(19) United States
(12) Patent Application Publication
(10) Pub. No.: US 2013/0039904 A1
(43) Pub. Date: Feb. 14, 2013

(54) GAMBOGIC ACID CYCLIZATION ANALOGUES, THEIR PREPARATION METHOD AND APPLICATION THEREOF

(71) Applicant: Lifeng Xu, San Diego, CA (US)

(72) Inventor: Lifeng Xu, San Diego, CA (US)

(73) Assignee: Liaoning Lifeng Scientific & Technology Development Company Ltd., Liaoning (CN)

(21) Appl. No.: 13/389,263

(22) PCT Filed: Aug. 2, 2010
(86) PCT No.: PCT/CN10/75625

§ 371 Date: Apr. 16, 2012

(30) Foreign Application Priority Data
Aug. 12, 2009 (CN) 2009101066738.4

(51) Int. Cl. A61K 31/352; A61K 31/7048; A61K 31/706; C07D 491/22; A61K 31/436; A61K 31/519; C07D 493/18; A61K 31/496; A61K 31/5377; C07D 498/22; A61K 31/5365; C07F 9/6561; A61K 31/675; C07D 513/22; A61K 31/554; A61P 35/00; A61P 35/02; A61K 33/24; A61K 31/704; A61K 39/395; A61K 31/437; A61K 33/36; A61P 31/12; A61P 31/04; A61P 31/10; C07H 15/26

(52) U.S. Cl. 424/133.1; 536/17.9; 514/25; 549/224; 536/17.4; 546/31; 514/453; 514/279; 544/245; 514/257; 544/378; 514/254.11; 544/363; 514/253.08; 544/150; 514/232.8; 544/95; 514/229.5; 544/244; 514/81; 540/546; 514/211.1; 424/649; 514/110; 514/34; 514/285; 514/27; 424/623

(57) ABSTRACT

The present invention discloses a gamboge acid cyclization analogs, their preparation methods and applications by semi-synthesis with the following structural formula I-III:

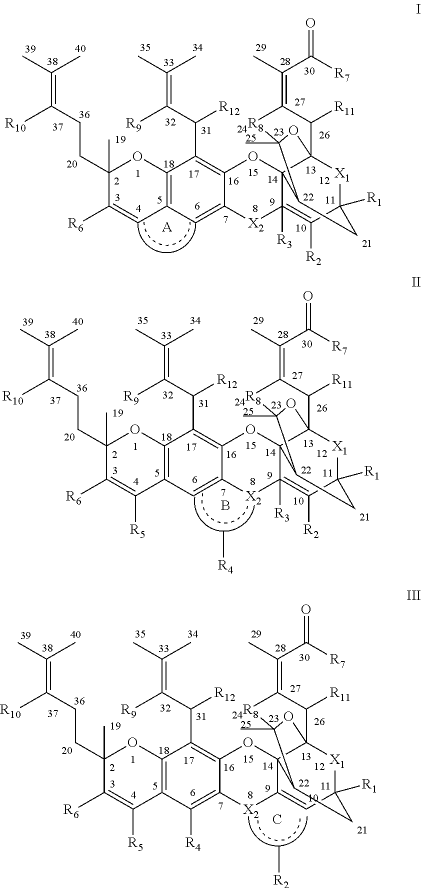

Where ring A, ring B or/and ring C is 4-10 membered saturated or/and unsaturated aliphatic ring aliphatic heterocycle or aryl heterocycle. R₁, R₂, R₃, R₄, R₅, R₆, R₇, R₈, R₉, R₁₀, R₁₁ or/and R₁₂ is, independently at each occurrence, optionally substituted substituent of glycosyl, multi-hydroxyl, amino acid, acyloxy, phosphoric acid oxy, sulfonyloxy, alkoxy, aryloxy, heterocyclic oxy, thiol, aliphatic or cyclic group containing oxygen, sulfur, nitrogen or phosphorus, one of the substituents or combinations thereof.

The present invention has antitumor activity management, antiviral, antibacterial and antifungal activity management, as anti-tumor, anti-viral, immune, antibacterial and antifungal agents, with other known anti-tumor, anti-viral, immune, together with the application of antibacterial and antifungal.

### THE FIELD OF INVENTION

This invention relates with anti-tumor activities, medicinal chemistry research and preparative methods of new cyclized gambogic acid derivatives and analogs thereof. The invention also relates with the medication applications of anti-tumor and other diseases by this kind of compounds.

### BACKGROUND OF THE INVENTION

This invention is in the field of medicinal chemistry. In particular, the invention relates with cyclized gambogic acid derivatives and analogs, and the discovery of these compounds is therapeutically effective anti-cancer agents.

### DESCRIPTION OF BACKGROUND ART

Medicinal plant Gamboge is a gel resin from trees of Hai teng, Yu huang, Yue huang and La huang, in India, Thailand, the islands of Southeast Asia, Cambodia, Thailand, Viet Nam and China (Ref. 1: Wang Ming, Feng Xu, Zhao Youyi, Fu Hui, Studies and Application of Gamboge, Chinese Medicine Research and Chinese Wild Plant Resources, 2003, 22 (1), 1-3).

Gambogic resin contains gambogic acid, neogambogic acid, allogambogic acid and other ingredients. Anti-cancer effects of gambogic acid was proved by the experiments of Chinese research group (Ref. 2: Xiang S. R., Chen T K, Huang, Y. C. et al, Effect on tumor S₁₈₀ and ascites by gambogic acid, J. Acta Acad Med Jiang xi, 1981, (1), 172211). Recent study showed the effect of gambogic acid on pancreatic cancer cells (Ref. 3: Qidong You, et al, Chinese patent CN1309125A). Shu Long, Wang reported a prodrug of gambogic acid reacted with multi-ethyleneglycol (Ref. 4: Shulong, Wang, A new gambogic acid derivative, Chinese Patent Application CN 1563014A). While Wen-Hu, Duan, et al, reported the structural modifications of C-4 and C-30 of gambogic acid [CN 1715283]. The patents, WO 06/44216, U.S. Pat. No. 7,176,234, U.S. Pat. No. 7,138,620, U.S. Pat. No. 7,138,428, U.S. Pat. No. 6,613,762, U.S. Pat. No. 6,462,041, US 2005/00040206, US 2004/0082066, US 2003/0078292 and US 2002/0076733 reported the structural modification of gambogic acid, chemical synthesis, preparation and study of anti-tumor activity at sites of C-10 and C-30.

To date there has been no report related with structural modification of gambogic acid with the introduction of ring structure to form cyclized gambogic acid derivatives and analogs at C-4, C-6, C-8 or C-10 site, nor structure-activity relationship studies by the introduction of ring system from all literature reported.

### BRIEF DESCRIPTION OF THE DRAWINGS

FIG. 1 shows the inhibition growth of sarcoma S₁₈₀ anatomy (Kunming mice inoculated with S₁₈₀ administered 7 days) of 11 compounds.

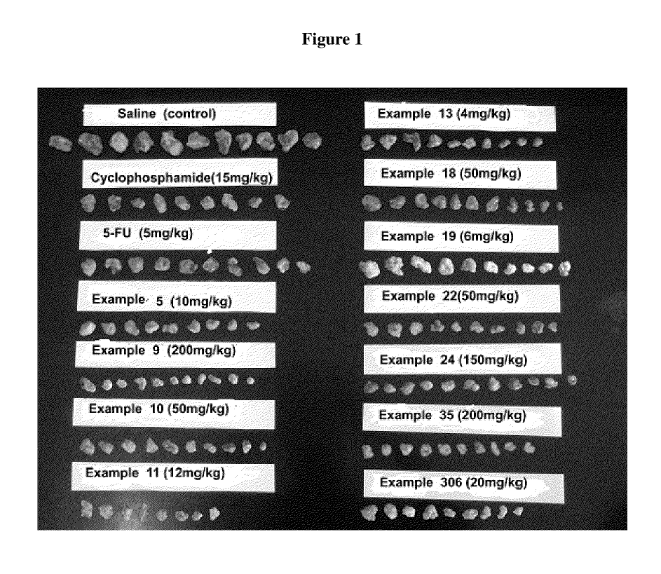

### SUMMARY OF THE INVENTION

This invention relates with the cyclized gambogic acid derivatives and analogs by the cyclization of gambogic acid, their preparation method, and the discovery of therapeutically effective anti-cancer. Their structures are formula I, II and III.

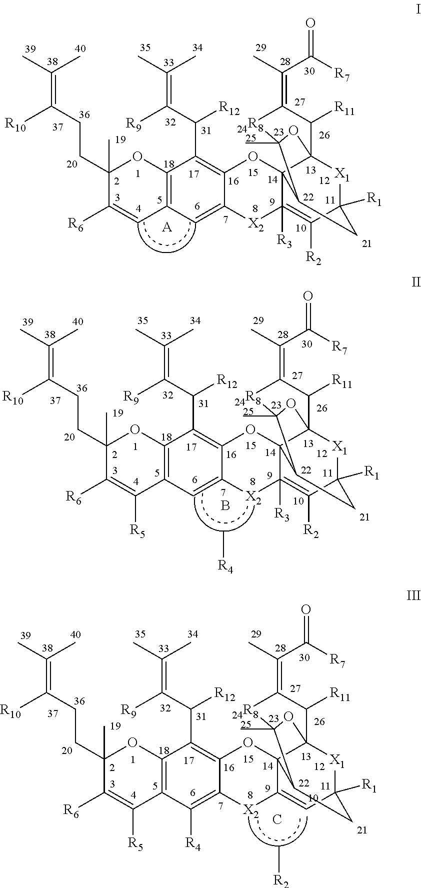

or stereoisomers, tautomers, prodrug, pharmaceutically acceptable salts, complex salts or solvates thereof, wherein:

The dotted lines are optionally substituted single bonds, optionally substituted double bond or a optionally substituted heterocyclic group containing carbon, oxygen, sulfur or nitrogen element;

Ring A, ring B or/and ring C is 4-10 membered saturated or/and unsaturated aliphatic ring aliphatic heterocycle or aryl heterocycle.

R₁, R₂, R₃, R₄, R₅, R₆, R₇, R₈, R₉, R₁₀, R₁₁ or/and R₁₂ is, independently at each occurrence, optionally substituted substituent of glycosyl, optionally substituted multi-hydroxyl, amino acid, acyloxy, phosphoric acid oxy, sulfonyloxy, alkoxy, aryloxy, heterocyclic oxy, thiol, substituted thiol, aliphatic or cyclic group containing primary amine secondary amine or/and tertiary amine or substituted primary amine, substituted secondary amine or substituted tertiary amine, where contains optionally substituted one or combination;

Substituent containing oxygen, sulfur, nitrogen or phosphorus element is, independently at each occurrence, optionally substituted one or combination of saturated, unsaturated C₁₋₁₀ alkyl, optionally substituted 1-4 double bond, optionally substituted triple bond, optional substituent of saturated or unsaturated C₁₋₁₀ alicyclic, arylcyclic and heterocyclic group, where contains a cycle or combination of oxygen, sulfur, nitrogen or phosphorus element, saturated or unsaturated 3-7 membered alicycle, aryl cycle, multi-cycle, aliphatic heterocycle, aryl heterocycle or fused heterocycle;

wherein:
X₁ and X₂ are, independently at each occurrence, C═O, C═Rb—Ra, CHOH, CHORb, CHRb or substituent, where Rb contains, independently at each occurrence, one or combination of C, N or P element; Ra is H, H₂, optionally substituted straight-alkyl, optionally substituted branched-alkyl, C₁₋₁₀ optionally substituted saturated alkyl, optionally substituted 1-4 double bond, optionally substituted 1-4 triple bond, optionally substituted unsaturated alkyl, optionally substituted saturated or unsaturated alicyclic, optionally substituted arylcyclic, optionally substituted aryl or optionally substituted 3-7 membered heterocyclic, optionally substituted aryheterocyclic, fused heterocyclic group where contains hydroxyl, halogen, oxygen, nitrogen, sulfur or phosphorus element;

Substituent is, independently at each occurrence, C₁₋₁₀ optionally substituted saturated Glycosyl is D- or L-configuration and its glycoside bond is C—C or C-hetero bond connection, including 1-8 optionally substituted glycosyl or optional substituent glycosyl group;

Multi-hydroxyl is, independently at each occurrence, 1-10 optionally substituted hydroxyl group of alkyl, aryl, cyclic or heterocyclic group, where contains optionally substituted or combination of amino acid, acyloxy, sulfonyloxy, phosphoric acid oxy, alkoxy, aryoxyl or heterocyclicoxyl, thiol, substituted thiol, or heteroatom contained alkyl, alicyclic, aryl ring, aliphatic heterocyclic or aryl heterocyclic group;

Cyclized cyclic substituent is, independently at each occurrence, formed a new one or a combination of A-ring between C-4 and C-6, B-ring between C-6 and C-8, C-ring between C-8 and C-10 positions;

R₁₂, R₁, R₂, R₅, R₆, R₈, R₉, R₁₀, R₁₁ or/and R₁₂ is, independently at each occurrence, H, halogen or XRa; where XRa is unsubstituted or substituted group containing C, O, S, Se, N, and/or P element.

R₃ is XaRa electrophilic substituent, where Xa is, independently at each occurrence, unsubstituted or substituted group containing C, S, P, and/or Si element;

R₄ is, independently at each occurrence, optional substituent of 1-8 glycosyl, multi-hydroxyl, substituted multi-hydroxyl, 1-5 amino acid, 1-4 phosphate, acyloxy, phosphoric, sulfonyloxy, alkoxy, aryloxy, heterocyclic oxy, alkyl, alicyclic, aryl cyclic, aliphatic heterocyclic oxyl or aryl heterocyclic oxyl containing oxygen, sulfur, nitrogen or phosphorus element, where glycosyl, multihydroxyl, amino acid, acryloxy, phosphoryloxy, sulfonyloxy, alkoxy, aryloxy, heterocyclic oxy and substituent is the same as above.

1-8 Glycosyl is, independently at each occurrence, optionally substituted C₃₋₈ saccharide, optionally substituted monosaccharide, optionally substituted disaccharide, optionally substituted trisaccharide and/or optionally substituted polysaccharide;

wherein:

C₃₋₈ Saccharide is independently at each occurrence, optionally substituted C₃ saccharide, optionally substituted C₄ saccharide, optionally substituted C₅ saccharide, optionally substituted C₆ saccharide, optionally substituted C₇ saccharide, optionally substituted C₈ saccharide, optionally substituted hydroxyl saccharide, optionally substituted amino saccharide, optionally substituted deoxysaccharide, optionally substituted sulfuric acid saccharide, optionally substituted hetero-element saccharide and/or its glycoside.

R₇ is H or XbRa; Xb is, independently at each occurrence, optional substituent containing H, C, O or N element.

wherein:

When X₁ and/or X₂ is C═O, C═Rb—Ra, CHOH, CHORb, or CHRb, X₁ and X₂ are the same or different substituents; when Rb is C, N or P element, Ra is, independently at each occurrence, optionally substituted formation of olefin, alkane, halogenated hydrocarbon, alcohol, ether, oxime, hydrazone or substituted said groups.

A bromo compound at 11-position is selected, independently at each occurrence, from: gambogic acid, methyl gambogate, ethyl gambogate, gambogyl morpholine, gambogyl.

A compound with A-ring, B-ring or/and C-ring cyclized respectively between 4- and 6-position, 6- and 8-position and 8- and 10-position of gamboge acid is selected, independently at each occurrence, from the example 1 to example 441 and the list (see claim 2) but not limiting, of the method and composition of the present invention:

fused A-ring was formed between 4- and 6-position of gambogate acid analogs; fused B-ring was formed between 6- and 8-position of gambogate acid analogs; fused C-ring was formed between 8- and 10-position of gambogate acid analogs.

A process for the manufacture of a compound of formula I, II, III comprises: for the preparation of compounds of formula I, II, III with A-ring, B-ring, C-ring and salts thereof in which the reaction of a gambogic acid or analog to introduce A-ring lactone at 4-, 6-position, B-ring at 6-, 8-position, and C-ring at 8-, 10-position forms a bond of C—C, C—O, C—S, C—N or C—P under catalysis at −78° C. to 90° C., wherein:

The reactant selected from 2-ethoxy-1-ethoxy carbon acyl-1,2-dihydroquinoline, 2-(7-azabenzotriazole)-N,N,N′,N′-tetramethyl urea hexafluorophosphate, benzotriazole N,N,N′,N′-tetramethyl urea hexafluoro phosphate, 6-chlorophenyl and triazole-1,1,3,3-tetramethyl urea hexafluoro phosphate, 1-hydroxy-7-azobis benzotriazole, 1-hydroxy-benzotriazole, 3-hydroxy-1,2,3-benzotriazin-4(3H)one, N-hydroxysuccinimide, and triethylamine, Fmoc chloride, acyl succinimide Fmoc, 9-fluorene methanol;

The catalyst selected from palladium, platinum, ruthenium, metal catalyst, organic base or inorganic base, 1-ethyl-3-(3-dimethylpropylamine)carbodiimide, ditertbutyl dicarbonate, bis(2-oxo-3-oxazoline alkyl) times phosphorus chloride, N,N′-carbonyl two pyrrolidine, N,N′-carbonyl bis (1,2,4-triazole), 6-chloro-1-hydroxybenzo triazole, N,N′-dicyclohexyl carbodiimide, 4,5-dicyano imidazole, 3-(diethoxyphosphoryl)oxy-1,2,3-benzotriazine-4-ketone, N,N′-bis isopropyl carbon imide, N,N′-diisopropyl ethylamine, 4-dimethylaminopyridine, 4,4′-dimethoxytriphenyl chloride, 4-(4,6-dimethoxytriazine)-4-methyl morpholine hydrochloride, N,N′-succinimidyl carbonate, 1-ethyl-(3-dimethylaminopropyl) carbodiimide hydrochloride;

The solvent selected from tetrahydrofuran, 1,4-dioxane, acetonitrile, N,N-dimethylformamide, N,N-dimethylacetamide, hexane, toluene, quinoline;

For the preparation of compounds of formula I, II, III with A-ring, B-ring, C-ring and salts, the X₁, X₂, R₁, R₂, R₃, R₄, R₅, R₆, R₇, R₈, R₉, R₁₀, R₁₁ or/and R₁₂ is an amino acid, acyloxy, phosphoric acid, phosphoryloxy, sulfonyloxy, alkoxy, aryloxy, heterocyclicoxy, hydrocarbons, alicyclic, glycosyl, multi-hydroxyl, carboxyl, glucosyl, multi-hydroxyl, alkane, aryl, alicyclic, heterocyclic, heteroarylcyclic or substituent modified by acylation, halogenation; electrophilic substituent at 9-position, nucleophilic substituent at 10-position accompanied by 1,4 addition reaction; allylation at 11-position, 26-position, 31-position or 36-position with a bond of C—C, C—O, C—S, C—N or C—P under catalysis at −78° C. to 90° C.

A method for treating cancer, comprising: administration to a above compound, in the range of 0.001 mg/kg-250 mg/kg, a pharmaceutically acceptable salt or prodrug from thereof; a cancer is selected from the lung cancer, stomach cancer, colon cancer, small cell lung cancer, thyroid cancer, esophageal cancer, pancreatic cancer, endometrial cancer, adrenal cortical carcinoma, head and neck cancer, Osteogenic sarcoma, breast cancer, ovarian cancer, Vail Williams tumors, cervical tumors, testicular cancer, genitourinary cancer, skin cancer, renal cell carcinoma, bladder cancer, primary brain cancer, prostate cancer, soft tissue sarcoma, neuroblastoma, rhabdomyosarcoma, Kaposi sarcoma, malignant melanoma, malignant pancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, or Hodgkin's disease.

The compound is administered together with at least one known cancer, chemotherapeutic and immune agent selected from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, tamoxifen, doxazocin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoromethyl ornithine, fenretinide, N-4-carboxyphenyl retinamide, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, Roscovitine, Olonoucine, Valecoxib.

The compound is selected from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier.

A compound for treating, preventing or slowing the progression of neoplasia and cancer, and infection diseases by virus, bacterial or fungi, including bacterial infections and fungal infections of the drug application, which comprises administration together with at least one known chemotherapeutic agent selected from the group consisting of antibacterial and antifungal drugs to a patient in need of such treatment.

The administration may be by oral route, parenteral, subcutaneous, intravenous, intramuscular, intra-peritoneal, transdermal, buccal, intrathecal, intracranial, intranasal or topical routes.

The invention has the following beneficial effects: the structure-activity relationship studies have shown that toxicity and activity of gambogic acid cyclization analogs are sensitive by the introduction of the A ring, B-ring and C-ring cyclization at the 4-, 6-position, 6-, 8-position and 8-, 10-position. The inhibition rate increased to 25-55% after modification of gambogic acid into cyclized gambogic acid analogs.

Gambogic acid preparation (see U.S. Patent Publication No. US2011/0038952A1)

### Chemical Synthesis

Synthesis of Gambogic Acid Analogies at 30-Position into the Ester, Anhydride and Amide:

gambogic acid was modified into the analogs of esters, anhydrides and amides by the catalyst to form C—O bond, C—S bond, C—N bond, C—P bond and the solvent selected from THF, 1,4-dioxane, Acetonitrile, dichloromethane, N,N-dimethylformamide, N,N-dimethylacetamide, n-hexane, toluene, quinoline, at −78 to 90° C.

The Introduction of the A-Ring Analogs:

cyclized gamboge acid analogs were modified by the introduction of the A-ring between 4- and 6-position by the catalyst to form C—C bond, C—O bond, C—S bond, C—N bond, C—P bond and the solvents selected from THF, 1,4-dioxane, acetonitrile, dichloromethane, N,N-dimethyl formamide, N,N-dimethylacetamide, n-hexane, toluene, quinoline, at −78 to 90° C.

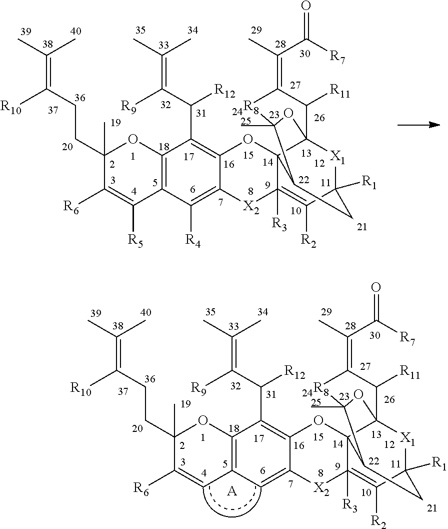

The Introduction of the B-Ring Analogs:

cyclized gamboge acid analogs were modified by the introduction of the A-ring between 6- and 8-position by the catalyst to form C—C bond, C—O bond, C—S bond, C—N bond, C—P bond and the solvents selected from THF, 1,4-dioxane, acetonitrile, dichloromethane, N,N-dimethylformamide, N,N-dimethylacetamide, n-hexane, toluene, quinoline at −78 to 90° C.

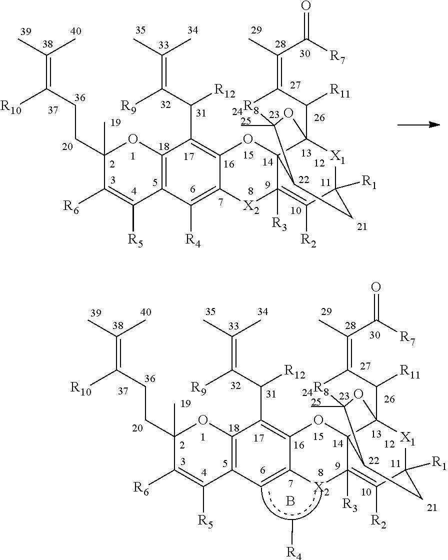

The Introduction of the C-Ring Analogs:

cyclized gamboge acid analogs were modified by the introduction of the C-ring between 8-, 10-position by the catalyst to form C—C bond, C—O bond, C—S bond, C—N bond, C—P bond and the solvents selected from THF, 1,4-dioxane, acetonitrile, dichloromethane, N,N-dimethylformamide, N,N-dimethylacetamide, n-hexane, toluene, quinoline at −78 to 90° C.

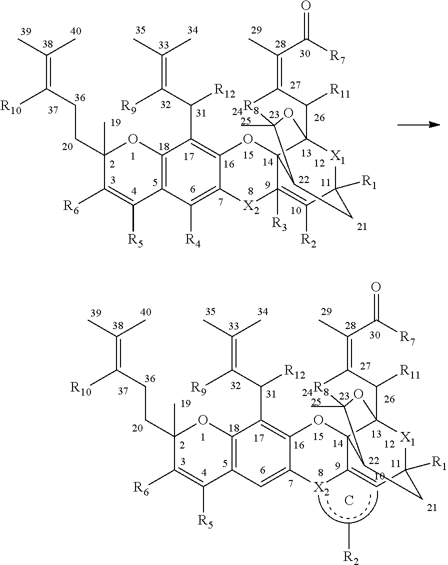

Modification of Gambogic Acid Analogs at 6-Position:

Modification of gambogic acid analogs at 6-position by introduction of ester, anhydride, amide, ether, sugar, substituted sugar or multi-hydroxyl groups to form C—C bond, C—O bond, C—S bond, C—N bond and C—P bond by the catalyst selected from silver-containing catalyst, Lewis acid, perchloric acid and molecular sieves.

The phenolic hydroxyl of gambogic acid analogs at 6-position can be made into a good leaving and then the cyclized gambogic acid analogs was modified by the introduction of a nucleophilic agent to form C-halogen bond, C—C bond, C—S bond, C—N bond and/or C—P bond analogs;

The following examples are illustrative, but not limiting, of the method and composition of the present invention.

### EXAMPLES

### Synthesis and Preparation

The following examples illustrate the present invention. If no mentioned otherwise, the reactions take place at room temperature.

### Example 1

### Compound 1.1 in Table 1

To a mixture of gamboge acid 12.56 g (20 mmol), catalyst volume DMAP in THF 80 ml and DMF 20 ml was added ethanolamine 1.22 g (20 mmol). The mixture was stirred at room temperature 8 h and added glacial acetic acid 1.3 ml, at 40° C., for 8 h. The reaction solution was distilled under reduced pressure and the aqueous phase was extracted three times with ethyl acetate. The crude was separated by silica gel column chromatography to give compound 1.1, IR (KBr, cm⁻¹): 3422, 2965, 2925, 2855, 1738, 1711, 1633, 1594, 1508, 1439, 1400, 1384, 1332, 1174, 1136, 1048, 957, 793, 772.

### Example 2

### Compound 1.2 in Table 1

To a mixture of compounds 1.1 7.13 g (10 mmol) and triethylamine 1.52 g (15 mmol) in CH₂Cl₂ 30 ml, in THF 80 ml and DMF 20 ml was added 4-O-D-allosylbenzoyl chloride 4.77 g (15 mmol). The mixture was stirred at room temperature for 8 h. The reaction solution was distilled under reduced pressure and the aqueous phase was extracted three times with ethyl acetate. The crude was separated by silica gel column chromatography to give compound 1.2, IR (KBr, cm⁻¹): 3420, 2925, 2855, 1738, 1633, 1594, 1508, 1457, 1438, 1383, 1332, 1175, 1136, 1048, 793, 771, 496.

### Example 3

### Compound 1 in Table 1

To a 125 ml round bottom flask was added 3.51 g (3 mmol) compound 1.2 in methanol 50 ml and the mixture was refluxed 14 hours. The reaction solution was distilled under reduced pressure and the aqueous phase was extracted three times with ethyl acetate. The crude was separated by silica gel column chromatography to give compound 1, IR (KBr, cm⁻¹): 3421, 2968, 2926, 1739, 1711, 1633, 1607, 1544, 1502, 1458, 1438, 1398, 1328, 1299, 1241, 1176, 1082, 1042, 906, 848, 768, 560; ¹H NMR (CDCl₃) δ 8.20 (d, J=8.4 Hz, 2H), 7.36 (d, J=7.2 Hz, 1H), 7.13 (d, J=8.4 Hz, 2H), 6.44 (d, J=10.2 Hz, 1H), 6.39 (t, J=6.0 Hz, 1H), 5.60 (d, J=9.6 Hz, 1H), 5.36 (d, J=7.2 Hz, 1H), 5.22 (m, 1H), 5.07 (m, 1H), 4.25 (m, 1H), 3.93 (d, J=11.4 Hz, 1H), 3.86 (m, 1H), 3.81 (m, 1H), 3.71˜3.66 (m, 5H), 3.43 (m, 1H), 3.38 (m, 1H), 3.37 (m, 1H), 2.64 (Br, 5H), 2.51 (d, J=9.0 Hz, 1H), 2.29 (m, 1H), 2.05 (m, 2H), 1.78 (s, 3H), 1.76 (m, 2H), 1.72 (s, 3H), 1.69 (s, 3H), 1.67 (s, 3H), 1.65 (s, 3H), 1.59 (s, 3H), 1.36 (s, 3H), 1.40 (m, 1H), 1.29 (s, 3H).

### Example 4

### Compound 2.1 in Table 1

To a mixture of methyl gambogate 12.84 g (20 mmol) and ammonium acetate 3.85 g (50 mmol) in DMF 60 ml, was added 1,3-indanedione 3.50 g (24 mmol). The mixture was stirred at 35° C. for 24 h. The reaction solution was distilled under reduced pressure and the aqueous phase was extracted three times with ethyl acetate. The crude was separated by silica gel column chromatography to give compound 2.1, IR (KBr, cm⁻¹): 3438, 2971, 2925, 2856, 1741, 1710, 1644, 1627, 1586, 1456, 1438, 1375, 1321, 1254, 1216, 1176, 1125, 965, 947, 906, 839, 754.

### Example 5

### Compound 2.2 in Table 1

To a mixture of compound 2.1 3.35 g (5 mmol) and triethylamine 0.71 g (7 mmol) in CH₂Cl₂ 20 ml was added 4-O-D-Allosylbenzoyl chloride 2.23 g (7 mmol). The mixture was stirred at rt for 24 h. The reaction solution was distilled under reduced pressure and the aqueous phase was extracted three times with ethyl acetate. The crude was separated by silica gel column chromatography to give compound 2.2, IR(KBr, cm⁻¹): 3437, 2964, 2928, 1752, 1712, 1676, 1632, 1606, 1508, 1462, 1435, 1373, 1321, 1229, 1173, 1092, 1065, 1045, 948, 910, 759, 690.

### Example 6

### Compound 2 in Table 1

To a compound 2.2 2.45 g (2 mmol) was added methanol 30 ml. The mixture was refluxed for 14 h. The reaction solution was distilled under reduced pressure and the aqueous phase was extracted three times with ethyl acetate. The crude was separated by silica gel column chromatography to give compound 2, IR (KBr, cm⁻¹): 3437, 2924, 2854, 1741, 1708, 1674, 1641, 1604, 1509, 1461, 1384, 1320, 1259, 1171, 1041, 906, 846, 804, 690, 621. ¹H NMR (CDCl₃) δ 7.97 (m, 2H), 7.79 (m, 2H), 7.66 (d, J=8.4 Hz, 2H), 7.55 (d, J=7.2 Hz, 1H), 7.13 (d, J=8.4 Hz, 2H), 6.57 (d, J=10.2 Hz, 1H), 6.39 (t, J=6.0 Hz, 1H), 5.38 (d, J=10.2 Hz, 1H), 5.22 (d, J=7.2 Hz, 1H), 5.02 (m, 1H), 4.92 (m, 1H), 4.25 (m, 1H), 4.19 (m, 2H), 3.78 (m, 2H), 3.66 (m, 1H), 3.63 (s, 1H), 3.59 (m, 3H), 3.40 (m, 1H), 3.20 (m, 1H), 3.19 (m, 1H), 2.75 (m, 2H), 2.51˜2.50 (br, 4H), 2.39 (d, J=8.4 Hz, 1H), 2.00 (m, 1H), 1.97 (m, 2H), 1.95 (s, 3H), 1.78 (s, 3H), 1.70 (m, 1H), 1.66 (m, 6H), 1.57 (s, 3H), 1.52 (m, 2H), 1.34 (s, 3H), 1.38 (s, 3H), 1.17 (s, 3H).

### Example 7

### Compound 3.1 in Table 1

To a mixture of 4-O-D-Allosylbenzoyl chloride 9.48 g (20 mmol), DMAP 1.22 g (10 mmol), THF 30 ml and DMF 10 ml, was added ethanolamine 2.44 g (40 mmol). The mixture was stirred at room temperature 4 h. The reaction solution was distilled under reduced pressure and the aqueous phase was extracted three times with ethyl acetate. The crude was separated by silica gel column chromatography to give compound 3.1, IR (KBr, cm⁻¹): 3432, 2919, 2850, 1734, 1699, 1628, 1603, 1583, 1565, 1506, 1468, 1413, 1384, 1306, 1283, 1229, 1166, 1144, 1097, 1037, 838, 720.

### Example 8

### Compound 3.2 in Table 1

To a mixture of compound 3.1 5.17 g (10 mmol) and gambogic acid 12.56 g (20 mmol) in THF 80 ml and DMF 20 ml, was added DMAP 0.61 g (5 mmol). The mixture was stirred at room temperature 8 h. The reaction solution was distilled under reduced pressure. The crude was separated by silica gel column chromatography to give compound 3.2, IR (KBr, cm⁻¹): 3445, 2925, 2855, 1748, 1663, 1633, 1606, 1508, 1459, 1384, 1320, 1227, 1173, 1141, 1089, 1044, 910, 851, 760, 617.

### Example 9

### Compound 3 in Table 1

To a compound 3.2 2.25 g (2 mmol) was added methanol 30 ml. The mixture was refluxed for 8 h. The reaction solution was distilled under reduced pressure and the aqueous phase was extracted three times with ethyl acetate. The crude was separated by silica gel column chromatography to give compound 3, IR (KBr, cm⁻¹): 3347, 2967, 2924, 2856, 1739, 1716, 1664, 1628, 1609, 1522, 1503, 1453, 1375, 1323, 1224, 1175, 1125, 1060, 1045, 955, 907, 830, 748, 599. ¹H NMR (CDCl₃) δ 11.92 (s, 1H), 7.68 (d, J=8.4 Hz, 2H), 7.55 (d, J=7.2 Hz, 1H), 7.13 (d, J=8.4 Hz, 2H), 6.57 (d, J=10.2 Hz, 1H), 6.49 (t, J=6.0 Hz, 1H), 5.38 (d, J=10.2 Hz, 1H), 5.22 (d, J=7.2 Hz, 1H), 5.02 (m, 1H), 4.92 (m, 1H), 4.25 (m, 1H), 4.19 (m, 2H), 3.78 (m, 2H), 3.66 (m, 1H), 3.61 (m, 3H), 3.40 (m, 1H), 3.20 (m, 1H), 3.19 (m, 1H), 2.75 (m, 2H), 2.51˜2.50 (m, 3H), 2.39 (d, J=8.4 Hz, 1H), 2.00 (m, 1H), 1.97 (m, 2H), 1.87 (s, 3H), 1.70 (m, 2H), 1.65 (s, 3H), 1.61 (s, 3H), 1.58 (s, 3H), 1.57 (s, 3H), 1.49 (s, 3H), 1.35 (m, 1H), 1.29 (s, 3H), 1.08 (s, 3H).

### Example 10

### Compound 4.1 in Table 1

To a compound of acetylated-4-O-D-allosylbenzoic acid 20 g (42.8 mmol) was added fuming nitric acid 16 ml. The mixture was stirred at −20° C., for 1 h. The reaction solution was added ice water 60 mL. The crude was separated by silica gel column chromatography to give compound 4.1, IR (KBr, cm⁻¹): 3436, 2926, 1753, 1617, 1541, 1701, 1617, 1541, 1500, 1428, 1373, 1231, 1168, 1086, 1066, 1048, 951, 917, 829, 769.

### Example 11

### Compound 4.2 in Table 1

To a compound of 4.1, 6 g (11.7 mmol) in methanol 30 ml was added H₂. The mixture was stirred at rt for 2 h. The reaction solution was distilled under reduced pressure and the crude was separated by silica gel column chromatography to give compound 4.2, IR (KBr, cm⁻¹): 3476, 3382, 2963, 1751, 1719, 1621, 1599, 1516, 1449, 1374, 1227, 1155, 1091, 1047, 951, 911, 887, 770, 646.

### Example 12

### Compound 4.3 in Table 1

To a mixture of compound of 4.2, 4.0 g (8.4 mmol) and pyridine 2 ml in methanol 30 ml and THF15 ml was added acetic anhydride 2 ml. The mixture was stirred at rt for 4 h. The ice water was added the reaction solution. The crude was separated by silica gel column chromatography to give compound 4.3, IR (KBr, cm⁻¹): 3391, 2963, 1757, 1714, 1660, 1599, 1544, 1484, 1442, 1375, 1250, 1227, 1078, 1044, 952, 911, 836, 805, 770, 645.

### Example 13

### Compound 4.4 in Table 1

To a mixture of compound of 4.3, 4.0 g (7.6 mmol), thionyl chloride 0.81 ml, pyridine 0.606 ml, DMAP 0.57 g and triethylamine 1.3 ml in CH₂Cl₂ 20 ml was added gamboge methyl 3 g. The mixture was stirred at rt for 0.5 h. The reaction solution was distilled under reduced pressure and the aqueous phase was extracted three times with ethyl acetate. The crude was separated by silica gel column chromatography to give compound 4.4, IR (KBr, cm⁻¹): 3435, 2964, 2928, 2847, 1751, 1709, 1663, 1632, 1606, 1537, 1501, 1458, 1431, 1384, 1321, 1226, 1187, 1137, 1091, 1046, 952, 909, 756, 599.

### Example 14

### Compound 4 in Table 1

To a compound 4.4 2.31 g (2 mmol) was added methanol 30 ml. The mixture was refluxed for 8 h. The reaction solution was distilled under reduced pressure and the crude was separated by silica gel column chromatography to give compound 4, IR (KBr, cm⁻¹): 3429, 2925, 2848, 1740, 1709, 1655, 1633, 1605, 1539, 1458, 1431, 1384, 1262, 1188, 1138, 1046, 805, 755, 502. ¹H NMR (CDCl₃) δ 8.86 (s, 1H), 8.56 (s, 1H), 7.96 (d, J=7.8 Hz, 1H), 7.35 (d, J=6.6 Hz, 1H), 7.24 (d, J=8.4 Hz, 2H), 6.42 (d, J=10.8 Hz, 1H), 6.00 (t, J=6.0 Hz, 1H), 5.58 (d, J=9.6 Hz, 1H), 5.21 (d, J=7.2 Hz, 1H), 5.07 (m 1H), 5.02 (m, 1H), 4.07 (m, 1H), 3.80˜3.58 (m, 5H), 3.49 (s, 3H), 3.34 (m, 2H), 2.98˜2.70 (m, 2H), 2.64 (m, 2H), 2.47 (d, J=8.4 Hz, 1H), 2.20 (m, 1H), 2.12 (s, 3H), 2.12 (m, 2H), 1.75 (s, 3H), 1.74 (m, 2H), 1.68 (s, 3H), 1.65 (s, 3H), 1.64 (s, 3H), 1.54 (s, 3H), 1.44 (s, 3H), 1.29 (m, 1H), 1.26 (s, 3H), 1.22 (s, 3H).

### Example 15

### Compound 5 in Table 1

To a compound of methyl-6-(4-oxo-D-glucosyl)benzoyl gambogate 2 g (2.15 mmol) in 20 ml acetone, was added p-toluenesulfonic acid 0.37 g (2.15 mmol). The mixture was stirred at rt, for 12 h. The reaction solution was distilled under reduced pressure and the crude was separated by silica gel column chromatography to give compound 5, IR (KBr, cm⁻¹): 3410, 2957, 2924, 2854, 1738, 1716, 1663, 1606, 1515, 1463, 1384, 1322, 1258, 1110, 1043, 849, 690, 606; ¹H NMR (CDCl₃) δ 8.00 (d, J=8.4 Hz, 2H), 7.41 (d, J=6 Hz, 1H), 6.85 (d, J=8.4 Hz, 1H), 6.70 (d, J=8.4 Hz, 2H), 6.37 (d, J=10.2 Hz, 1H), 5.94 (t, J=6.0 Hz, 1H), 5.58 (d, J=10.2 Hz, 1H), 5.11 (m, 1H), 5.05 (m, 1H), 3.70 (m, 1H), 3.52 (s, 3H), 3.42 (m, 2H), 3.28 (m, 1H), 3.25 (m, 1H), 3.00 (m, 2H), 2.53 (d, J=9.0 Hz, 1H), 2.29 (m, 1H), 2.04 (m, 2H), 1.81 (s, 3H), 1.79 (m, 2H), 1.77 (s, 3H), 1.74 (s, 3H), 1.71 (s, 3H), 1.68 (s, 3H), 1.67 (s, 3H), 1.66 (s, 3H), 1.60 (br, 4H), 1.58 (s, 3H), 1.56 (s, 3H), 1.39 (m, 1H), 1.29 (s, 3H), 1.26 (s, 3H).

### Example 16

### Compound 6.1 in Table 1

To a mixture of compound BOC-L-alanine 18.90 g (0.1 mol) and DMAP 6.10 g (0.05 mol), in THF 200 ml, was added N-methyl-naphthalene methylamine 20.50 g (0.12 mol). The mixture was stirred at room temperature 8 h. The reaction solution was distilled under reduced pressure. The crude was separated by silica gel column chromatography to give compound 6.1, IR (KBr, cm⁻¹): 3426, 2978, 2931, 1709, 1646, 1599, 1511, 1487, 1457, 1414, 1385, 1367, 1250, 1167, 1087, 1051, 1020, 866, 793, 778.

### Example 17

### Compound 6.2 in Table 1

To a mixture of compound 6.1, 17.10 g (50 mmol), DMAP 3.05 g (25 mmol) and L-alanine-N-methyl naphthalene formamide 55 mmol in THF 100 ml was add gambogic acid 31.4 g (50 mmol). The mixture was stirred at room temperature 6 h. The reaction solution was distilled under reduced pressure. The crude was separated by silica gel column chromatography to give compound 6.2, IR (KBr, cm⁻¹): 3429, 2963, 2925, 2856, 1740, 1639, 1605, 1575, 1508, 1461, 1432, 1384, 1321, 1244, 1172, 1100, 1082, 1043, 908, 850, 793, 760, 688.

### Example 18

### Compound 6.3 in Table 1

To a mixture of compound 6.2, 21.73 g (25 mmol), DMAP 1.59 g (13 mmol), triethylamine 7 ml in CH₂Cl₂ 20 ml was add acetylated-4-O-D-allosylbenzoic chloride 7.96 g (25 mmol), gambogic acid 31.4 g (50 mmol). The mixture was stirred at rt for 1 h. The reaction solution was distilled under reduced pressure. The crude was separated by silica gel column chromatography to give compound 6.3, IR (KBr, cm⁻¹): 3450, 2965, 2926, 2856, 1750, 1662, 1639, 1605, 1575, 1509, 1482, 1462, 1374, 1321, 1300, 1175, 1142, 1090, 1045, 949, 910, 852, 760, 687, 600.

### Example 19

### Compound 6 in Table 1

To a compound 6.3, 13.25 g (10 mmol) was added methanol 100 ml. The mixture was refluxed for 8 h. The reaction solution was distilled under reduced pressure and the crude was separated by silica gel column chromatography to give compound 6, IR (KBr, cm⁻¹): 3445, 2921, 2851, 1747, 1682, 1631, 1604, 1508, 1460, 1374, 1225, 1166, 1092, 1047, 909, 793, 780, 576. ¹H NMR (CDCl₃) δ 8.10˜6.95 (m, 13H), 6.34 (t, J=6.0 Hz, 1H), 5.50 (d, J=10.2 Hz, 1H), 5.31 (m, 1H), 5.23 (m, 1H), 5.08 (m, 1H), 4.98 (m, 1H), 4.78 (m, 2H), 4.59˜4.16 (m, 2H), 3.90-3.40 (br, 4H), 3.33 (m, 2H), 2.92 (m, 3H), 2.43 (d, J=9.0 Hz, 1H), 2.40-2.00 (br, 4H), 1.98 (m, 2H), 1.76 (s, 3H), 1.74 (m, 2H), 1.69 (s, 3H), 1.64 (s, 3H), 1.62 (s, 3H), 1.60 (s, 3H), 1.59 (s, 3H), 1.49 (s, 3H), 1.42 (s, 3H), 1.39 (m, 1H), 1.27 (s, 3H), 1.19 (s, 3H).

### Example 20

### Compound 7 in Table 1

To a mixture of malononitrile 132 mg, triethylamine 2 ml in ethanol 10 ml was added methylgambogate 642 mg. The mixture was stirred at rt for 2 h. The reaction solution was distilled under reduced pressure. The crude was separated by silica gel column chromatography to give compound 7, IR (KBr, cm⁻¹): 3434, 3290, 2974, 2928 2880, 2214, 1714, 1646, 1629, 1584, 1454, 1440, 1374, 1255, 1177, 1142, 1126, 1028, 956, 907. ¹H NMR (CDCl₃) δ 11.63 (s, 1H), 6.68 (s, 1H), 6.66 (d, J=10.2 Hz, 1H), 6.22 (m, 1H), 5.49 (d, J=10.2 Hz, 1H), 5.09 (m, 1H), 5.00 (m, 1H), 4.13 (m, 2H), 3.78 (s, 1H), 3.77 (s, 3H), 3.65 (m, 1H), 3.41 (m, 1H), 3.27 (m, 1H), 3.13 (m, 1H), 2.72 (d, J=5.4 Hz, 1H), 2.44 (m, 2H), 2.05 (s, 3H), 2.02 (s, 3H), 1.69 (s, 3H), 1.67 (br, 3H), 1.63 (br, 3H), 1.56 (m, 3H), 1.49 (s, 3H), 1.45 (s, 3H), 1.38 (s, 3H), 1.26 (t, J=7.2 Hz, 3H).

### Example 21

### Compound 8 in Table 1

To a mixture of methyl gambogate 1.28 g (2 mmol) and triethylamine 5 ml in ethanol 25 ml was added malononitrile 160 mg. The mixture was stirred at rt for 5 h. The reaction solution was distilled under reduced pressure. The crude was separated by silica gel column chromatography to give compound 8, IR (KBr, cm⁻¹): 3427, 2978, 2933, 2880, 2193, 1679, 1624, 1444, 1397, 1368, 1304, 1230, 1217, 1175, 1111, 1096, 1059, 965. ¹H NMR (CDCl₃) δ 11.72 (s, 1H), 6.66 (d, J=10.2 Hz, 1H), 5.47 (d, J=10.2 Hz, 1H), 5.15 (m, 2H), 5.10 (m, 2H), 4.23 (m, 2H), 3.41 (s, 3H), 3.22 (m, 2H), 3.08 (m, 1H), 2.86 (d, J=2.4 Hz, 1H), 2.49 (m, 1H), 2.39 (br, 1H), 2.23 (m, 2H), 2.07 (br, 3H), 1.73 (s, 3H), 1.66 (m, 8H), 1.61 (s, 2H), 1.56 (s, 3H), 1.41 (s, 3H), 1.39 (s, 6H), 1.22 (d, 3H).

### Example 22

### Compound 9 in Table 1

To a mixture of methyl gambogate 1.284 g (2 mmol) and triethylamine 1.1 g in ethanol 20 ml was added 2-aminoimidazole 320 mg (3.9 mmol). The mixture was stirred at rt for 7 h. The reaction solution was distilled under reduced pressure. The crude was separated by silica gel column chromatography to give compound 9, IR (KBr, cm⁻¹): 3445, 2968, 2926, 1728, 1646, 1626, 1584, 1553, 1455, 1383, 1334, 1297, 1260, 1216, 1172, 1125, 1060, 1034. ¹H NMR (CDCl₃) δ 11.75 (s, 1H), 6.65 (d, J=10.2 Hz, 1H), 6.60 (m, 2H), 5.45 (d, J=10.2 Hz, 1H), 5.11 (m, 3H), 4.76 (m, 1H), 4.08 (m, 1H), 3.69 (m, 1H), 3.46 (s, 1H), 3.42 (s, 3H), 3.25 (m, 1H), 3.16 (m, 2H), 2.35 (m, 3H), 2.08 (m, 5H), 1.77 (m, 1H), 1.71 (s, 1H), 1.66 (s, 3H), 1.62 (s, 3H), 1.56 (s, 3H), 1.44 (m, 4H), 1.41 (s, 3H), 1.39 (br, 2H), 1.37 (m, 3H).

### Example 23

### Compound 10 in Table 1

To a mixture of methyl gambogate 1.284 g (2 mmol) and triethylamine 1.1 g in ethanol 20 ml was added 2-aminobenzimidazole 320 mg (2.4 mmol). The mixture was stirred at rt for 10 h. The reaction solution was distilled under reduced pressure. The crude was separated by silica gel column chromatography to give compound 10, IR (KBr, cm⁻¹): 3459, 2966, 2926, 1727, 1625, 1584, 1550, 1445, 1397, 1383, 1329, 1299, 1285, 1262, 1235, 1170, 1125, 1093, 1063, 1035, 1007, 961, 920, 820, 740, 603, 547. ¹H NMR (CDCl₃) δ 11.81 (s, 1H), 7.30 (m, 2H), 7.04 (m, 2H), 6.65 (d, J=10.2 Hz, 1H), 5.44 (d, J=10.2 Hz, 1H), 5.11 (m, 2H), 4.90 (m, 1H), 3.37 (m, 1H), 3.20 (m, 1H), 3.08 (s, 3H), 2.65˜3.0 (br, 8H), 2.40 (m, 1H), 2.09 (br, 2H), 1.98 (m, 1H), 1.67 (s, 3H), 1.66 (s, 3H), 1.60 (s, 3H), 1.59 (m, 3H), 1.56 (s, 3H), 1.41 (s, 3H), 1.37 (s, 3H), 1.34 (s, 3H).

### Example 24

### Compound 11 in Table 1

To a mixture of methyl gambogate 1.284 g (2 mmol) and DMAP 250 mg in THF 20 ml and DMF 5 ml, was added piperazine acid 740 mg (5.1 mmol). The mixture was stirred at rt for 11 h. The reaction solution was distilled under reduced pressure. The crude was separated by silica gel column chromatography to give compound 11, IR (KBr, cm⁻¹): 3435, 2962, 2925, 2855, 2688, 2454, 2347, 1767, 1737, 1713, 1658, 1575, 1462, 1432, 1384, 1321, 1227, 1202, 1176, 1136, 1047, 978, 877, 804, 758; ¹H NMR (CDCl₃) δ 7.38 (d, J=6.6 Hz, 1H), 6.50 (d, J=10.2 Hz, 1H), 6.00 (br, 1H), 5.61 (m, J=10.2 Hz, 1H), 5.05 (m, 2H), 3.86 (br, 2H), 3.41 (m, 4H), 3.22 (br, 1H), 3.10 (br, 2H), 2.97 (br, 1H), 2.65˜3.0 (br, 8H), 2.60 (m, 3H), 2.53 (m, 1H), 2.30 (m, 2H), 2.02 (m, 3H), 1.75˜1.90 (m, 4H), 1.70˜1.60 (m, 12H), 1.56 (m, 3H), 1.47 (s, 3H), 1.36 (m, 2H), 1.31 (s, 3H).

### Example 25

### Compound 12 in Table 1

To a mixture of methyl gambogate 642 mg (1 mmol), triethylamine 0.5 ml, DMSO 20 ml, DMAP 250 mg in THF 20 ml and DMF 5 ml was added ciprofloxacin 440 mg (1.3 mmol). The mixture was stirred at rt for 20 h. The reaction solution was distilled under reduced pressure. The crude was separated by silica gel column chromatography to give compound 12, IR (KBr, cm⁻¹): 3451, 2970, 2925, 2853, 1737, 1627, 1584, 1548, 1505, 1454, 1383, 1325, 1302, 1257, 1216, 1176, 1124, 1006, 893, 835, 807, 747; ¹H NMR (CDCl₃) δ 15.05 (s, 1H, —COOH), δ 11.98 (s, 1H, —OH), δ 8.77 (s, 1H), δ 8.00 (d, 1H, J=13.2 Hz), 7.32 (d, 1H, J=7.2 Hz), 6.67 (d, 1H, J=10.2 Hz, 1H, 4-H), 6.64 (t, 1H, J=6.6 Hz), 5.47 (d, 1H, J=10.2 Hz, 1H, 3-H), 5.10 (m, 1H), 5.01 (m, 1H), 3.59 (s, 1H, COOCH 3), 3.53 (m, 1H), 3.42 (m, 1H), 3.30 (m, 4H), 3.18 (m, 4H), 2.84 (br, 3H), 2.67 (m, 2H), 2.56 (d, 1H, J=8.4 Hz), 2.09 (br, 2H), 2.03 (m, 2H), 1.95 (s, 3H), 1.70 (m, 4H), 1.68 (m, 9H), 1.57 (s, 3H), 1.41 (m, 2H), 1.36 (s, 6H), 1.10 (m, 2H), 1.17 (s, 3H).

### Example 26

### Compound 13 in Table 1

To a mixture of methyl-10-morpholine gambogate 1.46 g and DMAP 250 mg was added chloroacetic acid 500 mg (5.2 mmol). The mixture was stirred at rt for 4 h. The reaction solution was distilled under reduced pressure. The crude was separated by silica gel column chromatography to give compound 13, IR (KBr, cm⁻¹): 3436, 2965, 2925, 2855, 1791, 1739, 1714, 1678, 1642, 1605, 1573, 1460, 1384, 1320, 1276, 1234, 1176, 1133, 1050, 1021, 887, 842, 808, 744, 570.

### Example 27

### Compound 14.1 in Table 1

To a compound of methyl-6-O-(2-chloroacetyl) gambogate 1.440 mg (2 mmol) in THF 40 ml was added palladium acetate 50 mg. The mixture was stirred at rt for 7 h. The reaction solution was distilled under reduced pressure. The crude was separated by silica gel column chromatography to give compound 14.1, IR (KBr, cm⁻¹): 3448, 2971, 2925, 1737, 1715, 1632, 1594, 1436, 1401, 1382, 1175, 1135.

### Example 28

### Compound 14 in Table 1

To a compound 14.1 600 mg (1.2 mmol) and triethylamine 0.8 g in ethanol 20 ml was added 2-aminoimidazole 110 mg (1.3 mmol). The mixture was stirred at rt for 7 h. The reaction solution was distilled under reduced pressure. The crude was separated by silica gel column chromatography to give compound 14, ¹H NMR (CDCl₃) δ 6.65 (d, J=10.2 Hz, 1H), 6.60 (m, 2H), 5.45 (d, J=10.2 Hz, 1H), 5.11 (m, 3H), 4.76 (m, 1H), 4.08 (m, 1H), 3.69 (m, 1H), 3.42 (s, 3H), 3.25 (m, 1H), 3.16 (m, 2H), 2.91 (s, 2H), 2.35 (m, 3H), 2.08 (m, 5H), 1.77 (m, 1H), 1.71 (s, 1H), 1.66 (s, 3H), 1.62 (s, 3H), 1.56 (s, 3H), 1.44 (m, 4H), 1.41 (s, 3H), 1.39 (br, 2H), 1.37 (m, 3H).

### Example 29

### Compound 15.1 in Table 1

To a mixture of methylgambogate 321 mg (0.5 mmol), potassium carbonate 138 mg (1 mmol) and sodium iodide 78 mg (0.52 mmol) in DMF 10 ml was added 1,2-dibromomethane 90 mg (0.52 mmol). The mixture was stirred at rt for 3 h. The reaction solution was distilled under reduced pressure. The crude was separated by silica gel column chromatography to give compound 15.1.

### Example 30

### Compound 15 in Table 1

To a compound 15.1 642 mg (1 mmol) in THF 10 ml was added aminotriazole 100 mg (1.2 mmol). The mixture was stirred at rt for 12 h. The reaction solution was distilled under reduced pressure. The crude was separated by silica gel column chromatography to give compound 15, ¹HNMR (CDCl³) δ 7.27 (s, 1H), 6.65 (d, J=5.1 Hz, 1H), 6.15 (m, 2H), 5.47 (d, J=5.1 Hz, 1H), 5.20 (s, 1H), 5.10 (m, 2H), 4.36 (m, 2H), 3.80 (s, 3H), 3.34 (m, 1H), 3.18 (m, 1H), 3.09 (m, 1H), 2.85 (s, 1H), 2.54 (t, J=2.1 Hz, 1H), 2.41 (d, J=4.8 Hz, 1H), 2.35 (q, J=7.8 Hz, 1H), 2.26 (q, J=6.9 Hz, 1H), 2.08 (m, 3H), 1.76 (m, 6H), 1.72 (s, 3H), 1.66 (s, 3H), 1.64 (s, 3H), 1.56 (s, 3H), 1.42 (s, 3H), 1.38 (s, 3H), 0.85 (s, 3H).

### Example 31

### Compound 16 in Table 1

To a mixture of methyl-6-(2-bromoethyl) gambogate 224 mg (0.3 mmol), potassium carbonate 82.8 mg (0.6 mmol) in DMF 10 ml was added ethanolamine 20 mg (0.33 mmol). The mixture was stirred at rt for 8 h. The reaction solution was distilled under reduced pressure. The crude was separated by silica gel column chromatography to give compound 16, IR (KBr, cm-1): 3410, 2957, 2924, 2854, 1738, 1716, 1663, 1606, 1515, 1463, 1384, 1322, 1258, 1110, 1043, 849, 690, 606; ¹H NMR (CDCl₃) δ 7.46 (d, J=6.9 Hz, 1H), 6.73 (m, 1H), 5.95 (m, J=7.4 Hz, 1H), 5.67 (d, J=10.2 Hz, 1H), 5.09 (m, 2H), 3.44 (m, 6H), 3.00 (m, 2H), 2.54 (m, 1H), 2.30 (m, 1H), 2.06 (m, 2H), 1.84˜1.75 (br, 5H), 1.74˜1.59 (m, 14H), 1.58 (m, 4H), 1.55 (m, 4H), 1.46 (s, 4H), 1.30 (s, 6H).

### Example 32

### Compound 17 in Table 1

To a mixture of methyl gambogate 642 mg (1 mmol) in ethanol 10 ml was added aminotriazole 100 mg (1.2 mmol). The mixture was stirred at rt for 12 h. The reaction solution was distilled under reduced pressure. The crude was separated by silica gel column chromatography to give compound 17, IR (KBr, cm⁻¹): 3438, 2921, 1735, 1710, 1629, 1554, 1451, 1383, 1246, 1158, 1021, 789. ¹H NMR (CDCl₃) δ 11.60 (s, 1H), 7.27 (s, 1H), 6.65 (d, J=5.1 Hz, 1H), 5.47 (d, J=5.1 Hz, 1H), 5.20 (s, 1H), 5.10 (m, 2H), 4.36 (m, 2H), 3.80 (s, 3H), 3.34 (m, 1H), 3.18 (m, 1H), 3.09 (m, 1H), 2.85 (s, 1H), 2.54 (t, J=2.1 Hz, 1H), 2.41 (d, J=4.8 Hz, 1H), 2.35 (q, J=7.8 Hz, 1H), 2.26 (q, J=6.9 Hz, 1H), 2.08 (m, 3H), 1.76 (m, 6H), 1.72 (s, 3H), 1.66 (s, 3H), 1.64 (s, 3H), 1.56 (s, 3H), 1.42 (s, 3H), 1.38 (s, 3H), 0.85 (s, 3H).

### Example 33

### Compound 18 in Table 1

To a mixture of methyl gambogate 642 mg (1 mmol) in ethanol 10 ml was added aminotriazole 100 mg (1.2 mmol). The mixture was refluxed for 16 h. The reaction solution was distilled under reduced pressure. The crude was separated by silica gel column chromatography to give compound 18, IR (KBr, cm⁻¹): 3437, 2925, 1737, 1628, 1554, 1458, 1383, 1173, 1126, 1025, 752; ¹HNMR (CDCl₃) δ 12.79 (s, 1H), 7.26 (s, 1H), 6.67 (d, J=10.5 Hz, 1H), 6.37 (m, 1H), 5.45 (d, J=10.2 Hz, 1H), 5.10 (m, 2H), 3.65 (s, 3H), 3.50 (m, 2H), 3.26 (d, J=6.6 Hz, 2H), 2.62 (d, J=7.5 Hz, 2H), 2.53 (d, J=9.3 Hz, 1H), 2.34 (m, 1H), 2.04 (m, 2H), 1.74 (s, 3H), 1.71 (s, 3H), 1.65 (m, 7H), 1.56 (m, 5H), 1.43 (s, 3H), 1.37 (s, 3H), 1.30 (s, 3H).

### Example 34

### Compound 19.1 in Table 1

To a mixture of methyl gambogate 642 mg (1 mmol) in ethanol 10 ml was added malononitrile 330 mg (5 mmol). The mixture was refluxed for 3 h. The reaction solution was distilled under reduced pressure. The crude was separated by silica gel column chromatography to give compound 19.1.

### Example 35

### Compound 19 in Table 1

To a mixture of methyl-9,10-dihydro-O-adiponitrile gambogate 694 mg (1 mmol) and potassium carbonate 690 mg (5 mmol) in ethanol 10 ml was added hydroxylamine hydrochloride 350 mg (5 mmol). The mixture was refluxed for 18 h. The reaction solution was distilled under reduced pressure. The crude was separated by silica gel column chromatography to give compound 19, IR (KBr, cm⁻¹): 3435, 2976, 2938, 2739, 2678, 2530, 2492, 1647, 1476, 1398, 1383, 1172, 1143, 1073, 1036, 850, 806 ¹H NMR (CDCl₃) δ 12.76 (s, 1H), 7.49 (m, 1H), 5.90 (m, 1H), 5.10 (m, 1H), 4.59 (s, 1H), 4.28 (m, 1H), 3.56 (m, 1H), 3.46 (m, 4H), 3.43˜3.20 (m, 2H), 2.96 (m, 2H), 2.54˜2.21 (m, 4H), 1.93 (m, 5H), 1.84 (m, 2H), 1.77˜1.71 (m, 7H), 1.66˜1.64 (m, 8H), 1.58˜1.52 (m, 2H), 1.41˜1.31 (m, 6H), 1.28˜1.25 (m, 4H).

### Example 36

### Compound 20 in Table 1

To a mixture of methyl gambogate 694 mg (1 mmol), methylamine hydrochloride 135 mg (2 mmol) and triethylamine 404 mg (4 mmol) in DMF 10 ml was added indanedione 176 mg (1.2 mmol). The mixture was stirred at rt for 12 h. The reaction solution was distilled under reduced pressure. The crude was separated by silica gel column chromatography to give compound 20, IR (KBr, cm⁻¹): 3434, 2924, 2853, 1721, 1620, 1595, 1428, 1351, 1253, 1138, 1072, 991, 736, 681 ¹H NMR (CDCl₃) δ 11.81 (s, 1H), 7.94 (m, 2H), 7.87 (m, 2H), 6.62 (m, 2H), 5.45 (d, J=10.2 Hz, 1H), 5.08 (m, 2H), 4.26 (d, J=3.6 Hz, 1H), 3.65 (s, 3H), 3.37˜3.23 (m, 6H), 2.95 (m, 1H), 2.69 (d, J=11.4 Hz, 1H), 2.58 (d, J=8.7 Hz, 1H), 2.17˜2.05 (m, 4H), 1.95 (s, 3H), 1.76 (s, 3H), 1.66 (m, 6H), 1.57 (m, 5H), 1.39 (s, 3H), 1.38 (s, 3H), 1.17 (s, 3H).

### Example 37

### Compound 21 in Table 1

To a mixture of methyl gambogate 694 mg (1 mmol) and triethylamine 404 mg (4 mmol) in DMF 10 ml was added o-amino thiophenol 150 mg (1.2 mmol). The mixture was stirred at rt for 20 h. The reaction solution was distilled under reduced pressure. The crude was separated by silica gel column chromatography to give compound 21, IR (KBr, cm⁻¹): 3460, 3370, 2970, 2924, 2853, 1738, 1713, 1628, 1479, 1454, 1397, 1367, 1309, 1220, 1174, 1045, 987, 955; ¹H NMR (CDCl₃) δ 11.80 (s, 1H), 7.53 (d, J=3.9 Hz, 1H), 7.23 (t, J=3.6 Hz, 1H), 7.03 (m, 1H), 6.86 (t, J=3.6 Hz, 1H), 6.66 (d, J=5.1 Hz, 1H), 6.63 (m, 1H), 5.46 (d, J=5.1 Hz, 1H), 5.07 (m, 2H), 4.33 (s, 1H), 3.73 (s, 3H), 3.44˜3.15 (m, 5H), 2.51 (d, J=4.2 Hz, 1H), 2.48 (s, 1H), 2.09˜1.94 (m, 3H), 1.74 (s, 3H), 1.68˜1.62 (m, 9H), 1.57 (s, 3H), 1.37 (s, 3H), 1.35 (s, 3H), 1.27 (s, 3H), 1.11 (s, 3H).

### Example 38

### Compound 22 in Table 1

To a mixture of methyl gambogate 694 mg (1 mmol) and diamine 72 mg (1.2 mmol) in DMF 10 ml was added indanedione 176 mg (1.2 mmol). The mixture was stirred at rt for 12 h. The reaction solution was distilled under reduced pressure. The crude was separated by silica gel column chromatography to give compound 22, IR (KBr, cm⁻¹): 3431, 2925, 1739, 1709, 1626, 1523, 1453, 1383, 1321, 1253, 1217, 1175, 1125, 907, 840, 754, 620. 1H NMR (CDCl₃) δ 11.92 (s, 1H), 7.58 (m, 2H), 6.90 (m, 2H), 6.57 (d, J=5.1 Hz, 1H), 6.49 (m, 1H), 5.38 (d, J=5.1 Hz, 1H), 5.22 (d, J=3.6 Hz, 1H), 5.02˜4.92 (m, 3H), 4.25˜4.18 (m, 4H), 3.78 (m, 2H), 3.59 (m, 5H), 3.21˜3.18 (m, 4H), 2.75 (m, 2H), 2.40 (m, 1H), 2.00 (m, 2H), 1.85 (s, 3H), 1.65 (s, 3H), 1.58 (s, 3H), 1.57 (s, 3H), 1.55 (s, 3H), 1.50 (s, 3H), 1.28 (s, 3H), 1.03 (s, 3H).

TABLE 1
Embodiment 1-441

| Ex- | | | |
|---|---|---|---|
| am- | | | M. |
| ple | Chemical Structure | Formula | Weight |
| 1 | 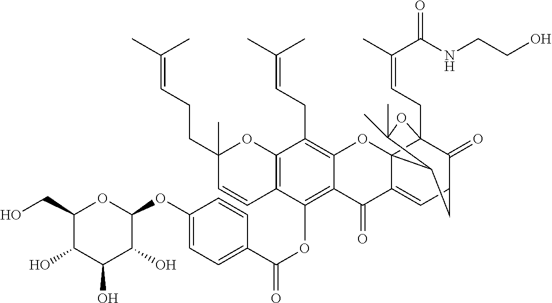 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C(=O)C2=CC4CC5C(C)(C)OC(C/C=C(/C)C(=O)NCCO)(C4=O)C25O3)O1] | C₅₃H₆₃NO₁₅ | 954.07 |
| 1.1 | 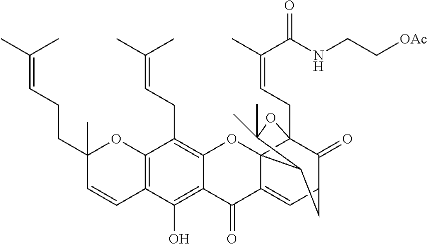 [SMILES: CC(=O)OCCNC(=O)/C(C)=C\CC12OC(C)(C)C3CC(C=C4C(=O)c5c(O)c6c(c(CC=C(C)C)c5OC431)OC(C)(CCC=C(C)C)C=C6)C2=O] | C₄₂H₅₁O₈ | 713.86 |
| 1.2 | 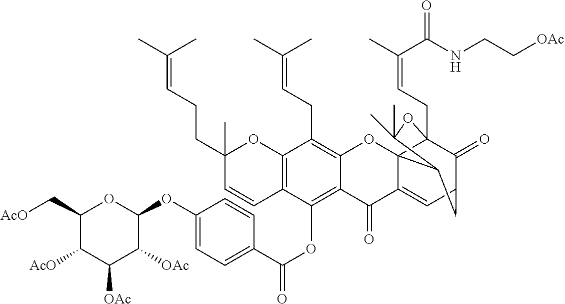 [SMILES: CC(=O)OCCNC(=O)/C(C)=C\CC12OC(C)(C)C3CC(C=C4C(=O)c5c(OC(=O)c6ccc(O[C@@H]7O[C@H](COC(C)=O)[C@@H](OC(C)=O)[C@H](OC(C)=O)[C@H]7OC(C)=O)cc6)c6c(c(CC=C(C)C)c5OC431)OC(C)(CCC=C(C)C)C=C6)C2=O] | C₆₃H₇₃NO₂₀ | 1164.25 |
| 2 | 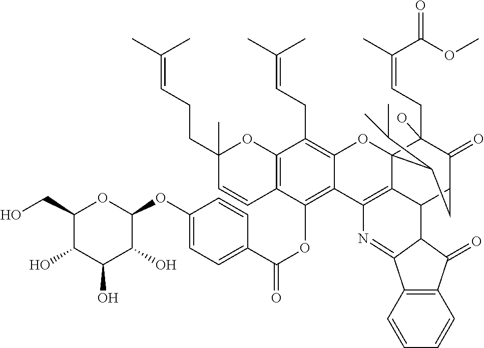 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C2C(=O)c3ccccc3C2=N1)C=CC(C)(CCC=C(C)C)O4] | C₆₁H₆₅NO₁₅ | 1052.17 |
| 2.1 | 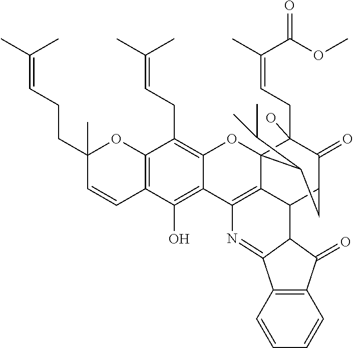 [SMILES: COC(=O)/C(C)=C\CC1(O)C(=O)C2CC(C(C)C)C13Oc1c(CC=C(C)C)c4c(c(O)c1C1=C3C2C2C(=O)c3ccccc3C2=N1)C=CC(C)(CCC=C(C)C)O4] | C₄₈H₅₁NO₈ | 769.92 |
| 2.2 | 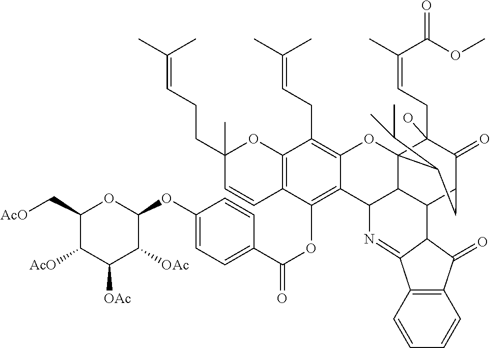 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](COC(C)=O)[C@@H](OC(C)=O)[C@H](OC(C)=O)[C@H]6OC(C)=O)cc5)c1C1N=C5c6ccccc6C(=O)C5C2C13)C=CC(C)(CCC=C(C)C)O4] | C₆₉H₇₅NO₁₉ | 1222.33 |
| 3 | 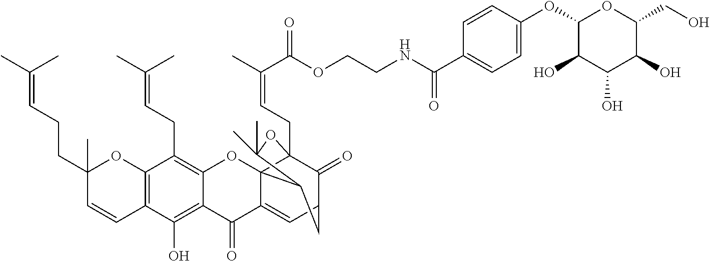 [SMILES: CC(C)=CCCC1(C)C=Cc2c(O)c3c(c(CC=C(C)C)c2O1)OC12C(=CC4CC1C(C)(C)OC2(C/C=C(/C)C(=O)OCCNC(=O)c1ccc(O[C@@H]2O[C@H](CO)[C@@H](O)[C@H](O)[C@H]2O)cc1)C4=O)C3=O] | C₅₃H₆₃NO₁₅ | 954.07 |
| 3.1 | 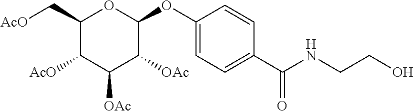 [SMILES: CC(=O)OC[C@H]1O[C@@H](Oc2ccc(C(=O)NCCO)cc2)[C@H](OC(C)=O)[C@@H](OC(C)=O)[C@@H]1OC(C)=O] | C₂₃H₂₉NO₁₂ | 511.48 |
| 3.2 | 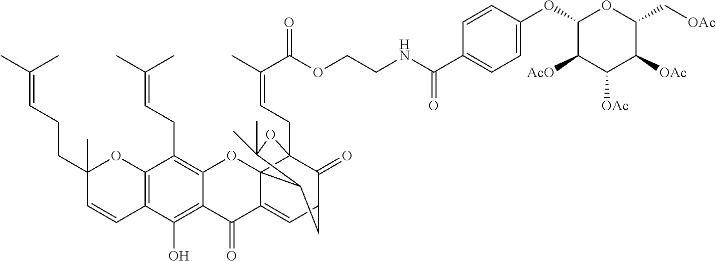 [SMILES: CC(=O)OC[C@H]1O[C@@H](Oc2ccc(C(=O)NCCOC(=O)/C(C)=C\CC34OC(C)(C)C5CC(C=C6C(=O)c7c(O)c8c(c(CC=C(C)C)c7OC653)OC(C)(CCC=C(C)C)C=C8)C4=O)cc2)[C@H](OC(C)=O)[C@@H](OC(C)=O)[C@@H]1OC(C)=O] | C₆₁H₇₁NO₁₉ | 1122.21 |
| 4 | 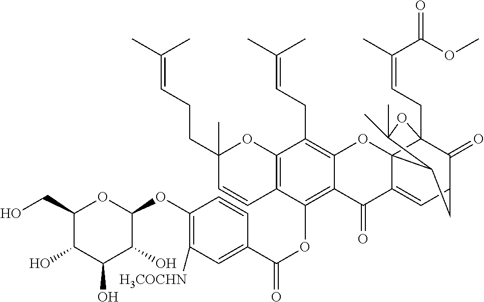 | C₅₄H₆₃NO₁₆ | 982.08 |
| 4.1 | 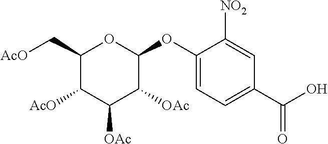 [SMILES: CC(=O)OC[C@H]1O[C@@H](Oc2ccc(C(=O)O)cc2[N+](=O)[O-])[C@H](OC(C)=O)[C@@H](OC(C)=O)[C@@H]1OC(C)=O] | C₂₁H₂₃NO₁₄ | 513.41 |
| 4.2 | 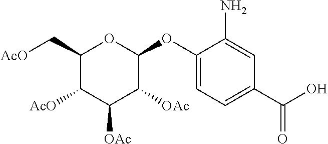 [SMILES: CC(=O)OC[C@H]1O[C@@H](Oc2ccc(C(=O)O)cc2N)[C@H](OC(C)=O)[C@@H](OC(C)=O)[C@@H]1OC(C)=O] | C₂₁H₂₅NO₁₂ | 483.42 |
| 4.3 | 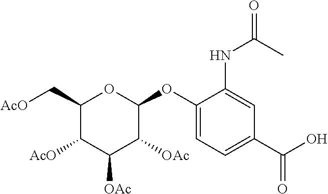 [SMILES: CC(=O)Nc1cc(C(=O)O)ccc1O[C@@H]1O[C@H](COC(C)=O)[C@@H](OC(C)=O)[C@H](OC(C)=O)[C@H]1OC(C)=O] | C₂₃H₂₇NO₁₃ | 525.46 |
| 4.4 | 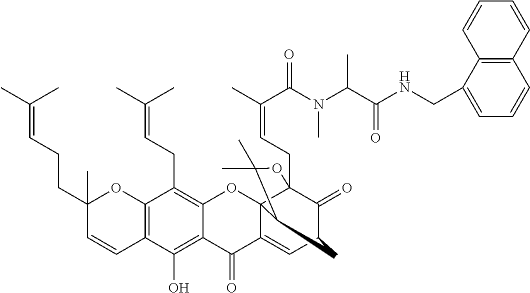 [SMILES: CC(C)=CCCC1(C)C=Cc2c(O)c3c(c(CC=C(C)C)c2O1)OC12C(=C[C@@H]4C[C@@H]1C(C)(C)OC2(C/C=C(/C)C(=O)N(C)C(C)C(=O)NCc1cccc2ccccc12)C4=O)C3=O] | C₅₃H₆₀N₂O₈ | 853.05 |
| 5 | 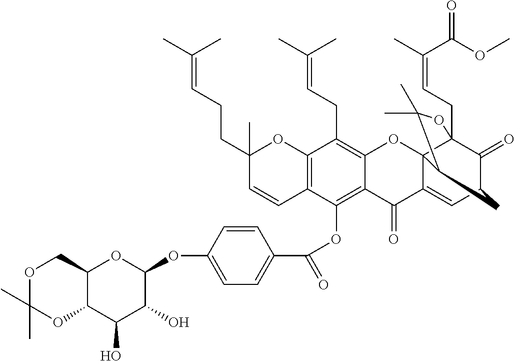 [SMILES: COC(=O)/C(C)=C\CC12OC(C)(C)[C@H]3C[C@@H](C=C4C(=O)c5c(OC(=O)c6ccc(O[C@@H]7O[C@@H]8COC(C)(C)O[C@H]8[C@H](O)[C@H]7O)cc6)c6c(c(CC=C(C)C)c5OC431)OC(C)(CCC=C(C)C)C=C6)C2=O] | C₅₅H₆₄NO₁₅ | 965.09 |
| 6 | 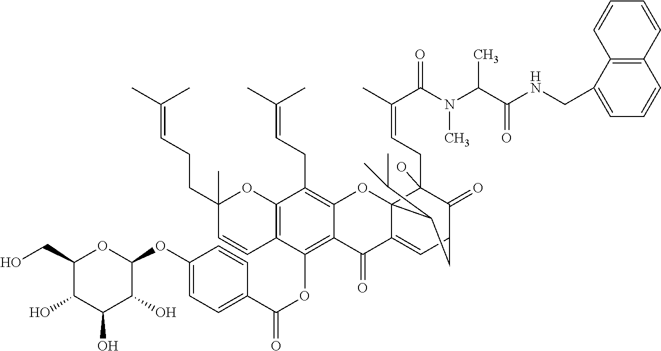 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C(=O)C2=CC4CC(C(C)C)C2(O3)C(O)(C/C=C(/C)C(=O)N(C)C(C)C(=O)NCc2cccc3ccccc23)C4=O)O1] | C₆₆H₇₄N₂O₁₅ | 1135.3 |
| 6.1 | 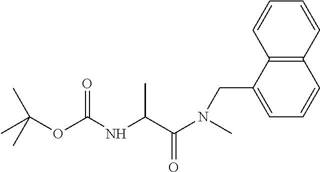 [SMILES: CC(NC(=O)OC(C)(C)C)C(=O)N(C)Cc1cccc2ccccc12] | C₂₀H₂₆N₂O₃ | 342.43 |
| 6.2 | 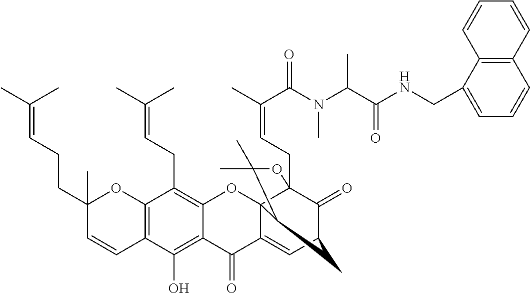 [SMILES: CC(C)=CCCC1(C)C=Cc2c(O)c3c(c(CC=C(C)C)c2O1)OC12C(=C[C@@H]4C[C@@H]1C(C)(C)OC2(C/C=C(/C)C(=O)N(C)C(C)C(=O)NCc1cccc2ccccc12)C4=O)C3=O] | C₅₃H₆₀N₂O₈ | 853.05 |
| 6.3 | 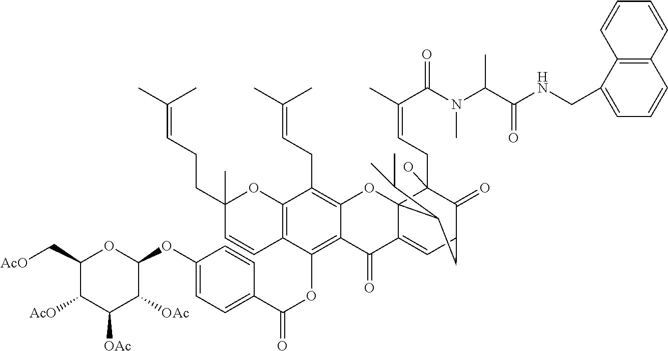 [SMILES: CC(=O)OC[C@H]1O[C@@H](Oc2ccc(C(=O)Oc3c4c(c(CC=C(C)C)c5c3C(=O)C3=CC6CC(C(C)C)C3(O5)C(O)(C/C=C(/C)C(=O)N(C)C(C)C(=O)NCc3cccc5ccccc35)C6=O)OC(C)(CCC=C(C)C)C=C4)cc2)[C@H](OC(C)=O)[C@@H](OC(C)=O)[C@@H]1OC(C)=O] | C₇₄H₈₂N₂O₁₉ | 1303.44 |
| 6.4 | 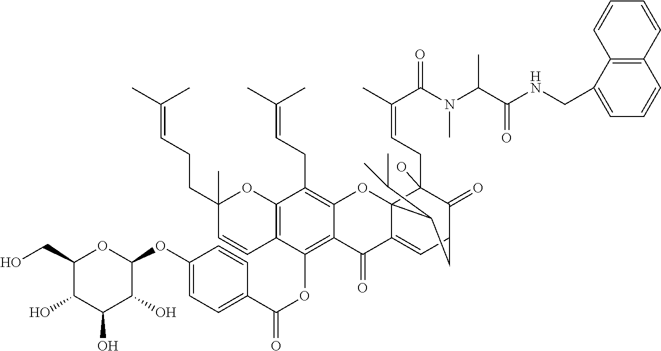 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C(=O)C2=CC4CC(C(C)C)C2(O3)C(O)(C/C=C(/C)C(=O)N(C)C(C)C(=O)NCc2cccc3ccccc23)C4=O)O1] | C₆₆H₇₄N₂O₁₅ | 1135.3 |
| 7 | 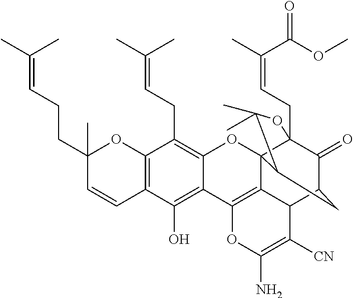 [SMILES: COC(=O)/C(C)=C\CC12OC(C)(C)C3CC(C1=O)C1C(C#N)=C(N)OC4=C1C32Oc1c(CC=C(C)C)c2c(c(O)c14)C=CC(C)(CCC=C(C)C)O2] | C₄₂H₄₈N₂O₈ | 708.84 |
| 8 | 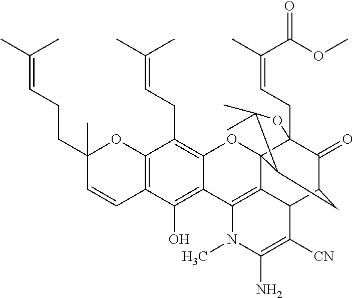 [SMILES: COC(=O)/C(C)=C\CC12OC(C)(C)C3CC(C1=O)C1C(C#N)=C(N)N(C)C4=C1C32Oc1c(CC=C(C)C)c2c(c(O)c14)C=CC(C)(CCC=C(C)C)O2] | C₄₃H₅₁N₃O₇ | 721.88 |
| 9 | 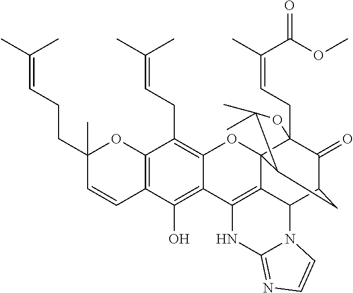 [SMILES: COC(=O)/C(C)=C\CC12OC(C)(C)C3CC(C1=O)C1C4=C(Nc5nccn51)c1c(O)c5c(c(CC=C(C)C)c1OC432)OC(C)(CCC=C(C)C)C=C5] | C₄₂H₄₉N₃O₇ | 707.85 |
| 10 | 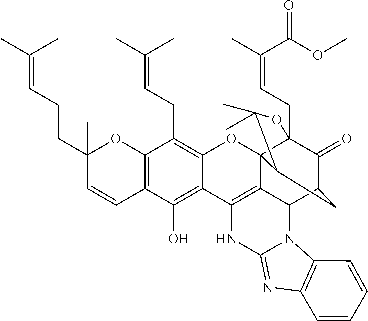 [SMILES: COC(=O)/C(C)=C\CC12OC(C)(C)C3CC(C1=O)C1C4=C(Nc5nc6ccccc6n51)c1c(O)c5c(c(CC=C(C)C)c1OC432)OC(C)(CCC=C(C)C)C=C5] | C₄₆H₅₁N₃O₇ | 757.91 |
| 11 | 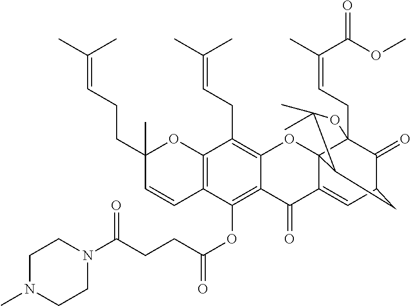 [SMILES: COC(=O)/C(C)=C\CC12OC(C)(C)C3CC(C=C4C(=O)c5c(OC(=O)CCC(=O)N6CCN(C)CC6)c6c(c(CC=C(C)C)c5OC431)OC(C)(CCC=C(C)C)C=C6)C2=O] | C₄₈H₆₀N₂O₁₀ | 825 |
| 12 | 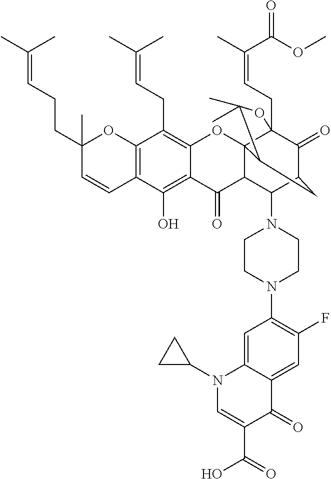 [SMILES: COC(=O)/C(C)=C\CC12OC(C)(C)C3CC(C1=O)C(N1CCN(c4cc5c(cc4F)c(=O)c(C(=O)O)cn5C4CC4)CC1)C1C(=O)c4c(O)c5c(c(CC=C(C)C)c4OC132)OC(C)(CCC=C(C)C)C=C5] | C₅₆H₆₄FN₃O₁₁ | 974.12 |
| 13 | 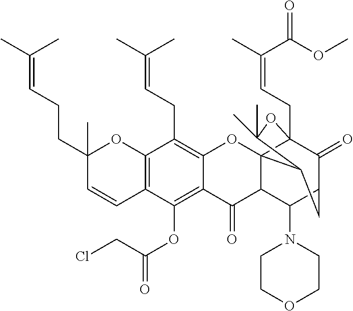 [SMILES: COC(=O)/C(C)=C\CC12OC(C)(C)C3CC(C1=O)C(N1CCOCC1)C1C(=O)c4c(OC(=O)CCl)c5c(c(CC=C(C)C)c4OC132)OC(C)(CCC=C(C)C)C=C5] | C₄₃H₅₆ClNO₁₀ | 806.38 |
| 14 | 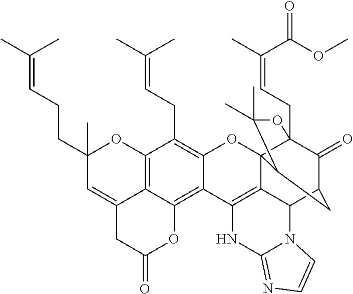 [SMILES: COC(=O)/C(C)=C\CC12OC(C)(C)C3CC(C1=O)C1C4=C(Nc5nccn51)c1c(c(CC=C(C)C)c5c6c1OC(=O)CC6=CC(C)(CCC=C(C)C)O5)OC432] | C₄₄H₄₉N₃O₈ | 747.88 |
| 15 | 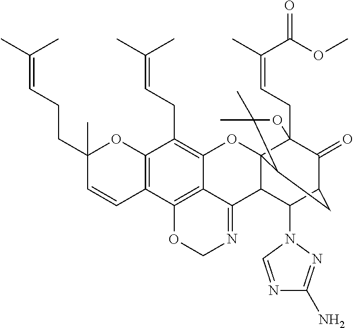 [SMILES: COC(=O)/C(C)=C\CC12OC(C)(C)C3CC(C1=O)C(n1cnc(N)n1)C1C4=NCOc5c6c(c(CC=C(C)C)c(c54)OC132)OC(C)(CCC=C(C)C)C=C6] | C₄₂H₅₁N₅O₇ | 737.88 |
| 16 | 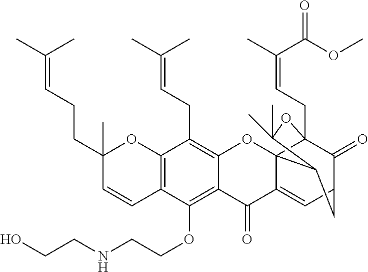 [SMILES: COC(=O)/C(C)=C\CC12OC(C)(C)C3CC(C=C4C(=O)c5c(OCCNCCO)c6c(c(CC=C(C)C)c5OC431)OC(C)(CCC=C(C)C)C=C6)C2=O] | C₄₃H₅₅NO₉ | 729.9 |
| 17 | 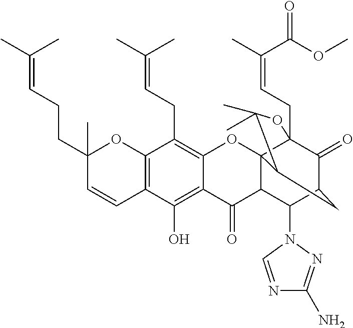 [SMILES: COC(=O)/C(C)=C\CC12OC(C)(C)C3CC(C1=O)C(n1cnc(N)n1)C1C(=O)c4c(O)c5c(c(CC=C(C)C)c4OC132)OC(C)(CCC=C(C)C)C=C5] | C₄₁H₅₀N₄O₈ | 726.86 |
| 18 | 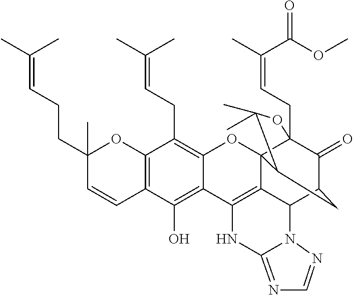 [SMILES: COC(=O)/C(C)=C\CC12OC(C)(C)C3CC(C1=O)C1C4=C(Nc5ncnn51)c1c(O)c5c(c(CC=C(C)C)c1OC432)OC(C)(CCC=C(C)C)C=C5] | C₄₁H₄₈N₄O₇ | 708.84 |
| 19 | 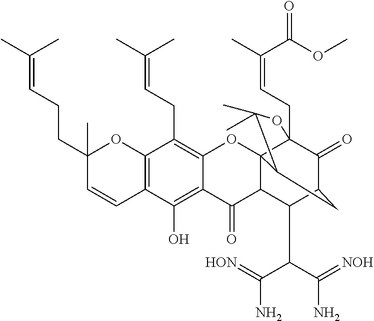 | C₄₂H₅₄N₄O₁₀ | 774.9 |
| 19.1 | 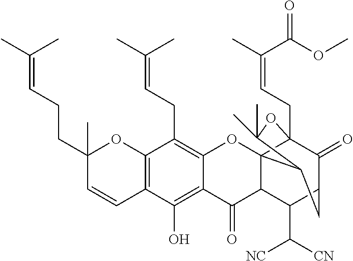 [SMILES: COC(=O)/C(C)=C\CC12OC(C)(C)C3CC(C1=O)C(C(C#N)C#N)C1C(=O)c4c(O)c5c(c(CC=C(C)C)c4OC132)OC(C)(CCC=C(C)C)C=C5] | C₄₂H₄₈N₂O₈ | 708.84 |
| 20 | 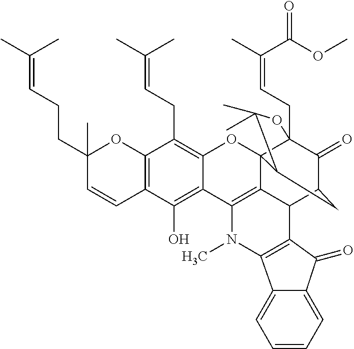 [SMILES: COC(=O)/C(C)=C\CC12OC(C)(C)C3CC(C1=O)C1C4=C(c5ccccc5C4=O)N(C)C4=C1C32Oc1c(CC=C(C)C)c2c(c(O)c14)C=CC(C)(CCC=C(C)C)O2] | C₄₉H₅₃NO₈ | 783.95 |
| 21 | 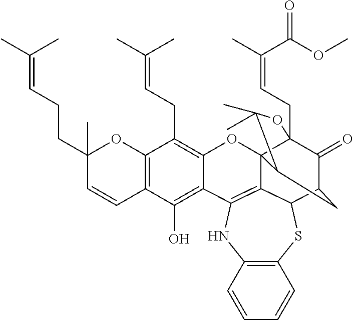 [SMILES: COC(=O)/C(C)=C\CC12OC(C)(C)C3CC(C1=O)C1Sc4ccccc4NC4=C1C32Oc1c(CC=C(C)C)c2c(c(O)c14)C=CC(C)(CCC=C(C)C)O2] | C₄₅H₅₁NO₇S | 749.95 |
| 22 | 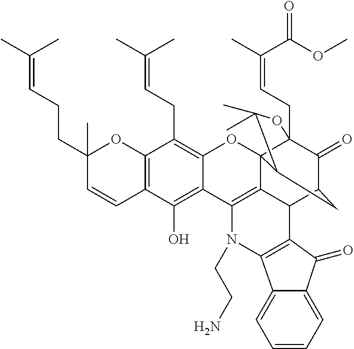 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(O)c14)C=CC(C)(CCC=C(C)C)O2] | C₅₀H₅₆N₂O₈ | 812.99 |
| 23 | 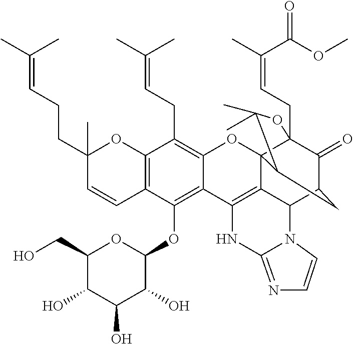 [SMILES: COC(=O)/C(C)=C\CC12OC(C)(C)C3CC(C1=O)C1C4=C(Nc5nccn51)c1c(O[C@@H]5O[C@H](CO)[C@@H](O)[C@H](O)[C@H]5O)c5c(c(CC=C(C)C)c1OC432)OC(C)(CCC=C(C)C)C=C5] | C₄₈H₅₉N₃O₁₂ | 869.99 |
| 24 | 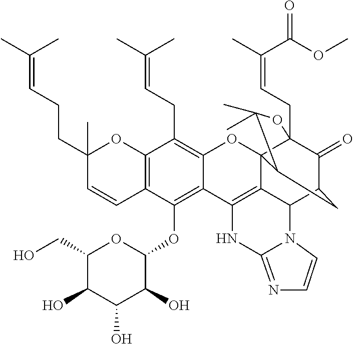 [SMILES: COC(=O)/C(C)=C\CC12OC(C)(C)C3CC(C1=O)C1C4=C(Nc5nccn51)c1c(O[C@H]5O[C@@H](CO)[C@H](O)[C@@H](O)[C@@H]5O)c5c(c(CC=C(C)C)c1OC432)OC(C)(CCC=C(C)C)C=C5] | C₄₈H₅₉N₃O₁₂ | 869.99 |
| 25 | 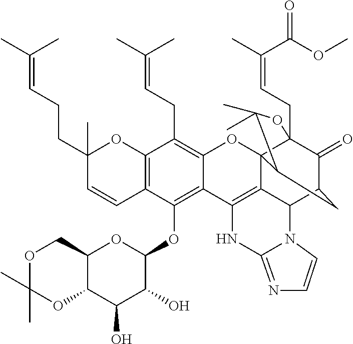 [SMILES: COC(=O)/C(C)=C\CC12OC(C)(C)C3CC(C1=O)C1C4=C(Nc5nccn51)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₅₁H₆₃N₃O₁₂ | 910.06 |
| 26 | 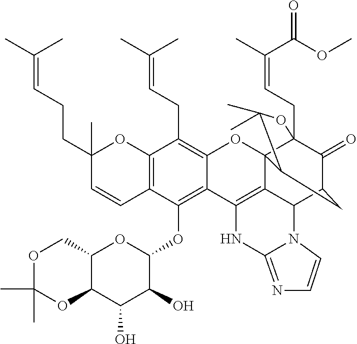 [SMILES: COC(=O)/C(C)=C\CC12OC(C)(C)C3CC(C1=O)C1C4=C(Nc5nccn51)c1c(O[C@H]5O[C@H]6COC(C)(C)O[C@@H]6[C@@H](O)[C@@H]5O)c5c(c(CC=C(C)C)c1OC432)OC(C)(CCC=C(C)C)C=C5] | C₅₁H₆₃N₃O₁₂ | 910.06 |
| 27 | 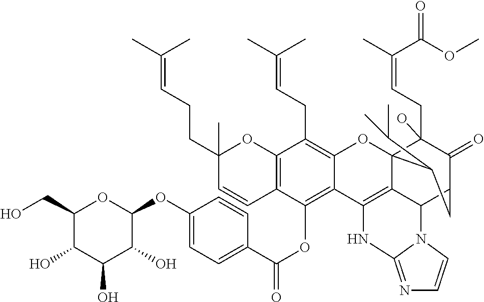 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2n2ccnc2N1)C=CC(C)(CCC=C(C)C)O4] | C₅₅H₆₃N₃O₁₄ | 990.1 |
| 28 | 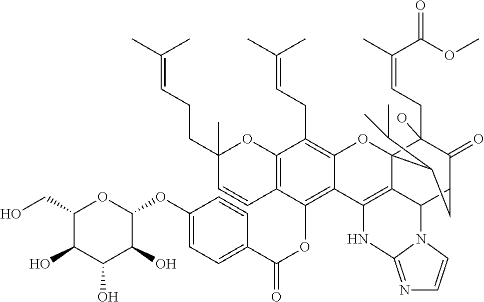 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2n2ccnc2N1)C=CC(C)(CCC=C(C)C)O4] | C₅₅H₆₃N₃O₁₄ | 990.1 |
| 29 | 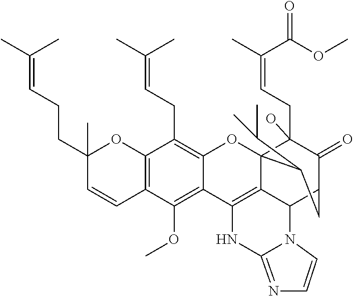 [SMILES: COC(=O)/C(C)=C\CC1(O)C(=O)C2CC(C(C)C)C13Oc1c(CC=C(C)C)c4c(c(OC)c1C1=C3C2n2ccnc2N1)C=CC(C)(CCC=C(C)C)O4] | C₄₃H₅₁N₃O₇ | 721.88 |
| 30 | 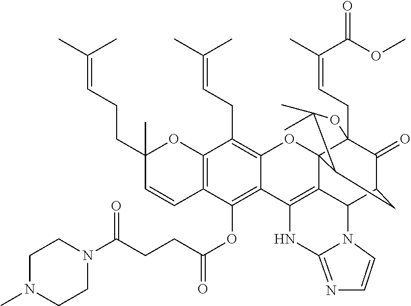 [SMILES: COC(=O)/C(C)=C\CC12OC(C)(C)C3CC(C1=O)C1C4=C(Nc5nccn51)c1c(OC(=O)CCC(=O)N5CCN(C)CC5)c5c(c(CC=C(C)C)c1OC432)OC(C)(CCC=C(C)C)C=C5] | C₅₀H₆₃N₅O₉ | 890.07 |
| 31 | 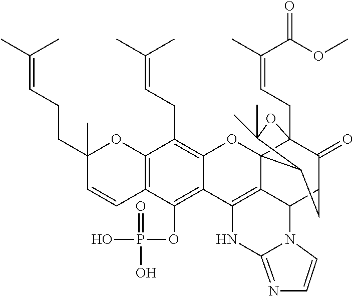 [SMILES: COC(=O)/C(C)=C\CC12OC(C)(C)C3CC(C1=O)C1C4=C(Nc5nccn51)c1c(OP(=O)(O)O)c5c(c(CC=C(C)C)c1OC432)OC(C)(CCC=C(C)C)C=C5] | C₄₂H₅₀N₃O₁₀P | 787.83 |
| 32 | 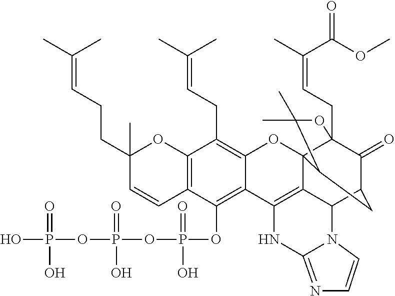 [SMILES: COC(=O)/C(C)=C\CC12OC(C)(C)C3CC(C1=O)C1C4=C(Nc5nccn51)c1c(OP(=O)(O)OP(=O)(O)OP(=O)(O)O)c5c(c(CC=C(C)C)c1OC432)OC(C)(CCC=C(C)C)C=C5] | C₄₂H₅₂N₃O₁₆P₃ | 947.79 |
| 33 | 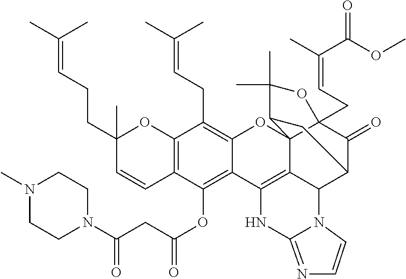 | C₅₀H₆₁N₅O₉ | 876.05 |
| 34 | 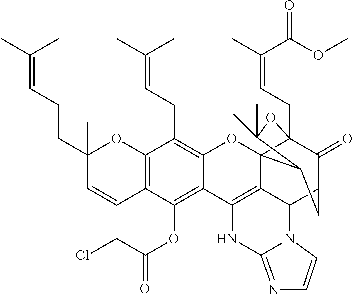 [SMILES: COC(=O)/C(C)=C\CC12OC(C)(C)C3CC(C1=O)C1C4=C(Nc5nccn51)c1c(OC(=O)CCl)c5c(c(CC=C(C)C)c1OC432)OC(C)(CCC=C(C)C)C=C5] | C₄₄H₅₀ClN₃O₈ | 784.34 |
| 35 | 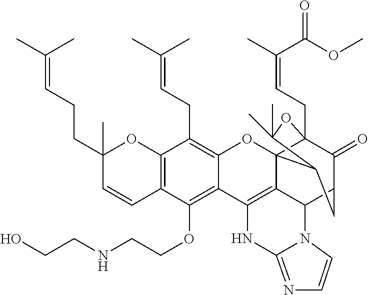 [SMILES: COC(=O)/C(C)=C\CC12OC(C)(C)C3CC(C1=O)C1C4=C(Nc5nccn51)c1c(OCCNCCO)c5c(c(CC=C(C)C)c1OC432)OC(C)(CCC=C(C)C)C=C5] | C₄₆H₅₈N₄O₈ | 794.97 |
| 36 | 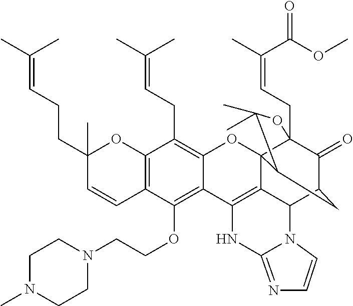 [SMILES: COC(=O)/C(C)=C\CC12OC(C)(C)C3CC(C1=O)C1C4=C(Nc5nccn51)c1c(OCCN5CCN(C)CC5)c5c(c(CC=C(C)C)c1OC432)OC(C)(CCC=C(C)C)C=C5] | C₄₉H₆₃N₅O₇ | 834.05 |
| 37 | 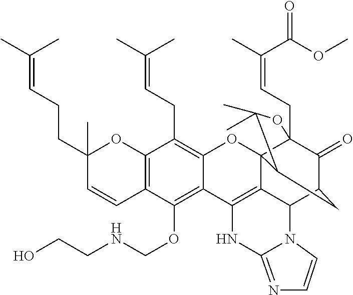 [SMILES: COC(=O)/C(C)=C\CC12OC(C)(C)C3CC(C1=O)C1C4=C(Nc5nccn51)c1c(OCNCCO)c5c(c(CC=C(C)C)c1OC432)OC(C)(CCC=C(C)C)C=C5] | C₄₉H₆₃N₅O₇ | 780.95 |
| 38 | 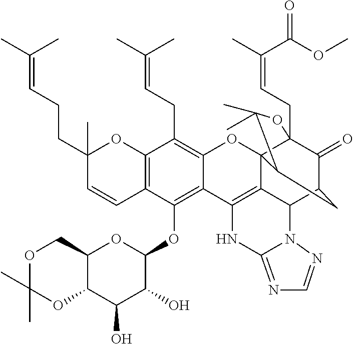 [SMILES: COC(=O)/C(C)=C\CC12OC(C)(C)C3CC(C1=O)C1C4=C(Nc5ncnn51)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₄₃H₅₀BrN₃O₇ | 800.78 |
| 39 | 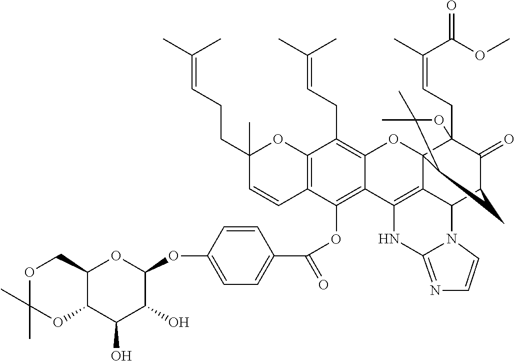 [SMILES: COC(=O)/C(C)=C\CC12OC(C)(C)[C@H]3C[C@H](C1=O)C1C4=C(Nc5nccn51)c1c(OC(=O)c5ccc(O[C@@H]6O[C@@H]7COC(C)(C)O[C@H]7[C@H](O)[C@H]6O)cc5)c5c(c(CC=C(C)C)c1OC432)OC(C)(CCC=C(C)C)C=C5] | C₅₈H₆₇N₃O₁₄ | 1030.16 |
| 40 | 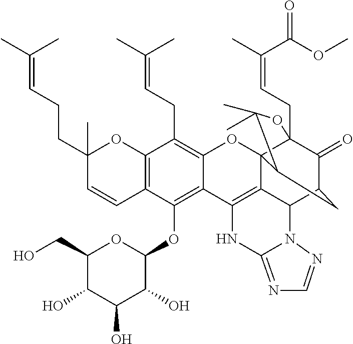 [SMILES: COC(=O)/C(C)=C\CC12OC(C)(C)C3CC(C1=O)C1C4=C(Nc5ncnn51)c1c(O[C@@H]5O[C@H](CO)[C@@H](O)[C@H](O)[C@H]5O)c5c(c(CC=C(C)C)c1OC432)OC(C)(CCC=C(C)C)C=C5] | C₄₇H₅₈N₄O₁₂ | 870.98 |
| 41 | 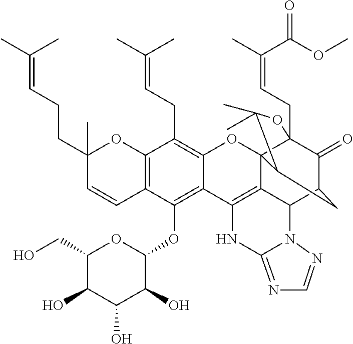 [SMILES: COC(=O)/C(C)=C\CC12OC(C)(C)C3CC(C1=O)C1C4=C(Nc5ncnn51)c1c(O[C@H]5O[C@@H](CO)[C@H](O)[C@@H](O)[C@@H]5O)c5c(c(CC=C(C)C)c1OC432)OC(C)(CCC=C(C)C)C=C5] | C₄₇H₅₈N₄O₁₂ | 870.98 |
| 42 | 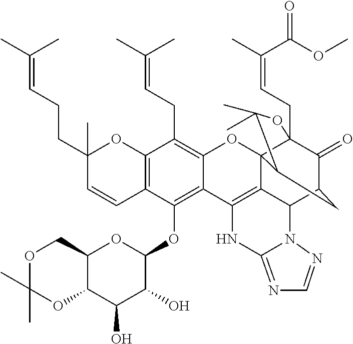 [SMILES: COC(=O)/C(C)=C\CC12OC(C)(C)C3CC(C1=O)C1C4=C(Nc5ncnn51)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₅₀H₆₂N₄O₁₂ | 911.05 |
| 43 | 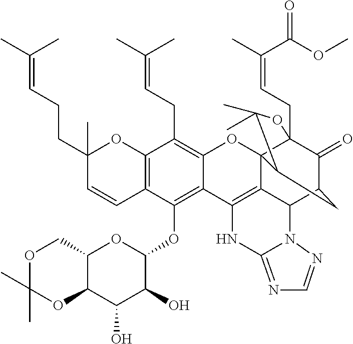 [SMILES: COC(=O)/C(C)=C\CC12OC(C)(C)C3CC(C1=O)C1C4=C(Nc5ncnn51)c1c(O[C@H]5O[C@H]6COC(C)(C)O[C@@H]6[C@@H](O)[C@@H]5O)c5c(c(CC=C(C)C)c1OC432)OC(C)(CCC=C(C)C)C=C5] | C₅₀H₆₂N₄O₁₂ | 911.05 |
| 44 | 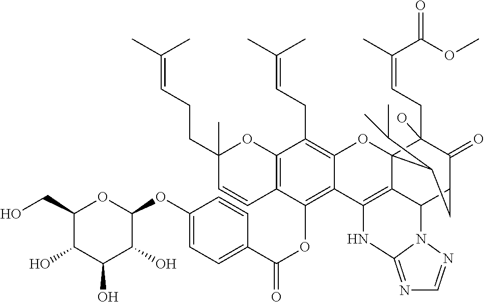 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2n2ncnc2N1)C=CC(C)(CCC=C(C)C)O4] | C₅₄H₆₂N₄O₁₄ | 991.09 |
| 45 | 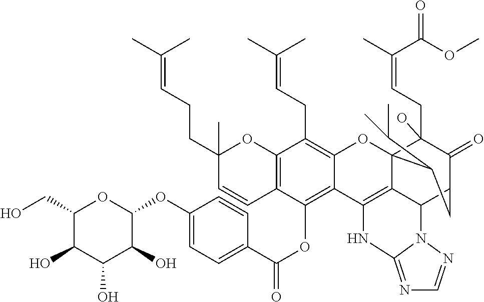 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2n2ncnc2N1)C=CC(C)(CCC=C(C)C)O4] | C₅₄H₆₂N₄O₁₄ | 991.09 |
| 46 | 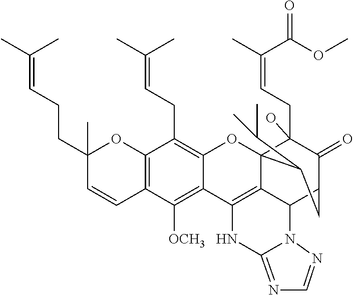 [SMILES: COC(=O)/C(C)=C\CC1(O)C(=O)C2CC(C(C)C)C13Oc1c(CC=C(C)C)c4c(c(OC)c1C1=C3C2n2ncnc2N1)C=CC(C)(CCC=C(C)C)O4] | C₄₂H₅₀N₄O₇ | 722.87 |
| 47 | 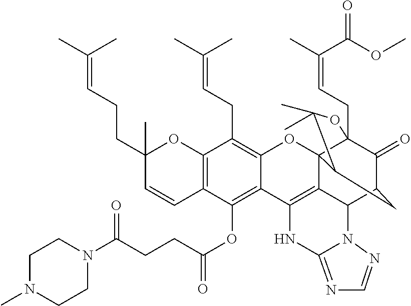 [SMILES: COC(=O)/C(C)=C\CC12OC(C)(C)C3CC(C1=O)C1C4=C(Nc5ncnn51)c1c(OC(=O)CCC(=O)N5CCN(C)CC5)c5c(c(CC=C(C)C)c1OC432)OC(C)(CCC=C(C)C)C=C5] | C₅₀H₆₂N₆O₉ | 891.06 |
| 48 | 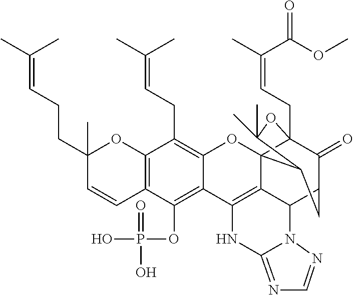 [SMILES: COC(=O)/C(C)=C\CC12OC(C)(C)C3CC(C1=O)C1C4=C(Nc5ncnn51)c1c(OP(=O)(O)O)c5c(c(CC=C(C)C)c1OC432)OC(C)(CCC=C(C)C)C=C5] | C₄₁H₄₉N₄O₁₀P | 788.82 |
| 49 | 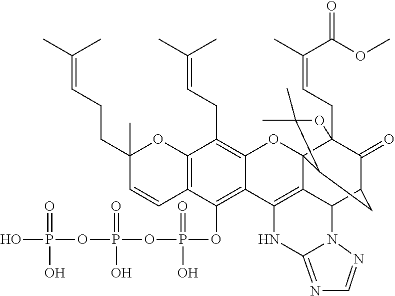 [SMILES: COC(=O)/C(C)=C\CC12OC(C)(C)C3CC(C1=O)C1C4=C(Nc5ncnn51)c1c(OP(=O)(O)OP(=O)(O)OP(=O)(O)O)c5c(c(CC=C(C)C)c1OC432)OC(C)(CCC=C(C)C)C=C5] | C₄₁H₅₁N₄O₁₆P₃ | 948.78 |
| 50 | 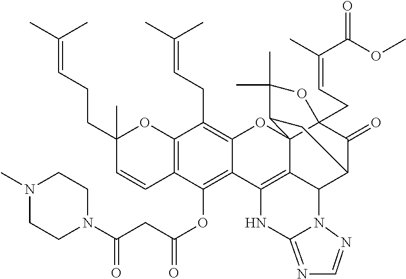 | C₄₉H₆₀N₆O₉ | 877.04 |
| 51 | 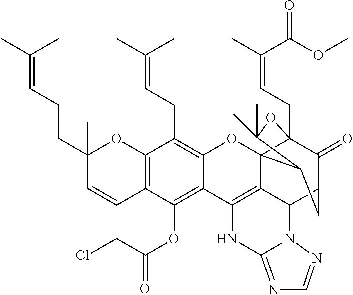 [SMILES: COC(=O)/C(C)=C\CC12OC(C)(C)C3CC(C1=O)C1C4=C(Nc5ncnn51)c1c(OC(=O)CCl)c5c(c(CC=C(C)C)c1OC432)OC(C)(CCC=C(C)C)C=C5] | C₄₃H₄₉ClN₄O₈ | 785.32 |
| 52 | 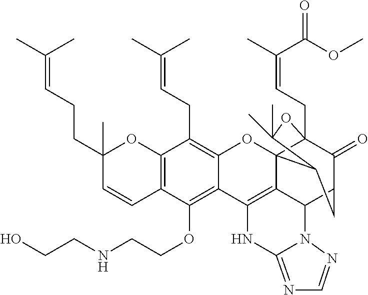 [SMILES: COC(=O)/C(C)=C\CC12OC(C)(C)C3CC(C1=O)C1C4=C(Nc5ncnn51)c1c(OCCNCCO)c5c(c(CC=C(C)C)c1OC432)OC(C)(CCC=C(C)C)C=C5] | C₄₅H₅₇N₅O₈ | 795.96 |
| 53 | 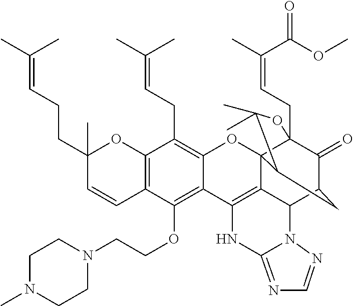 [SMILES: COC(=O)/C(C)=C\CC12OC(C)(C)C3CC(C1=O)C1C4=C(Nc5ncnn51)c1c(OCCN5CCN(C)CC5)c5c(c(CC=C(C)C)c1OC432)OC(C)(CCC=C(C)C)C=C5] | C₄₈H₆₂N₆O₇ | 835.04 |
| 54 | 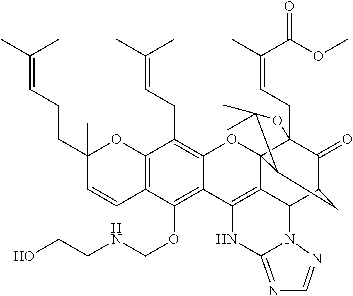 [SMILES: COC(=O)/C(C)=C\CC12OC(C)(C)C3CC(C1=O)C1C4=C(Nc5ncnn51)c1c(OCNCCO)c5c(c(CC=C(C)C)c1OC432)OC(C)(CCC=C(C)C)C=C5] | C₄₄H₅₅N₅O₈ | 781.94 |
| 55 | 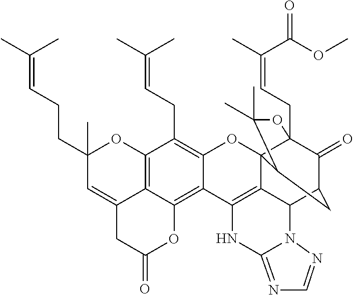 [SMILES: COC(=O)/C(C)=C\CC12OC(C)(C)C3CC(C1=O)C1C4=C(Nc5ncnn51)c1c(c(CC=C(C)C)c5c6c1OC(=O)CC6=CC(C)(CCC=C(C)C)O5)OC432] | C₄₃H₄₈N₄O₈ | 748.86 |
| 56 | 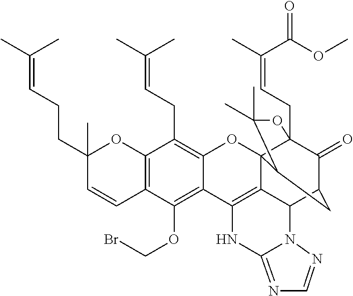 [SMILES: COC(=O)/C(C)=C\CC12OC(C)(C)C3CC(C1=O)C1C4=C(Nc5ncnn51)c1c(OCBr)c5c(c(CC=C(C)C)c1OC432)OC(C)(CCC=C(C)C)C=C5] | C₄₂H₄₉BrN₄O₇ | 801.77 |
| 57 | 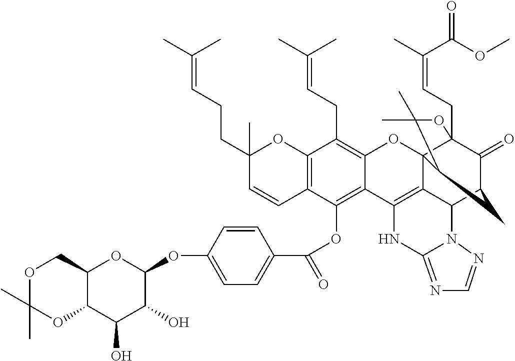 [SMILES: COC(=O)/C(C)=C\CC12OC(C)(C)[C@H]3C[C@H](C1=O)C1C4=C(Nc5ncnn51)c1c(OC(=O)c5ccc(O[C@@H]6O[C@@H]7COC(C)(C)O[C@H]7[C@H](O)[C@H]6O)cc5)c5c(c(CC=C(C)C)c1OC432)OC(C)(CCC=C(C)C)C=C5] | C₅₇H₆₆N₄O₁₄ | 1031.15 |
| 58 | 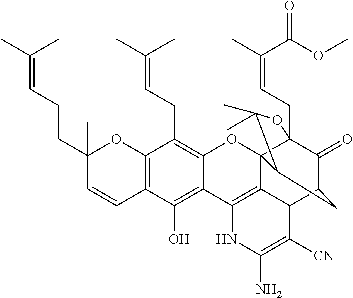 [SMILES: COC(=O)/C(C)=C\CC12OC(C)(C)C3CC(C1=O)C1C(C#N)=C(N)NC4=C1C32Oc1c(CC=C(C)C)c2c(c(O)c14)C=CC(C)(CCC=C(C)C)O2] | C₄₂H₄₉N₃O₇ | 707.85 |
| 59 | 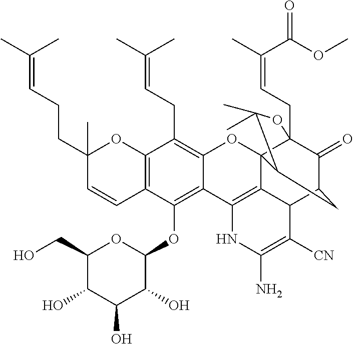 [SMILES: COC(=O)/C(C)=C\CC12OC(C)(C)C3CC(C1=O)C1C(C#N)=C(N)NC4=C1C32Oc1c(CC=C(C)C)c2c(c(O[C@@H]3O[C@H](CO)[C@@H](O)[C@H](O)[C@H]3O)c14)C=CC(C)(CCC=C(C)C)O2] | C₄₈H₅₉N₃O₁₂ | 869.99 |
| 60 | 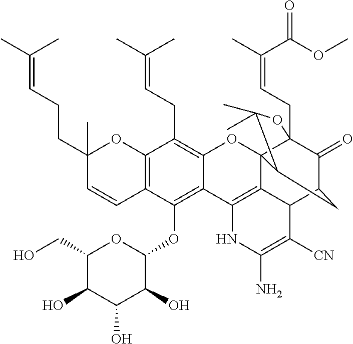 [SMILES: COC(=O)/C(C)=C\CC12OC(C)(C)C3CC(C1=O)C1C(C#N)=C(N)NC4=C1C32Oc1c(CC=C(C)C)c2c(c(O[C@H]3O[C@@H](CO)[C@H](O)[C@@H](O)[C@@H]3O)c14)C=CC(C)(CCC=C(C)C)O2] | C₄₈H₅₉N₃O₁₂ | 869.99 |
| 61 | 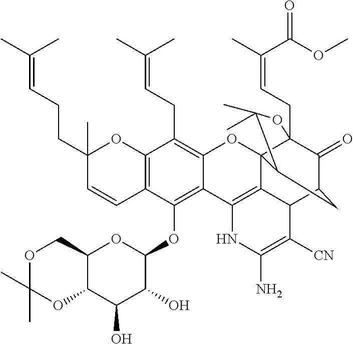 [SMILES: COC(=O)/C(C)=C\CC12OC(C)(C)C3CC(C1=O)C1C(C#N)=C(N)NC4=C1C32Oc1c(CC=C(C)C)c2c(c(O[C@@H]3O[C@@H]5COC(C)(C)O[C@H]5[C@H](O)[C@H]3O)c14)C=CC(C)(CCC=C(C)C)O2] | C₅₁H₆₃N₃O₁₂ | 910.06 |
| 62 | 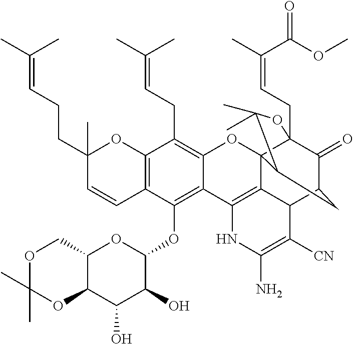 [SMILES: COC(=O)/C(C)=C\CC12OC(C)(C)C3CC(C1=O)C1C(C#N)=C(N)NC4=C1C32Oc1c(CC=C(C)C)c2c(c(O[C@H]3O[C@H]5COC(C)(C)O[C@@H]5[C@@H](O)[C@@H]3O)c14)C=CC(C)(CCC=C(C)C)O2] | C₅₁H₆₃N₃O₁₂ | 910.06 |
| 63 | 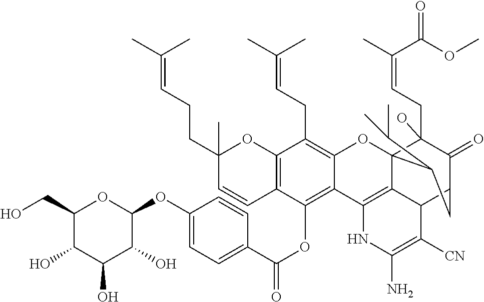 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C(C#N)=C(N)N1)C=CC(C)(CCC=C(C)C)O4] | C₅₅H₆₃N₃O₁₄ | 990.1 |
| 64 | 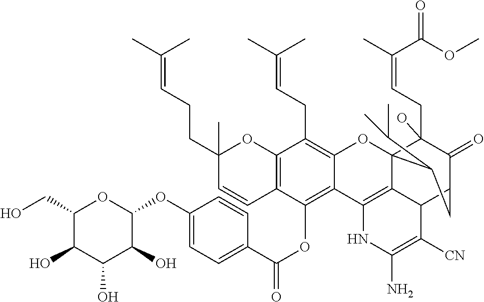 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C(C#N)=C(N)N1)C=CC(C)(CCC=C(C)C)O4] | C₅₅H₆₃N₃O₁₄ | 990.1 |
| 65 | 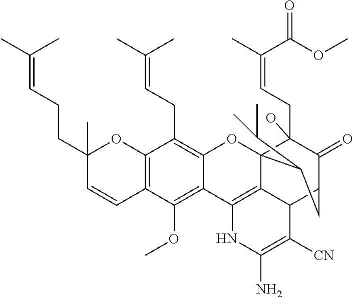 [SMILES: COC(=O)/C(C)=C\CC1(O)C(=O)C2CC(C(C)C)C13Oc1c(CC=C(C)C)c4c(c(OC)c1C1=C3C2C(C#N)=C(N)N1)C=CC(C)(CCC=C(C)C)O4] | C₄₃H₅₁N₃O₇ | 721.88 |
| 66 | 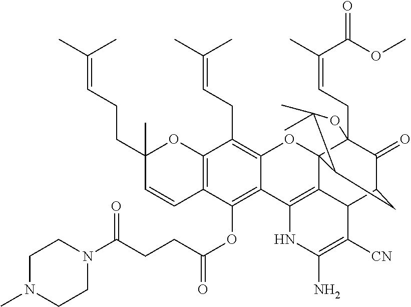 [SMILES: COC(=O)/C(C)=C\CC12OC(C)(C)C3CC(C1=O)C1C(C#N)=C(N)NC4=C1C32Oc1c(CC=C(C)C)c2c(c(OC(=O)CCC(=O)N3CCN(C)CC3)c14)C=CC(C)(CCC=C(C)C)O2] | C₅₁H₆₃N₅O₉ | 890.07 |
| 67 | 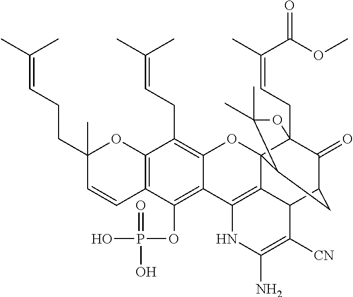 [SMILES: COC(=O)/C(C)=C\CC12OC(C)(C)C3CC(C1=O)C1C(C#N)=C(N)NC4=C1C32Oc1c(CC=C(C)C)c2c(c(OP(=O)(O)O)c14)C=CC(C)(CCC=C(C)C)O2] | C₄₂H₅₀N₃O₁₀P | 787.83 |
| 68 | 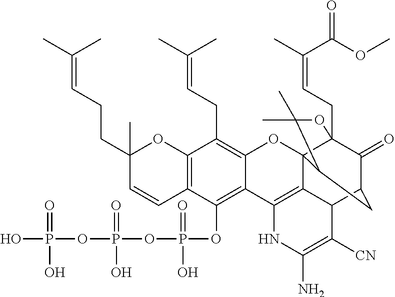 [SMILES: COC(=O)/C(C)=C\CC12OC(C)(C)C3CC(C1=O)C1C(C#N)=C(N)NC4=C1C32Oc1c(CC=C(C)C)c2c(c(OP(=O)(O)OP(=O)(O)OP(=O)(O)O)c14)C=CC(C)(CCC=C(C)C)O2] | C₄₂H₅₂N₃O₁₆P₃ | 947.79 |
| 69 | 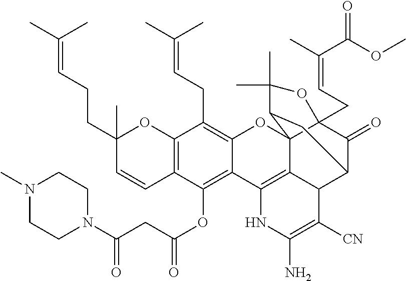 | C₅₀H₆₁N₅O₉ | 876.05 |
| 70 | 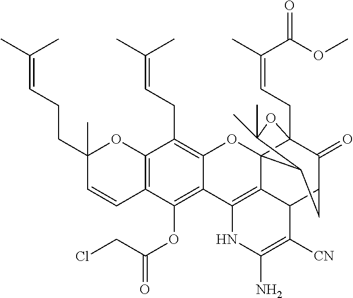 [SMILES: COC(=O)/C(C)=C\CC12OC(C)(C)C3CC(C1=O)C1C(C#N)=C(N)NC4=C1C32Oc1c(CC=C(C)C)c2c(c(OC(=O)CCl)c14)C=CC(C)(CCC=C(C)C)O2] | C₄₄H₅₀ClN₃O₈ | 784.34 |
| 71 | 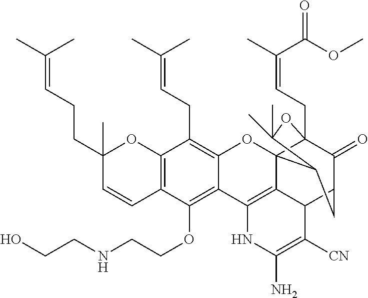 [SMILES: COC(=O)/C(C)=C\CC12OC(C)(C)C3CC(C1=O)C1C(C#N)=C(N)NC4=C1C32Oc1c(CC=C(C)C)c2c(c(OCCNCCO)c14)C=CC(C)(CCC=C(C)C)O2] | C₄₆H₅₈N₄O₈ | 794.97 |
| 72 | 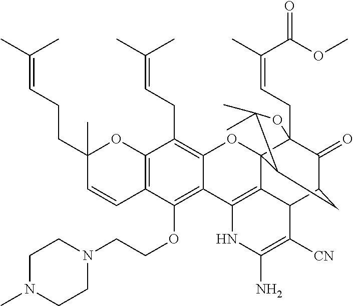 [SMILES: COC(=O)/C(C)=C\CC12OC(C)(C)C3CC(C1=O)C1C(C#N)=C(N)NC4=C1C32Oc1c(CC=C(C)C)c2c(c(OCCN3CCN(C)CC3)c14)C=CC(C)(CCC=C(C)C)O2] | C₄₉H₆₃N₅O₇ | 834.05 |
| 73 | 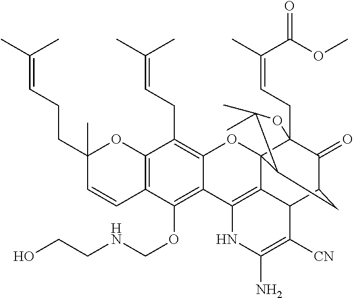 [SMILES: COC(=O)/C(C)=C\CC12OC(C)(C)C3CC(C1=O)C1C(C#N)=C(N)NC4=C1C32Oc1c(CC=C(C)C)c2c(c(OCNCCO)c14)C=CC(C)(CCC=C(C)C)O2] | C₄₅H₅₆N₄O₈ | 780.95 |
| 74 | 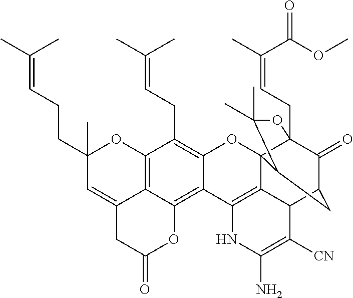 [SMILES: COC(=O)/C(C)=C\CC12OC(C)(C)C3CC(C1=O)C1C(C#N)=C(N)NC4=C1C32Oc1c(CC=C(C)C)c2c3c(c14)OC(=O)CC3=CC(C)(CCC=C(C)C)O2] | C₄₄H₄₉N₃O₈ | 747.88 |
| 75 | 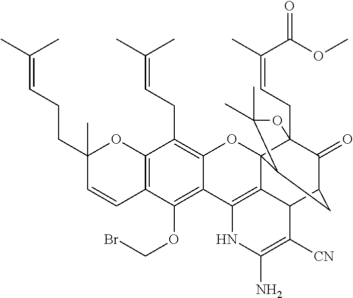 [SMILES: COC(=O)/C(C)=C\CC12OC(C)(C)C3CC(C1=O)C1C(C#N)=C(N)NC4=C1C32Oc1c(CC=C(C)C)c2c(c(OCBr)c14)C=CC(C)(CCC=C(C)C)O2] | C₄₃H₅₀BrN₃O₇ | 800.78 |
| 76 | 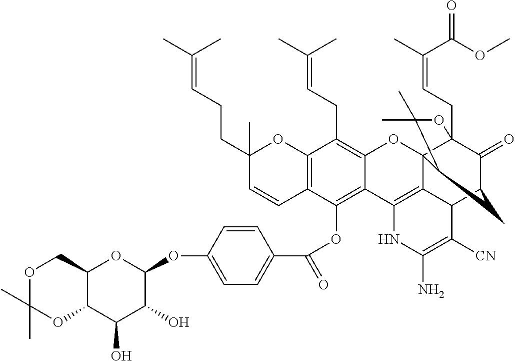 [SMILES: COC(=O)/C(C)=C\CC12OC(C)(C)[C@H]3C[C@H](C1=O)C1C(C#N)=C(N)NC4=C1C32Oc1c(CC=C(C)C)c2c(c(OC(=O)c3ccc(O[C@@H]5O[C@@H]6COC(C)(C)O[C@H]6[C@H](O)[C@H]5O)cc3)c14)C=CC(C)(CCC=C(C)C)O2] | C₅₈H₆₇N₃O₁₄ | 1030.16 |
| 77 | 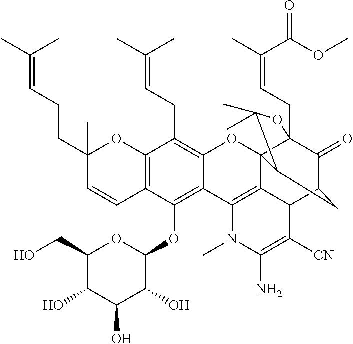 [SMILES: COC(=O)/C(C)=C\CC12OC(C)(C)C3CC(C1=O)C1C(C#N)=C(N)N(C)C4=C1C32Oc1c(CC=C(C)C)c2c(c(O[C@@H]3O[C@H](CO)[C@@H](O)[C@H](O)[C@H]3O)c14)C=CC(C)(CCC=C(C)C)O2] | C₄₉H₆₁N₃O₁₂ | 884.02 |
| 78 | 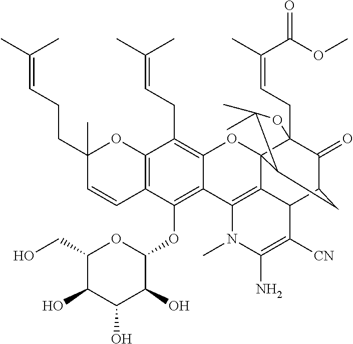 [SMILES: COC(=O)/C(C)=C\CC12OC(C)(C)C3CC(C1=O)C1C(C#N)=C(N)N(C)C4=C1C32Oc1c(CC=C(C)C)c2c(c(O[C@H]3O[C@@H](CO)[C@H](O)[C@@H](O)[C@@H]3O)c14)C=CC(C)(CCC=C(C)C)O2] | C₄₉H₆₁N₃O₁₂ | 884.02 |
| 79 | 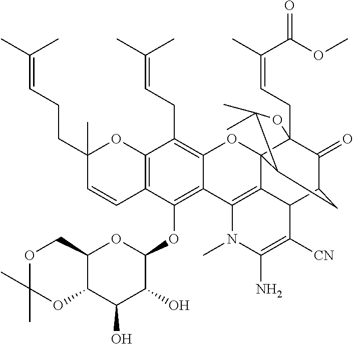 [SMILES: COC(=O)/C(C)=C\CC12OC(C)(C)C3CC(C1=O)C1C(C#N)=C(N)N(C)C4=C1C32Oc1c(CC=C(C)C)c2c(c(O[C@@H]3O[C@@H]5COC(C)(C)O[C@H]5[C@H](O)[C@H]3O)c14)C=CC(C)(CCC=C(C)C)O2] | C₅₂H₆₅N₃O₁₂ | 924.09 |
| 80 | 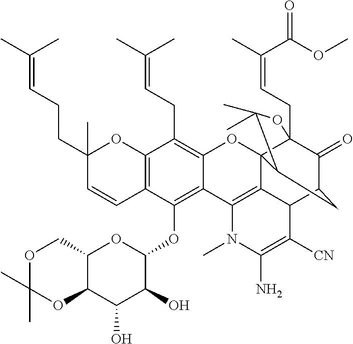 [SMILES: COC(=O)/C(C)=C\CC12OC(C)(C)C3CC(C1=O)C1C(C#N)=C(N)N(C)C4=C1C32Oc1c(CC=C(C)C)c2c(c(O[C@H]3O[C@H]5COC(C)(C)O[C@@H]5[C@@H](O)[C@@H]3O)c14)C=CC(C)(CCC=C(C)C)O2] | C₅₂H₆₅N₃O₁₂ | 924.09 |
| 81 | 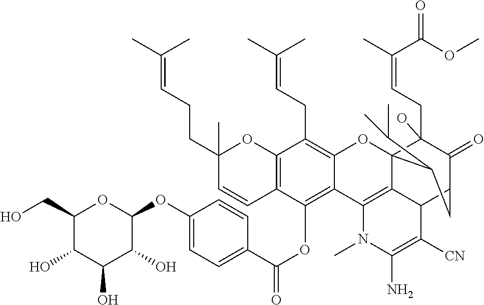 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C(C#N)=C(N)N1C)C=CC(C)(CCC=C(C)C)O4] | C₅₆H₆₅N₃O₁₄ | 1004.13 |
| 82 | 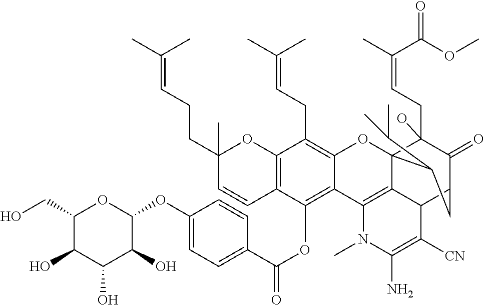 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C(C#N)=C(N)N1C)C=CC(C)(CCC=C(C)C)O4] | C₅₃H₆₅N₃O₁₄ | 1004.13 |
| 83 | 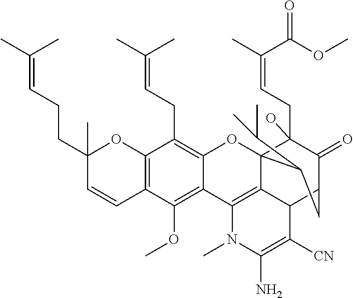 [SMILES: COC(=O)/C(C)=C\CC1(O)C(=O)C2CC(C(C)C)C13Oc1c(CC=C(C)C)c4c(c(OC)c1C1=C3C2C(C#N)=C(N)N1C)C=CC(C)(CCC=C(C)C)O4] | C₄₄H₅₃N₃O₇ | 735.91 |
| 84 | 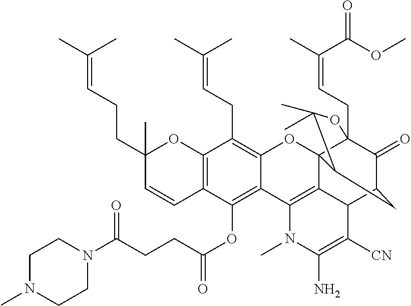 [SMILES: COC(=O)/C(C)=C\CC12OC(C)(C)C3CC(C1=O)C1C(C#N)=C(N)N(C)C4=C1C32Oc1c(CC=C(C)C)c2c(c(OC(=O)CCC(=O)N3CCN(C)CC3)c14)C=CC(C)(CCC=C(C)C)O2] | C₅₂H₆₅N₅O₉ | 904.1 |
| 85 | 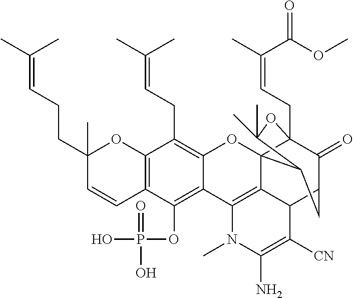 [SMILES: COC(=O)/C(C)=C\CC12OC(C)(C)C3CC(C1=O)C1C(C#N)=C(N)N(C)C4=C1C32Oc1c(CC=C(C)C)c2c(c(OP(=O)(O)O)c14)C=CC(C)(CCC=C(C)C)O2] | C₄₃H₅₂N₃O₁₀P | 801.86 |
| 86 | 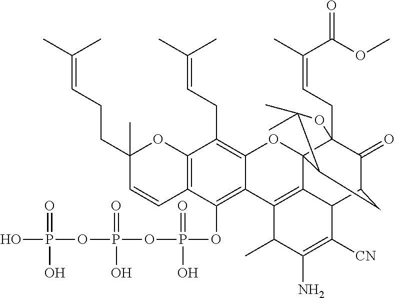 [SMILES: COC(=O)/C(C)=C\CC12OC(C)(C)C3CC(C1=O)C1C(C#N)=C(N)C(C)C4=C1C32Oc1c(CC=C(C)C)c2c(c(OP(=O)(O)OP(=O)(O)OP(=O)(O)O)c14)C=CC(C)(CCC=C(C)C)O2] | C₄₄H₅₅N₂O₁₆P₃ | 960.83 |
| 87 | 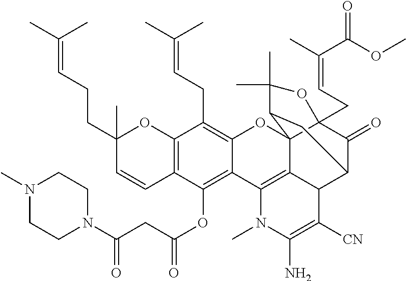 | C₅₁H₆₃N₅O₉ | 890.07 |
| 88 | 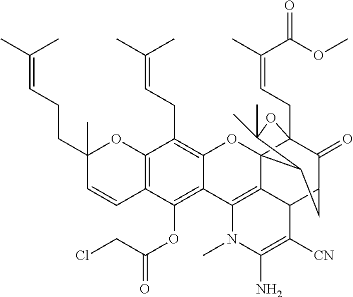 [SMILES: COC(=O)/C(C)=C\CC12OC(C)(C)C3CC(C1=O)C1C(C#N)=C(N)N(C)C4=C1C32Oc1c(CC=C(C)C)c2c(c(OC(=O)CCl)c14)C=CC(C)(CCC=C(C)C)O2] | C₄₅H₅₂ClN₃O₈ | 798.36 |
| 89 | 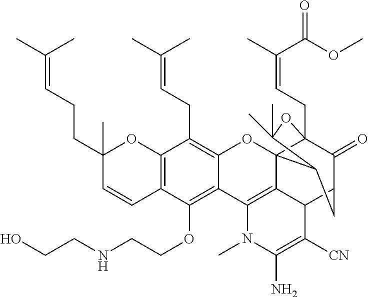 [SMILES: COC(=O)/C(C)=C\CC12OC(C)(C)C3CC(C1=O)C1C(C#N)=C(N)N(C)C4=C1C32Oc1c(CC=C(C)C)c2c(c(OCCNCCO)c14)C=CC(C)(CCC=C(C)C)O2] | C₄₇H₆₀N₄O₈ | 809 |
| 90 | 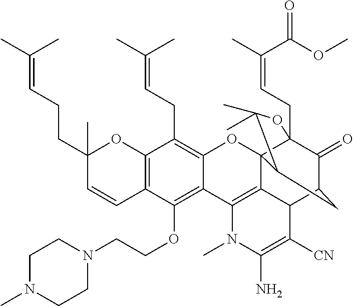 [SMILES: COC(=O)/C(C)=C\CC12OC(C)(C)C3CC(C1=O)C1C(C#N)=C(N)N(C)C4=C1C32Oc1c(CC=C(C)C)c2c(c(OCCN3CCN(C)CC3)c14)C=CC(C)(CCC=C(C)C)O2] | C₅₀H₆₅N₅O₇ | 848.08 |
| 91 | 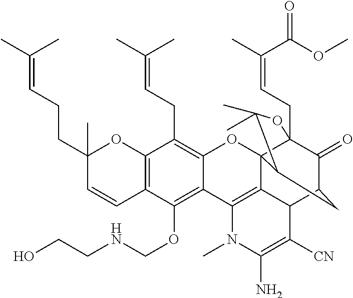 | C₄₆H₅₈N₄O₈ | 794.97 |
| 92 | 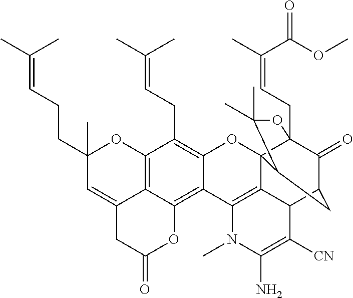 [SMILES: COC(=O)/C(C)=C\CC12OC(C)(C)C3CC(C1=O)C1C(C#N)=C(N)N(C)C4=C1C32Oc1c(CC=C(C)C)c2c3c(c14)OC(=O)CC3=CC(C)(CCC=C(C)C)O2] | C₄₅H₅₁N₃O₈ | 761.9 |
| 93 | 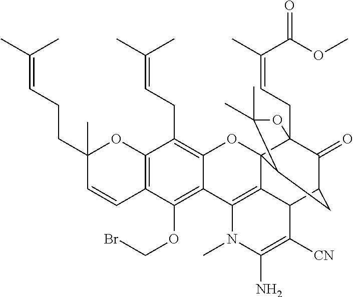 [SMILES: COC(=O)/C(C)=C\CC12OC(C)(C)C3CC(C1=O)C1C(C#N)=C(N)N(C)C4=C1C32Oc1c(CC=C(C)C)c2c(c(OCBr)c14)C=CC(C)(CCC=C(C)C)O2] | C₄₄H₅₂BrN₃O₇ | 814.8 |
| 94 | 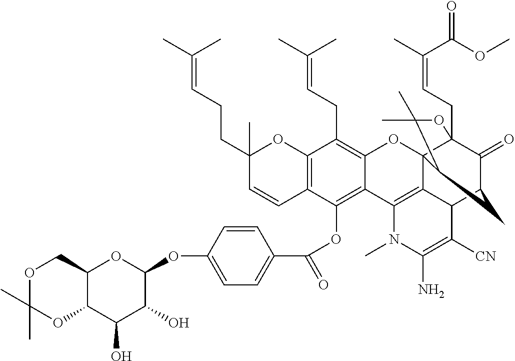 [SMILES: COC(=O)/C(C)=C\CC12OC(C)(C)[C@H]3C[C@H](C1=O)C1C(C#N)=C(N)N(C)C4=C1C32Oc1c(CC=C(C)C)c2c(c(OC(=O)c3ccc(O[C@@H]5O[C@@H]6COC(C)(C)O[C@H]6[C@H](O)[C@H]5O)cc3)c14)C=CC(C)(CCC=C(C)C)O2] | C₅₉H₆₉N₃O₁₄ | 1044.19 |
| 95 | 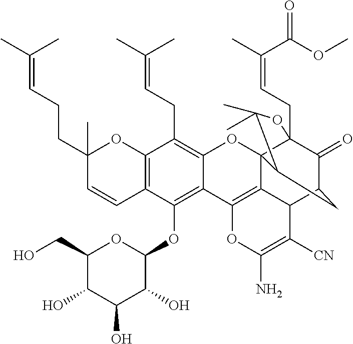 [SMILES: COC(=O)/C(C)=C\CC12OC(C)(C)C3CC(C1=O)C1C(C#N)=C(N)OC4=C1C32Oc1c(CC=C(C)C)c2c(c(O[C@@H]3O[C@H](CO)[C@@H](O)[C@H](O)[C@H]3O)c14)C=CC(C)(CCC=C(C)C)O2] | C₄₈H₅₈N₂O₁₃ | 870.98 |
| 96 | 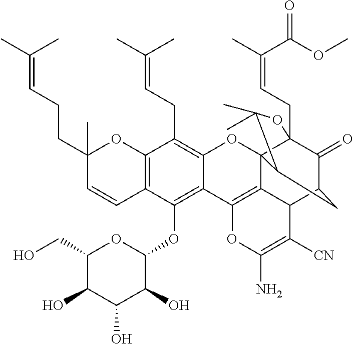 [SMILES: COC(=O)/C(C)=C\CC12OC(C)(C)C3CC(C1=O)C1C(C#N)=C(N)OC4=C1C32Oc1c(CC=C(C)C)c2c(c(O[C@H]3O[C@@H](CO)[C@H](O)[C@@H](O)[C@@H]3O)c14)C=CC(C)(CCC=C(C)C)O2] | C₄₈H₅₈N₂O₁₃ | 870.98 |
| 97 | 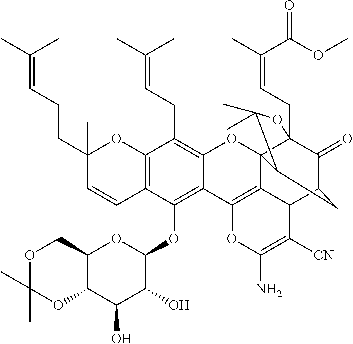 [SMILES: COC(=O)/C(C)=C\CC12OC(C)(C)C3CC(C1=O)C1C(C#N)=C(N)OC4=C1C32Oc1c(CC=C(C)C)c2c(c(O[C@@H]3O[C@@H]5COC(C)(C)O[C@H]5[C@H](O)[C@H]3O)c14)C=CC(C)(CCC=C(C)C)O2] | C₅₁H₆₂N₂O₁₃ | 911.04 |
| 98 | 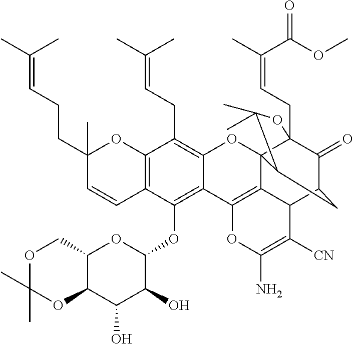 [SMILES: COC(=O)/C(C)=C\CC12OC(C)(C)C3CC(C1=O)C1C(C#N)=C(N)OC4=C1C32Oc1c(CC=C(C)C)c2c(c(O[C@H]3O[C@H]5COC(C)(C)O[C@@H]5[C@@H](O)[C@@H]3O)c14)C=CC(C)(CCC=C(C)C)O2] | C₅₁H₆₂N₂O₁₃ | 911.04 |
| 99 | 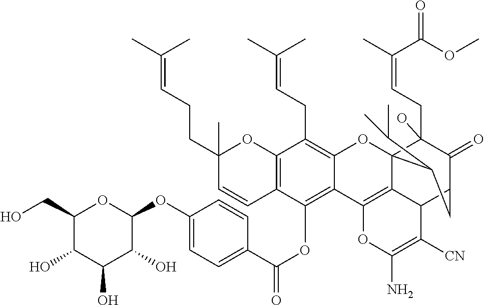 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C(C#N)=C(N)O1)C=CC(C)(CCC=C(C)C)O4] | C₅₅H₆₂N₂O₁₅ | 991.09 |
| 100 | 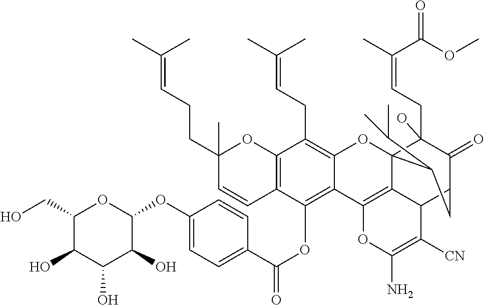 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C(C#N)=C(N)O1)C=CC(C)(CCC=C(C)C)O4] | C₅₅H₆₂N₂O₁₅ | 991.09 |
| 101 | 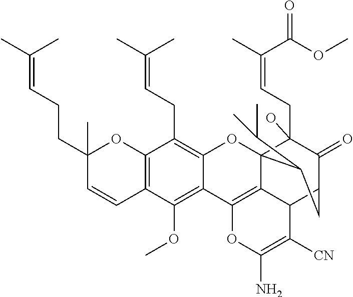 [SMILES: COC(=O)/C(C)=C\CC1(O)C(=O)C2CC(C(C)C)C13Oc1c(CC=C(C)C)c4c(c(OC)c1C1=C3C2C(C#N)=C(N)O1)C=CC(C)(CCC=C(C)C)O4] | C₄₃H₅₀N₂O₈ | 722.87 |
| 102 | 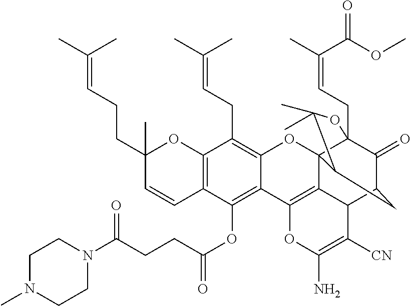 [SMILES: COC(=O)/C(C)=C\CC12OC(C)(C)C3CC(C1=O)C1C(C#N)=C(N)OC4=C1C32Oc1c(CC=C(C)C)c2c(c(OC(=O)CCC(=O)N3CCN(C)CC3)c14)C=CC(C)(CCC=C(C)C)O2] | C₅₁H₆₂N₄O₁₀ | 891.06 |
| 103 | 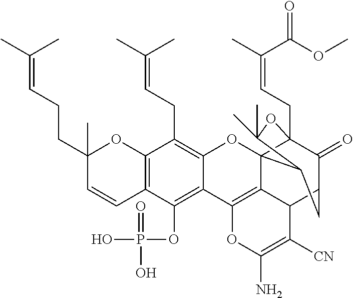 [SMILES: COC(=O)/C(C)=C\CC12OC(C)(C)C3CC(C1=O)C1C(C#N)=C(N)OC4=C1C32Oc1c(CC=C(C)C)c2c(c(OP(=O)(O)O)c14)C=CC(C)(CCC=C(C)C)O2] | C₄₂H₄₉N₂O₁₁P | 788.82 |
| 104 | 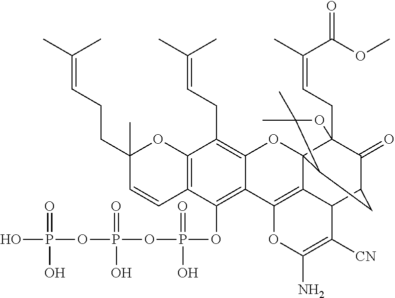 [SMILES: COC(=O)/C(C)=C\CC12OC(C)(C)C3CC(C1=O)C1C(C#N)=C(N)OC4=C1C32Oc1c(CC=C(C)C)c2c(c(OP(=O)(O)OP(=O)(O)OP(=O)(O)O)c14)C=CC(C)(CCC=C(C)C)O2] | C₄₂H₅₁N₂O₁₇P₃ | 948.78 |
| 105 | 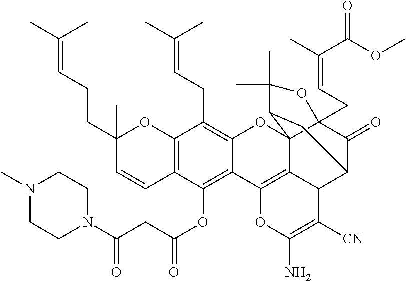 | C₅₀H₆₀N₄O₁₀ | 877.03 |
| 106 | 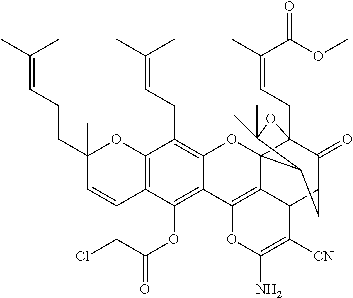 [SMILES: COC(=O)/C(C)=C\CC12OC(C)(C)C3CC(C1=O)C1C(C#N)=C(N)OC4=C1C32Oc1c(CC=C(C)C)c2c(c(OC(=O)CCl)c14)C=CC(C)(CCC=C(C)C)O2] | C₄₄H₄₉ClN₂O₉ | 785.32 |
| 107 | 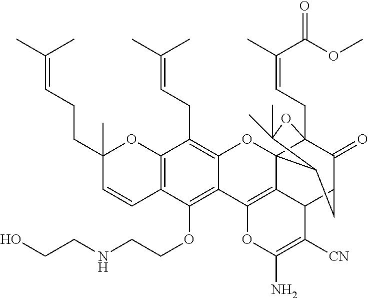 [SMILES: COC(=O)/C(C)=C\CC12OC(C)(C)C3CC(C1=O)C1C(C#N)=C(N)OC4=C1C32Oc1c(CC=C(C)C)c2c(c(OCCNCCO)c14)C=CC(C)(CCC=C(C)C)O2] | C₄₆H₅₇N₃O₉ | 795.96 |
| 108 | 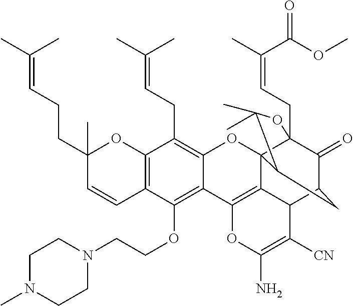 [SMILES: COC(=O)/C(C)=C\CC12OC(C)(C)C3CC(C1=O)C1C(C#N)=C(N)OC4=C1C32Oc1c(CC=C(C)C)c2c(c(OCCN3CCN(C)CC3)c14)C=CC(C)(CCC=C(C)C)O2] | C₄₉H₆₂N₄O₈ | 835.04 |
| 109 | 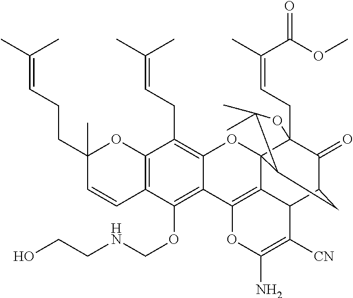 [SMILES: COC(=O)/C(C)=C\CC12OC(C)(C)C3CC(C1=O)C1C(C#N)=C(N)OC4=C1C32Oc1c(CC=C(C)C)c2c(c(OCNCCO)c14)C=CC(C)(CCC=C(C)C)O2] | C₄₅H₅₅N₃O₉ | 781.93 |
| 110 | 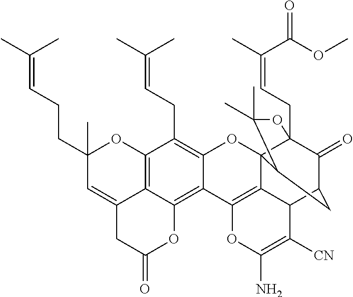 [SMILES: COC(=O)/C(C)=C\CC12OC(C)(C)C3CC(C1=O)C1C(C#N)=C(N)OC4=C1C32Oc1c(CC=C(C)C)c2c3c(c14)OC(=O)CC3=CC(C)(CCC=C(C)C)O2] | C₄₄H₄₈N₂O₉ | 748.86 |
| 111 | 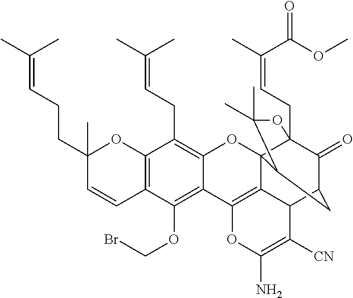 [SMILES: COC(=O)/C(C)=C\CC12OC(C)(C)C3CC(C1=O)C1C(C#N)=C(N)OC4=C1C32Oc1c(CC=C(C)C)c2c(c(OCBr)c14)C=CC(C)(CCC=C(C)C)O2] | C₄₃H₄₉BrN₂O8 | 801.76 |
| 112 | 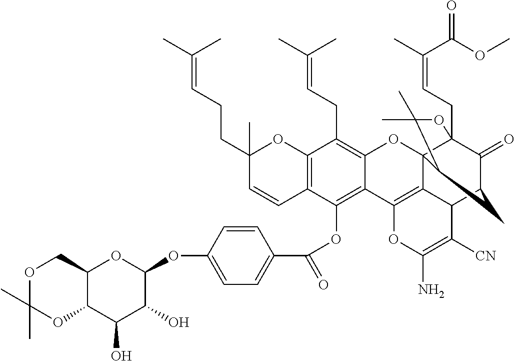 [SMILES: COC(=O)/C(C)=C\CC12OC(C)(C)[C@H]3C[C@H](C1=O)C1C(C#N)=C(N)OC4=C1C32Oc1c(CC=C(C)C)c2c(c(OC(=O)c3ccc(O[C@@H]5O[C@@H]6COC(C)(C)O[C@H]6[C@H](O)[C@H]5O)cc3)c14)C=CC(C)(CCC=C(C)C)O2] | C₅₈H₆₆N₂O₁₅ | 1031.15 |
| 113 | 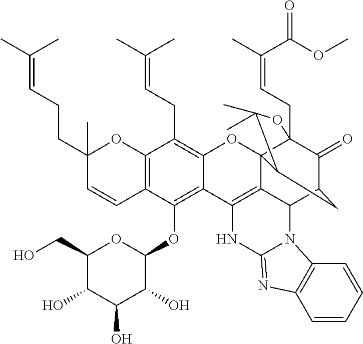 [SMILES: COC(=O)/C(C)=C\CC12OC(C)(C)C3CC(C1=O)C1C4=C(Nc5nc6ccccc6n51)c1c(O[C@@H]5O[C@H](CO)[C@@H](O)[C@H](O)[C@H]5O)c5c(c(CC=C(C)C)c1OC432)OC(C)(CCC=C(C)C)C=C5] | C₅₂H₆₁N₃O₁₂ | 920.05 |
| 114 | 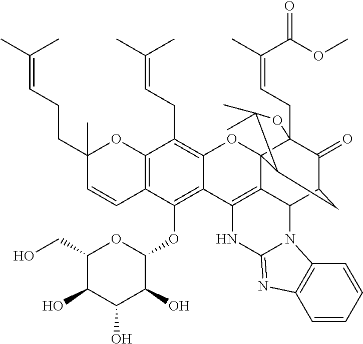 [SMILES: COC(=O)/C(C)=C\CC12OC(C)(C)C3CC(C1=O)C1C4=C(Nc5nc6ccccc6n51)c1c(O[C@H]5O[C@@H](CO)[C@H](O)[C@@H](O)[C@@H]5O)c5c(c(CC=C(C)C)c1OC432)OC(C)(CCC=C(C)C)C=C5] | C₅₂H₆₁N₃O₁₂ | 920.05 |
| 115 | 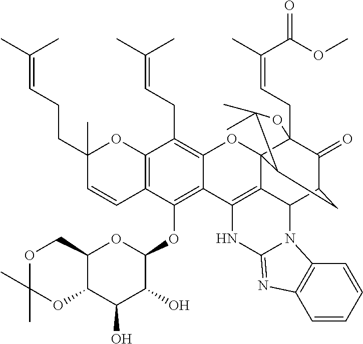 [SMILES: COC(=O)/C(C)=C\CC12OC(C)(C)C3CC(C1=O)C1C4=C(Nc5nc6ccccc6n51)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₅₅H₆₅N₃O₁₂ | 960.12 |
| 116 | 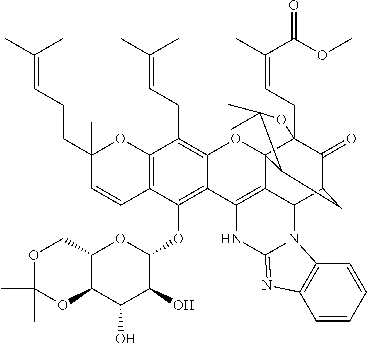 [SMILES: COC(=O)/C(C)=C\CC12OC(C)(C)C3CC(C1=O)C1C4=C(Nc5nc6ccccc6n51)c1c(O[C@H]5O[C@H]6COC(C)(C)O[C@@H]6[C@@H](O)[C@@H]5O)c5c(c(CC=C(C)C)c1OC432)OC(C)(CCC=C(C)C)C=C5] | C₅₅H₆₅N₃O₁₂ | 960.12 |
| 117 | 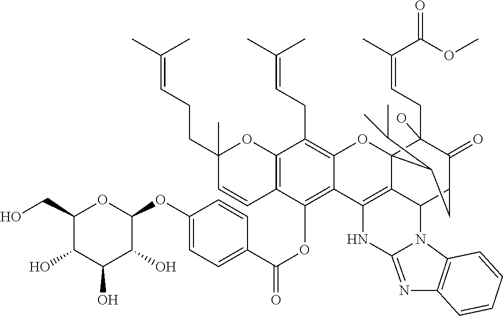 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2n2c(nc3ccccc32)N1)C=CC(C)(CCC=C(C)C)O4] | C₅₉H₆₅N₃O₁₄ | 1040.16 |
| 118 | 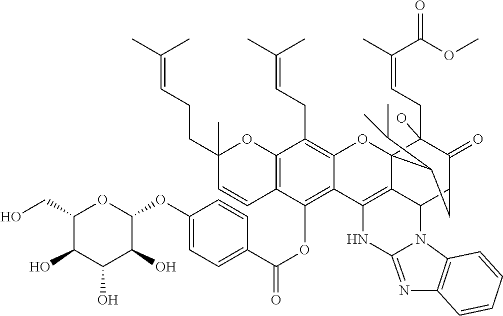 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2n2c(nc3ccccc32)N1)C=CC(C)(CCC=C(C)C)O4] | C₅₉H₆₅N₃O₁₄ | 1040.16 |
| 119 | 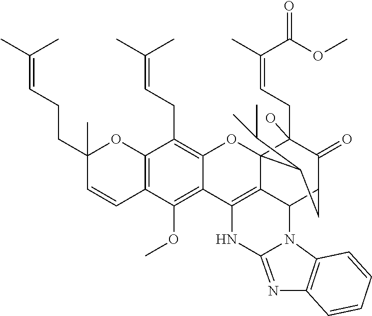 [SMILES: COC(=O)/C(C)=C\CC1(O)C(=O)C2CC(C(C)C)C13Oc1c(CC=C(C)C)c4c(c(OC)c1C1=C3C2n2c(nc3ccccc32)N1)C=CC(C)(CCC=C(C)C)O4] | C₄₇H₅₃N₃O₇ | 771.94 |
| 120 | 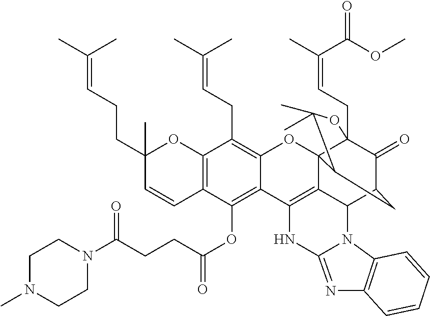 [SMILES: COC(=O)/C(C)=C\CC12OC(C)(C)C3CC(C1=O)C1C4=C(Nc5nc6ccccc6n51)c1c(OC(=O)CCC(=O)N5CCN(C)CC5)c5c(c(CC=C(C)C)c1OC432)OC(C)(CCC=C(C)C)C=C5] | C₅₅H₆₅N₅O₉ | 940.13 |
| 121 | 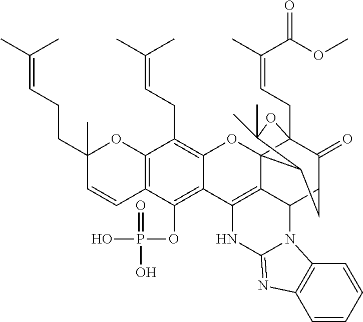 [SMILES: COC(=O)/C(C)=C\CC12OC(C)(C)C3CC(C1=O)C1C4=C(Nc5nc6ccccc6n51)c1c(OP(=O)(O)O)c5c(c(CC=C(C)C)c1OC432)OC(C)(CCC=C(C)C)C=C5] | C₄₆H₅₂N₃O₁₀P | 837.89 |
| 122 | 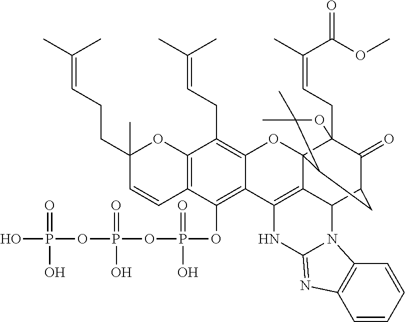 [SMILES: COC(=O)/C(C)=C\CC12OC(C)(C)C3CC(C1=O)C1C4=C(Nc5nc6ccccc6n51)c1c(OP(=O)(O)OP(=O)(O)OP(=O)(O)O)c5c(c(CC=C(C)C)c1OC432)OC(C)(CCC=C(C)C)C=C5] | C₄₆H₅₄N₃O₁₆P₃ | 997.85 |
| 123 | 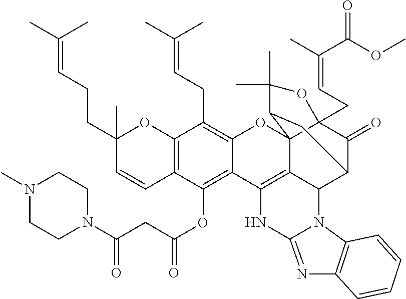 | C₅₄H₆₃N₅O₉ | 926.11 |
| 124 | 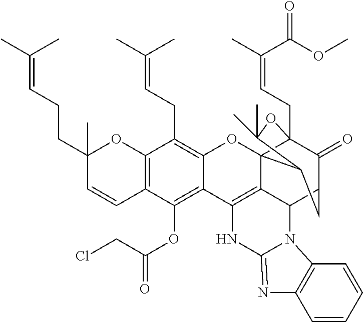 [SMILES: COC(=O)/C(C)=C\CC12OC(C)(C)C3CC(C1=O)C1C4=C(Nc5nc6ccccc6n51)c1c(OC(=O)CCl)c5c(c(CC=C(C)C)c1OC432)OC(C)(CCC=C(C)C)C=C5] | C₄₈H₅₂ClN₃O₈ | 834.39 |
| 125 | 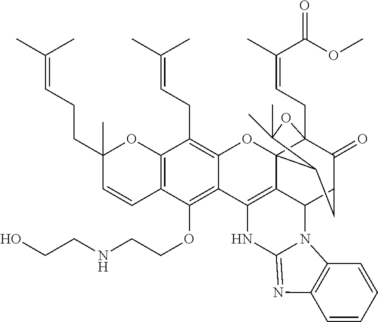 [SMILES: COC(=O)/C(C)=C\CC12OC(C)(C)C3CC(C1=O)C1C4=C(Nc5nc6ccccc6n51)c1c(OCCNCCO)c5c(c(CC=C(C)C)c1OC432)OC(C)(CCC=C(C)C)C=C5] | C₅₀H₆₀N₄O₈ | 845.03 |
| 126 | 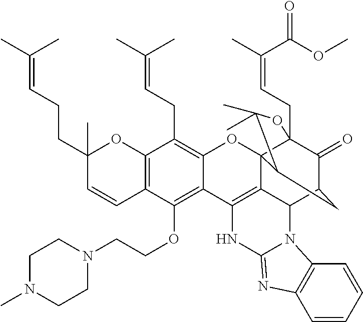 [SMILES: COC(=O)/C(C)=C\CC12OC(C)(C)C3CC(C1=O)C1C4=C(Nc5nc6ccccc6n51)c1c(OCCN5CCN(C)CC5)c5c(c(CC=C(C)C)c1OC432)OC(C)(CCC=C(C)C)C=C5] | C₅₃H₆₅N₅O₇ | 884.11 |
| 127 | 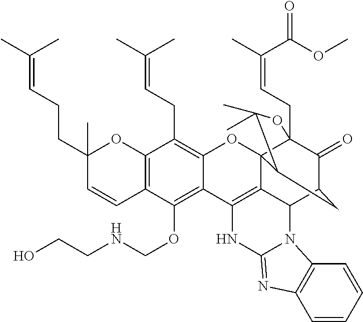 [SMILES: COC(=O)/C(C)=C\CC12OC(C)(C)C3CC(C1=O)C1C4=C(Nc5nc6ccccc6n51)c1c(OCNCCO)c5c(c(CC=C(C)C)c1OC432)OC(C)(CCC=C(C)C)C=C5] | C₄₉H₅₈N₄O₈ | 831.01 |
| 128 | 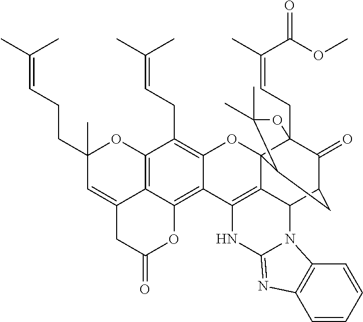 [SMILES: COC(=O)/C(C)=C\CC12OC(C)(C)C3CC(C1=O)C1C4=C(Nc5nc6ccccc6n51)c1c(c(CC=C(C)C)c5c6c1OC(=O)CC6=CC(C)(CCC=C(C)C)O5)OC432] | C₄₈H₅₁N₃O₈ | 797.93 |
| 129 | 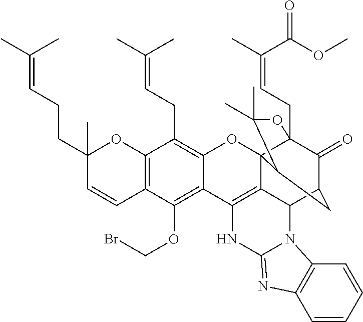 [SMILES: COC(=O)/C(C)=C\CC12OC(C)(C)C3CC(C1=O)C1C4=C(Nc5nc6ccccc6n51)c1c(OCBr)c5c(c(CC=C(C)C)c1OC432)OC(C)(CCC=C(C)C)C=C5] | C₄₇H₅₂BrN₃O₇ | 850.84 |
| 130 | 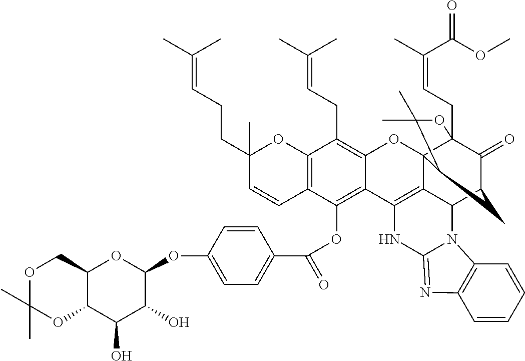 [SMILES: COC(=O)/C(C)=C\CC12OC(C)(C)[C@H]3C[C@H](C1=O)C1C4=C(Nc5nc6ccccc6n51)c1c(OC(=O)c5ccc(O[C@@H]6O[C@@H]7COC(C)(C)O[C@H]7[C@H](O)[C@H]6O)cc5)c5c(c(CC=C(C)C)c1OC432)OC(C)(CCC=C(C)C)C=C5] | C₆₂H₆₉N₃O₁₄ | 1080.22 |
| 131 | 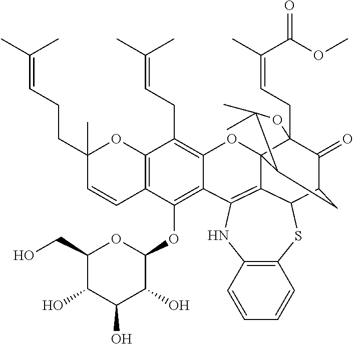 [SMILES: COC(=O)/C(C)=C\CC12OC(C)(C)C3CC(C1=O)C1Sc4ccccc4NC4=C1C32Oc1c(CC=C(C)C)c2c(c(O[C@@H]3O[C@H](CO)[C@@H](O)[C@H](O)[C@H]3O)c14)C=CC(C)(CCC=C(C)C)O2] | C₅₁H₆₁NO₁₂S | 912.09 |
| 132 | 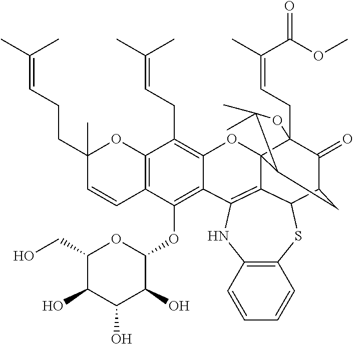 [SMILES: COC(=O)/C(C)=C\CC12OC(C)(C)C3CC(C1=O)C1Sc4ccccc4NC4=C1C32Oc1c(CC=C(C)C)c2c(c(O[C@H]3O[C@@H](CO)[C@H](O)[C@@H](O)[C@@H]3O)c14)C=CC(C)(CCC=C(C)C)O2] | C₅₁H₆₁NO₁₂S | 912.09 |
| 133 | 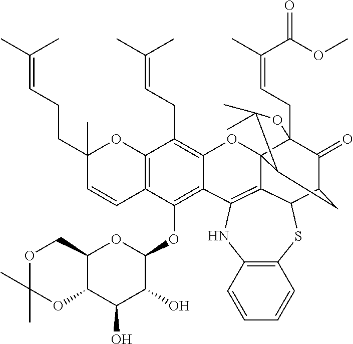 [SMILES: COC(=O)/C(C)=C\CC12OC(C)(C)C3CC(C1=O)C1Sc4ccccc4NC4=C1C32Oc1c(CC=C(C)C)c2c(c(O[C@@H]3O[C@@H]5COC(C)(C)O[C@H]5[C@H](O)[C@H]3O)c14)C=CC(C)(CCC=C(C)C)O2] | C₅₄H₆₅NO₁₂S | 952.16 |
| 134 | 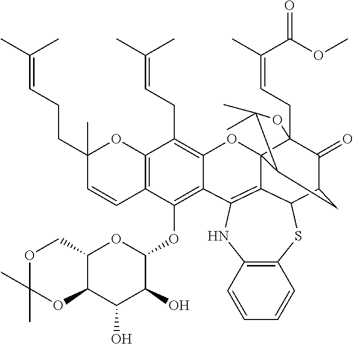 [SMILES: COC(=O)/C(C)=C\CC12OC(C)(C)C3CC(C1=O)C1Sc4ccccc4NC4=C1C32Oc1c(CC=C(C)C)c2c(c(O[C@H]3O[C@H]5COC(C)(C)O[C@@H]5[C@@H](O)[C@@H]3O)c14)C=CC(C)(CCC=C(C)C)O2] | C₅₄H₆₅NO₁₂S | 952.16 |
| 135 | 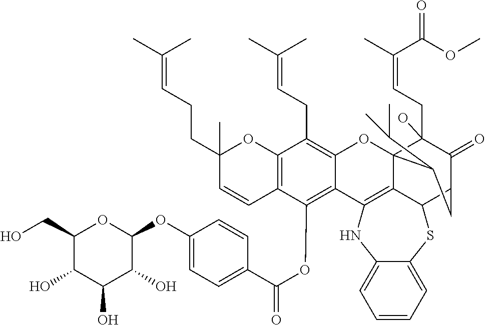 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2Sc2ccccc2N1)C=CC(C)(CCC=C(C)C)O4] | C₅₈H₆₅NO₁₄S | 1032.2 |
| 136 | 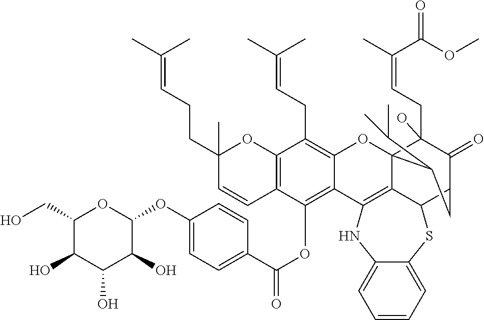 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2Sc2ccccc2N1)C=CC(C)(CCC=C(C)C)O4] | C₅₈H₆₅NO₁₄S | 1032.2 |
| 137 | 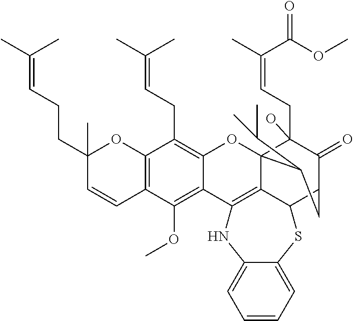 [SMILES: COC(=O)/C(C)=C\CC1(O)C(=O)C2CC(C(C)C)C13Oc1c(CC=C(C)C)c4c(c(OC)c1C1=C3C2Sc2ccccc2N1)C=CC(C)(CCC=C(C)C)O4] | C₄₆H₅₃NO₇S | 763.98 |
| 138 | 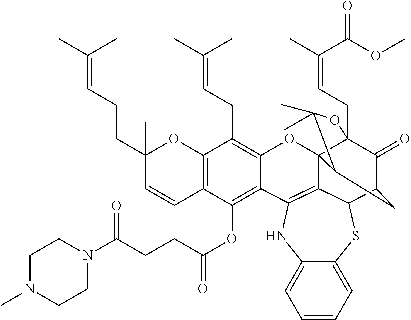 [SMILES: COC(=O)/C(C)=C\CC12OC(C)(C)C3CC(C1=O)C1Sc4ccccc4NC4=C1C32Oc1c(CC=C(C)C)c2c(c(OC(=O)CCC(=O)N3CCN(C)CC3)c14)C=CC(C)(CCC=C(C)C)O2] | C₅₄H₆₅N₃O₉S | 932.17 |
| 139 | 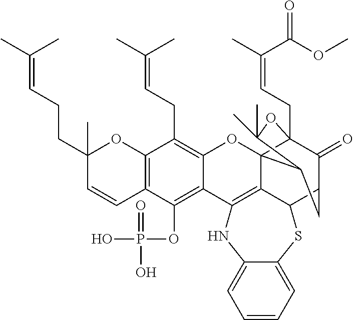 [SMILES: COC(=O)/C(C)=C\CC12OC(C)(C)C3CC(C1=O)C1Sc4ccccc4NC4=C1C32Oc1c(CC=C(C)C)c2c(c(OP(=O)(O)O)c14)C=CC(C)(CCC=C(C)C)O2] | C₄₅H₅₂NO₁₀PS | 829.93 |
| 140 | 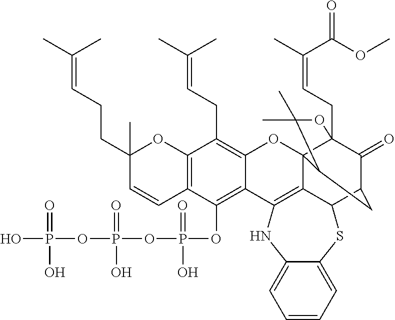 [SMILES: COC(=O)/C(C)=C\CC12OC(C)(C)C3CC(C1=O)C1Sc4ccccc4NC4=C1C32Oc1c(CC=C(C)C)c2c(c(OP(=O)(O)OP(=O)(O)OP(=O)(O)O)c14)C=CC(C)(CCC=C(C)C)O2] | C₄₅H₅₄NO₁₆P₃S | 989.89 |
| 141 | 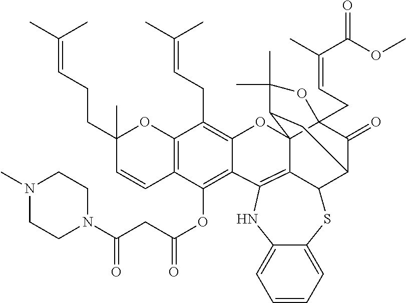 | C₅₃H₆₃N₃O₉S | 918.15 |
| 142 | 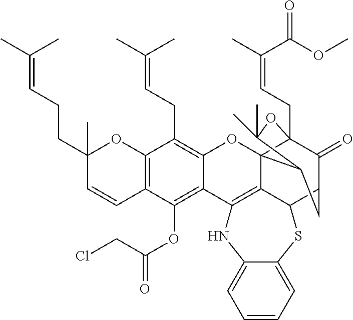 [SMILES: COC(=O)/C(C)=C\CC12OC(C)(C)C3CC(C1=O)C1Sc4ccccc4NC4=C1C32Oc1c(CC=C(C)C)c2c(c(OC(=O)CCl)c14)C=CC(C)(CCC=C(C)C)O2] | C₄₇H₅₂ClNO₈S | 826.44 |
| 143 | 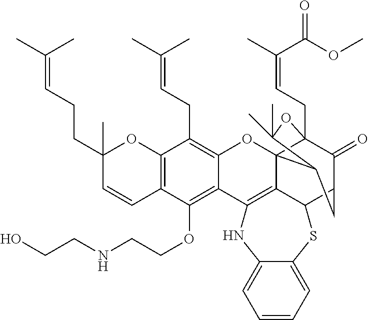 [SMILES: COC(=O)/C(C)=C\CC12OC(C)(C)C3CC(C1=O)C1Sc4ccccc4NC4=C1C32Oc1c(CC=C(C)C)c2c(c(OCCNCCO)c14)C=CC(C)(CCC=C(C)C)O2] | C₄₉H₆₀N₂O₈S | 837.07 |
| 144 | 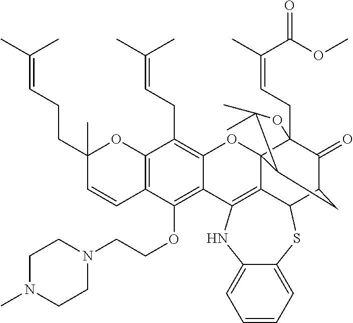 [SMILES: COC(=O)/C(C)=C\CC12OC(C)(C)C3CC(C1=O)C1Sc4ccccc4NC4=C1C32Oc1c(CC=C(C)C)c2c(c(OCCN3CCN(C)CC3)c14)C=CC(C)(CCC=C(C)C)O2] | C₅₂H₆₅N₃O₇S | 876.15 |
| 145 | 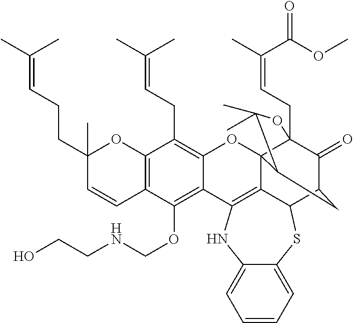 [SMILES: COC(=O)/C(C)=C\CC12OC(C)(C)C3CC(C1=O)C1Sc4ccccc4NC4=C1C32Oc1c(CC=C(C)C)c2c(c(OCNCCO)c14)C=CC(C)(CCC=C(C)C)O2] | C₄₈H₅₈N₂O₈S | 823.05 |
| 146 | 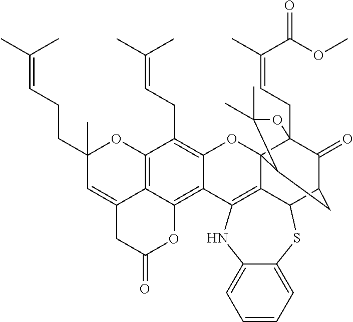 [SMILES: COC(=O)/C(C)=C\CC12OC(C)(C)C3CC(C1=O)C1Sc4ccccc4NC4=C1C32Oc1c(CC=C(C)C)c2c3c(c14)OC(=O)CC3=CC(C)(CCC=C(C)C)O2] | C₄₇H₅₁NO₈S | 789.97 |
| 147 | 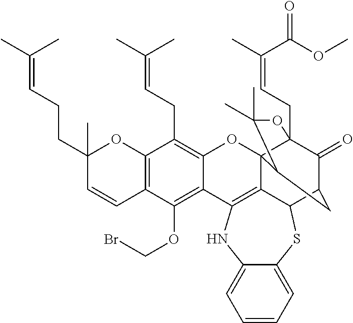 [SMILES: COC(=O)/C(C)=C\CC12OC(C)(C)C3CC(C1=O)C1Sc4ccccc4NC4=C1C32Oc1c(CC=C(C)C)c2c(c(OCBr)c14)C=CC(C)(CCC=C(C)C)O2] | C₄₆H₅₂BrNO₇S | 842.88 |
| 148 | 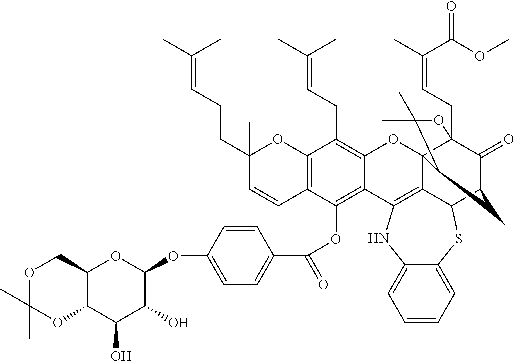 [SMILES: COC(=O)/C(C)=C\CC12OC(C)(C)[C@H]3C[C@H](C1=O)C1Sc4ccccc4NC4=C1C32Oc1c(CC=C(C)C)c2c(c(OC(=O)c3ccc(O[C@@H]5O[C@@H]6COC(C)(C)O[C@H]6[C@H](O)[C@H]5O)cc3)c14)C=CC(C)(CCC=C(C)C)O2] | C₆₁H₆₉NO₁₄S | 1072.26 |
| 149 | 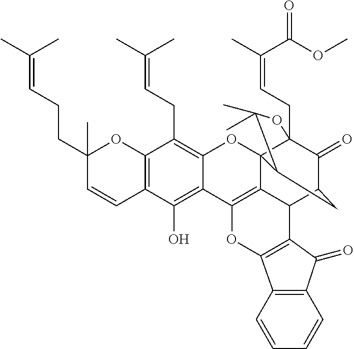 [SMILES: COC(=O)/C(C)=C\CC12OC(C)(C)C3CC(C1=O)C1C4=C(OC5=C1C32Oc1c(CC=C(C)C)c2c(c(O)c15)C=CC(C)(CCC=C(C)C)O2)c1ccccc1C4=O] | C₄₈H₅₀O₉ | 770.91 |
| 150 | 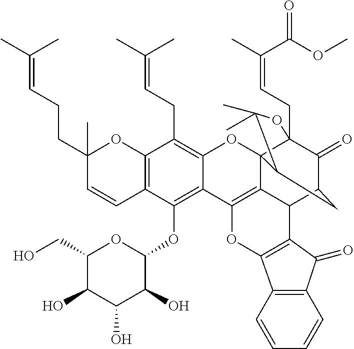 [SMILES: COC(=O)/C(C)=C\CC12OC(C)(C)C3CC(C1=O)C1C4=C(OC5=C1C32Oc1c(CC=C(C)C)c2c(c(O[C@H]3O[C@@H](CO)[C@H](O)[C@@H](O)[C@@H]3O)c15)C=CC(C)(CCC=C(C)C)O2)c1ccccc1C4=O] | C₅₄H₆₀O₁₄ | 933.05 |
| 151 | 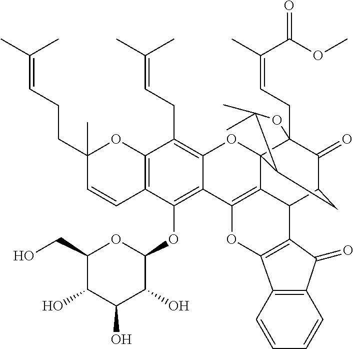 [SMILES: COC(=O)/C(C)=C\CC12OC(C)(C)C3CC(C1=O)C1C4=C(OC5=C1C32Oc1c(CC=C(C)C)c2c(c(O[C@@H]3O[C@H](CO)[C@@H](O)[C@H](O)[C@H]3O)c15)C=CC(C)(CCC=C(C)C)O2)c1ccccc1C4=O] | C₅₄H₆₀O₁₄ | 933.05 |
| 152 | 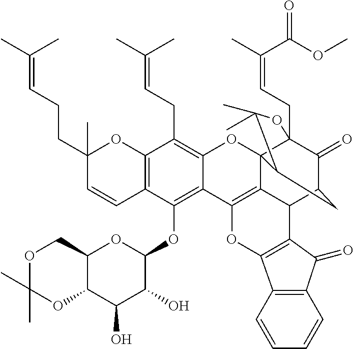 [SMILES: COC(=O)/C(C)=C\CC12OC(C)(C)C3CC(C1=O)C1C4=C(OC5=C1C32Oc1c(CC=C(C)C)c2c(c(O[C@@H]3O[C@@H]6COC(C)(C)O[C@H]6[C@H](O)[C@H]3O)c15)C=CC(C)(CCC=C(C)C)O2)c1ccccc1C4=O] | C₅₇H₆₄O₁₄ | 973.11 |
| 153 | 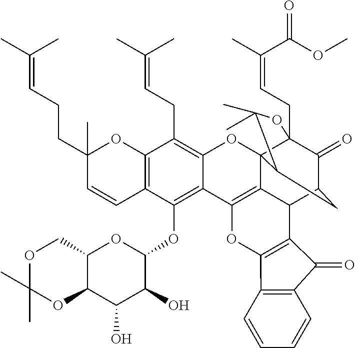 [SMILES: COC(=O)/C(C)=C\CC12OC(C)(C)C3CC(C1=O)C1C4=C(OC5=C1C32Oc1c(CC=C(C)C)c2c(c(O[C@H]3O[C@H]6COC(C)(C)O[C@@H]6[C@@H](O)[C@@H]3O)c15)C=CC(C)(CCC=C(C)C)O2)c1ccccc1C4=O] | C₅₇H₆₆O₁₄ | 973.11 |
| 154 | 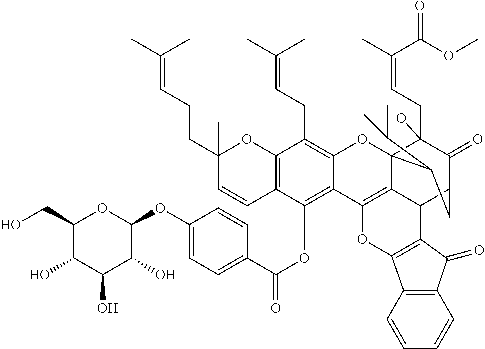 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C2=C(O1)c1ccccc1C2=O)C=CC(C)(CCC=C(C)C)O4] | C₆₁H₆₆O₁₆ | 1053.15 |
| 155 | 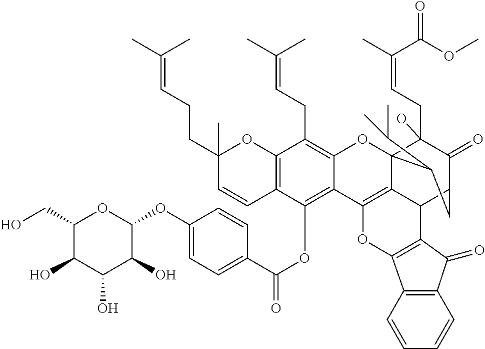 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C2=C(O1)c1ccccc1C2=O)C=CC(C)(CCC=C(C)C)O4] | C₆₁H₆₆O₁₆ | 1053.15 |
| 156 | 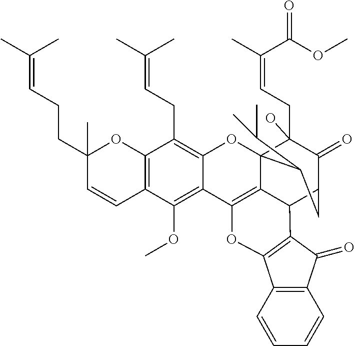 [SMILES: COC(=O)/C(C)=C\CC1(O)C(=O)C2CC(C(C)C)C13Oc1c(CC=C(C)C)c4c(c(OC)c1C1=C3C2C2=C(O1)c1ccccc1C2=O)C=CC(C)(CCC=C(C)C)O4] | C₄₉H₅₂O₉ | 784.93 |
| 157 | 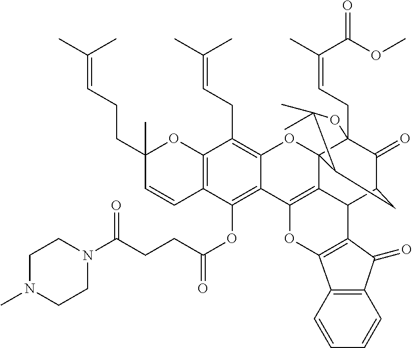 [SMILES: COC(=O)/C(C)=C\CC12OC(C)(C)C3CC(C1=O)C1C4=C(OC5=C1C32Oc1c(CC=C(C)C)c2c(c(OC(=O)CCC(=O)N3CCN(C)CC3)c15)C=CC(C)(CCC=C(C)C)O2)c1ccccc1C4=O] | C₅₇H₆₄N₂O₁₁ | 953.12 |
| 158 | 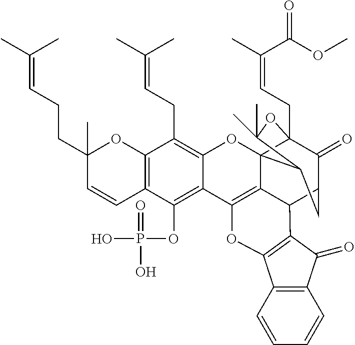 [SMILES: COC(=O)/C(C)=C\CC12OC(C)(C)C3CC(C1=O)C1C4=C(OC5=C1C32Oc1c(CC=C(C)C)c2c(c(OP(=O)(O)O)c15)C=CC(C)(CCC=C(C)C)O2)c1ccccc1C4=O] | C₄₈H₅₁O₁₂P | 850.89 |
| 159 | 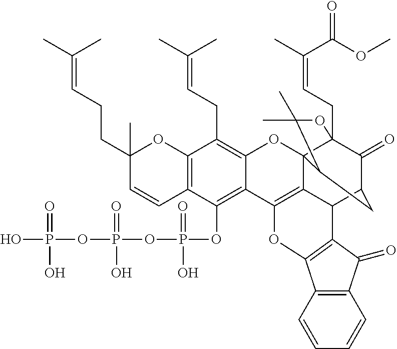 [SMILES: COC(=O)/C(C)=C\CC12OC(C)(C)C3CC(C1=O)C1C4=C(OC5=C1C32Oc1c(CC=C(C)C)c2c(c(OP(=O)(O)OP(=O)(O)OP(=O)(O)O)c15)C=CC(C)(CCC=C(C)C)O2)c1ccccc1C4=O] | C₄₈H₅₃O₁₈P₃ | 1010.84 |
| 160 | 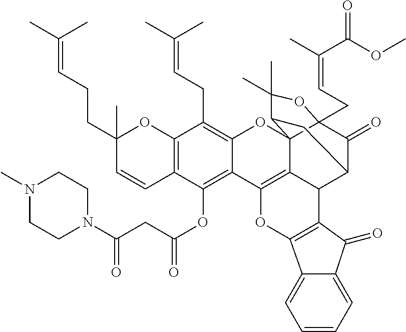 | C₅₆H₆₂N₂O₁₁ | 939.1 |
| 161 | 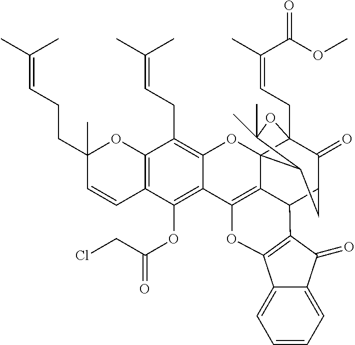 [SMILES: COC(=O)/C(C)=C\CC12OC(C)(C)C3CC(C1=O)C1C4=C(OC5=C1C32Oc1c(CC=C(C)C)c2c(c(OC(=O)CCl)c15)C=CC(C)(CCC=C(C)C)O2)c1ccccc1C4=O] | C₅₀H₅₁ClO₁₀ | 847.39 |
| 162 | 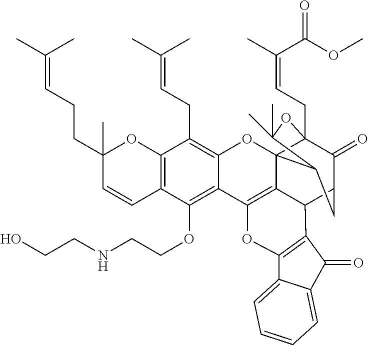 [SMILES: COC(=O)/C(C)=C\CC12OC(C)(C)C3CC(C1=O)C1C4=C(OC5=C1C32Oc1c(CC=C(C)C)c2c(c(OCCNCCO)c15)C=CC(C)(CCC=C(C)C)O2)c1ccccc1C4=O] | C₅₂H₅₉NO₁₀ | 858.03 |
| 163 | 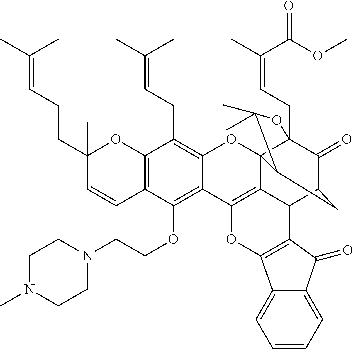 [SMILES: COC(=O)/C(C)=C\CC12OC(C)(C)C3CC(C1=O)C1C4=C(OC5=C1C32Oc1c(CC=C(C)C)c2c(c(OCCN3CCN(C)CC3)c15)C=CC(C)(CCC=C(C)C)O2)c1ccccc1C4=O] | C₅₅H₆₄N₂O₉ | 897.1 |
| 164 | 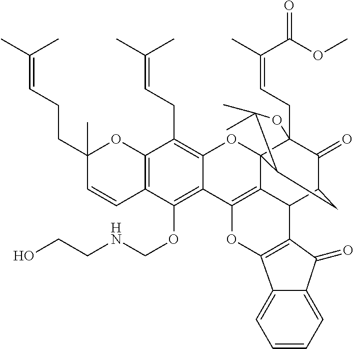 [SMILES: COC(=O)/C(C)=C\CC12OC(C)(C)C3CC(C1=O)C1C4=C(OC5=C1C32Oc1c(CC=C(C)C)c2c(c(OCNCCO)c15)C=CC(C)(CCC=C(C)C)O2)c1ccccc1C4=O] | C₅₁H₅₇NO₁₀ | 844 |
| 165 | 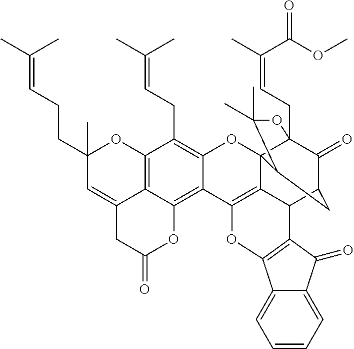 [SMILES: COC(=O)/C(C)=C\CC12OC(C)(C)C3CC(C1=O)C1C4=C(OC5=C1C32Oc1c(CC=C(C)C)c2c3c(c15)OC(=O)CC3=CC(C)(CCC=C(C)C)O2)c1ccccc1C4=O] | C₅₀H₅₀O₁₀ | 810.93 |
| 166 | 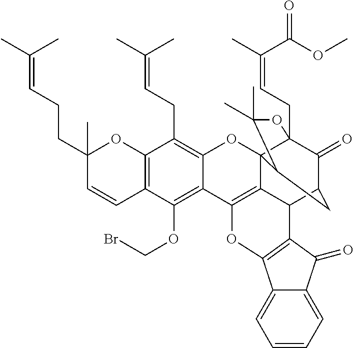 [SMILES: COC(=O)/C(C)=C\CC12OC(C)(C)C3CC(C1=O)C1C4=C(OC5=C1C32Oc1c(CC=C(C)C)c2c(c(OCBr)c15)C=CC(C)(CCC=C(C)C)O2)c1ccccc1C4=O] | C₄₉H₅₁BrO₉ | 863.93 |
| 167 | 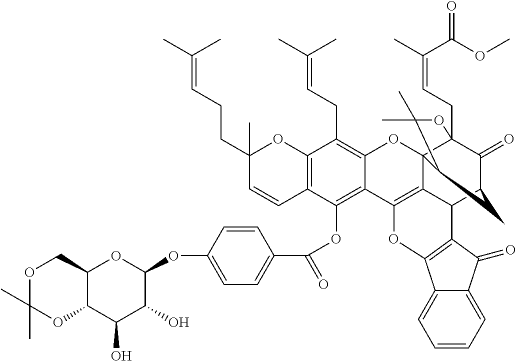 [SMILES: COC(=O)/C(C)=C\CC12OC(C)(C)[C@H]3C[C@H](C1=O)C1C4=C(OC5=C1C32Oc1c(CC=C(C)C)c2c(c(OC(=O)c3ccc(O[C@@H]6O[C@@H]7COC(C)(C)O[C@H]7[C@H](O)[C@H]6O)cc3)c15)C=CC(C)(CCC=C(C)C)O2)c1ccccc1C4=O] | C₆₄H₆₈O₁₆ | 1093.22 |
| 168 | 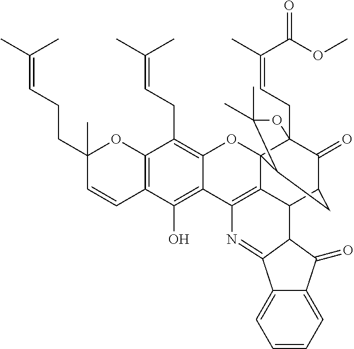 [SMILES: COC(=O)/C(C)=C\CC12OC(C)(C)C3CC(C1=O)C1C4=C(N=C5c6ccccc6C(=O)C51)c1c(O)c5c(c(CC=C(C)C)c1OC432)OC(C)(CCC=C(C)C)C=C5] | C₄₈H₅₁O₈ | 769.92 |
| 169 | 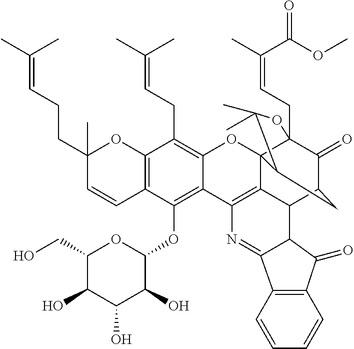 [SMILES: COC(=O)/C(C)=C\CC12OC(C)(C)C3CC(C1=O)C1C4=C(N=C5c6ccccc6C(=O)C51)c1c(O[C@H]5O[C@@H](CO)[C@H](O)[C@@H](O)[C@@H]5O)c5c(c(CC=C(C)C)c1OC432)OC(C)(CCC=C(C)C)C=C5] | C₅₄H₆₁NO₁₃ | 932.06 |
| 170 | 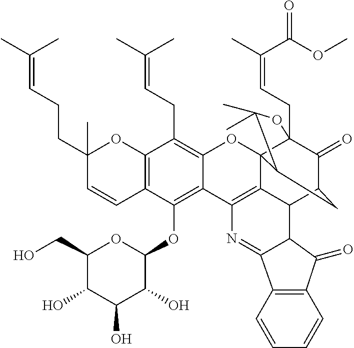 [SMILES: COC(=O)/C(C)=C\CC12OC(C)(C)C3CC(C1=O)C1C4=C(N=C5c6ccccc6C(=O)C51)c1c(O[C@@H]5O[C@H](CO)[C@@H](O)[C@H](O)[C@H]5O)c5c(c(CC=C(C)C)c1OC432)OC(C)(CCC=C(C)C)C=C5] | C₅₄H₆₁NO₁₃ | 932.06 |
| 171 | 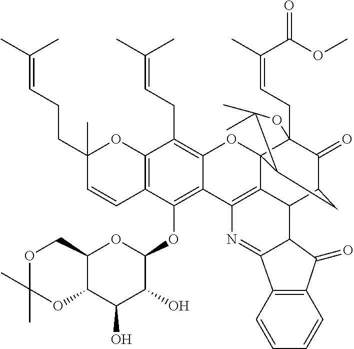 [SMILES: COC(=O)/C(C)=C\CC12OC(C)(C)C3CC(C1=O)C1C4=C(N=C5c6ccccc6C(=O)C51)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₅₇H₆₅NO₁₃ | 972.12 |
| 172 | 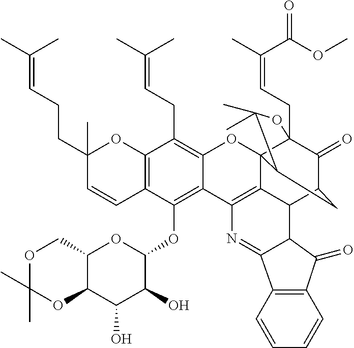 [SMILES: COC(=O)/C(C)=C\CC12OC(C)(C)C3CC(C1=O)C1C4=C(N=C5c6ccccc6C(=O)C51)c1c(O[C@H]5O[C@H]6COC(C)(C)O[C@@H]6[C@@H](O)[C@@H]5O)c5c(c(CC=C(C)C)c1OC432)OC(C)(CCC=C(C)C)C=C5] | C₅₇H₆₅NO₁₃ | 972.12 |
| 173 | 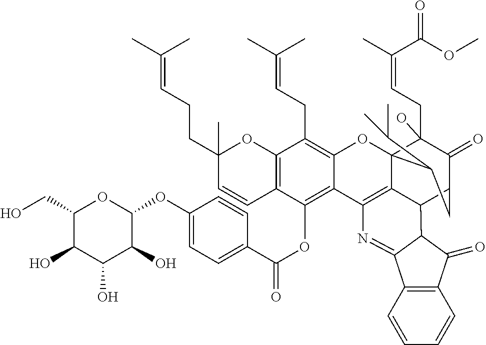 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C2C(=O)c3ccccc3C2=N1)C=CC(C)(CCC=C(C)C)O4] | C₆₁H₆₅NO₁₅ | 1052.17 |
| 174 | 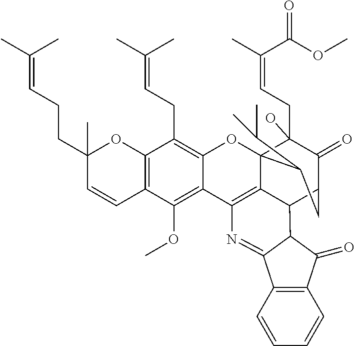 [SMILES: COC(=O)/C(C)=C\CC1(O)C(=O)C2CC(C(C)C)C13Oc1c(CC=C(C)C)c4c(c(OC)c1C1=C3C2C2C(=O)c3ccccc3C2=N1)C=CC(C)(CCC=C(C)C)O4] | C₄₉H₅₃NO₈ | 783.95 |
| 175 | 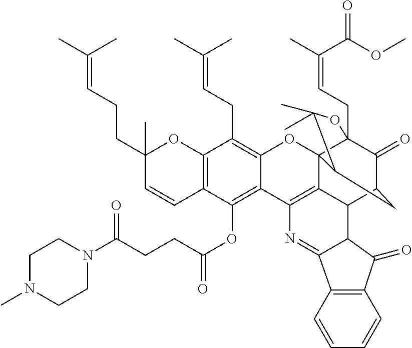 [SMILES: COC(=O)/C(C)=C\CC12OC(C)(C)C3CC(C1=O)C1C4=C(N=C5c6ccccc6C(=O)C51)c1c(OC(=O)CCC(=O)N5CCN(C)CC5)c5c(c(CC=C(C)C)c1OC432)OC(C)(CCC=C(C)C)C=C5] | C₅₇H₆₅N₃O₁₀ | 952.14 |
| 176 | 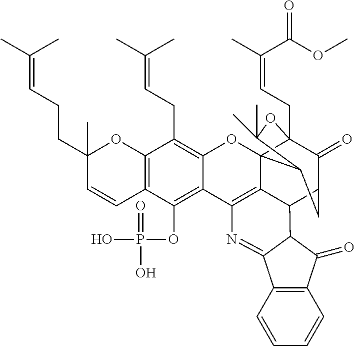 [SMILES: COC(=O)/C(C)=C\CC12OC(C)(C)C3CC(C1=O)C1C4=C(N=C5c6ccccc6C(=O)C51)c1c(OP(=O)(O)O)c5c(c(CC=C(C)C)c1OC432)OC(C)(CCC=C(C)C)C=C5] | C₄₈H₅₂NO₁₁P | 849.9 |
| 177 | 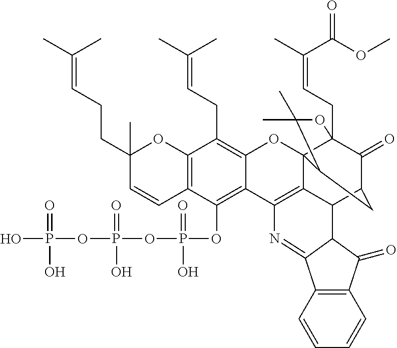 [SMILES: COC(=O)/C(C)=C\CC12OC(C)(C)C3CC(C1=O)C1C4=C(N=C5c6ccccc6C(=O)C51)c1c(OP(=O)(O)OP(=O)(O)OP(=O)(O)O)c5c(c(CC=C(C)C)c1OC432)OC(C)(CCC=C(C)C)C=C5] | C₄₈H₅₄NO₁₇P₃ | 1009.86 |
| 178 | 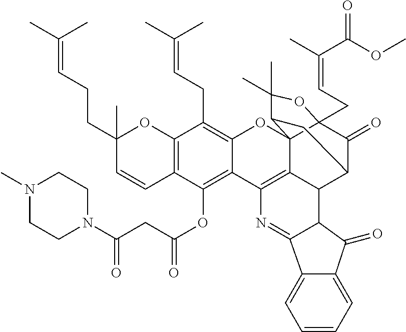 | C₅₆H₆₃N₃O₁₀ | 938.11 |
| 179 | 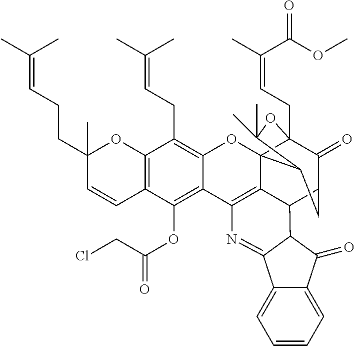 [SMILES: COC(=O)/C(C)=C\CC12OC(C)(C)C3CC(C1=O)C1C4=C(N=C5c6ccccc6C(=O)C51)c1c(OC(=O)CCl)c5c(c(CC=C(C)C)c1OC432)OC(C)(CCC=C(C)C)C=C5] | C₅₀H₅₂ClNO₉ | 846.4 |
| 180 | 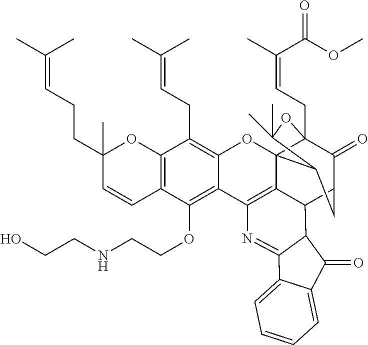 [SMILES: COC(=O)/C(C)=C\CC12OC(C)(C)C3CC(C1=O)C1C4=C(N=C5c6ccccc6C(=O)C51)c1c(OCCNCCO)c5c(c(CC=C(C)C)c1OC432)OC(C)(CCC=C(C)C)C=C5] | C₅₂H₆₀N₂O₉ | 857.04 |
| 181 | 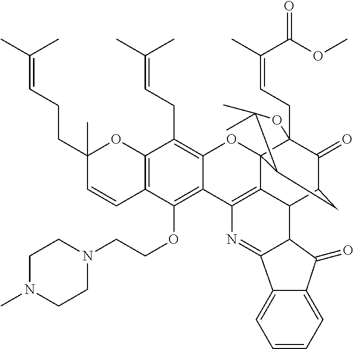 [SMILES: COC(=O)/C(C)=C\CC12OC(C)(C)C3CC(C1=O)C1C4=C(N=C5c6ccccc6C(=O)C51)c1c(OCCN5CCN(C)CC5)c5c(c(CC=C(C)C)c1OC432)OC(C)(CCC=C(C)C)C=C5] | C₅₅H₆₅N₃O₈ | 896.12 |
| 182 | 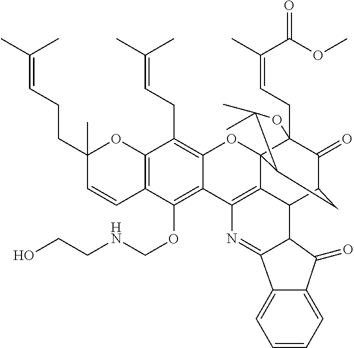 [SMILES: COC(=O)/C(C)=C\CC12OC(C)(C)C3CC(C1=O)C1C4=C(N=C5c6ccccc6C(=O)C51)c1c(OCNCCO)c5c(c(CC=C(C)C)c1OC432)OC(C)(CCC=C(C)C)C=C5] | C₅₁H₅₈N₂O₉ | 843.01 |
| 183 | 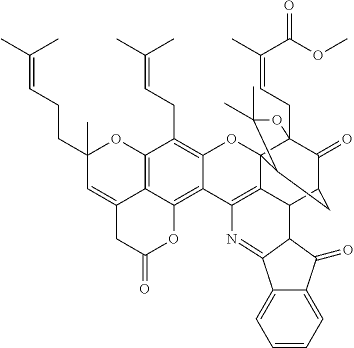 [SMILES: COC(=O)/C(C)=C\CC12OC(C)(C)C3CC(C1=O)C1C4=C(N=C5c6ccccc6C(=O)C51)c1c(c(CC=C(C)C)c5c6c1OC(=O)CC6=CC(C)(CCC=C(C)C)O5)OC432] | C₅₀H₅₁NO₉ | 809.94 |
| 184 | 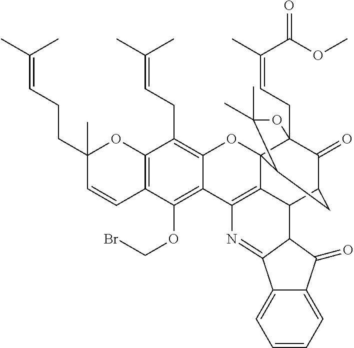 [SMILES: COC(=O)/C(C)=C\CC12OC(C)(C)C3CC(C1=O)C1C4=C(N=C5c6ccccc6C(=O)C51)c1c(OCBr)c5c(c(CC=C(C)C)c1OC432)OC(C)(CCC=C(C)C)C=C5] | C₄₉H₅₂BrNO₈ | 862.84 |
| 185 | 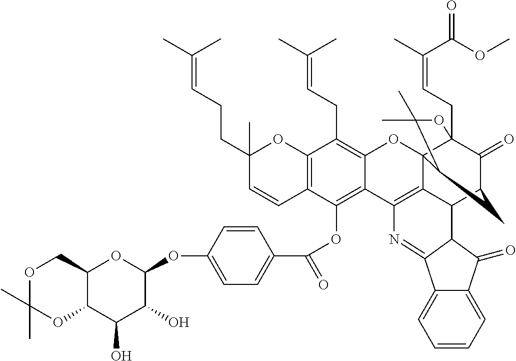 [SMILES: COC(=O)/C(C)=C\CC12OC(C)(C)[C@H]3C[C@H](C1=O)C1C4=C(N=C5c6ccccc6C(=O)C51)c1c(OC(=O)c5ccc(O[C@@H]6O[C@@H]7COC(C)(C)O[C@H]7[C@H](O)[C@H]6O)cc5)c5c(c(CC=C(C)C)c1OC432)OC(C)(CCC=C(C)C)C=C5] | C₆₄H₆₉NO₁₅ | 1092.23 |
| 186 | 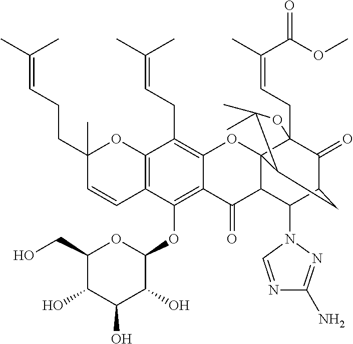 [SMILES: COC(=O)/C(C)=C\CC12OC(C)(C)C3CC(C1=O)C(n1cnc(N)n1)C1C(=O)c4c(O[C@@H]5O[C@H](CO)[C@@H](O)[C@H](O)[C@H]5O)c5c(c(CC=C(C)C)c4OC132)OC(C)(CCC=C(C)C)C=C5] | C₄₇H₆₀N₄O₁₃ | 889 |
| 187 | 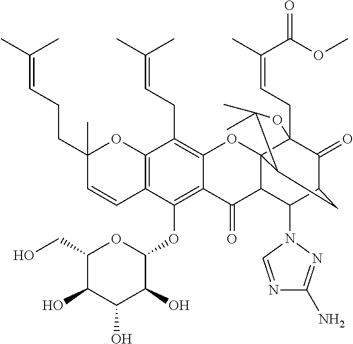 [SMILES: COC(=O)/C(C)=C\CC12OC(C)(C)C3CC(C1=O)C(n1cnc(N)n1)C1C(=O)c4c(O[C@H]5O[C@@H](CO)[C@H](O)[C@@H](O)[C@@H]5O)c5c(c(CC=C(C)C)c4OC132)OC(C)(CCC=C(C)C)C=C5] | C₄₇H₆₀N₄O₁₃ | 889 |
| 188 | 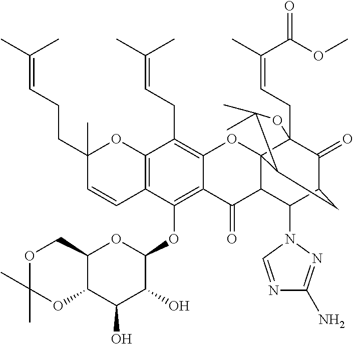 [SMILES: COC(=O)/C(C)=C\CC12OC(C)(C)C3CC(C1=O)C(n1cnc(N)n1)C1C(=O)c4c(O[C@@H]5O[C@@H]6COC(C)(C)O[C@H]6[C@H](O)[C@H]5O)c5c(c(CC=C(C)C)c4OC132)OC(C)(CCC=C(C)C)C=C5] | C₅₀H₆₄N₄O₁₃ | 929.06 |
| 189 | 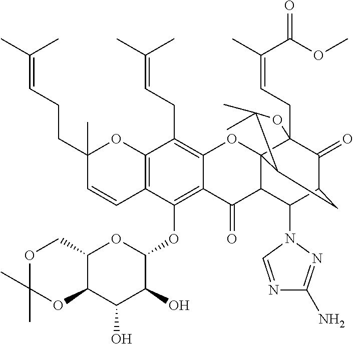 [SMILES: COC(=O)/C(C)=C\CC12OC(C)(C)C3CC(C1=O)C(n1cnc(N)n1)C1C(=O)c4c(O[C@H]5O[C@H]6COC(C)(C)O[C@@H]6[C@@H](O)[C@@H]5O)c5c(c(CC=C(C)C)c4OC132)OC(C)(CCC=C(C)C)C=C5] | C₅₀H₆₄N₄O₁₃ | 929.06 |
| 190 | 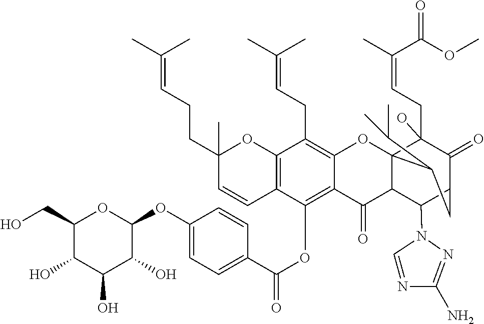 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C(=O)C3C2n1cnc(N)n1)C=CC(C)(CCC=C(C)C)O4] | C₅₄H₆₄N₄O₁₅ | 1009.1 |
| 191 | 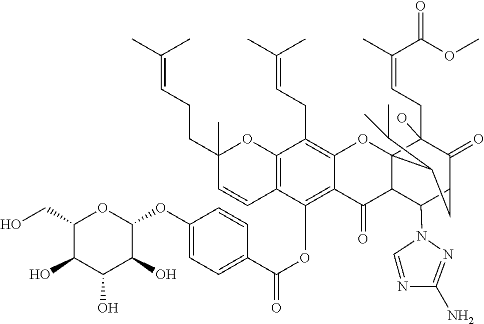 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C(=O)C3C2n1cnc(N)n1)C=CC(C)(CCC=C(C)C)O4] | C₅₄H₆₄N₄O₁₅ | 1009.1 |
| 192 | 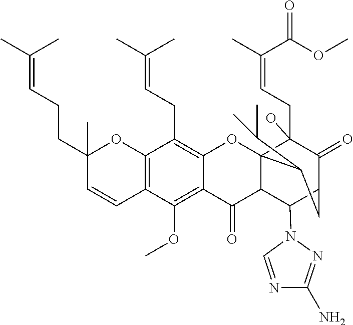 [SMILES: COC(=O)/C(C)=C\CC1(O)C(=O)C2CC(C(C)C)C13Oc1c(CC=C(C)C)c4c(c(OC)c1C(=O)C3C2n1cnc(N)n1)C=CC(C)(CCC=C(C)C)O4] | C₄₂H₅₂N₄O₈ | 740.88 |
| 193 | 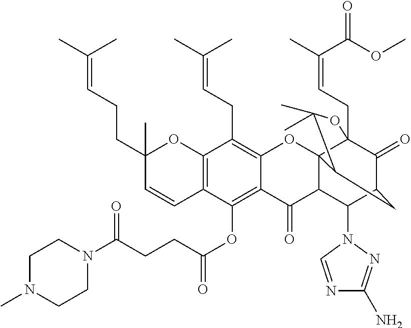 [SMILES: COC(=O)/C(C)=C\CC12OC(C)(C)C3CC(C1=O)C(n1cnc(N)n1)C1C(=O)c4c(OC(=O)CCC(=O)N5CCN(C)CC5)c5c(c(CC=C(C)C)c4OC132)OC(C)(CCC=C(C)C)C=C5] | C₅₀H₆₄N₆O₁₀ | 909.08 |
| 194 | 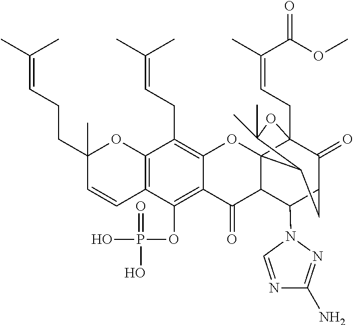 [SMILES: COC(=O)/C(C)=C\CC12OC(C)(C)C3CC(C1=O)C(n1cnc(N)n1)C1C(=O)c4c(OP(=O)(O)O)c5c(c(CC=C(C)C)c4OC132)OC(C)(CCC=C(C)C)C=C5] | C₄₁H₅₁N₄O₁₁P | 806.84 |
| 195 | 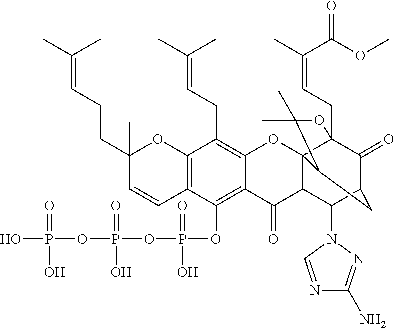 [SMILES: COC(=O)/C(C)=C\CC12OC(C)(C)C3CC(C1=O)C(n1cnc(N)n1)C1C(=O)c4c(OP(=O)(O)OP(=O)(O)OP(=O)(O)O)c5c(c(CC=C(C)C)c4OC132)OC(C)(CCC=C(C)C)C=C5] | C₄₁H₅₃N₄O₁₇P₃ | 966.8 |
| 196 | 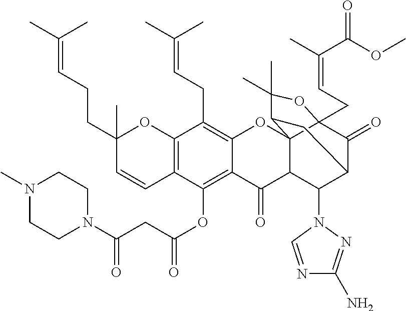 | C₄₉H₆₂N₆O₁₀ | 895.05 |
| 197 | 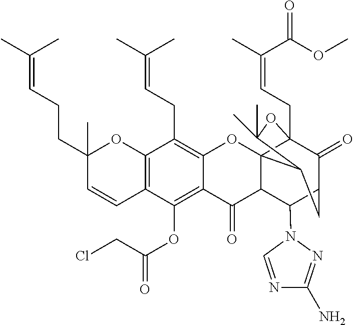 [SMILES: COC(=O)/C(C)=C\CC12OC(C)(C)C3CC(C1=O)C(n1cnc(N)n1)C1C(=O)c4c(OC(=O)CCl)c5c(c(CC=C(C)C)c4OC132)OC(C)(CCC=C(C)C)C=C5] | C₄₃H₅₁ClN₄O₉ | 803.34 |
| 198 | 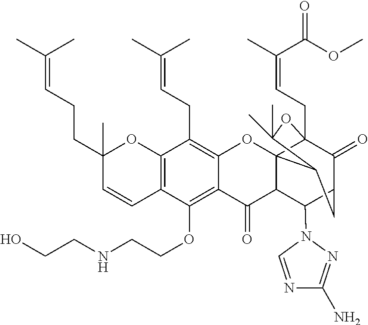 [SMILES: COC(=O)/C(C)=C\CC12OC(C)(C)C3CC(C1=O)C(n1cnc(N)n1)C1C(=O)c4c(OCCNCCO)c5c(c(CC=C(C)C)c4OC132)OC(C)(CCC=C(C)C)C=C5] | C₄₆H₆₀N₄O₉ | 812.99 |
| 199 | 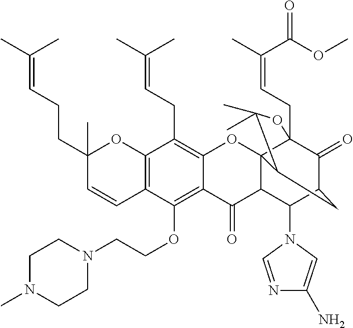 [SMILES: COC(=O)/C(C)=C\CC12OC(C)(C)C3CC(C1=O)C(n1cnc(N)c1)C1C(=O)c4c(OCCN5CCN(C)CC5)c5c(c(CC=C(C)C)c4OC132)OC(C)(CCC=C(C)C)C=C5] | C₄₉H₆₅N₅O₈ | 852.07 |
| 200 | 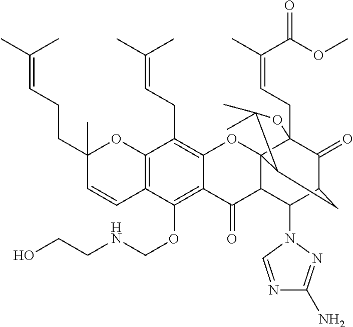 [SMILES: COC(=O)/C(C)=C\CC12OC(C)(C)C3CC(C1=O)C(n1cnc(N)n1)C1C(=O)c4c(OCNCCO)c5c(c(CC=C(C)C)c4OC132)OC(C)(CCC=C(C)C)C=C5] | C₄₄H₅₇N₅O₉ | 799.95 |
| 201 | 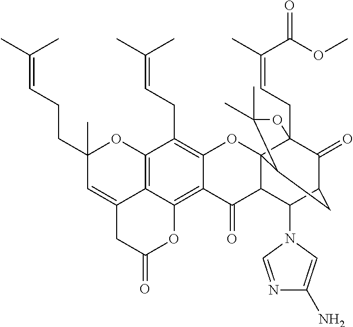 [SMILES: COC(=O)/C(C)=C\CC12OC(C)(C)C3CC(C1=O)C(n1cnc(N)c1)C1C(=O)c4c(c(CC=C(C)C)c5c6c4OC(=O)CC6=CC(C)(CCC=C(C)C)O5)OC132] | C₄₄H₅₁N₃O₉ | 765.89 |
| 202 | 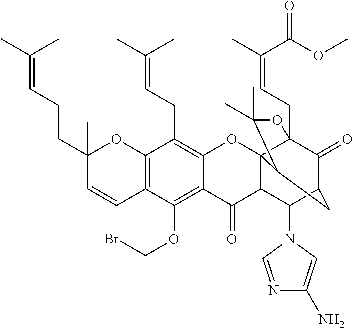 [SMILES: COC(=O)/C(C)=C\CC12OC(C)(C)C3CC(C1=O)C(n1cnc(N)c1)C1C(=O)c4c(OCBr)c5c(c(CC=C(C)C)c4OC132)OC(C)(CCC=C(C)C)C=C5] | C₄₃H₅₂BrN₃O₈ | 818.79 |
| 203 | 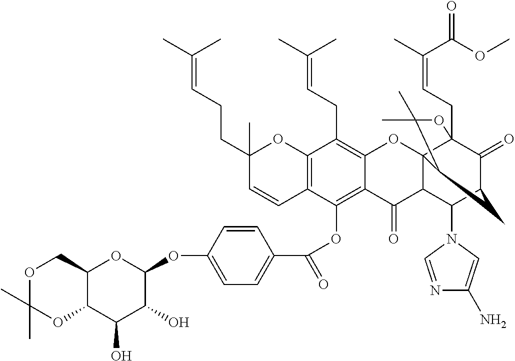 [SMILES: COC(=O)/C(C)=C\CC12OC(C)(C)[C@H]3C[C@H](C1=O)C(n1cnc(N)c1)C1C(=O)c4c(OC(=O)c5ccc(O[C@@H]6O[C@@H]7COC(C)(C)O[C@H]7[C@H](O)[C@H]6O)cc5)c5c(c(CC=C(C)C)c4OC132)OC(C)(CCC=C(C)C)C=C5] | C₅₈H₆₉N₃O₁₅ | 1048.18 |
| 204 | 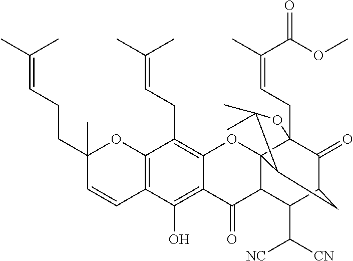 [SMILES: COC(=O)/C(C)=C\CC12OC(C)(C)C3CC(C1=O)C(C(C#N)C#N)C1C(=O)c4c(O)c5c(c(CC=C(C)C)c4OC132)OC(C)(CCC=C(C)C)C=C5] | C₄₂H₄₈N₂O₈ | 708.84 |
| 205 | 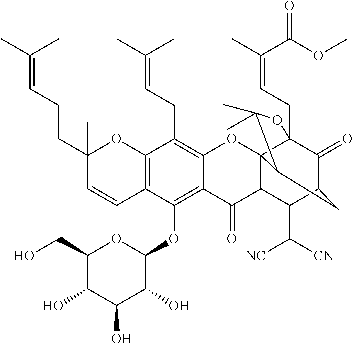 [SMILES: COC(=O)/C(C)=C\CC12OC(C)(C)C3CC(C1=O)C(C(C#N)C#N)C1C(=O)c4c(O[C@@H]5O[C@H](CO)[C@@H](O)[C@H](O)[C@H]5O)c5c(c(CC=C(C)C)c4OC132)OC(C)(CCC=C(C)C)C=C5] | C₄₈H₅₈N₂O₁₃ | 870.98 |
| 206 | 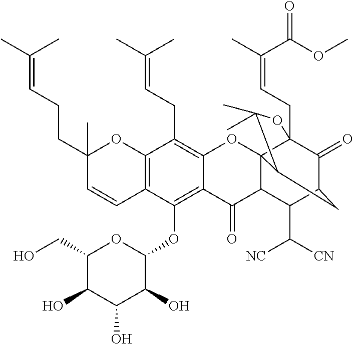 [SMILES: COC(=O)/C(C)=C\CC12OC(C)(C)C3CC(C1=O)C(C(C#N)C#N)C1C(=O)c4c(O[C@H]5O[C@@H](CO)[C@H](O)[C@@H](O)[C@@H]5O)c5c(c(CC=C(C)C)c4OC132)OC(C)(CCC=C(C)C)C=C5] | C₄₈H₅₈N₂O₁₃ | 870.98 |
| 207 | 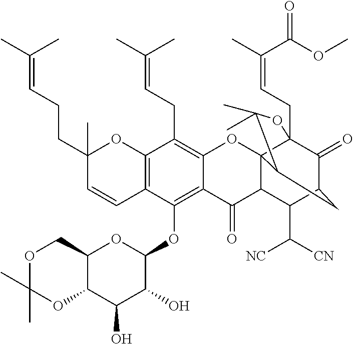 [SMILES: COC(=O)/C(C)=C\CC12OC(C)(C)C3CC(C1=O)C(C(C#N)C#N)C1C(=O)c4c(O[C@@H]5O[C@@H]6COC(C)(C)O[C@H]6[C@H](O)[C@H]5O)c5c(c(CC=C(C)C)c4OC132)OC(C)(CCC=C(C)C)C=C5] | C₅₁H₆₂N₂O₁₃ | 911.04 |
| 208 | 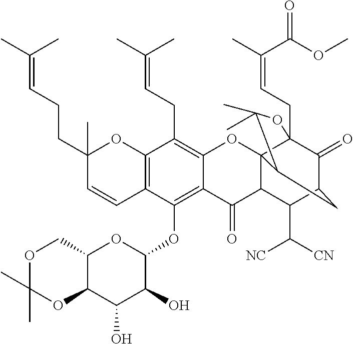 [SMILES: COC(=O)/C(C)=C\CC12OC(C)(C)C3CC(C1=O)C(C(C#N)C#N)C1C(=O)c4c(O[C@H]5O[C@H]6COC(C)(C)O[C@@H]6[C@@H](O)[C@@H]5O)c5c(c(CC=C(C)C)c4OC132)OC(C)(CCC=C(C)C)C=C5] | C₅₁H₆₂N₂O₁₃ | 911.04 |
| 209 | 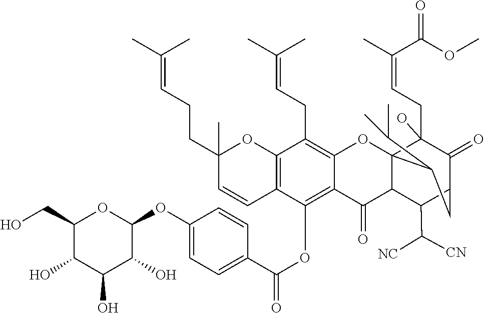 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C(=O)C3C2C(C#N)C#N)C=CC(C)(CCC=C(C)C)O4] | C₅₅H₆₂N₂O₁₅ | 991.09 |
| 210 | 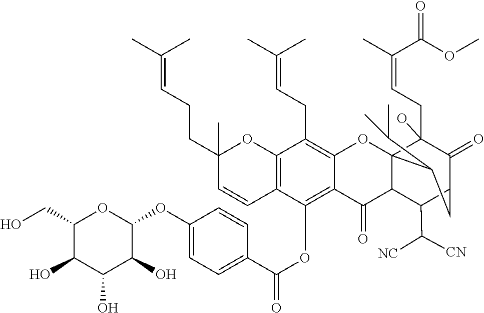 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C(=O)C3C2C(C#N)C#N)C=CC(C)(CCC=C(C)C)O4] | C₅₅H₆₂N₂O₁₅ | 991.09 |
| 211 | 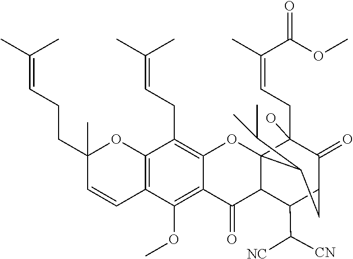 [SMILES: COC(=O)/C(C)=C\CC1(O)C(=O)C2CC(C(C)C)C13Oc1c(CC=C(C)C)c4c(c(OC)c1C(=O)C3C2C(C#N)C#N)C=CC(C)(CCC=C(C)C)O4] | C₄₃H₅₀N₂O₈ | 722.87 |
| 212 | 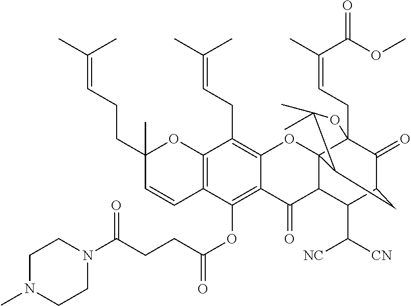 [SMILES: COC(=O)/C(C)=C\CC12OC(C)(C)C3CC(C1=O)C(C(C#N)C#N)C1C(=O)c4c(OC(=O)CCC(=O)N5CCN(C)CC5)c5c(c(CC=C(C)C)c4OC132)OC(C)(CCC=C(C)C)C=C5] | C₅₁H₆₂N₄O₁₀ | 891.06 |
| 213 | 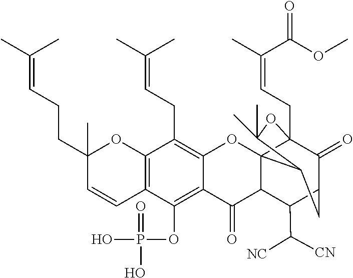 [SMILES: COC(=O)/C(C)=C\CC12OC(C)(C)C3CC(C1=O)C(C(C#N)C#N)C1C(=O)c4c(OP(=O)(O)O)c5c(c(CC=C(C)C)c4OC132)OC(C)(CCC=C(C)C)C=C5] | C₄₂H₄₉N₂O₁₁P | 788.82 |
| 214 | 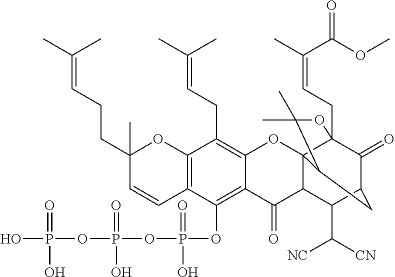 [SMILES: COC(=O)/C(C)=C\CC12OC(C)(C)C3CC(C1=O)C(C(C#N)C#N)C1C(=O)c4c(OP(=O)(O)OP(=O)(O)OP(=O)(O)O)c5c(c(CC=C(C)C)c4OC132)OC(C)(CCC=C(C)C)C=C5] | C₄₂H₅₁N₂O₁₇P₃ | 948.78 |
| 215 | 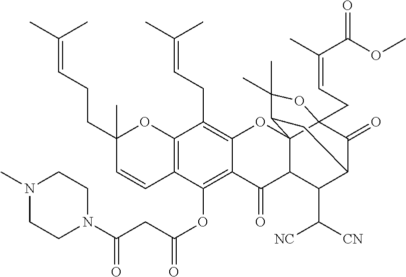 | C₅₀H₆₀N₄O₁₀ | 877.03 |
| 216 | 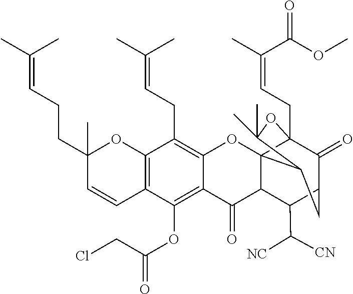 [SMILES: COC(=O)/C(C)=C\CC12OC(C)(C)C3CC(C1=O)C(C(C#N)C#N)C1C(=O)c4c(OC(=O)CCl)c5c(c(CC=C(C)C)c4OC132)OC(C)(CCC=C(C)C)C=C5] | C₄₄H₄₉ClN₂O₉ | 785.32 |
| 217 | 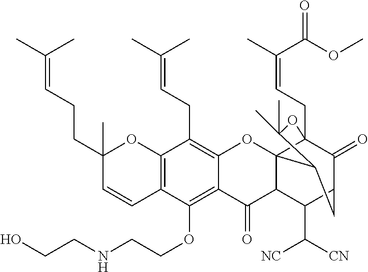 [SMILES: COC(=O)/C(C)=C\CC12OC(C)(C)C3CC(C1=O)C(C(C#N)C#N)C1C(=O)c4c(OCCNCCO)c5c(c(CC=C(C)C)c4OC132)OC(C)(CCC=C(C)C)C=C5] | C₄₆H₅₇N₃O₉ | 795.96 |
| 218 | 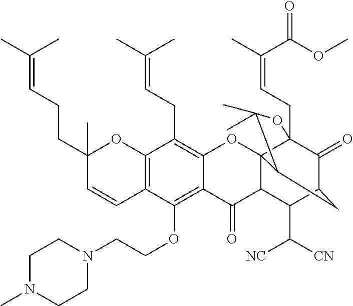 [SMILES: COC(=O)/C(C)=C\CC12OC(C)(C)C3CC(C1=O)C(C(C#N)C#N)C1C(=O)c4c(OCCN5CCN(C)CC5)c5c(c(CC=C(C)C)c4OC132)OC(C)(CCC=C(C)C)C=C5] | C₄₉H₆₂N₄O₈ | 835.04 |
| 219 | 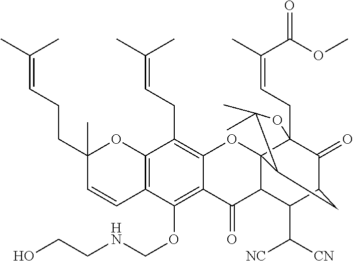 [SMILES: COC(=O)/C(C)=C\CC12OC(C)(C)C3CC(C1=O)C(C(C#N)C#N)C1C(=O)c4c(OCNCCO)c5c(c(CC=C(C)C)c4OC132)OC(C)(CCC=C(C)C)C=C5] | C₄₅H₅₅N₃O₉ | 781.93 |
| 220 | 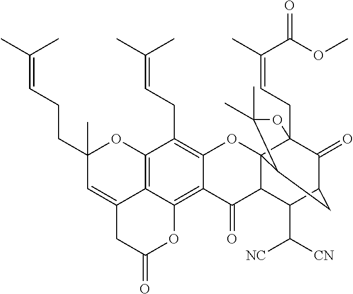 [SMILES: COC(=O)/C(C)=C\CC12OC(C)(C)C3CC(C1=O)C(C(C#N)C#N)C1C(=O)c4c(c(CC=C(C)C)c5c6c4OC(=O)CC6=CC(C)(CCC=C(C)C)O5)OC132] | C₄₄H₄₈N₂O₉ | 748.86 |
| 221 | 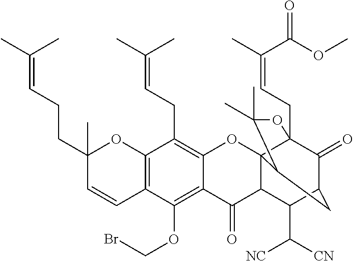 [SMILES: COC(=O)/C(C)=C\CC12OC(C)(C)C3CC(C1=O)C(C(C#N)C#N)C1C(=O)c4c(OCBr)c5c(c(CC=C(C)C)c4OC132)OC(C)(CCC=C(C)C)C=C5] | C₄₃H₄₉BrN₂O₈ | 801.76 |
| 222 | 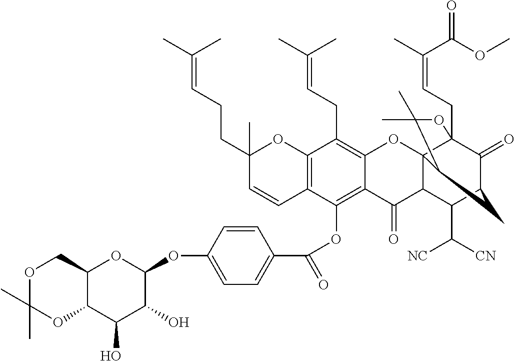 [SMILES: COC(=O)/C(C)=C\CC12OC(C)(C)[C@H]3C[C@H](C1=O)C(C(C#N)C#N)C1C(=O)c4c(OC(=O)c5ccc(O[C@@H]6O[C@@H]7COC(C)(C)O[C@H]7[C@H](O)[C@H]6O)cc5)c5c(c(CC=C(C)C)c4OC132)OC(C)(CCC=C(C)C)C=C5] | C₅₈H₆₆N₂O₁₅ | 1031.15 |
| 223 | 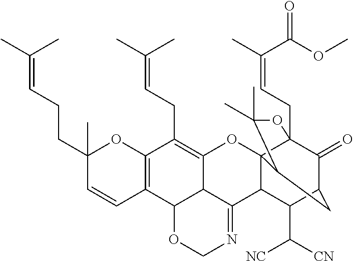 [SMILES: COC(=O)/C(C)=C\CC12OC(C)(C)C3CC(C1=O)C(C(C#N)C#N)C1C4=NCOC5C6=C(OC(C)(CCC=C(C)C)C=C6)C(CC=C(C)C)=C(OC132)C45] | C₄₃H₅₁N₃O₇ | 721.88 |
| 224 | 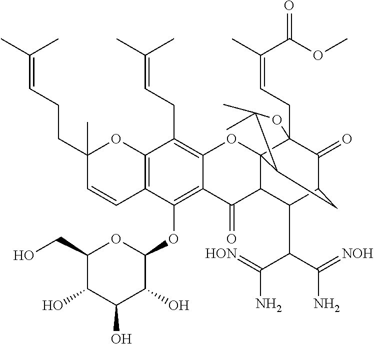 | C₄₈H₆₄N₄O₁₅ | 937.04 |
| 225 | 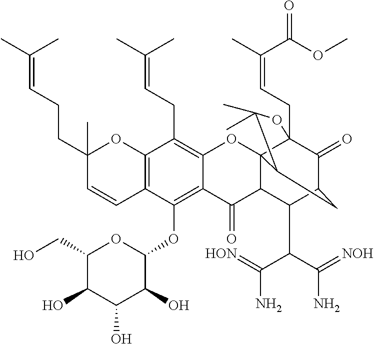 | C₄₉H₆₆N₄O₁₄ | 935.07 |
| 226 | 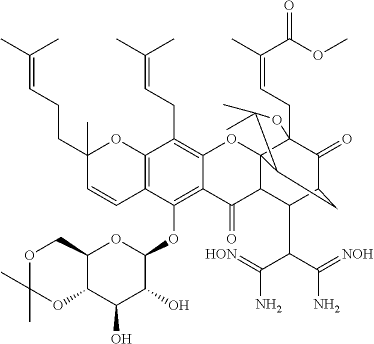 | C₅₁H₆₈N₄O₁₅ | 977.1 |
| 227 | 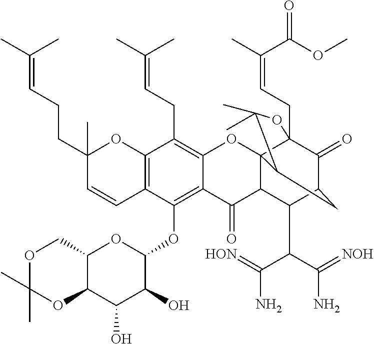 | C₅₁H₆₈N₄O₁₅ | 977.1 |
| 228 | 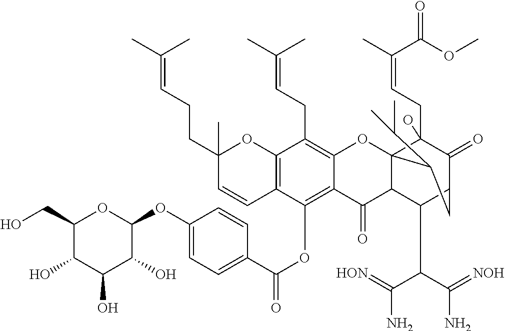 | C₅₅H₆₈N₄O₁₇ | 1057.15 |
| 229 | 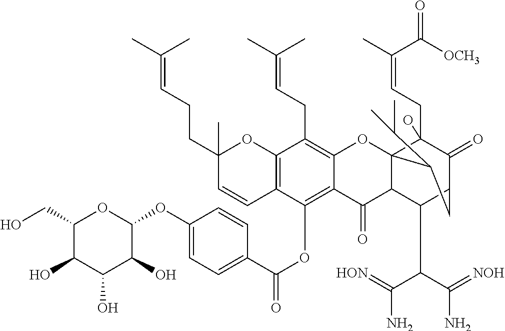 | C₅₅H₆₈N₄O₁₇ | 1057.15 |
| 230 | 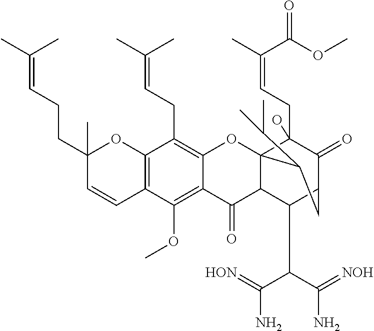 | C₄₃H₅₆N₄O₁₀ | 788.93 |
| 231 | 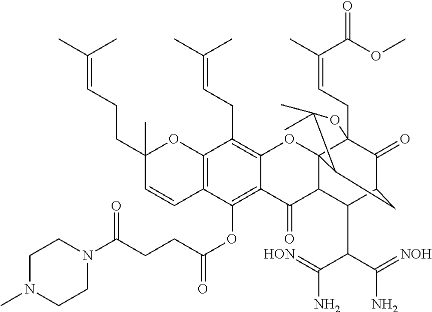 | C₅₁H₆₈N₂O₁₂ | 957.12 |
| 232 | 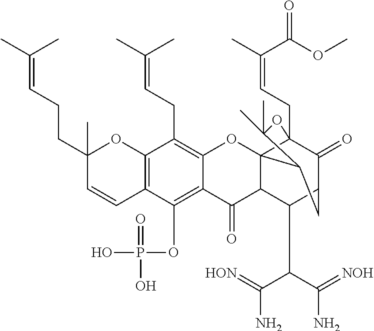 | C₄₂H₅₅N₄O₁₃P | 854.88 |
| 233 | 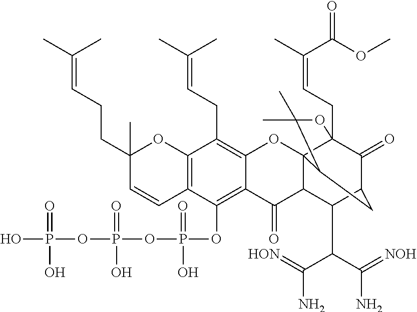 | C₄₂H₅₇N₄O₁₉P₃ | 1014.84 |
| 234 | 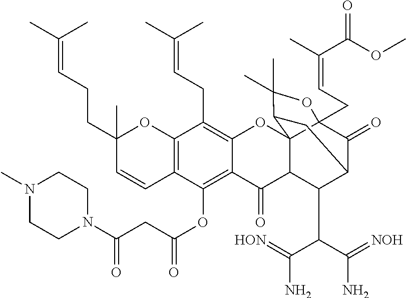 | C₅₀H₆₆N₆O₁₂ | 943.09 |
| 235 | 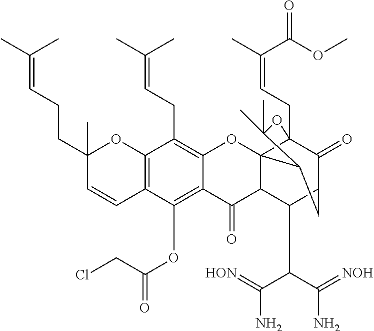 | C₄₄H₅₅ClN₄O₁₁ | 851.38 |
| 236 | 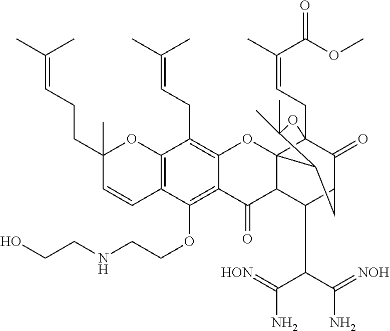 | C₄₆H₆₃N₅O₁₁ | 862.02 |
| 237 | 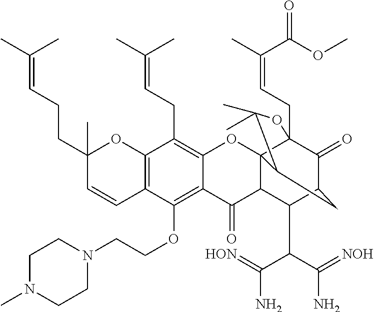 | C₄₉H₆₈N₆O₁₀ | 901.1 |
| 238 | 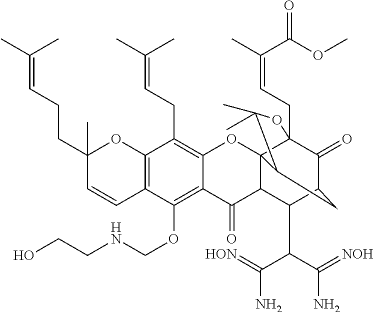 | C₄₅H₆₁N₅O₁₁ | 847.99 |
| 239 | 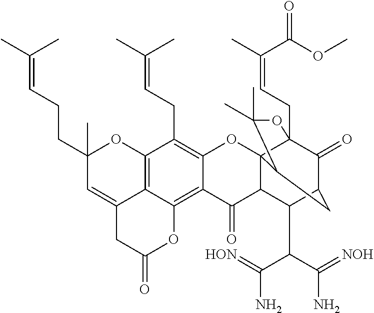 | C₄₄H₅₄N₄O₁₁ | 814.92 |
| 240 | 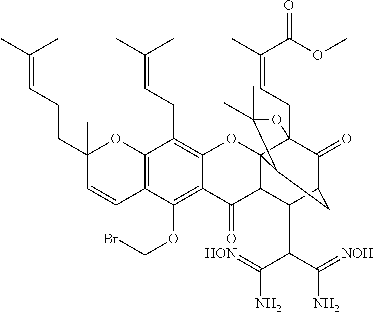 | C₄₃H₅₅BrN₄O₁₀ | 867.82 |
| 241 | 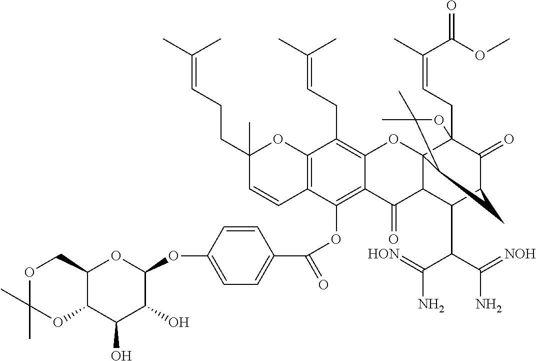 | C₅₈H₇₂N₄O₁₇ | 1097.21 |
| 242 | 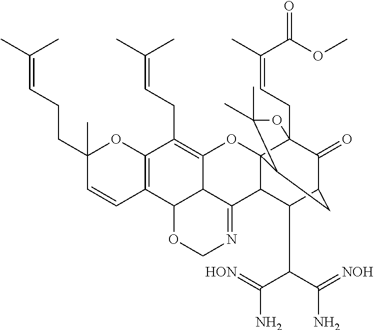 | C₄₃H₅₇N₅O₉ | 787.94 |
| 243 | 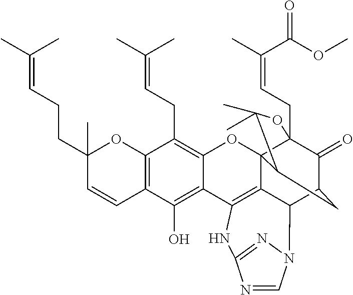 [SMILES: COC(=O)/C(C)=C\CC12OC(C)(C)C3CC(C1=O)C1C4=C(Nc5ncn1n5)c1c(O)c5c(c(CC=C(C)C)c1OC432)OC(C)(CCC=C(C)C)C=C5] | C₄₁H₄₈N₄O₇ | 708.84 |
| 244 | 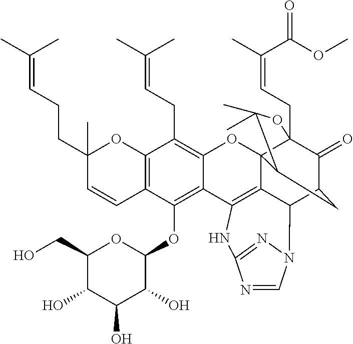 [SMILES: COC(=O)/C(C)=C\CC12OC(C)(C)C3CC(C1=O)C1C4=C(Nc5ncn1n5)c1c(O[C@@H]5O[C@H](CO)[C@@H](O)[C@H](O)[C@H]5O)c5c(c(CC=C(C)C)c1OC432)OC(C)(CCC=C(C)C)C=C5] | C₄₇H₅₈N₄O₁₂ | 870.98 |
| 245 | 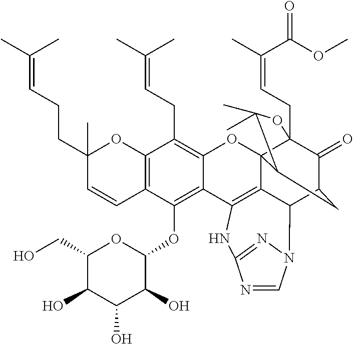 [SMILES: COC(=O)/C(C)=C\CC12OC(C)(C)C3CC(C1=O)C1C4=C(Nc5ncn1n5)c1c(O[C@H]5O[C@@H](CO)[C@H](O)[C@@H](O)[C@@H]5O)c5c(c(CC=C(C)C)c1OC432)OC(C)(CCC=C(C)C)C=C5] | C₄₇H₅₈N₄O₁₂ | 870.98 |
| 246 | 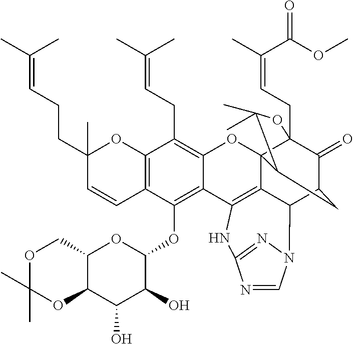 [SMILES: COC(=O)/C(C)=C\CC12OC(C)(C)C3CC(C1=O)C1C4=C(Nc5ncn1n5)c1c(O[C@H]5O[C@H]6COC(C)(C)O[C@@H]6[C@@H](O)[C@@H]5O)c5c(c(CC=C(C)C)c1OC432)OC(C)(CCC=C(C)C)C=C5] | C₅₀H₆₂N₄O₁₂ | 911.05 |
| 247 | 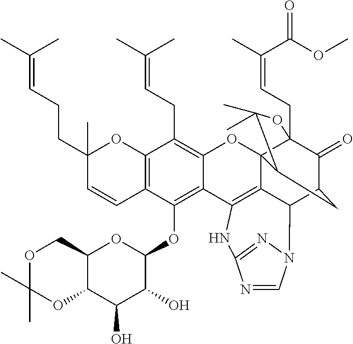 [SMILES: COC(=O)/C(C)=C\CC12OC(C)(C)C3CC(C1=O)C1C4=C(Nc5ncn1n5)c1c(O[C@@H]5O[C@@H]6COC(C)(C)O[C@H]6[C@H](O)[C@H]5O)c5c(c(CC=C(C)C)c1OC432)OC(C)(CCC=C(C)C)C=C5] | C₅₀H₆₂N₄O₁₂ | 911.05 |
| 248 | 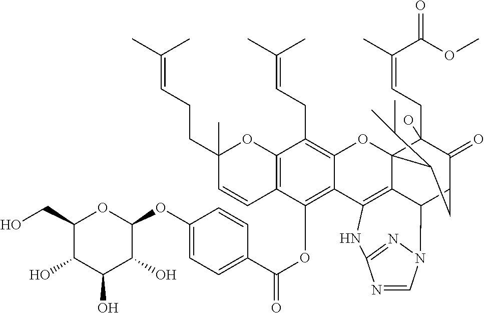 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2n2cnc(n2)N1)C=CC(C)(CCC=C(C)C)O4] | C₅₄H₆₂N₄O₁₄ | 991.09 |
| 249 | 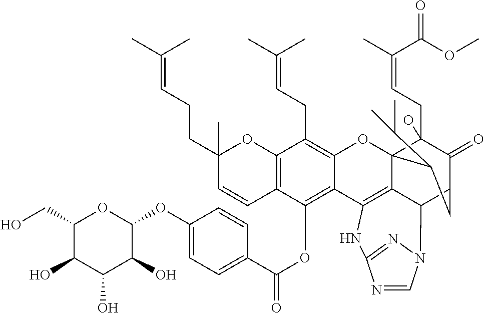 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2n2cnc(n2)N1)C=CC(C)(CCC=C(C)C)O4] | C₅₄H₆₂N₄O₁₄ | 991.09 |
| 250 | 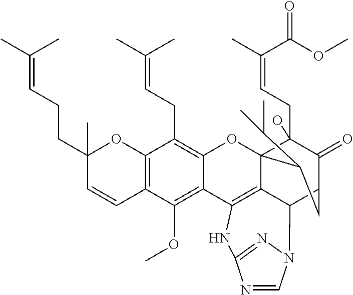 [SMILES: COC(=O)/C(C)=C\CC1(O)C(=O)C2CC(C(C)C)C13Oc1c(CC=C(C)C)c4c(c(OC)c1C1=C3C2n2cnc(n2)N1)C=CC(C)(CCC=C(C)C)O4] | C₄₂H₅₀N₄O₇ | 722.87 |
| 251 | 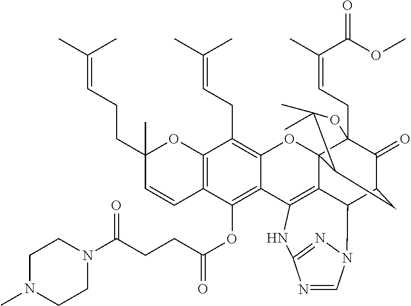 [SMILES: COC(=O)/C(C)=C\CC12OC(C)(C)C3CC(C1=O)C1C4=C(Nc5ncn1n5)c1c(OC(=O)CCC(=O)N5CCN(C)CC5)c5c(c(CC=C(C)C)c1OC432)OC(C)(CCC=C(C)C)C=C5] | C₅₀H₆₂N₆O₉ | 891.06 |
| 252 | 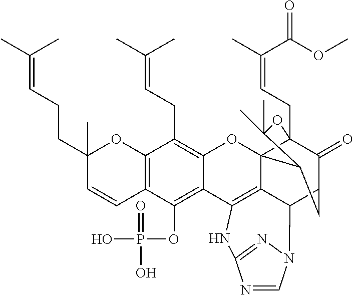 [SMILES: COC(=O)/C(C)=C\CC12OC(C)(C)C3CC(C1=O)C1C4=C(Nc5ncn1n5)c1c(OP(=O)(O)O)c5c(c(CC=C(C)C)c1OC432)OC(C)(CCC=C(C)C)C=C5] | C₄₁H₄₉N₄O₁₀P | 788.82 |
| 253 | 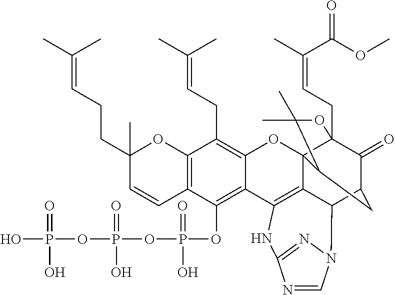 [SMILES: COC(=O)/C(C)=C\CC12OC(C)(C)C3CC(C1=O)C1C4=C(Nc5ncn1n5)c1c(OP(=O)(O)OP(=O)(O)OP(=O)(O)O)c5c(c(CC=C(C)C)c1OC432)OC(C)(CCC=C(C)C)C=C5] | C₄₁H₅₁N₄O₁₆P₃ | 948.78 |
| 254 | 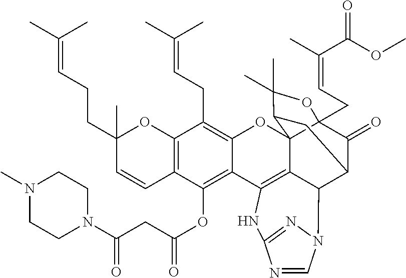 | C₄₉H₆₀N₆O₉ | 877.04 |
| 255 | 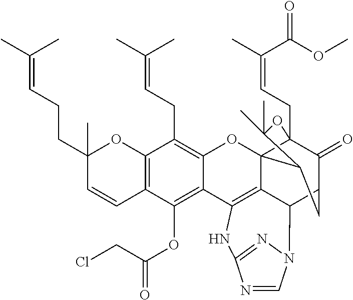 [SMILES: COC(=O)/C(C)=C\CC12OC(C)(C)C3CC(C1=O)C1C4=C(Nc5ncn1n5)c1c(OC(=O)CCl)c5c(c(CC=C(C)C)c1OC432)OC(C)(CCC=C(C)C)C=C5] | C₄₃H₄₉ClN₄O₈ | 785.32 |
| 256 | 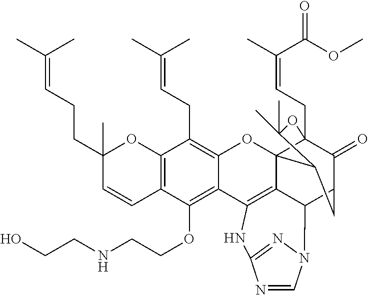 [SMILES: COC(=O)/C(C)=C\CC12OC(C)(C)C3CC(C1=O)C1C4=C(Nc5ncn1n5)c1c(OCCNCCO)c5c(c(CC=C(C)C)c1OC432)OC(C)(CCC=C(C)C)C=C5] | C₄₅H₅₇N₅O₈ | 795.96 |
| 257 | 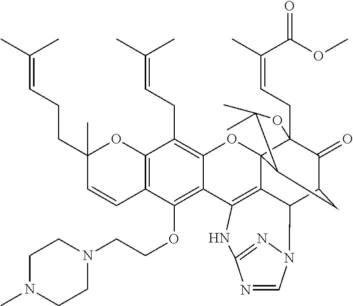 [SMILES: COC(=O)/C(C)=C\CC12OC(C)(C)C3CC(C1=O)C1C4=C(Nc5ncn1n5)c1c(OCCN5CCN(C)CC5)c5c(c(CC=C(C)C)c1OC432)OC(C)(CCC=C(C)C)C=C5] | C₄₈H₆₂N₆O₇ | 835.04 |
| 258 | 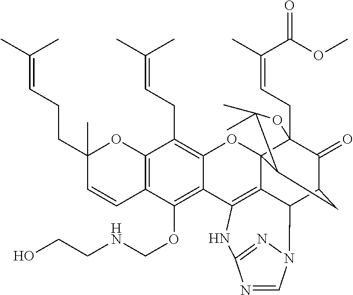 [SMILES: COC(=O)/C(C)=C\CC12OC(C)(C)C3CC(C1=O)C1C4=C(Nc5ncn1n5)c1c(OCNCCO)c5c(c(CC=C(C)C)c1OC432)OC(C)(CCC=C(C)C)C=C5] | C₄₄H₅₅N₅O₈ | 781.94 |
| 259 | 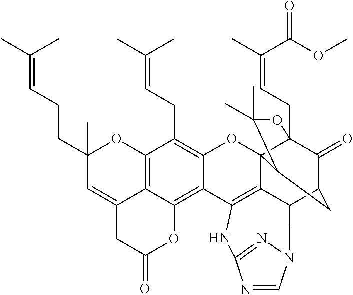 [SMILES: COC(=O)/C(C)=C\CC12OC(C)(C)C3CC(C1=O)C1C4=C(Nc5ncn1n5)c1c(c(CC=C(C)C)c5c6c1OC(=O)CC6=CC(C)(CCC=C(C)C)O5)OC432] | C₄₄H₅₀N₄O₈ | 762.89 |
| 260 | 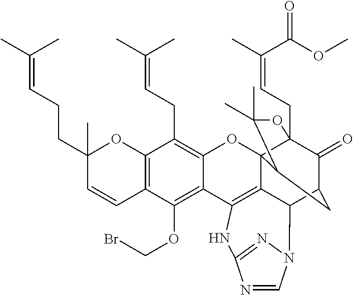 [SMILES: COC(=O)/C(C)=C\CC12OC(C)(C)C3CC(C1=O)C1C4=C(Nc5ncn1n5)c1c(OCBr)c5c(c(CC=C(C)C)c1OC432)OC(C)(CCC=C(C)C)C=C5] | C₄₂H₄₉BrN₄O₇ | 801.77 |
| 261 | 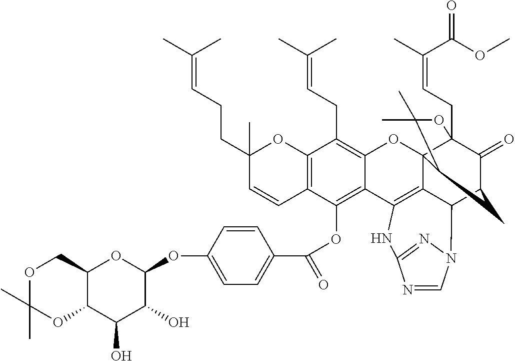 [SMILES: COC(=O)/C(C)=C\CC12OC(C)(C)[C@H]3C[C@H](C1=O)C1C4=C(Nc5ncn1n5)c1c(OC(=O)c5ccc(O[C@@H]6O[C@@H]7COC(C)(C)O[C@H]7[C@H](O)[C@H]6O)cc5)c5c(c(CC=C(C)C)c1OC432)OC(C)(CCC=C(C)C)C=C5] | C₅₇H₆₆N₄O₁₄ | 1031.15 |
| 262 | 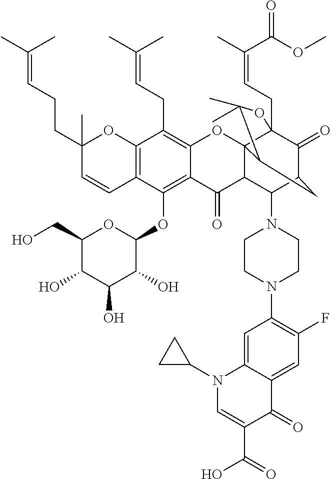 [SMILES: COC(=O)/C(C)=C\CC12OC(C)(C)C3CC(C1=O)C(N1CCN(c4cc5c(cc4F)c(=O)c(C(=O)O)cn5C4CC4)CC1)C1C(=O)c4c(O[C@@H]5O[C@H](CO)[C@@H](O)[C@H](O)[C@H]5O)c5c(c(CC=C(C)C)c4OC132)OC(C)(CCC=C(C)C)C=C5] | C₆₂H₇₄FN₃O₁₆ | 1136.26 |
| 263 | 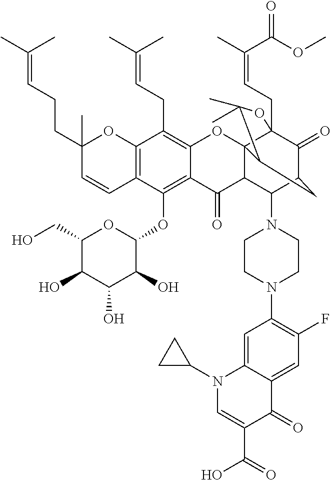 [SMILES: COC(=O)/C(C)=C\CC12OC(C)(C)C3CC(C1=O)C(N1CCN(c4cc5c(cc4F)c(=O)c(C(=O)O)cn5C4CC4)CC1)C1C(=O)c4c(O[C@H]5O[C@@H](CO)[C@H](O)[C@@H](O)[C@@H]5O)c5c(c(CC=C(C)C)c4OC132)OC(C)(CCC=C(C)C)C=C5] | C₆₂H₇₄FN₃O₁₆ | 1136.26 |
| 264 | 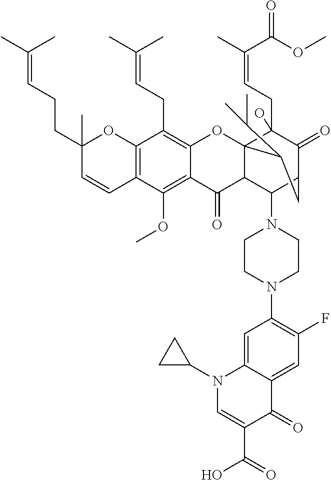 [SMILES: COC(=O)/C(C)=C\CC1(O)C(=O)C2CC(C(C)C)C13Oc1c(CC=C(C)C)c4c(c(OC)c1C(=O)C3C2N1CCN(c2cc3c(cc2F)c(=O)c(C(=O)O)cn3C2CC2)CC1)C=CC(C)(CCC=C(C)C)O4] | C₅₇H₆₆FN₃O₁₁ | 988.15 |
| 265 | 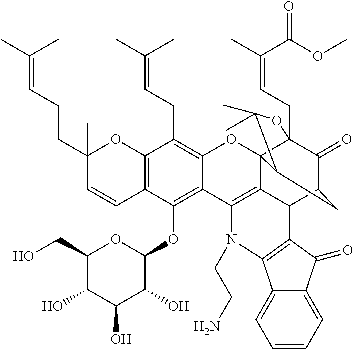 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(O[C@@H]3O[C@H](CO)[C@@H](O)[C@H](O)[C@H]3O)c14)C=CC(C)(CCC=C(C)C)O2] | C₅₅H₆₆N₂O₁₃ | 975.13 |
| 266 | 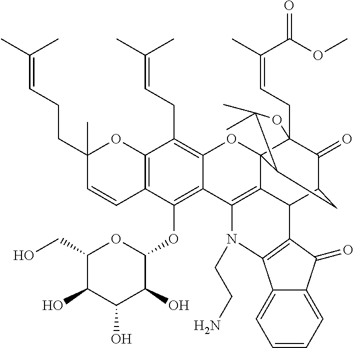 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(O[C@H]3O[C@@H](CO)[C@H](O)[C@@H](O)[C@@H]3O)c14)C=CC(C)(CCC=C(C)C)O2] | C₅₅H₆₆N₂O₁₃ | 975.13 |
| 267 | 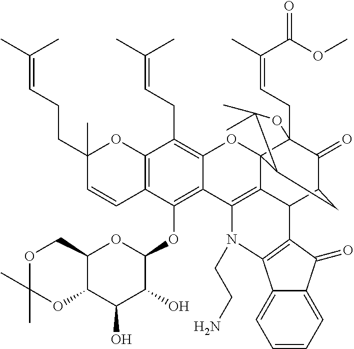 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(O[C@@H]3O[C@@H]5COC(C)(C)O[C@H]5[C@H](O)[C@H]3O)c14)C=CC(C)(CCC=C(C)C)O2] | C₅₉H₇₀N₂O₁₃ | 1015.19 |
| 268 | 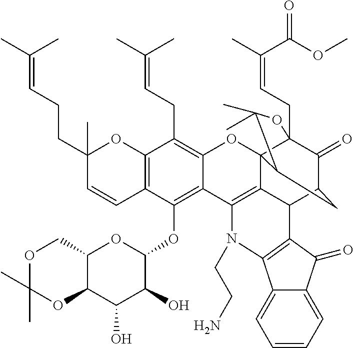 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(O[C@H]3O[C@H]5COC(C)(C)O[C@@H]5[C@@H](O)[C@@H]3O)c14)C=CC(C)(CCC=C(C)C)O2] | C₅₉H₇₀N₂O₁₃ | 1015.19 |
| 269 | 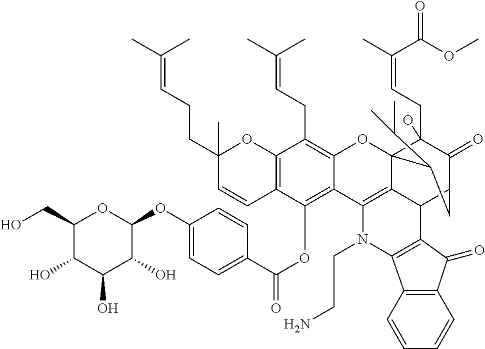 [SMILES: COC(=O)/C(C)=C\CC1(O)C(=O)C2CC(C(C)C)C13Oc1c(CC=C(C)C)c4c(c(OC(=O)c5ccc(O[C@@H]6O[C@H](CO)[C@@H](O)[C@H](O)[C@H]6O)cc5)c1C1=C3C2C2=C(c3ccccc3C2=O)N1CCN)C=CC(C)(CCC=C(C)C)O4] | C₆₃H₇₀N₂O₁₅ | 1095.23 |
| 270 | 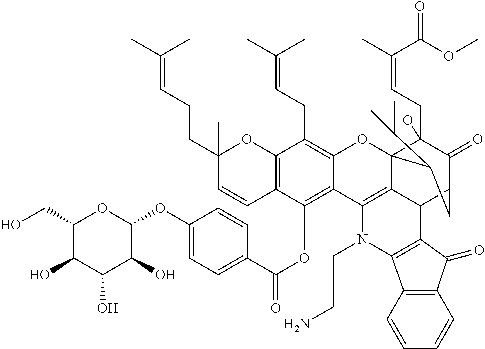 [SMILES: COC(=O)/C(C)=C\CC1(O)C(=O)C2CC(C(C)C)C13Oc1c(CC=C(C)C)c4c(c(OC(=O)c5ccc(O[C@H]6O[C@@H](CO)[C@H](O)[C@@H](O)[C@@H]6O)cc5)c1C1=C3C2C2=C(c3ccccc3C2=O)N1CCN)C=CC(C)(CCC=C(C)C)O4] | C₆₃H₇₀N₂O₁₅ | 1095.23 |
| 271 | 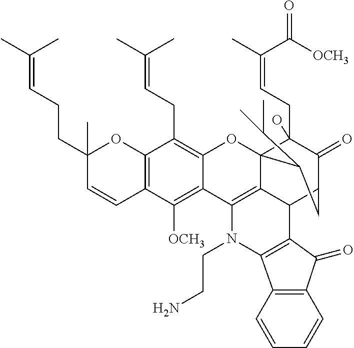 [SMILES: COC(=O)/C(C)=C\CC1(O)C(=O)C2CC(C(C)C)C13Oc1c(CC=C(C)C)c4c(c(OC)c1C1=C3C2C2=C(c3ccccc3C2=O)N1CCN)C=CC(C)(CCC=C(C)C)O4] | C₅₁H₅₈N₂O₈ | 827.01 |
| 272 | 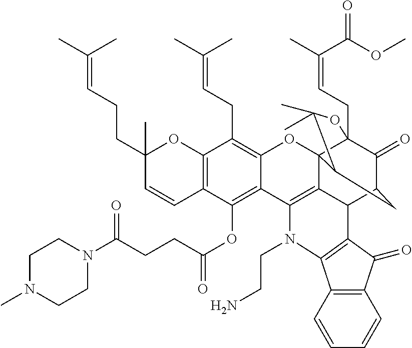 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OC(=O)CCC(=O)N3CCN(C)CC3)c14)C=CC(C)(CCC=C(C)C)O2] | C₅₉H₇₀N₄O₁₀ | 995.21 |
| 273 | 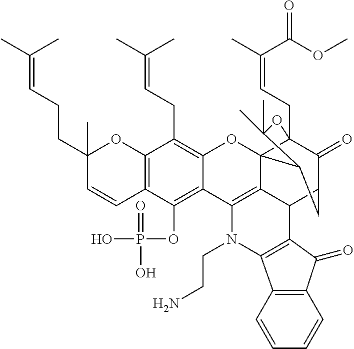 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OP(=O)(O)O)c14)C=CC(C)(CCC=C(C)C)O2] | C₅₀H₅₇N₂O₁₁P | 892.97 |
| 274 | 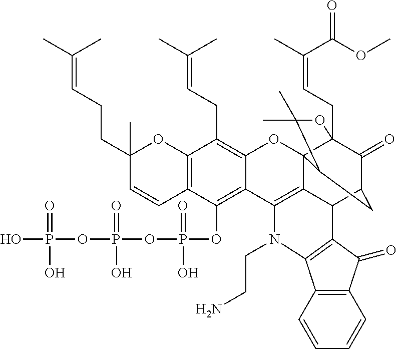 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OP(=O)(O)OP(=O)(O)OP(=O)(O)O)c14)C=CC(C)(CCC=C(C)C)O2] | C₅₀H₅₉N₂O₁₇P₃ | 1052.93 |
| 275 | 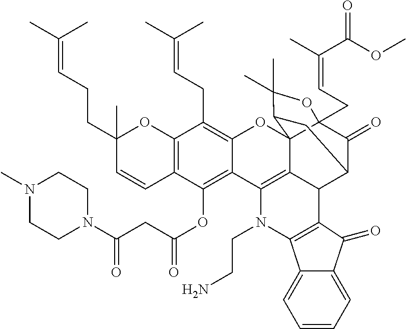 | C₅₈H₆₈N₄O₁₀ | 981.18 |
| 276 | 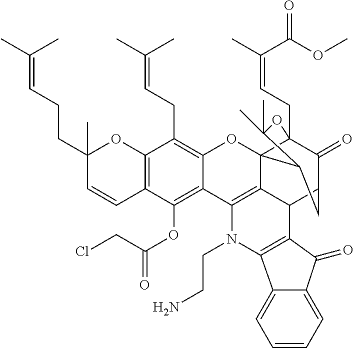 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OC(=O)CCl)c14)C=CC(C)(CCC=C(C)C)O2] | C₅₂H₅₇ClN₂O₉ | 889.47 |
| 277 | 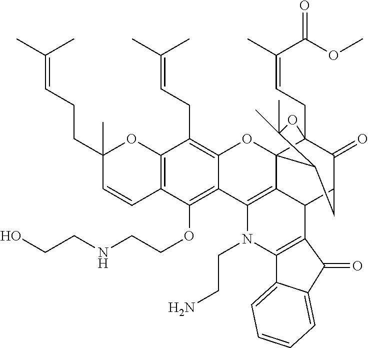 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OCCNCCO)c14)C=CC(C)(CCC=C(C)C)O2] | C₅₄H₆₅N₃O₉ | 900.11 |
| 278 | 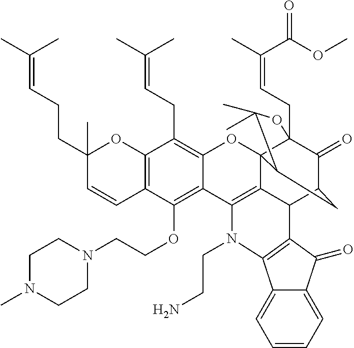 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OCCN3CCN(C)CC3)c14)C=CC(C)(CCC=C(C)C)O2] | C₅₇H₇₀N₄O₈ | 939.19 |
| 279 | 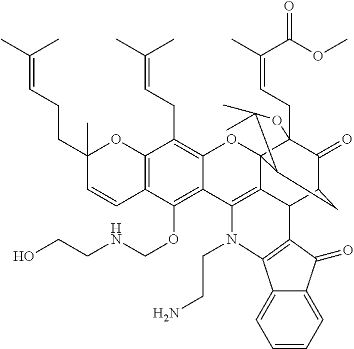 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OCNCCO)c14)C=CC(C)(CCC=C(C)C)O2] | C₅₃H₆₃N₃O₉ | 886.08 |
| 280 | 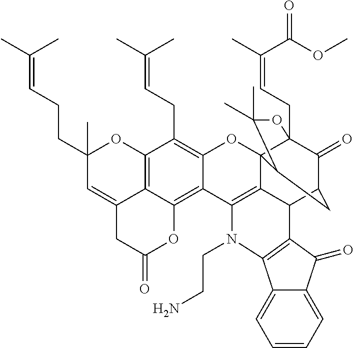 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c3c(c14)OC(=O)CC3=CC(C)(CCC=C(C)C)O2] | C₅₂H₅₆N₂O₉ | 853.01 |
| 281 | 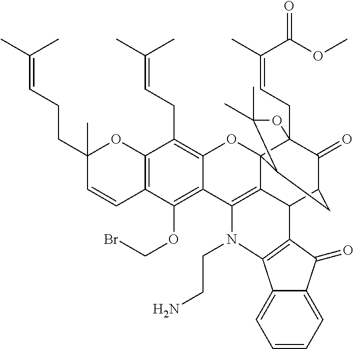 [SMILES: COC(=O)/C(C)=C\CC12OC(C)(C)C3CC(C1=O)C1C4=C(c5ccccc5C4=O)N(CCN)C4=C1C32Oc1c(CC=C(C)C)c2c(c(OCBr)c14)C=CC(C)(CCC=C(C)C)O2] | C₅₃H₅₇BrN₂O₈ | 905.91 |
| 282 | 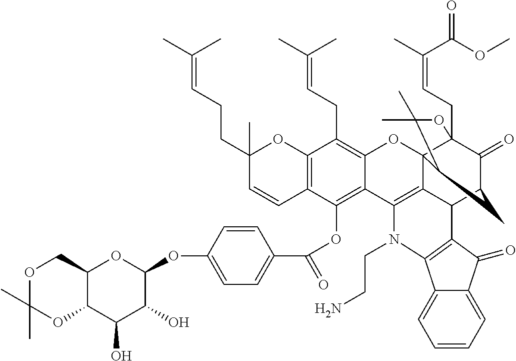 | C₆₆H₇₄N₂O₁₅ | 1135.3 |
| 283 | 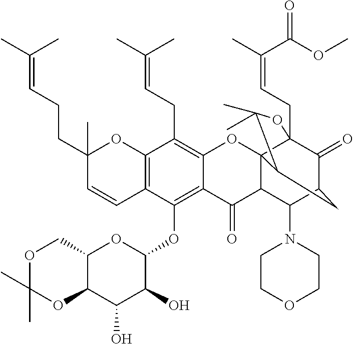 [SMILES: COC(=O)/C(C)=C\CC12OC(C)(C)C3CC(C1=O)C(N1CCOCC1)C1C(=O)c4c(O[C@H]5O[C@H]6COC(C)(C)O[C@@H]6[C@@H](O)[C@@H]5O)c5c(c(CC=C(C)C)c4OC132)OC(C)(CCC=C(C)C)C=C5] | C₅₂H₆₉NO₁₄ | 932.1 |
| 284 | 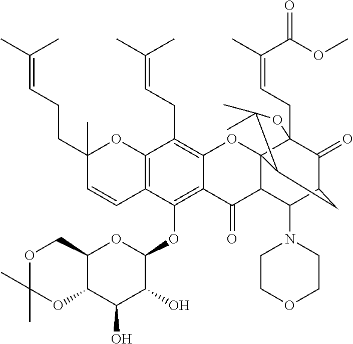 [SMILES: COC(=O)/C(C)=C\CC12OC(C)(C)C3CC(C1=O)C(N1CCOCC1)C1C(=O)c4c(O[C@@H]5O[C@@H]6COC(C)(C)O[C@H]6[C@H](O)[C@H]5O)c5c(c(CC=C(C)C)c4OC132)OC(C)(CCC=C(C)C)C=C5] | C₅₂H₆₉NO₁₄ | 932.1 |
| 285 | 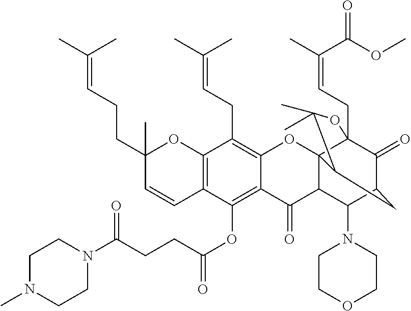 [SMILES: COC(=O)/C(C)=C\CC12OC(C)(C)C3CC(C1=O)C(N1CCOCC1)C1C(=O)c4c(OC(=O)CCC(=O)N5CCN(C)CC5)c5c(c(CC=C(C)C)c4OC132)OC(C)(CCC=C(C)C)C=C5] | C₅₂H₆₉N₃O₁₁ | 912.12 |
| 286 | 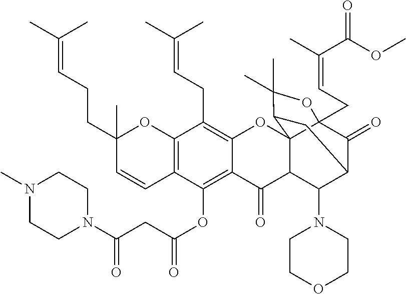 | C₅₁H₆₇N₃O₁₁ | 898.09 |
| 287 | 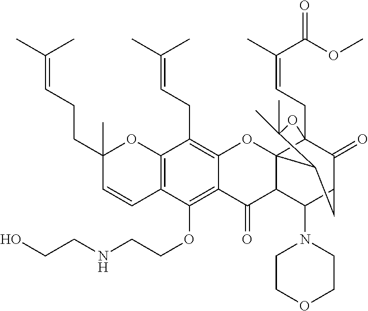 [SMILES: COC(=O)/C(C)=C\CC12OC(C)(C)C3CC(C1=O)C(N1CCOCC1)C1C(=O)c4c(OCCNCCO)c5c(c(CC=C(C)C)c4OC132)OC(C)(CCC=C(C)C)C=C5] | C₄₇H₆₄N₂O₁₀ | 817.02 |
| 288 | 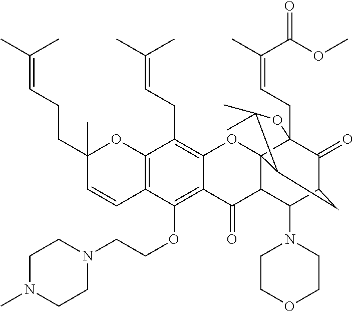 [SMILES: COC(=O)/C(C)=C\CC12OC(C)(C)C3CC(C1=O)C(N1CCOCC1)C1C(=O)c4c(OCCN5CCN(C)CC5)c5c(c(CC=C(C)C)c4OC132)OC(C)(CCC=C(C)C)C=C5] | C₅₀H₆₉N₃O₉ | 856.1 |
| 289 | 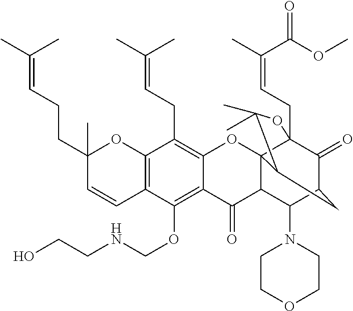 [SMILES: COC(=O)/C(C)=C\CC12OC(C)(C)C3CC(C1=O)C(N1CCOCC1)C1C(=O)c4c(OCNCCO)c5c(c(CC=C(C)C)c4OC132)OC(C)(CCC=C(C)C)C=C5] | C₄₆H₆₂N₂O₁₀ | 802.99 |
| 290 | 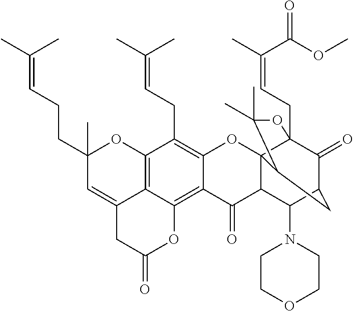 [SMILES: COC(=O)/C(C)=C\CC12OC(C)(C)C3CC(C1=O)C(N1CCOCC1)C1C(=O)c4c(c(CC=C(C)C)c5c6c4OC(=O)CC6=CC(C)(CCC=C(C)C)O5)OC132] | C₄₅H₅₅NO₁₀ | 769.92 |
| 291 | 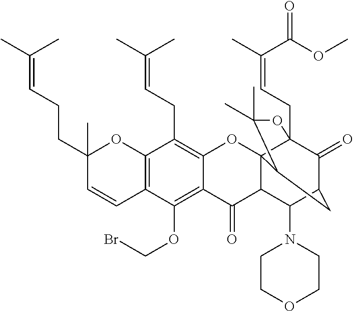 [SMILES: COC(=O)/C(C)=C\CC12OC(C)(C)C3CC(C1=O)C(N1CCOCC1)C1C(=O)c4c(OCBr)c5c(c(CC=C(C)C)c4OC132)OC(C)(CCC=C(C)C)C=C5] | C₄₄H₅₆BrNO₉ | 822.82 |
| 292 | 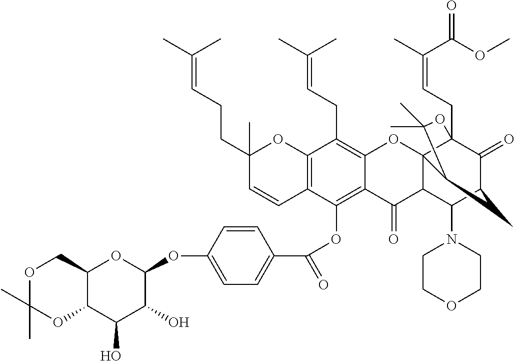 [SMILES: COC(=O)/C(C)=C\CC12OC(C)(C)[C@H]3C[C@H](C1=O)C(N1CCOCC1)C1C(=O)c4c(OC(=O)c5ccc(O[C@@H]6O[C@@H]7COC(C)(C)O[C@H]7[C@H](O)[C@H]6O)cc5)c5c(c(CC=C(C)C)c4OC132)OC(C)(CCC=C(C)C)C=C5] | C₅₉H₇₃NO₁₆ | 1052.21 |
| 293 | 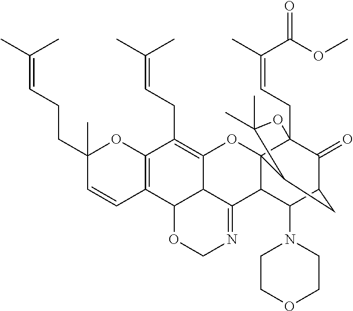 [SMILES: COC(=O)/C(C)=C\CC12OC(C)(C)C3CC(C1=O)C(N1CCOCC1)C1C4=NCOC5C6=C(OC(C)(CCC=C(C)C)C=C6)C(CC=C(C)C)=C(OC132)C45] | C₄₄H₅₈N₂O₈ | 742.94 |
| 294 | 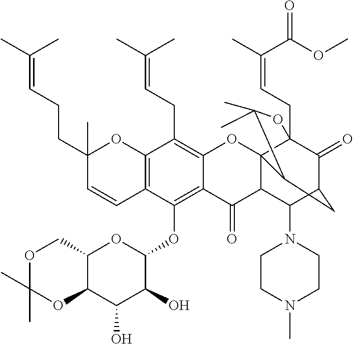 [SMILES: COC(=O)/C(C)=C\CC12OC(C)(C)C3CC(C1=O)C(N1CCN(C)CC1)C1C(=O)c4c(O[C@H]5O[C@H]6COC(C)(C)O[C@@H]6[C@@H](O)[C@@H]5O)c5c(c(CC=C(C)C)c4OC132)OC(C)(CCC=C(C)C)C=C5] | C₅₃H₇₂N₂O₁₃ | 945.14 |
| 295 | 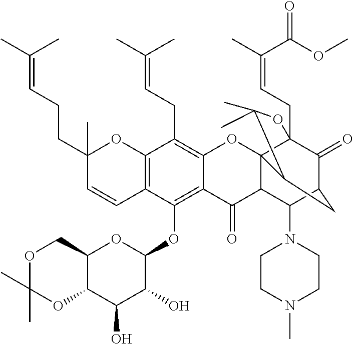 [SMILES: COC(=O)/C(C)=C\CC12OC(C)(C)C3CC(C1=O)C(N1CCN(C)CC1)C1C(=O)c4c(O[C@@H]5O[C@@H]6COC(C)(C)O[C@H]6[C@H](O)[C@H]5O)c5c(c(CC=C(C)C)c4OC132)OC(C)(CCC=C(C)C)C=C5] | C₅₃H₇₂N₂O₁₃ | 945.14 |
| 296 | 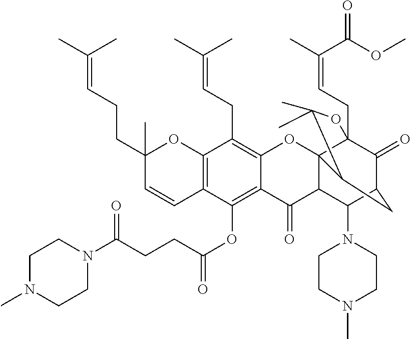 [SMILES: COC(=O)/C(C)=C\CC12OC(C)(C)C3CC(C1=O)C(N1CCN(C)CC1)C1C(=O)c4c(OC(=O)CCC(=O)N5CCN(C)CC5)c5c(c(CC=C(C)C)c4OC132)OC(C)(CCC=C(C)C)C=C5] | C₅₃H₇₂N₄O₁₀ | 925.16 |
| 297 | 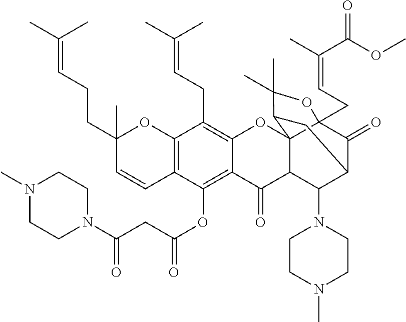 | C₅₂H₇₀N₄O₁₀ | 911.13 |
| 298 | 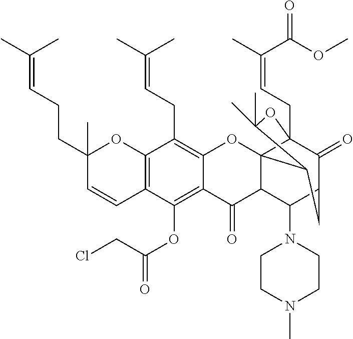 [SMILES: COC(=O)/C(C)=C\CC12OC(C)(C)C3CC(C1=O)C(N1CCN(C)CC1)C1C(=O)c4c(OC(=O)CCl)c5c(c(CC=C(C)C)c4OC132)OC(C)(CCC=C(C)C)C=C5] | C₄₆H₅₉ClN₂O₉ | 819.42 |
| 299 | 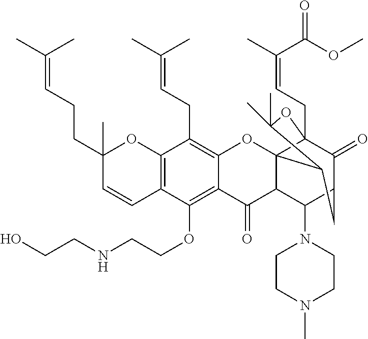 [SMILES: COC(=O)/C(C)=C\CC12OC(C)(C)C3CC(C1=O)C(N1CCN(C)CC1)C1C(=O)c4c(OCCNCCO)c5c(c(CC=C(C)C)c4OC132)OC(C)(CCC=C(C)C)C=C5] | C₄₈H₆₇N₃O₉ | 830.06 |
| 300 | 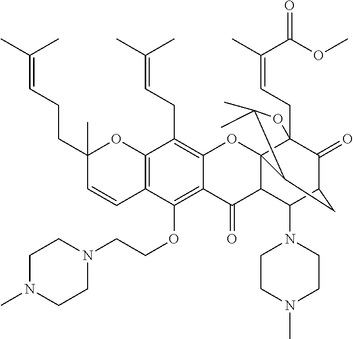 [SMILES: COC(=O)/C(C)=C\CC12OC(C)(C)C3CC(C1=O)C(N1CCN(C)CC1)C1C(=O)c4c(OCCN5CCN(C)CC5)c5c(c(CC=C(C)C)c4OC132)OC(C)(CCC=C(C)C)C=C5] | C₅₁H₇₂N₄O₈ | 869.14 |
| 301 | 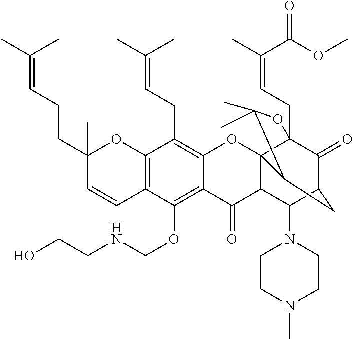 [SMILES: COC(=O)/C(C)=C\CC12OC(C)(C)C3CC(C1=O)C(N1CCN(C)CC1)C1C(=O)c4c(OCNCCO)c5c(c(CC=C(C)C)c4OC132)OC(C)(CCC=C(C)C)C=C5] | C₄₇H₆₅N₃O₉ | 816.03 |
| 302 | 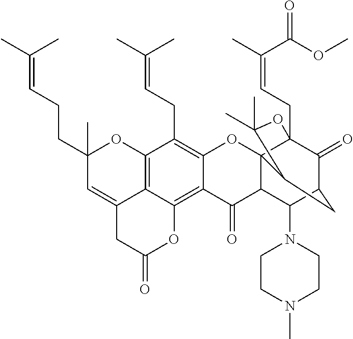 [SMILES: COC(=O)/C(C)=C\CC12OC(C)(C)C3CC(C1=O)C(N1CCN(C)CC1)C1C(=O)c4c(c(CC=C(C)C)c5c6c4OC(=O)CC6=CC(C)(CCC=C(C)C)O5)OC132] | C₄₆H₅₈N₂O₉ | 782.96 |
| 303 | 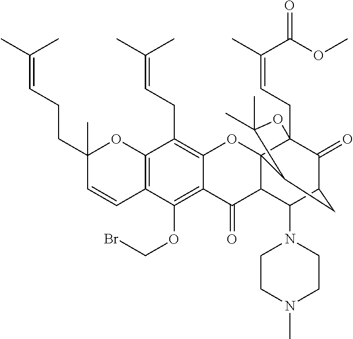 [SMILES: COC(=O)/C(C)=C\CC12OC(C)(C)C3CC(C1=O)C(N1CCN(C)CC1)C1C(=O)c4c(OCBr)c5c(c(CC=C(C)C)c4OC132)OC(C)(CCC=C(C)C)C=C5] | C₄₅H₅₉BrN₂O₈ | 835.86 |
| 304 | 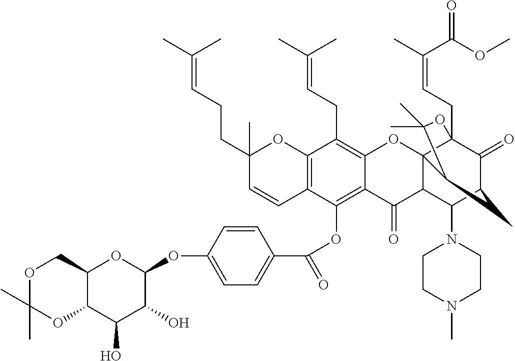 [SMILES: COC(=O)/C(C)=C\CC12OC(C)(C)[C@H]3C[C@H](C1=O)C(N1CCN(C)CC1)C1C(=O)c4c(OC(=O)c5ccc(O[C@@H]6O[C@@H]7COC(C)(C)O[C@H]7[C@H](O)[C@H]6O)cc5)c5c(c(CC=C(C)C)c4OC132)OC(C)(CCC=C(C)C)C=C5] | C₆₀H₇₆N₂O₁₅ | 1065.25 |
| 305 | 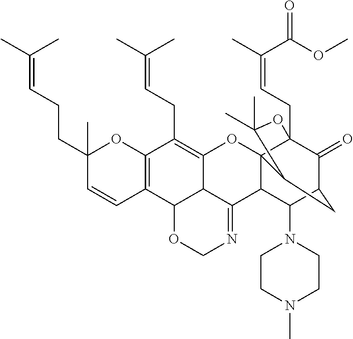 [SMILES: COC(=O)/C(C)=C\CC12OC(C)(C)C3CC(C1=O)C(N1CCN(C)CC1)C1C4=NCOC5C6=C(OC(C)(CCC=C(C)C)C=C6)C(CC=C(C)C)=C(OC132)C45] | C₄₅H₆₁N₃O₇ | 755.98 |
| 306 | 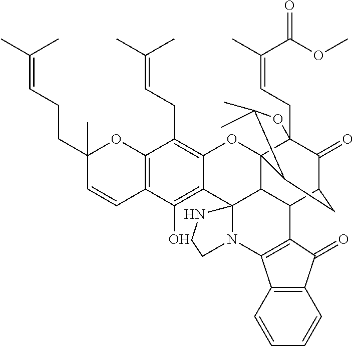 [SMILES: COC(=O)/C(C)=C\CC12OC(C)(C)C3CC(C1=O)C1C4=C(c5ccccc5C4=O)N4CCNC45c4c(O)c6c(c(CC=C(C)C)c4OC32C15)OC(C)(CCC=C(C)C)C=C6] | C₅₀H₅₆N₂O₈ | 812.99 |
| 307 | 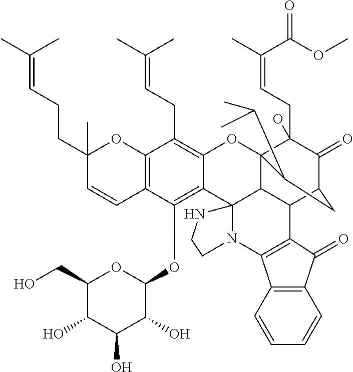 [SMILES: COC(=O)/C(C)=C\CC1(O)C(=O)C2CC(C(C)C)C13Oc1c(CC=C(C)C)c4c(c(O[C@@H]5O[C@H](CO)[C@@H](O)[C@H](O)[C@H]5O)c1C15NCCN1C1=C(C(=O)c6ccccc61)C2C53)C=CC(C)(CCC=C(C)C)O4] | C₅₆H₆₆N₂O₁₃ | 975.13 |
| 308 | 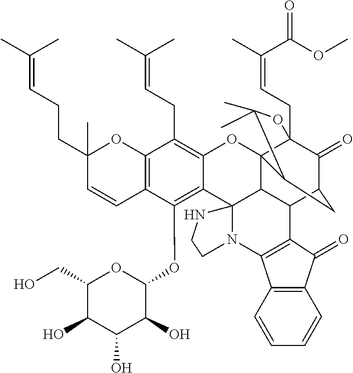 [SMILES: COC(=O)/C(C)=C\CC12OC(C)(C)C3CC(C1=O)C1C4=C(c5ccccc5C4=O)N4CCNC45c4c(O[C@H]6O[C@@H](CO)[C@H](O)[C@@H](O)[C@@H]6O)c6c(c(CC=C(C)C)c4OC32C15)OC(C)(CCC=C(C)C)C=C6] | C₅₆H₆₆N₂O₁₃ | 975.13 |
| 309 | 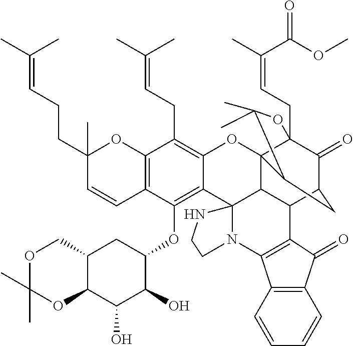 [SMILES: COC(=O)/C(C)=C\CC12OC(C)(C)C3CC(C1=O)C1C4=C(c5ccccc5C4=O)N4CCNC45c4c(O[C@H]6C[C@H]7COC(C)(C)O[C@@H]7[C@@H](O)[C@@H]6O)c6c(c(CC=C(C)C)c4OC32C15)OC(C)(CCC=C(C)C)C=C6] | C₆₀H₇₂N₂O₁₂ | 1013.22 |
| 310 | 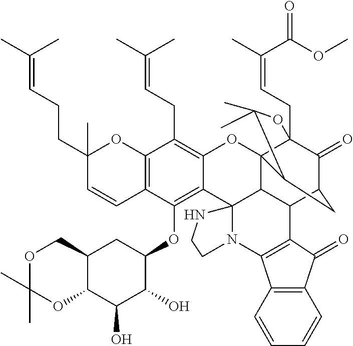 [SMILES: COC(=O)/C(C)=C\CC12OC(C)(C)C3CC(C1=O)C1C4=C(c5ccccc5C4=O)N4CCNC45c4c(O[C@@H]6C[C@@H]7COC(C)(C)O[C@H]7[C@H](O)[C@H]6O)c6c(c(CC=C(C)C)c4OC32C15)OC(C)(CCC=C(C)C)C=C6] | C₆₀H₇₂N₂O₁₂ | 1013.22 |
| 311 | 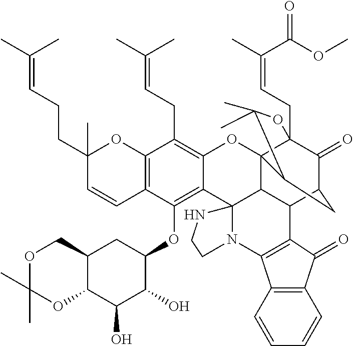 [SMILES: COC(=O)/C(C)=C\CC12OC(C)(C)C3CC(C1=O)C1C4=C(c5ccccc5C4=O)N4CCNC45c4c(O[C@@H]6C[C@@H]7COC(C)(C)O[C@H]7[C@H](O)[C@H]6O)c6c(c(CC=C(C)C)c4OC32C15)OC(C)(CCC=C(C)C)C=C6] | C₆₃H₇₀N₂O₁₅ | 1095.23 |
| 312 | 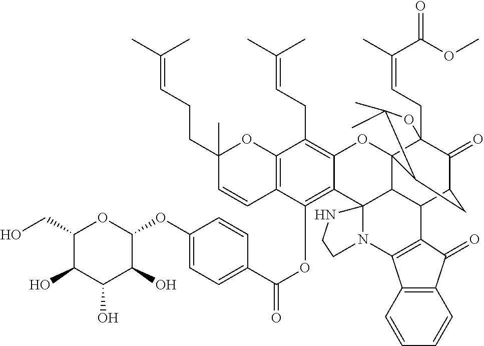 [SMILES: COC(=O)/C(C)=C\CC12OC(C)(C)C3CC(C1=O)C1C4=C(c5ccccc5C4=O)N4CCNC45c4c(OC(=O)c6ccc(O[C@H]7O[C@@H](CO)[C@H](O)[C@@H](O)[C@@H]7O)cc6)c6c(c(CC=C(C)C)c4OC32C15)OC(C)(CCC=C(C)C)C=C6] | C₆₃H₇₀N₂O₁₅ | 1095.23 |
| 313 | 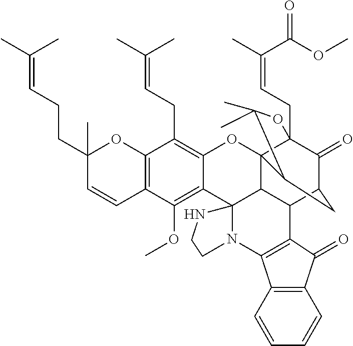 [SMILES: COC(=O)/C(C)=C\CC12OC(C)(C)C3CC(C1=O)C1C4=C(c5ccccc5C4=O)N4CCNC45c4c(OC)c6c(c(CC=C(C)C)c4OC32C15)OC(C)(CCC=C(C)C)C=C6] | C₅₁H₅₈N₂O₈ | 827.01 |
| 314 | 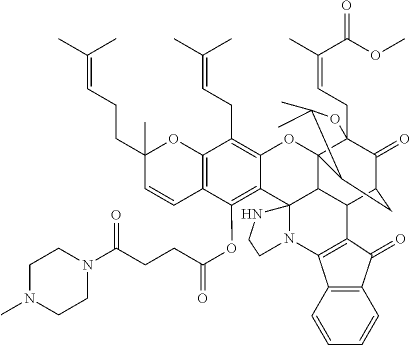 [SMILES: COC(=O)/C(C)=C\CC12OC(C)(C)C3CC(C1=O)C1C4=C(c5ccccc5C4=O)N4CCNC45c4c(OC(=O)CCC(=O)N6CCN(C)CC6)c6c(c(CC=C(C)C)c4OC32C15)OC(C)(CCC=C(C)C)C=C6] | C₅₉H₇₀N₂O₁₀ | 995.21 |
| 315 | 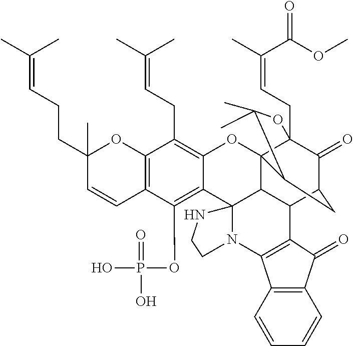 [SMILES: COC(=O)/C(C)=C\CC12OC(C)(C)C3CC(C1=O)C1C4=C(c5ccccc5C4=O)N4CCNC45c4c(OP(=O)(O)O)c6c(c(CC=C(C)C)c4OC32C15)OC(C)(CCC=C(C)C)C=C6] | C₅₀H₅₇N₂O₁₁P | 892.97 |
| 316 | 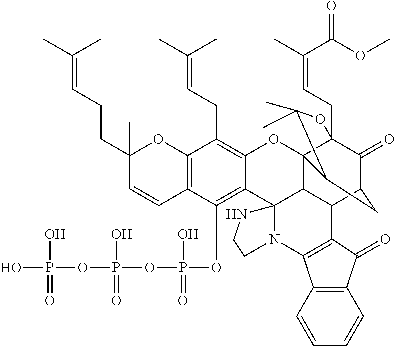 [SMILES: COC(=O)/C(C)=C\CC12OC(C)(C)C3CC(C1=O)C1C4=C(c5ccccc5C4=O)N4CCNC45c4c(OP(=O)(O)OP(=O)(O)OP(=O)(O)O)c6c(c(CC=C(C)C)c4OC32C15)OC(C)(CCC=C(C)C)C=C6] | C₅₀H₅₉N₂O₁₇P₃ | 1052.93 |
| 317 | 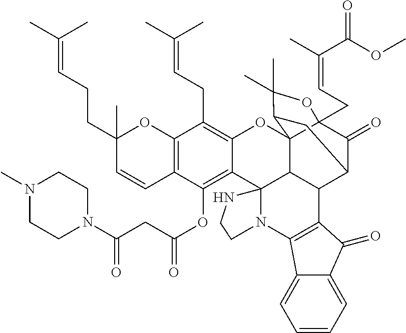 | C₅₈H₆₈N₄O₁₀ | 981.18 |
| 318 | 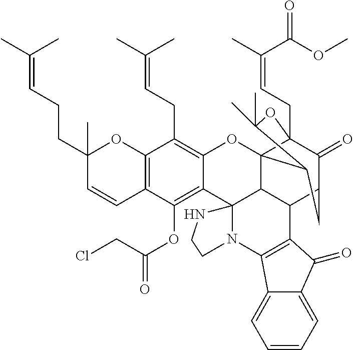 [SMILES: COC(=O)/C(C)=C\CC12OC(C)(C)C3CC(C1=O)C1C4=C(c5ccccc5C4=O)N4CCNC45c4c(OC(=O)CCl)c6c(c(CC=C(C)C)c4OC32C15)OC(C)(CCC=C(C)C)C=C6] | C₅₂H₅₇ClN₂O₉ | 889.47 |
| 319 | 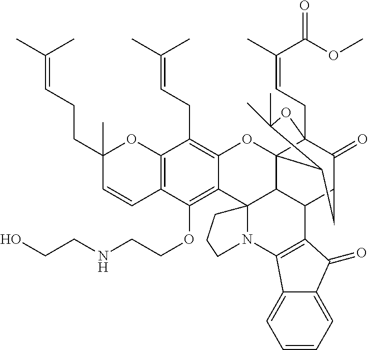 [SMILES: COC(=O)/C(C)=C\CC12OC(C)(C)C3CC(C1=O)C1C4=C(c5ccccc5C4=O)N4CCCC45c4c(OCCNCCO)c6c(c(CC=C(C)C)c4OC32C15)OC(C)(CCC=C(C)C)C=C6] | C₅₅H₆₆N₂O₉ | 899.12 |
| 320 | 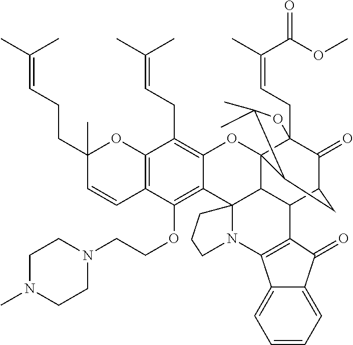 [SMILES: COC(=O)/C(C)=C\CC12OC(C)(C)C3CC(C1=O)C1C4=C(c5ccccc5C4=O)N4CCCC45c4c(OCCN6CCN(C)CC6)c6c(c(CC=C(C)C)c4OC32C15)OC(C)(CCC=C(C)C)C=C6] | C₅₈H₇₁N₃O₈ | 938.2 |
| 321 | 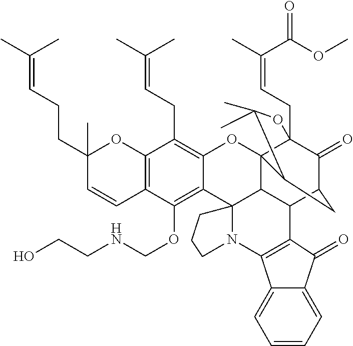 [SMILES: COC(=O)/C(C)=C\CC12OC(C)(C)C3CC(C1=O)C1C4=C(c5ccccc5C4=O)N4CCCC45c4c(OCNCCO)c6c(c(CC=C(C)C)c4OC32C15)OC(C)(CCC=C(C)C)C=C6] | C₅₄H₆₄N₂O₉ | 885.09 |
| 322 | 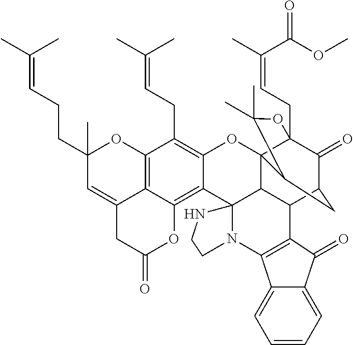 [SMILES: COC(=O)/C(C)=C\CC12OC(C)(C)C3CC(C1=O)C1C4=C(c5ccccc5C4=O)N4CCNC45c4c(c(CC=C(C)C)c6c7c4OC(=O)CC7=CC(C)(CCC=C(C)C)O6)OC32C15] | C₅₂H₅₆N₂O₉ | 853.01 |
| 323 | 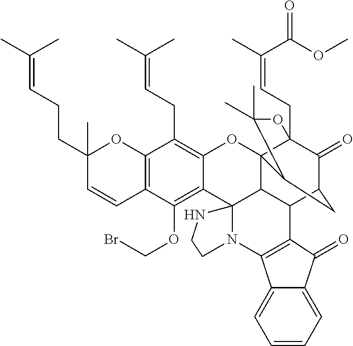 [SMILES: COC(=O)/C(C)=C\CC12OC(C)(C)C3CC(C1=O)C1C4=C(c5ccccc5C4=O)N4CCNC45c4c(OCBr)c6c(c(CC=C(C)C)c4OC32C15)OC(C)(CCC=C(C)C)C=C6] | C₅₁H₅₇BrN₂O₈ | 905.91 |
| 324 | 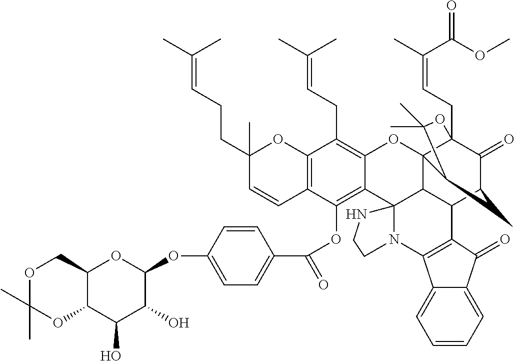 [SMILES: COC(=O)/C(C)=C\CC12OC(C)(C)[C@H]3C[C@H](C1=O)C1C4=C(c5ccccc5C4=O)N4CCNC45c4c(OC(=O)c6ccc(O[C@@H]7O[C@@H]8COC(C)(C)O[C@H]8[C@H](O)[C@H]7O)cc6)c6c(c(CC=C(C)C)c4OC32C15)OC(C)(CCC=C(C)C)C=C6] | C₆₆H₇₄N₂O₁₅ | 1135.3 |
| 325 | 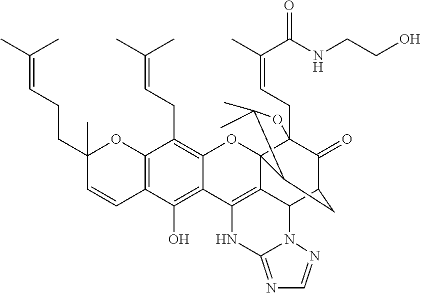 [SMILES: CC(C)=CCCC1(C)C=Cc2c(O)c3c(c(CC=C(C)C)c2O1)OC12C4=C3Nc3ncnn3C4C3CC1C(C)(C)OC2(C/C=C(/C)C(=O)NCCO)C3=O] | C₄₂H₅₁N₅O₇ | 737.88 |
| 326 | 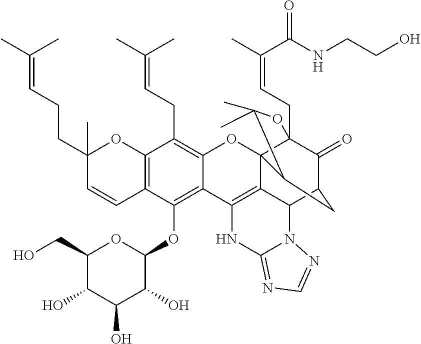 [SMILES: CC(C)=CCCC1(C)C=Cc2c(c(CC=C(C)C)c3c(c2O[C@@H]2O[C@H](CO)[C@@H](O)[C@H](O)[C@H]2O)C2=C4C(C5CC6C(C)(C)OC(C/C=C(/C)C(=O)NCCO)(C5=O)C46O3)n3ncnc3N2)O1] | C₄₈H₆₁N₅O₁₂ | 900.02 |
| 327 | 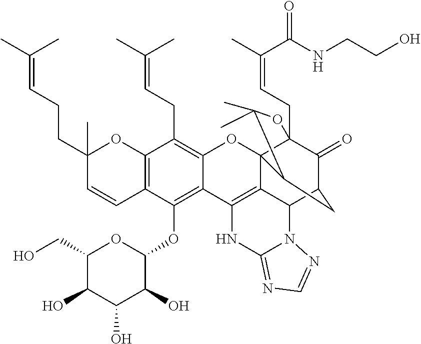 [SMILES: CC(C)=CCCC1(C)C=Cc2c(c(CC=C(C)C)c3c(c2O[C@H]2O[C@@H](CO)[C@H](O)[C@@H](O)[C@@H]2O)C2=C4C(C5CC6C(C)(C)OC(C/C=C(/C)C(=O)NCCO)(C5=O)C46O3)n3ncnc3N2)O1] | C₄₈H₆₁N₅O₁₂ | 900.02 |
| 328 | 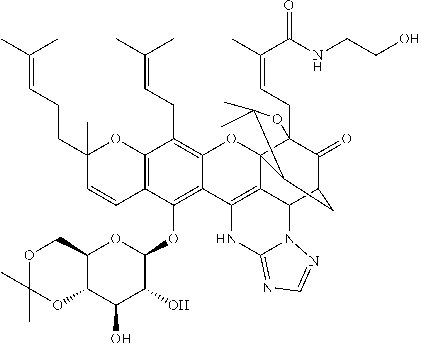 [SMILES: CC(C)=CCCC1(C)C=Cc2c(c(CC=C(C)C)c3c(c2O[C@@H]2O[C@@H]4COC(C)(C)O[C@H]4[C@H](O)[C@H]2O)C2=C4C(C5CC6C(C)(C)OC(C/C=C(/C)C(=O)NCCO)(C5=O)C46O3)n3ncnc3N2)O1] | C₅₁H₆₅N₅O₁₂ | 940.09 |
| 329 | 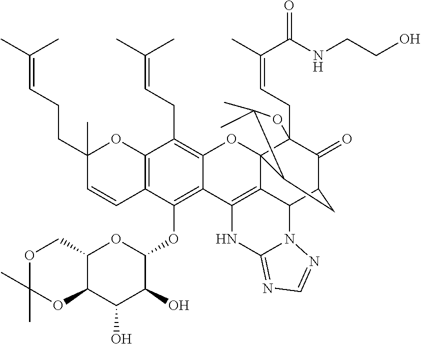 [SMILES: CC(C)=CCCC1(C)C=Cc2c(c(CC=C(C)C)c3c(c2O[C@H]2O[C@H]4COC(C)(C)O[C@@H]4[C@@H](O)[C@@H]2O)C2=C4C(C5CC6C(C)(C)OC(C/C=C(/C)C(=O)NCCO)(C5=O)C46O3)n3ncnc3N2)O1] | C₅₁H₆₅N₅O₁₂ | 940.09 |
| 330 | 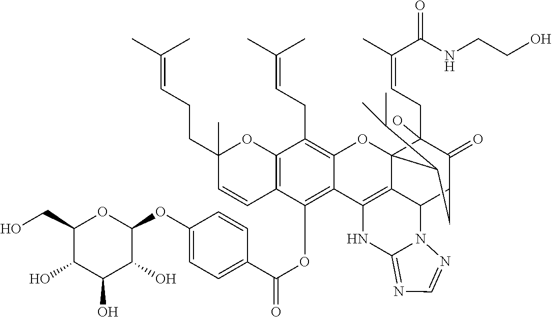 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C2=C4C(C5CC(C(C)C)C4(O3)C(O)(C/C=C(/C)C(=O)NCCO)C5=O)n3ncnc3N2)O1] | C₅₅H₆₅N₅O₁₄ | 1020.13 |
| 331 | 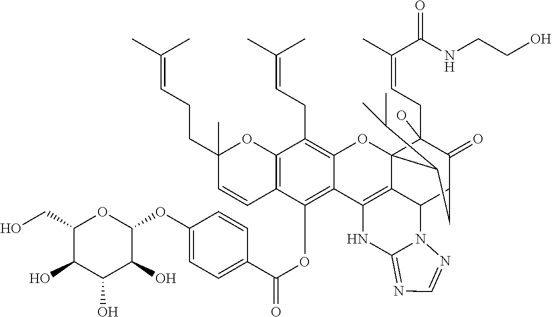 [SMILES: CC(C)=CCCC1(C)C=Cc2c(c(CC=C(C)C)c3c(c2OC(=O)c2ccc(O[C@H]4O[C@@H](CO)[C@H](O)[C@@H](O)[C@@H]4O)cc2)C2=C4C(C5CC(C(C)C)C4(O3)C(O)(C/C=C(/C)C(=O)NCCO)C5=O)n3ncnc3N2)O1] | C₅₅H₆₅N₅O₁₄ | 1020.13 |
| 332 | 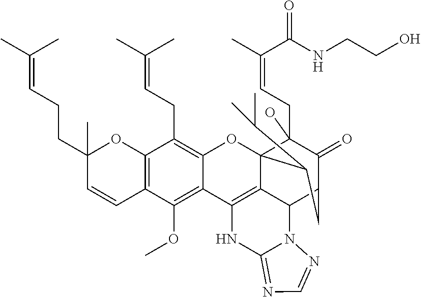 [SMILES: COc1c2c(c(CC=C(C)C)c3c1C1=C4C(C5CC(C(C)C)C4(O3)C(O)(C/C=C(/C)C(=O)NCCO)C5=O)n3ncnc3N1)OC(C)(CCC=C(C)C)C=C2] | C₄₃H₅₃N₅O₇ | 751.91 |
| 333 | 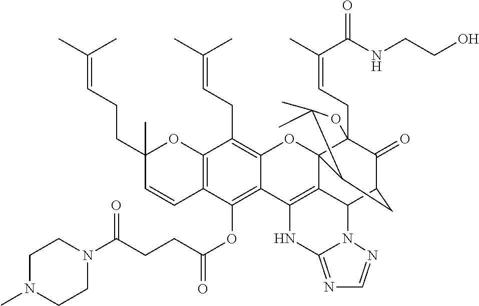 [SMILES: CC(C)=CCCC1(C)C=Cc2c(c(CC=C(C)C)c3c(c2OC(=O)CCC(=O)N2CCN(C)CC2)C2=C4C(C5CC6C(C)(C)OC(C/C=C(/C)C(=O)NCCO)(C5=O)C46O3)n3ncnc3N2)O1] | C₅₁H₆₅N₇O₉ | 920.1 |
| 334 | 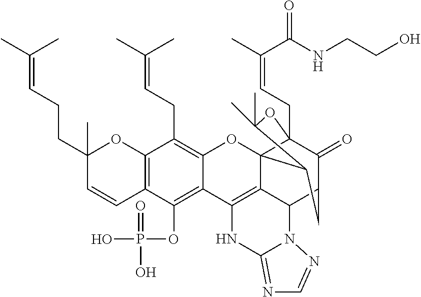 [SMILES: CC(C)=CCCC1(C)C=Cc2c(c(CC=C(C)C)c3c(c2OP(=O)(O)O)C2=C4C(C5CC6C(C)(C)OC(C/C=C(/C)C(=O)NCCO)(C5=O)C46O3)n3ncnc3N2)O1] | C₄₂H₅₂N₅O₁₀P | 817.86 |
| 335 | 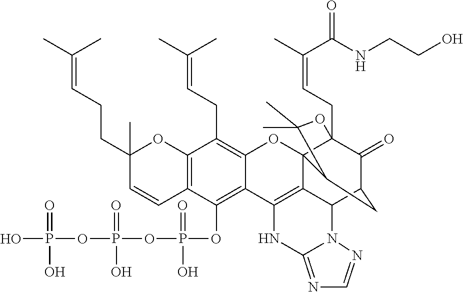 [SMILES: CC(C)=CCCC1(C)C=Cc2c(c(CC=C(C)C)c3c(c2OP(=O)(O)OP(=O)(O)OP(=O)(O)O)C2=C4C(C5CC6C(C)(C)OC(C/C=C(/C)C(=O)NCCO)(C5=O)C46O3)n3ncnc3N2)O1] | C₄₂H₅₄N₅O₁₆P₃ | 977.82 |
| 336 | 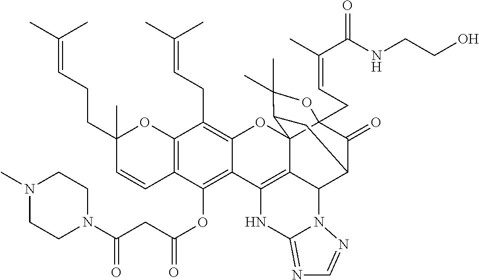 [SMILES: CC(C)=CCCC1(C)C=Cc2c(c(CC=C(C)C)c3c(c2OC(=O)CC(=O)N2CCN(C)CC2)C2=C4C(C5CC6C(C)(C)OC(C/C=C(/C)C(=O)NCCO)(C5=O)C46O3)n3ncnc3N2)O1] | C₅₀H₆₃N₇O₉ | 906.08 |
| 337 | 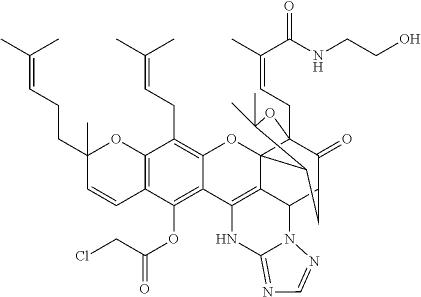 [SMILES: CC(C)=CCCC1(C)C=Cc2c(c(CC=C(C)C)c3c(c2OC(=O)CCl)C2=C4C(C5CC6C(C)(C)OC(C/C=C(/C)C(=O)NCCO)(C5=O)C46O3)n3ncnc3N2)O1] | C₄₄H₅₂ClN₅O₈ | 814.37 |
| 338 | 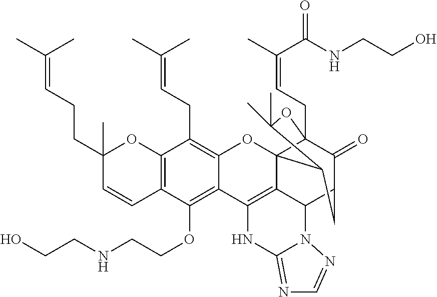 [SMILES: CC(C)=CCCC1(C)C=Cc2c(c(CC=C(C)C)c3c(c2OCCNCCO)C2=C4C(C5CC6C(C)(C)OC(C/C=C(/C)C(=O)NCCO)(C5=O)C46O3)n3ncnc3N2)O1] | C₄₆H₆₀N₆O₈ | 825 |
| 339 | 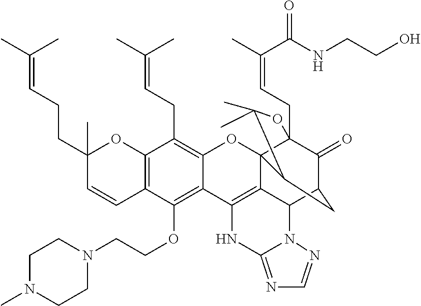 [SMILES: CC(C)=CCCC1(C)C=Cc2c(c(CC=C(C)C)c3c(c2OCCN2CCN(C)CC2)C2=C4C(C5CC6C(C)(C)OC(C/C=C(/C)C(=O)NCCO)(C5=O)C46O3)n3ncnc3N2)O1] | C₄₉H₆₅N₇O₇ | 864.08 |
| 340 | 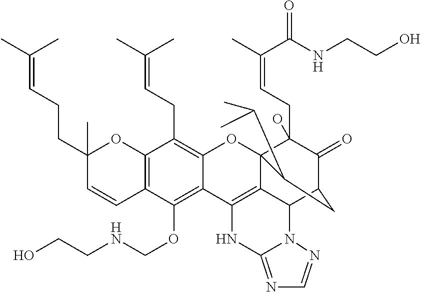 [SMILES: CC(C)=CCCC1(C)C=Cc2c(c(CC=C(C)C)c3c(c2OCNCCO)C2=C4C(C5CC(C(C)C)C4(O3)C(O)(C/C=C(/C)C(=O)NCCO)C5=O)n3ncnc3N2)O1] | C₄₅H₅₈N₆O₈ | 810.98 |
| 341 | 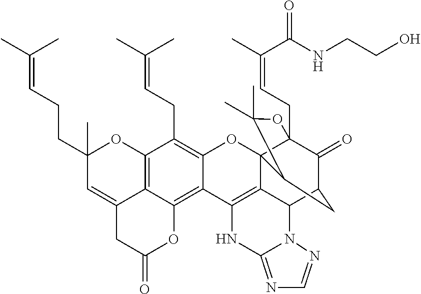 [SMILES: CC(C)=CCCC1(C)C=C2CC(=O)Oc3c2c(c(CC=C(C)C)c2c3C3=C4C(C5CC6C(C)(C)OC(C/C=C(/C)C(=O)NCCO)(C5=O)C46O2)n2ncnc2N3)O1] | C₄₄H₅₁N₅O₈ | 777.9 |
| 342 | 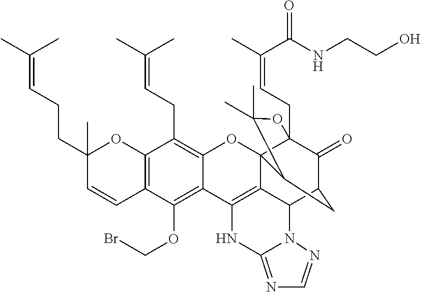 [SMILES: CC(C)=CCCC1(C)C=Cc2c(c(CC=C(C)C)c3c(c2OCBr)C2=C4C(C5CC6C(C)(C)OC(C/C=C(/C)C(=O)NCCO)(C5=O)C46O3)n3ncnc3N2)O1] | C₄₃H₅₂BrN₅O₇ | 830.81 |
| 343 | 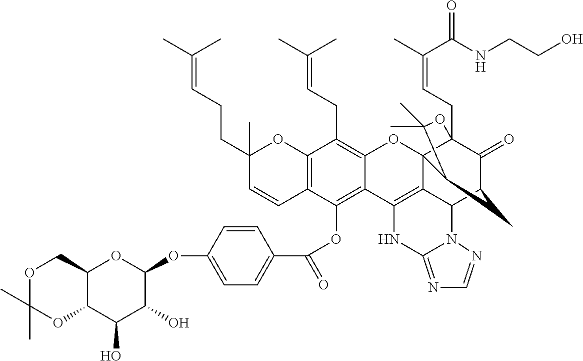 [SMILES: CC(C)=CCCC1(C)C=Cc2c(c(CC=C(C)C)c3c(c2OC(=O)c2ccc(O[C@@H]4O[C@@H]5COC(C)(C)O[C@H]5[C@H](O)[C@H]4O)cc2)C2=C4C([C@@H]5C[C@@H]6C(C)(C)OC(C/C=C(/C)C(=O)NCCO)(C5=O)C46O3)n3ncnc3N2)O1] | C₅₈H₆₉N₅O₁₄ | 1060.19 |
| 344 | 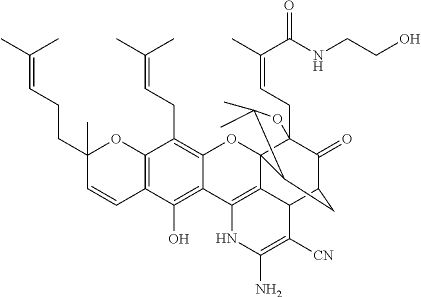 [SMILES: CC(C)=CCCC1(C)C=Cc2c(O)c3c(c(CC=C(C)C)c2O1)OC12C4=C3NC(N)=C(C#N)C4C3CC1C(C)(C)OC2(C/C=C(/C)C(=O)NCCO)C3=O] | C₄₃H₅₂N₄O₇ | 736.9 |
| 345 | 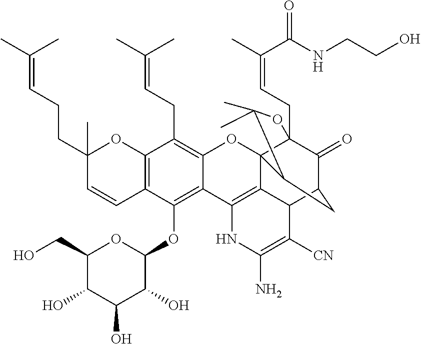 [SMILES: CC(C)=CCCC1(C)C=Cc2c(c(CC=C(C)C)c3c(c2O[C@@H]2O[C@H](CO)[C@@H](O)[C@H](O)[C@H]2O)C2=C4C(C(C#N)=C(N)N2)C2CC5C(C)(C)OC(C/C=C(/C)C(=O)NCCO)(C2=O)C45O3)O1] | C₄₉H₆₂N₄O₁₂ | 899.04 |
| 346 | 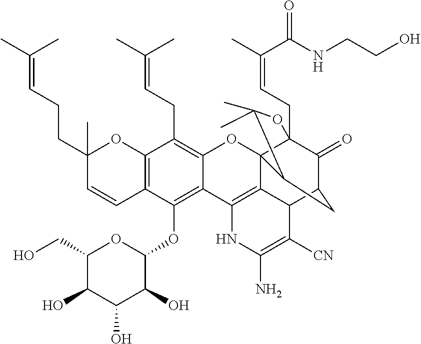 [SMILES: CC(C)=CCCC1(C)C=Cc2c(c(CC=C(C)C)c3c(c2O[C@H]2O[C@@H](CO)[C@H](O)[C@@H](O)[C@@H]2O)C2=C4C(C(C#N)=C(N)N2)C2CC5C(C)(C)OC(C/C=C(/C)C(=O)NCCO)(C2=O)C45O3)O1] | C₄₉H₆₂N₄O₁₂ | 899.04 |
| 347 | 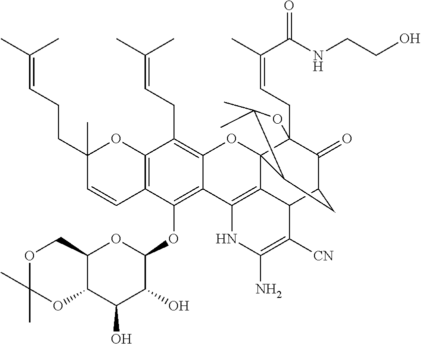 [SMILES: CC(C)=CCCC1(C)C=Cc2c(c(CC=C(C)C)c3c(c2O[C@@H]2O[C@@H]4COC(C)(C)O[C@H]4[C@H](O)[C@H]2O)C2=C4C(C(C#N)=C(N)N2)C2CC5C(C)(C)OC(C/C=C(/C)C(=O)NCCO)(C2=O)C45O3)O1] | C₅₂H₆₆N₄O₁₂ | 939.1 |
| 348 | 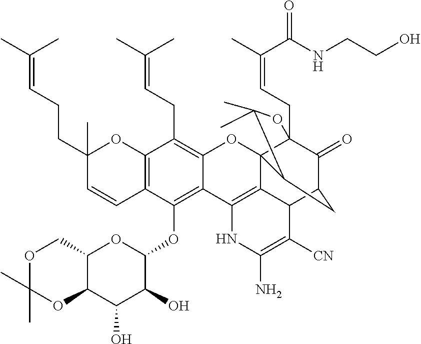 [SMILES: CC(C)=CCCC1(C)C=Cc2c(c(CC=C(C)C)c3c(c2O[C@H]2O[C@H]4COC(C)(C)O[C@@H]4[C@@H](O)[C@@H]2O)C2=C4C(C(C#N)=C(N)N2)C2CC5C(C)(C)OC(C/C=C(/C)C(=O)NCCO)(C2=O)C45O3)O1] | C₅₂H₆₆N₄O₁₂ | 939.1 |
| 349 | 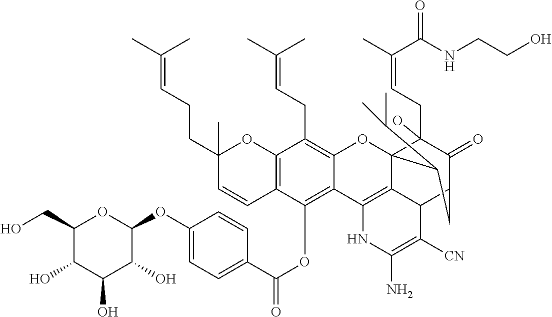 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C2=C4C(C(C#N)=C(N)N2)C2CC(C(C)C)C4(O3)C(O)(C/C=C(/C)C(=O)NCCO)C2=O)O1] | C₅₆H₆₆N₄O₁₄ | 1019.14 |
| 350 | 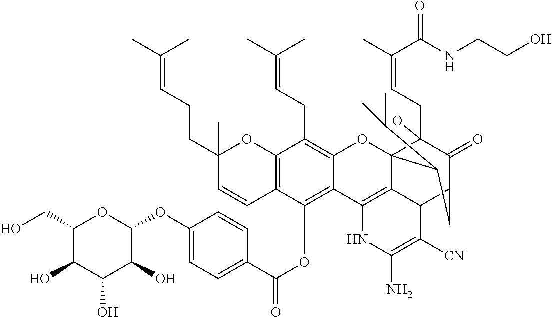 [SMILES: CC(C)=CCCC1(C)C=Cc2c(c(CC=C(C)C)c3c(c2OC(=O)c2ccc(O[C@H]4O[C@@H](CO)[C@H](O)[C@@H](O)[C@@H]4O)cc2)C2=C4C(C(C#N)=C(N)N2)C2CC(C(C)C)C4(O3)C(O)(C/C=C(/C)C(=O)NCCO)C2=O)O1] | C₅₆H₆₆N₄O₁₄ | 1019.14 |
| 351 | 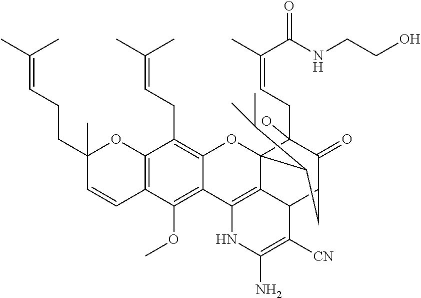 [SMILES: COc1c2c(c(CC=C(C)C)c3c1C1=C4C(C(C#N)=C(N)N1)C1CC(C(C)C)C4(O3)C(O)(C/C=C(/C)C(=O)NCCO)C1=O)OC(C)(CCC=C(C)C)C=C2] | C₄₄H₅₄N₄O₇ | 750.92 |
| 352 | 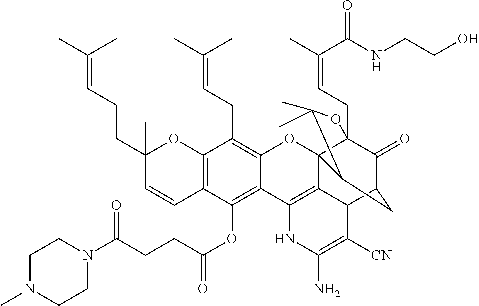 [SMILES: CC(C)=CCCC1(C)C=Cc2c(c(CC=C(C)C)c3c(c2OC(=O)CCC(=O)N2CCN(C)CC2)C2=C4C(C(C#N)=C(N)N2)C2CC5C(C)(C)OC(C/C=C(/C)C(=O)NCCO)(C2=O)C45O3)O1] | C₅₂H₆₆N₆O₉ | 919.12 |
| 353 | 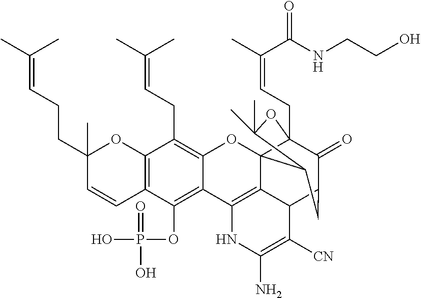 [SMILES: CC(C)=CCCC1(C)C=Cc2c(c(CC=C(C)C)c3c(c2OP(=O)(O)O)C2=C4C(C(C#N)=C(N)N2)C2CC5C(C)(C)OC(C/C=C(/C)C(=O)NCCO)(C2=O)C45O3)O1] | C₄₃H₅₃N₄O₁₀P | 816.88 |
| 354 | 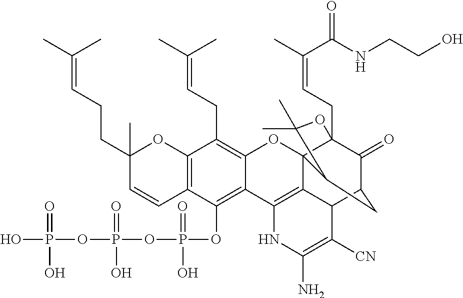 [SMILES: CC(C)=CCCC1(C)C=Cc2c(c(CC=C(C)C)c3c(c2OP(=O)(O)OP(=O)(O)OP(=O)(O)O)C2=C4C(C(C#N)=C(N)N2)C2CC5C(C)(C)OC(C/C=C(/C)C(=O)NCCO)(C2=O)C45O3)O1] | C₄₃H₅₅N₄O₁₆P₃ | 976.84 |
| 355 | 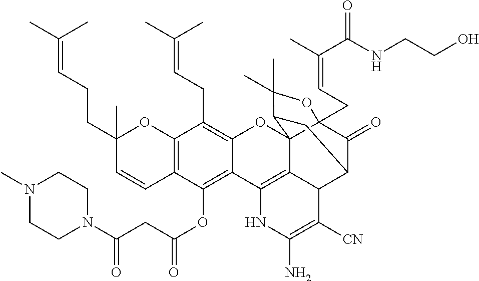 [SMILES: CC(C)=CCCC1(C)C=Cc2c(c(CC=C(C)C)c3c(c2OC(=O)CC(=O)N2CCN(C)CC2)C2=C4C(C(C#N)=C(N)N2)C2CC5C(C)(C)OC(C/C=C(/C)C(=O)NCCO)(C2=O)C45O3)O1] | C₅₁H₆₄N₆O₉ | 905.09 |
| 356 | 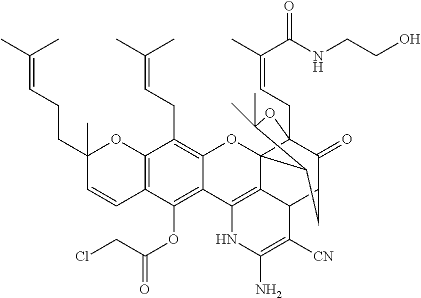 [SMILES: CC(C)=CCCC1(C)C=Cc2c(c(CC=C(C)C)c3c(c2OC(=O)CCl)C2=C4C(C(C#N)=C(N)N2)C2CC5C(C)(C)OC(C/C=C(/C)C(=O)NCCO)(C2=O)C45O3)O1] | C₄₅H₅₃ClN₄O₈ | 813.38 |
| 357 | 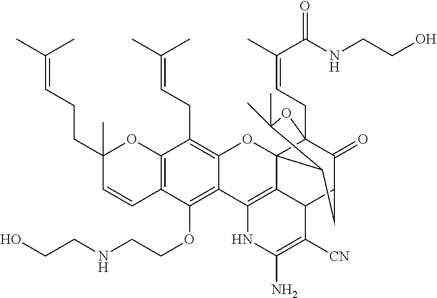 [SMILES: CC(C)=CCCC1(C)C=Cc2c(c(CC=C(C)C)c3c(c2OCCNCCO)C2=C4C(C(C#N)=C(N)N2)C2CC5C(C)(C)OC(C/C=C(/C)C(=O)NCCO)(C2=O)C45O3)O1] | C₄₇H₆₁N₅O₈ | 824.02 |
| 358 | 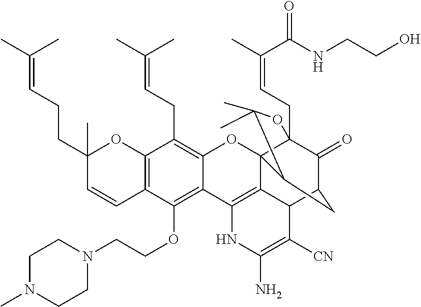 [SMILES: CC(C)=CCCC1(C)C=Cc2c(c(CC=C(C)C)c3c(c2OCCN2CCN(C)CC2)C2=C4C(C(C#N)=C(N)N2)C2CC5C(C)(C)OC(C/C=C(/C)C(=O)NCCO)(C2=O)C45O3)O1] | C₅₀H₆₆N₆O₇ | 863.1 |
| 359 | 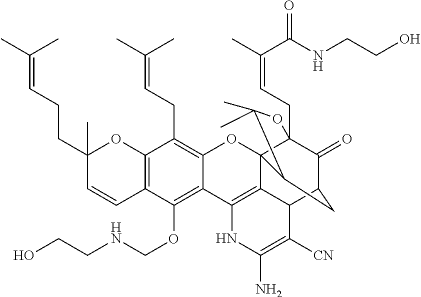 [SMILES: CC(C)=CCCC1(C)C=Cc2c(c(CC=C(C)C)c3c(c2OCNCCO)C2=C4C(C(C#N)=C(N)N2)C2CC5C(C)(C)OC(C/C=C(/C)C(=O)NCCO)(C2=O)C45O3)O1] | C₄₆H₅₉N₅O₈ | 809.99 |
| 360 | 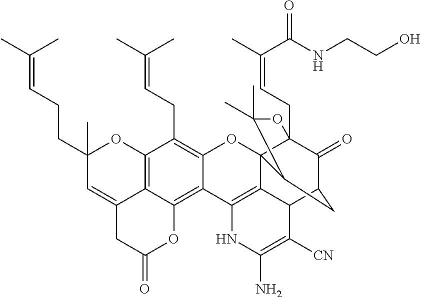 [SMILES: CC(C)=CCCC1(C)C=C2CC(=O)Oc3c2c(c(CC=C(C)C)c2c3C3=C4C(C(C#N)=C(N)N3)C3CC5C(C)(C)OC(C/C=C(/C)C(=O)NCCO)(C3=O)C45O2)O1] | C₄₅H₅₂N₄O₈ | 776.92 |
| 361 | 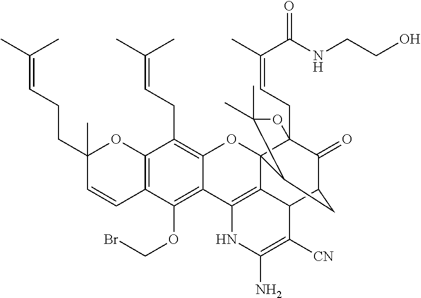 [SMILES: CC(C)=CCCC1(C)C=Cc2c(c(CC=C(C)C)c3c(c2OCBr)C2=C4C(C(C#N)=C(N)N2)C2CC5C(C)(C)OC(C/C=C(/C)C(=O)NCCO)(C2=O)C45O3)O1] | C₄₄H₅₃BrN₄O₇ | 829.82 |
| 362 | 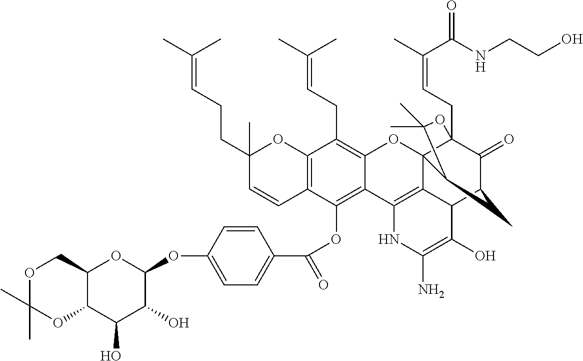 [SMILES: CC(C)=CCCC1(C)C=Cc2c(c(CC=C(C)C)c3c(c2OC(=O)c2ccc(O[C@@H]4O[C@@H]5COC(C)(C)O[C@H]5[C@H](O)[C@H]4O)cc2)C2=C4C(C(O)=C(N)N2)[C@@H]2C[C@@H]5C(C)(C)OC(C/C=C(/C)C(=O)NCCO)(C2=O)C45O3)O1] | C₅₉H₇₀N₄O₇ | 1059.21 |
| 363 | 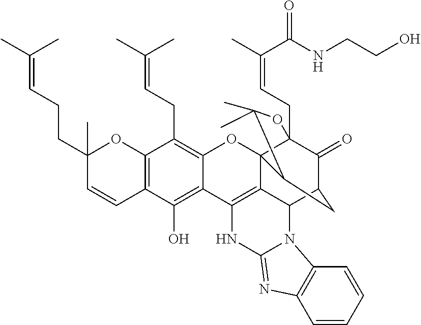 [SMILES: CC(C)=CCCC1(C)C=Cc2c(O)c3c(c(CC=C(C)C)c2O1)OC12C4=C3Nc3nc5ccccc5n3C4C3CC1C(C)(C)OC2(C/C=C(/C)C(=O)NCCO)C3=O] | C₄₇H₅₄N₄O₇ | 786.95 |
| 364 | 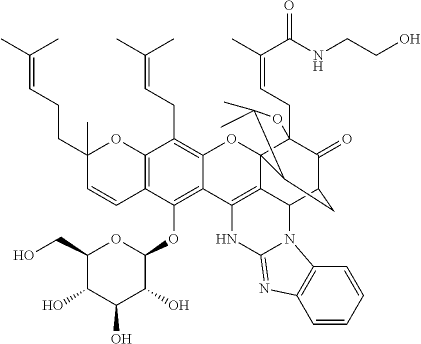 [SMILES: CC(C)=CCCC1(C)C=Cc2c(c(CC=C(C)C)c3c(c2O[C@@H]2O[C@H](CO)[C@@H](O)[C@H](O)[C@H]2O)C2=C4C(C5CC6C(C)(C)OC(C/C=C(/C)C(=O)NCCO)(C5=O)C46O3)n3c(nc4ccccc43)N2)O1] | C₅₃H₆₄N₄O₁₂ | 949.09 |
| 365 | 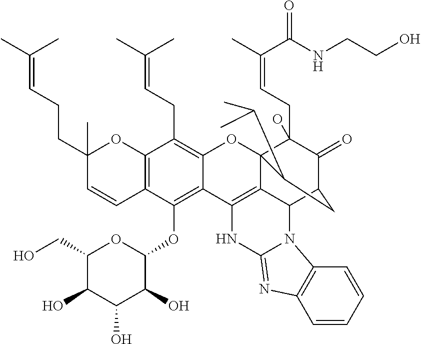 [SMILES: CC(C)=CCCC1(C)C=Cc2c(c(CC=C(C)C)c3c(c2O[C@H]2O[C@@H](CO)[C@H](O)[C@@H](O)[C@@H]2O)C2=C4C(C5CC(C(C)C)C4(O3)C(O)(C/C=C(/C)C(=O)NCCO)C5=O)n3c(nc4ccccc43)N2)O1] | C₅₃H₆₄N₄O₁₂ | 949.09 |
| 366 | 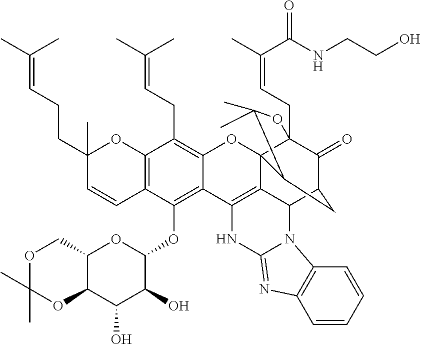 [SMILES: CC(C)=CCCC1(C)C=Cc2c(c(CC=C(C)C)c3c(c2O[C@H]2O[C@H]4COC(C)(C)O[C@@H]4[C@@H](O)[C@@H]2O)C2=C4C(C5CC6C(C)(C)OC(C/C=C(/C)C(=O)NCCO)(C5=O)C46O3)n3c(nc4ccccc43)N2)O1] | C₅₆H₆₈N₄O₁₂ | 989.16 |
| 367 | 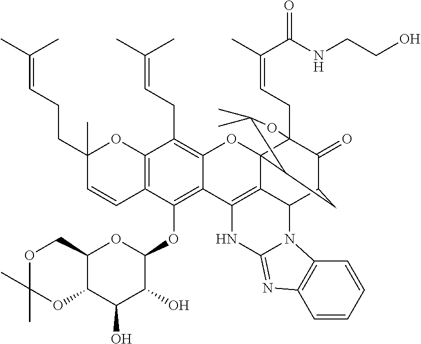 [SMILES: CC(C)=CCCC1(C)C=Cc2c(c(CC=C(C)C)c3c(c2O[C@@H]2O[C@@H]4COC(C)(C)O[C@H]4[C@H](O)[C@H]2O)C2=C4C(C5CC6C(C)(C)OC(C/C=C(/C)C(=O)NCCO)(C5=O)C46O3)n3c(nc4ccccc43)N2)O1] | C₅₆H₆₈N₄O₁₂ | 989.16 |
| 368 | 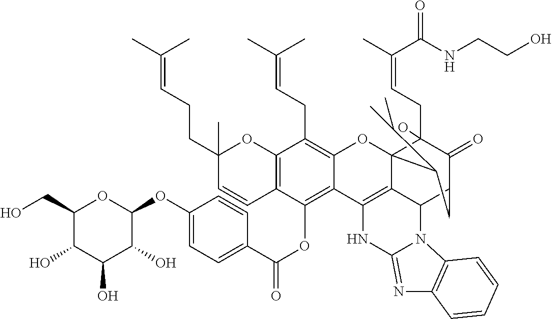 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C2=C4C(C5CC(C(C)C)C4(O3)C(O)(C/C=C(/C)C(=O)NCCO)C5=O)n3c(nc4ccccc43)N2)O1] | C₆₀H₆₈N₄O₁₄ | 1069.2 |
| 369 | 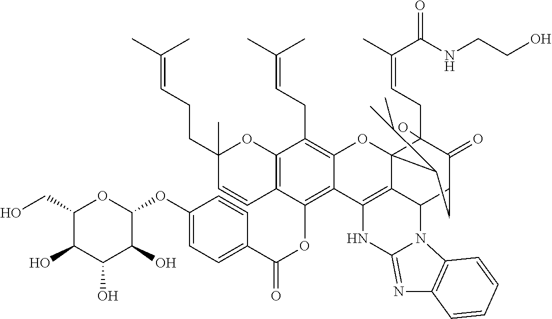 [SMILES: CC(C)=CCCC1(C)C=Cc2c(c(CC=C(C)C)c3c(c2OC(=O)c2ccc(O[C@H]4O[C@@H](CO)[C@H](O)[C@@H](O)[C@@H]4O)cc2)C2=C4C(C5CC(C(C)C)C4(O3)C(O)(C/C=C(/C)C(=O)NCCO)C5=O)n3c(nc4ccccc43)N2)O1] | C₆₀H₆₈N₄O₁₄ | 1069.2 |
| 370 | 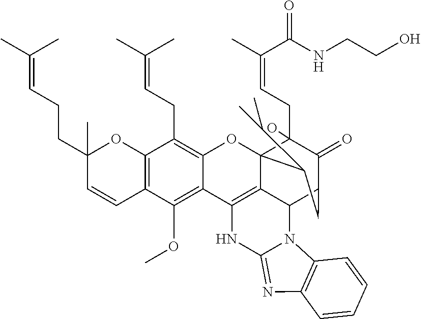 [SMILES: COc1c2c(c(CC=C(C)C)c3c1C1=C4C(C5CC(C(C)C)C4(O3)C(O)(C/C=C(/C)C(=O)NCCO)C5=O)n3c(nc4ccccc43)N1)OC(C)(CCC=C(C)C)C=C2] | C₄₈H₅₆N₄O₇ | 800.98 |
| 371 | 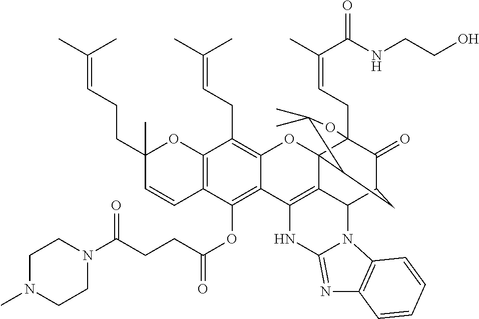 [SMILES: CC(C)=CCCC1(C)C=Cc2c(c(CC=C(C)C)c3c(c2OC(=O)CCC(=O)N2CCN(C)CC2)C2=C4C(C5CC6C(C)(C)OC(C/C=C(/C)C(=O)NCCO)(C5=O)C46O3)n3c(nc4ccccc43)N2)O1] | C₅₆H₆₈N₆O₉ | 969.17 |
| 372 | 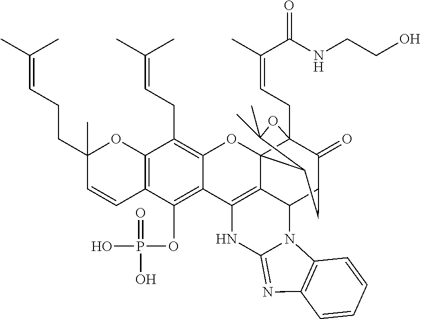 [SMILES: CC(C)=CCCC1(C)C=Cc2c(c(CC=C(C)C)c3c(c2OP(=O)(O)O)C2=C4C(C5CC6C(C)(C)OC(C/C=C(/C)C(=O)NCCO)(C5=O)C46O3)n3c(nc4ccccc43)N2)O1] | C₄₇H₅₅N₄O₁₀P | 866.93 |
| 373 | 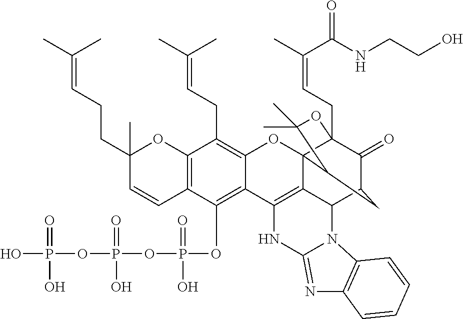 [SMILES: CC(C)=CCCC1(C)C=Cc2c(c(CC=C(C)C)c3c(c2OP(=O)(O)OP(=O)(O)OP(=O)(O)O)C2=C4C(C5CC6C(C)(C)OC(C/C=C(/C)C(=O)NCCO)(C5=O)C46O3)n3c(nc4ccccc43)N2)O1] | C₄₇H₅₇N₄O₁₆P₃ | 1026.89 |
| 374 | 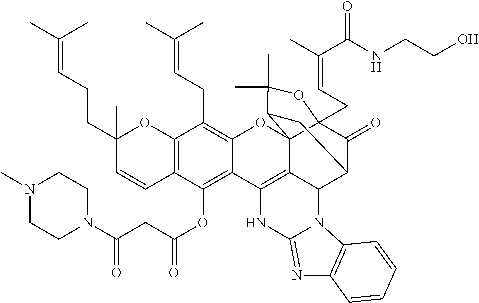 [SMILES: CC(C)=CCCC1(C)C=Cc2c(c(CC=C(C)C)c3c(c2OC(=O)CC(=O)N2CCN(C)CC2)C2=C4C(C5CC6C(C)(C)OC(C/C=C(/C)C(=O)NCCO)(C5=O)C46O3)n3c(nc4ccccc43)N2)O1] | C₅₅H₆₆N₆O₉ | 955.15 |
| 375 | 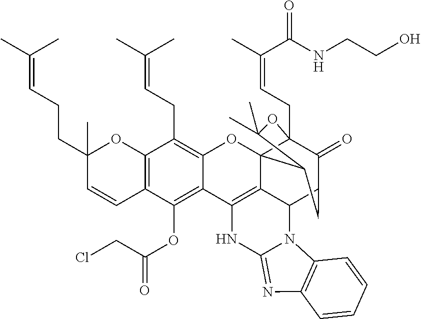 [SMILES: CC(C)=CCCC1(C)C=Cc2c(c(CC=C(C)C)c3c(c2OC(=O)CCl)C2=C4C(C5CC6C(C)(C)OC(C/C=C(/C)C(=O)NCCO)(C5=O)C46O3)n3c(nc4ccccc43)N2)O1] | C₄₉H₅₅ClN₄O₈ | 863.44 |
| 376 | 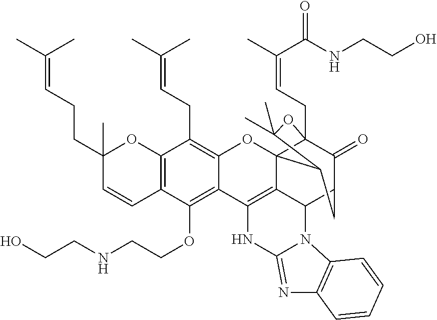 [SMILES: CC(C)=CCCC1(C)C=Cc2c(c(CC=C(C)C)c3c(c2OCCNCCO)C2=C4C(C5CC6C(C)(C)OC(C/C=C(/C)C(=O)NCCO)(C5=O)C46O3)n3c(nc4ccccc43)N2)O1] | C₅₁H₆₃N₅O₈ | 874.07 |
| 377 | 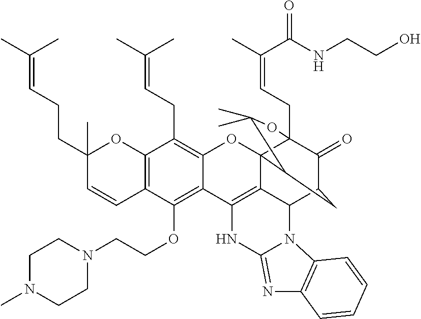 [SMILES: CC(C)=CCCC1(C)C=Cc2c(c(CC=C(C)C)c3c(c2OCCN2CCN(C)CC2)C2=C4C(C5CC6C(C)(C)OC(C/C=C(/C)C(=O)NCCO)(C5=O)C46O3)n3c(nc4ccccc43)N2)O1] | C₅₄H₆₈N₆O₇ | 913.15 |
| 378 | 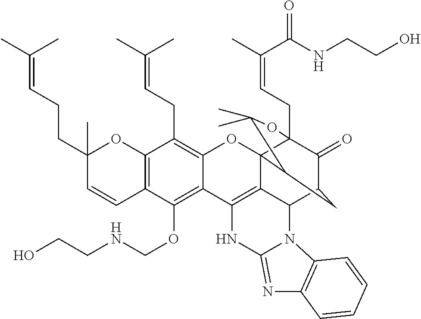 [SMILES: CC(C)=CCCC1(C)C=Cc2c(c(CC=C(C)C)c3c(c2OCNCCO)C2=C4C(C5CC6C(C)(C)OC(C/C=C(/C)C(=O)NCCO)(C5=O)C46O3)n3c(nc4ccccc43)N2)O1] | C₅₀H₆₁N₅O₈ | 860.05 |
| 379 | 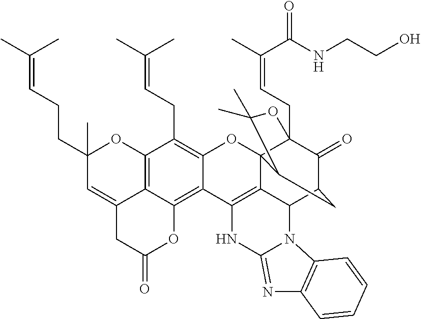 [SMILES: CC(C)=CCCC1(C)C=C2CC(=O)Oc3c2c(c(CC=C(C)C)c2c3C3=C4C(C5CC6C(C)(C)OC(C/C=C(/C)C(=O)NCCO)(C5=O)C46O2)n2c(nc4ccccc42)N3)O1] | C₄₉H₅₄N₄O₈ | 826.98 |
| 380 | 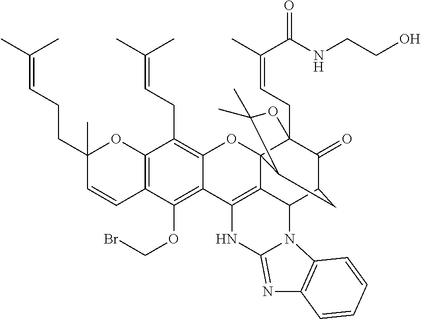 [SMILES: CC(C)=CCCC1(C)C=Cc2c(c(CC=C(C)C)c3c(c2OCBr)C2=C4C(C5CC6C(C)(C)OC(C/C=C(/C)C(=O)NCCO)(C5=O)C46O3)n3c(nc4ccccc43)N2)O1] | C₄₈H₅₅BrN₄O₇ | 879.88 |
| 381 | 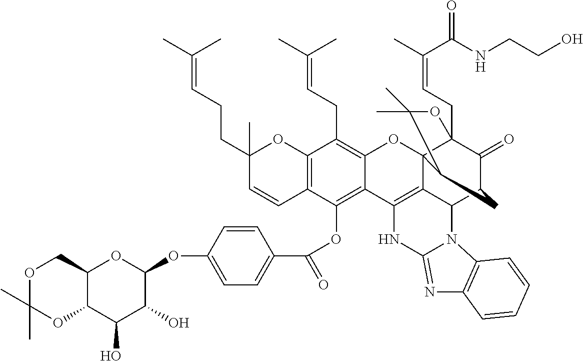 [SMILES: CC(C)=CCCC1(C)C=Cc2c(c(CC=C(C)C)c3c(c2OC(=O)c2ccc(O[C@@H]4O[C@@H]5COC(C)(C)O[C@H]5[C@H](O)[C@H]4O)cc2)C2=C4C([C@@H]5C[C@@H]6C(C)(C)OC(C/C=C(/C)C(=O)NCCO)(C5=O)C46O3)n3c(nc4ccccc43)N2)O1] | C₆₃H₇₂N₄O₁₄ | 1109.26 |
| 382 | 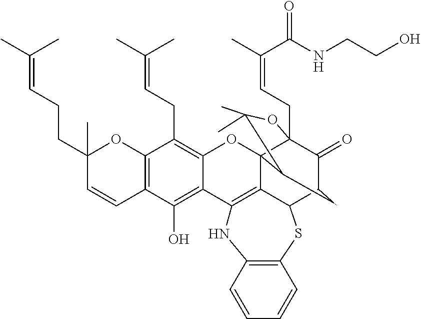 [SMILES: CC(C)=CCCC1(C)C=Cc2c(O)c3c(c(CC=C(C)C)c2O1)OC12C4=C3Nc3ccccc3SC4C3CC1C(C)(C)OC2(C/C=C(/C)C(=O)NCCO)C3=O] | C₄₆H₅₄N₂O₇S | 779 |
| 383 | 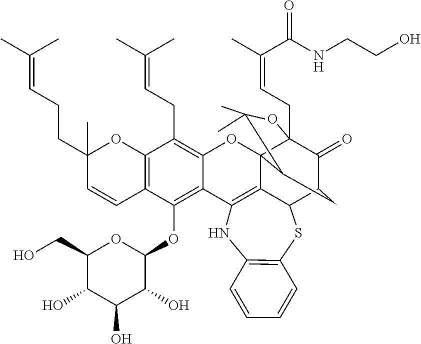 [SMILES: CC(C)=CCCC1(C)C=Cc2c(c(CC=C(C)C)c3c(c2O[C@@H]2O[C@H](CO)[C@@H](O)[C@H](O)[C@H]2O)C2=C4C(Sc5ccccc5N2)C2CC5C(C)(C)OC(C/C=C(/C)C(=O)NCCO)(C2=O)C45O3)O1] | C₅₂H₆₄N₂O₁₂S | 941.14 |
| 384 | 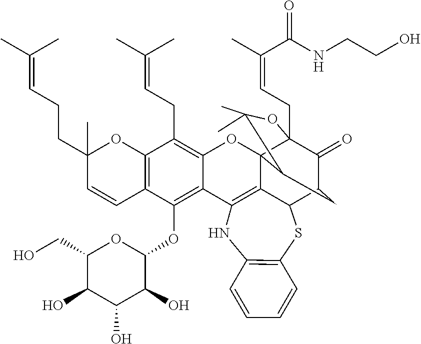 [SMILES: CC(C)=CCCC1(C)C=Cc2c(c(CC=C(C)C)c3c(c2O[C@H]2O[C@@H](CO)[C@H](O)[C@@H](O)[C@@H]2O)C2=C4C(Sc5ccccc5N2)C2CC5C(C)(C)OC(C/C=C(/C)C(=O)NCCO)(C2=O)C45O3)O1] | C₅₂H₆₄N₂O₁₂S | 941.14 |
| 385 | 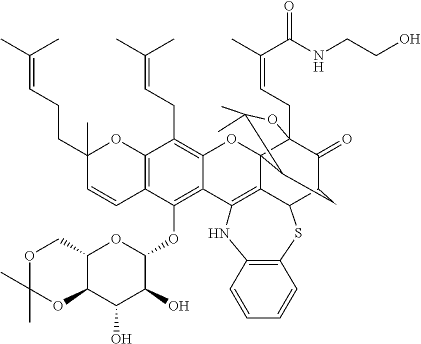 [SMILES: CC(C)=CCCC1(C)C=Cc2c(c(CC=C(C)C)c3c(c2O[C@H]2O[C@H]4COC(C)(C)O[C@@H]4[C@@H](O)[C@@H]2O)C2=C4C(Sc5ccccc5N2)C2CC5C(C)(C)OC(C/C=C(/C)C(=O)NCCO)(C2=O)C45O3)O1] | C₅₅H₆₈N₂O₁₂S | 981.2 |
| 386 | 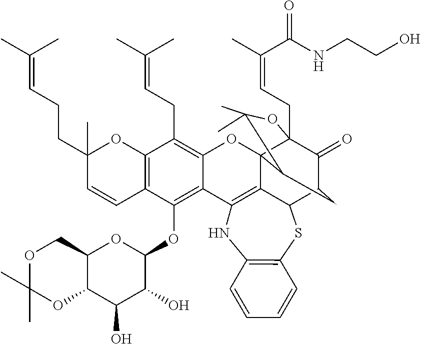 [SMILES: CC(C)=CCCC1(C)C=Cc2c(c(CC=C(C)C)c3c(c2O[C@@H]2O[C@@H]4COC(C)(C)O[C@H]4[C@H](O)[C@H]2O)C2=C4C(Sc5ccccc5N2)C2CC5C(C)(C)OC(C/C=C(/C)C(=O)NCCO)(C2=O)C45O3)O1] | C₅₅H₆₈N₂O₁₂S | 981.2 |
| 387 | 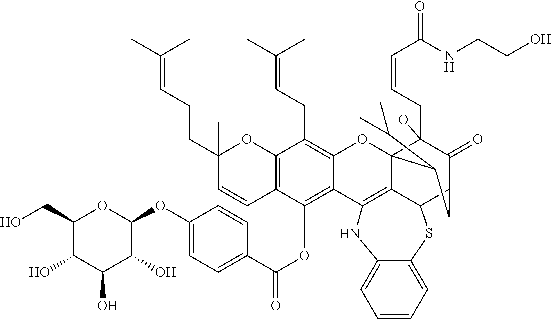 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C2=C4C(Sc5ccccc5N2)C2CC(C(C)C)C4(O3)C(O)(C/C=C\C(=O)NCCO)C2=O)O1] | C₅₈H₆₆N₂O₁₄S | 1047.21 |
| 388 | 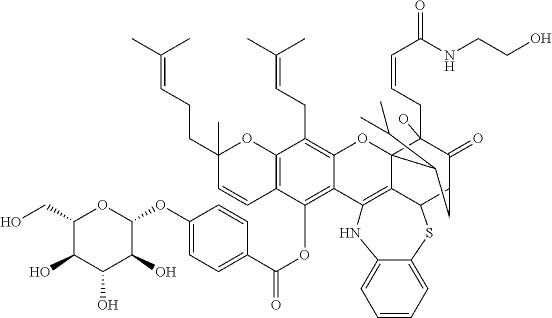 [SMILES: CC(C)=CCCC1(C)C=Cc2c(c(CC=C(C)C)c3c(c2OC(=O)c2ccc(O[C@H]4O[C@@H](CO)[C@H](O)[C@@H](O)[C@@H]4O)cc2)C2=C4C(Sc5ccccc5N2)C2CC(C(C)C)C4(O3)C(O)(C/C=C\C(=O)NCCO)C2=O)O1] | C₅₈H₆₆N₂O₁₄S | 1047.21 |
| 389 | 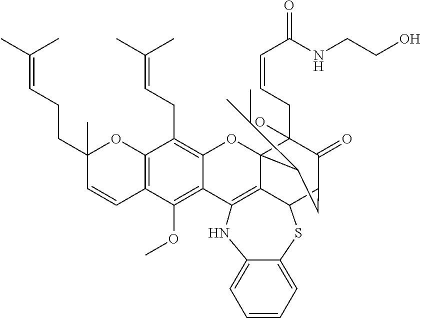 [SMILES: COc1c2c(c(CC=C(C)C)c3c1C1=C4C(Sc5ccccc5N1)C1CC(C(C)C)C4(O3)C(O)(C/C=C\C(=O)NCCO)C1=O)OC(C)(CCC=C(C)C)C=C2] | C₄₆H₅₄N₂O₇S | 779 |
| 390 | 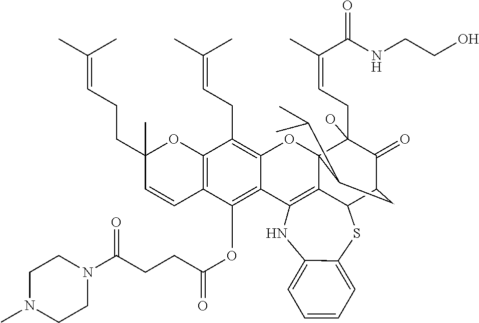 [SMILES: CC(C)=CCCC1(C)C=Cc2c(c(CC=C(C)C)c3c(c2OC(=O)CCC(=O)N2CCN(C)CC2)C2=C4C(Sc5ccccc5N2)C2CC(C(C)C)C4(O3)C(O)(C/C=C(/C)C(=O)NCCO)C2=O)O1] | C₅₅H₆₈N₄O₉S | 961.21 |
| 391 | 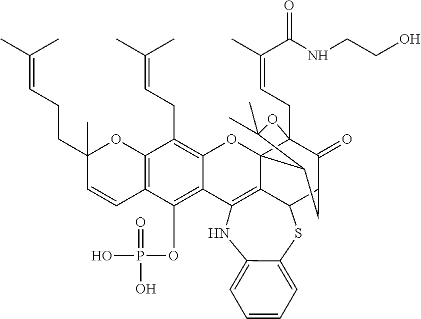 [SMILES: CC(C)=CCCC1(C)C=Cc2c(c(CC=C(C)C)c3c(c2OP(=O)(O)O)C2=C4C(Sc5ccccc5N2)C2CC5C(C)(C)OC(C/C=C(/C)C(=O)NCCO)(C2=O)C45O3)O1] | C₄₆H₅₅N₂O₁₀PS | 858.98 |
| 392 | 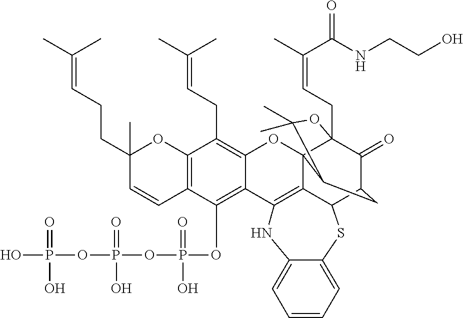 [SMILES: CC(C)=CCCC1(C)C=Cc2c(c(CC=C(C)C)c3c(c2OP(=O)(O)OP(=O)(O)OP(=O)(O)O)C2=C4C(Sc5ccccc5N2)C2CC5C(C)(C)OC(C/C=C(/C)C(=O)NCCO)(C2=O)C45O3)O1] | C₄₆H₅₇N₂O₁₆P₃S | 1018.93 |
| 393 | 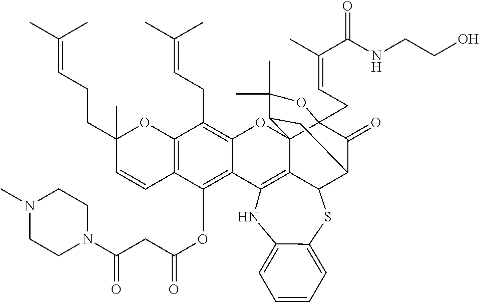 [SMILES: CC(C)=CCCC1(C)C=Cc2c(c(CC=C(C)C)c3c(c2OC(=O)CC(=O)N2CCN(C)CC2)C2=C4C(Sc5ccccc5N2)C2CC5C(C)(C)OC(C/C=C(/C)C(=O)NCCO)(C2=O)C45O3)O1] | C₅₄H₆₆N₄O₉S | 947.19 |
| 394 | 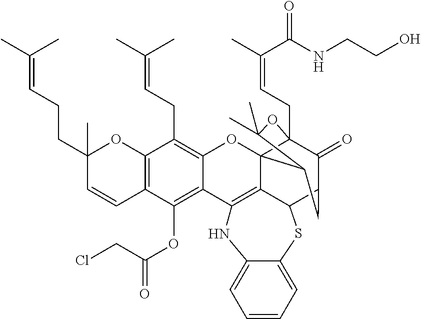 [SMILES: CC(C)=CCCC1(C)C=Cc2c(c(CC=C(C)C)c3c(c2OC(=O)CCl)C2=C4C(Sc5ccccc5N2)C2CC5C(C)(C)OC(C/C=C(/C)C(=O)NCCO)(C2=O)C45O3)O1] | C₄₈H₅₅ClN₂O₈S | 855.48 |
| 395 | 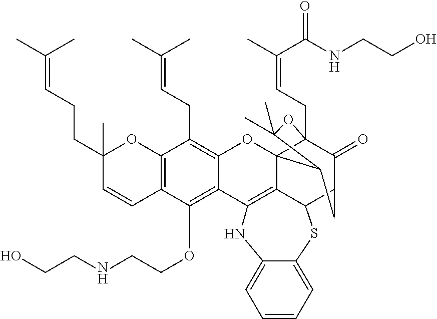 [SMILES: CC(C)=CCCC1(C)C=Cc2c(c(CC=C(C)C)c3c(c2OCCNCCO)C2=C4C(Sc5ccccc5N2)C2CC5C(C)(C)OC(C/C=C(/C)C(=O)NCCO)(C2=O)C45O3)O1] | C₅₀H₆₃N₃O₈S | 866.12 |
| 396 | 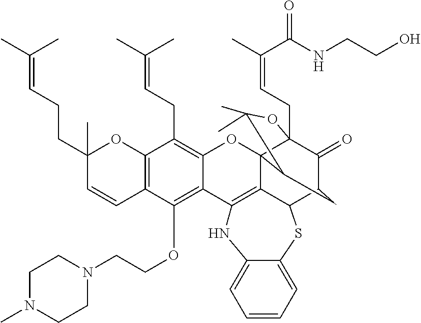 [SMILES: CC(C)=CCCC1(C)C=Cc2c(c(CC=C(C)C)c3c(c2OCCN2CCN(C)CC2)C2=C4C(Sc5ccccc5N2)C2CC5C(C)(C)OC(C/C=C(/C)C(=O)NCCO)(C2=O)C45O3)O1] | C₅₃H₆₈N₄O₇S | 905.19 |
| 397 | 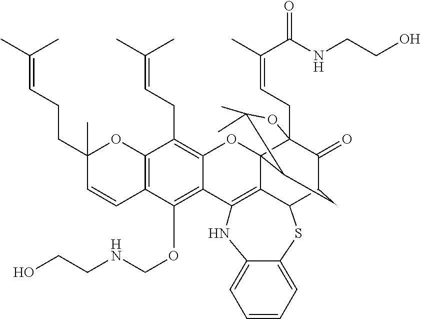 [SMILES: CC(C)=CCCC1(C)C=Cc2c(c(CC=C(C)C)c3c(c2OCNCCO)C2=C4C(Sc5ccccc5N2)C2CC5C(C)(C)OC(C/C=C(/C)C(=O)NCCO)(C2=O)C45O3)O1] | C₄₉H₆₁N₃O₈S | 852.09 |
| 398 | 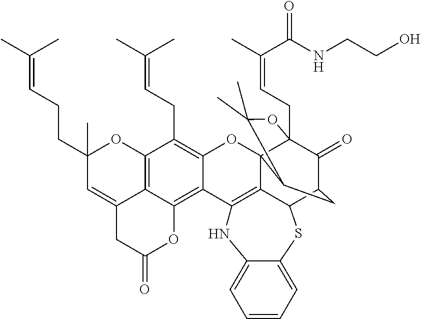 [SMILES: CC(C)=CCCC1(C)C=C2CC(=O)Oc3c2c(c(CC=C(C)C)c2c3C3=C4C(Sc5ccccc5N3)C3CC5C(C)(C)OC(C/C=C(/C)C(=O)NCCO)(C3=O)C45O2)O1] | C₄₈H₅₄N₂O₈S | 819.02 |
| 399 | 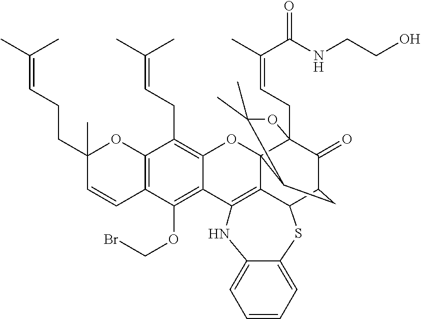 [SMILES: CC(C)=CCCC1(C)C=Cc2c(c(CC=C(C)C)c3c(c2OCBr)C2=C4C(Sc5ccccc5N2)C2CC5C(C)(C)OC(C/C=C(/C)C(=O)NCCO)(C2=O)C45O3)O1] | C₄₇H₅₅BrN₂O₇S | 871.92 |
| 400 | 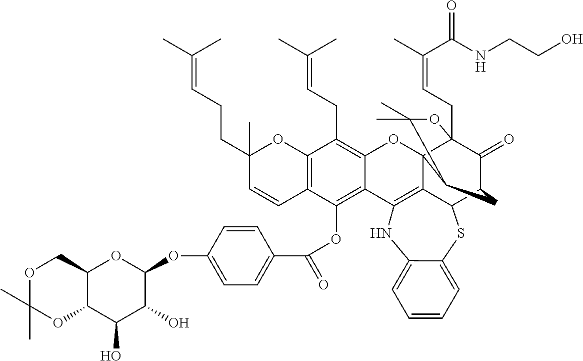 [SMILES: CC(C)=CCCC1(C)C=Cc2c(c(CC=C(C)C)c3c(c2OC(=O)c2ccc(O[C@@H]4O[C@@H]5COC(C)(C)O[C@H]5[C@H](O)[C@H]4O)cc2)C2=C4C(Sc5ccccc5N2)[C@@H]2C[C@@H]5C(C)(C)OC(C/C=C(/C)C(=O)NCCO)(C2=O)C45O3)O1] | C₆₂H₇₂N₂O₁₄S | 1101.31 |
| 401 | 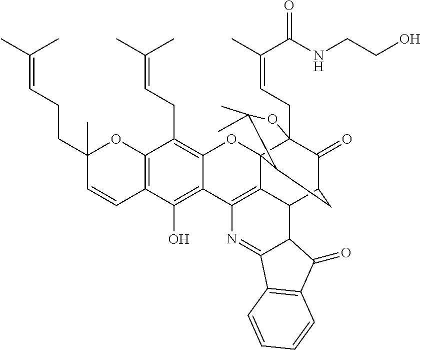 [SMILES: CC(C)=CCCC1(C)C=Cc2c(O)c3c(c(CC=C(C)C)c2O1)OC12C4=C3N=C3c5ccccc5C(=O)C3C4C3CC1C(C)(C)OC2(C/C=C(/C)C(=O)NCCO)C3=O] | C₄₉H₅₄N₂O₈ | 798.96 |
| 402 | 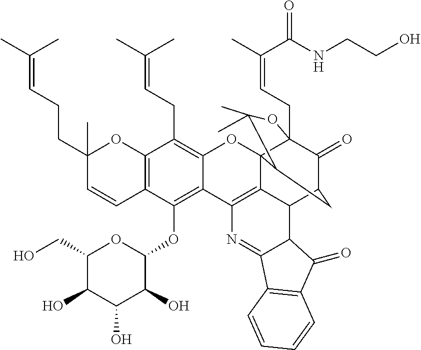 [SMILES: CC(C)=CCCC1(C)C=Cc2c(c(CC=C(C)C)c3c(c2O[C@H]2O[C@@H](CO)[C@H](O)[C@@H](O)[C@@H]2O)C2=C4C(C5CC6C(C)(C)OC(C/C=C(/C)C(=O)NCCO)(C5=O)C46O3)C3C(=O)c4ccccc4C3=N2)O1] | C₅₅H₆₄N₂O₁₃ | 961.1 |
| 403 | 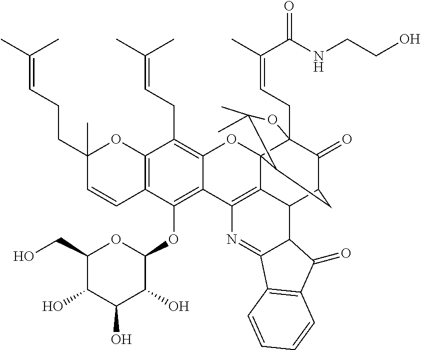 [SMILES: CC(C)=CCCC1(C)C=Cc2c(c(CC=C(C)C)c3c(c2O[C@@H]2O[C@H](CO)[C@@H](O)[C@H](O)[C@H]2O)C2=C4C(C5CC6C(C)(C)OC(C/C=C(/C)C(=O)NCCO)(C5=O)C46O3)C3C(=O)c4ccccc4C3=N2)O1] | C₅₅H₆₄N₂O₁₃ | 961.1 |
| 404 | 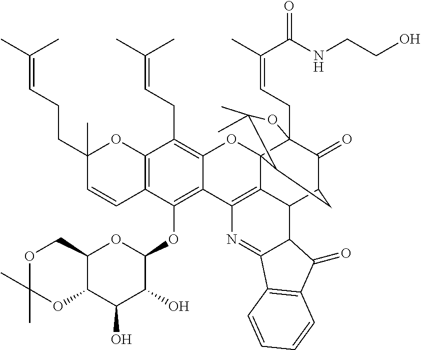 [SMILES: CC(C)=CCCC1(C)C=Cc2c(c(CC=C(C)C)c3c(c2O[C@@H]2O[C@@H]4COC(C)(C)O[C@H]4[C@H](O)[C@H]2O)C2=C4C(C5CC6C(C)(C)OC(C/C=C(/C)C(=O)NCCO)(C5=O)C46O3)C3C(=O)c4ccccc4C3=N2)O1] | C₅₈H₆₈N₂O₁₃ | 1001.17 |
| 405 | 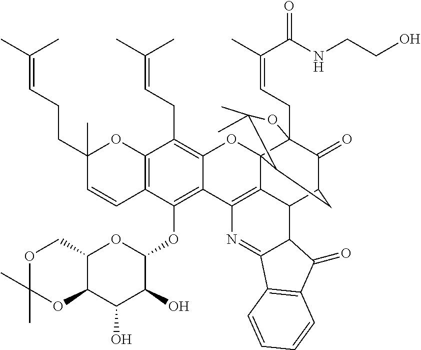 [SMILES: CC(C)=CCCC1(C)C=Cc2c(c(CC=C(C)C)c3c(c2O[C@H]2O[C@H]4COC(C)(C)O[C@@H]4[C@@H](O)[C@@H]2O)C2=C4C(C5CC6C(C)(C)OC(C/C=C(/C)C(=O)NCCO)(C5=O)C46O3)C3C(=O)c4ccccc4C3=N2)O1] | C₅₈H₆₈N₂O₁₃ | 1001.17 |
| 406 | 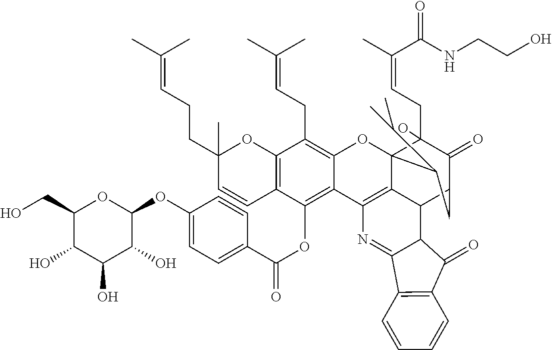 [SMILES: CC(C)=CCCC1(C)C=Cc2c(c(CC=C(C)C)c3c(c2OC(=O)c2ccc(O[C@@H]4O[C@H](CO)[C@@H](O)[C@H](O)[C@H]4O)cc2)C2=C4C(C5CC(C(C)C)C4(O3)C(O)(C/C=C(/C)C(=O)NCCO)C5=O)C3C(=O)c4ccccc4C3=N2)O1] | C₆₂H₆₈N₂O₁₅ | 1081.21 |
| 407 | 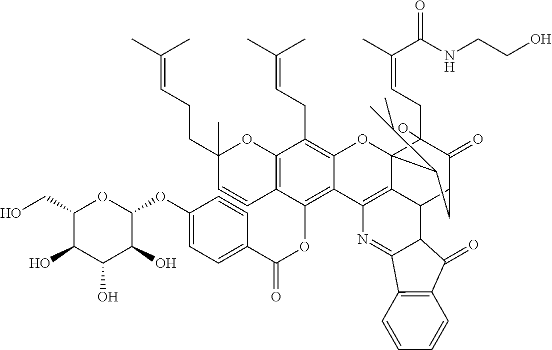 [SMILES: CC(C)=CCCC1(C)C=Cc2c(c(CC=C(C)C)c3c(c2OC(=O)c2ccc(O[C@H]4O[C@@H](CO)[C@H](O)[C@@H](O)[C@@H]4O)cc2)C2=C4C(C5CC(C(C)C)C4(O3)C(O)(C/C=C(/C)C(=O)NCCO)C5=O)C3C(=O)c4ccccc4C3=N2)O1] | C₆₂H₆₈N₂O₁₅ | 1081.21 |
| 408 | 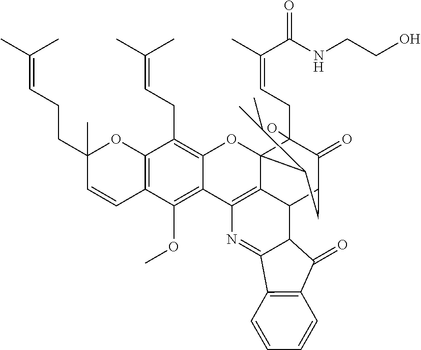 [SMILES: COc1c2c(c(CC=C(C)C)c3c1C1=C4C(C5CC(C(C)C)C4(O3)C(O)(C/C=C(/C)C(=O)NCCO)C5=O)C3C(=O)c4ccccc4C3=N1)OC(C)(CCC=C(C)C)C=C2] | C₅₀H₅₆N₂O₈ | 812.99 |
| 409 | 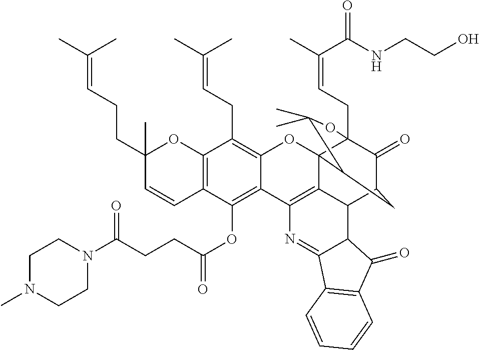 [SMILES: CC(C)=CCCC1(C)C=Cc2c(c(CC=C(C)C)c3c(c2OC(=O)CCC(=O)N2CCN(C)CC2)C2=C4C(C5CC6C(C)(C)OC(C/C=C(/C)C(=O)NCCO)(C5=O)C46O3)C3C(=O)c4ccccc4C3=N2)O1] | C₅₈H₆₈N₄O₁₀ | 981.18 |
| 410 | 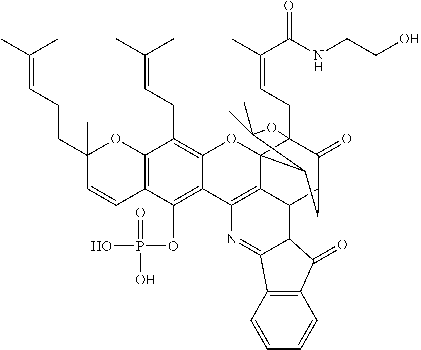 [SMILES: CC(C)=CCCC1(C)C=Cc2c(c(CC=C(C)C)c3c(c2OP(=O)(O)O)C2=C4C(C5CC6C(C)(C)OC(C/C=C(/C)C(=O)NCCO)(C5=O)C46O3)C3C(=O)c4ccccc4C3=N2)O1] | C₄₉H₅₅N₂O₁₁P | 878.94 |
| 411 | 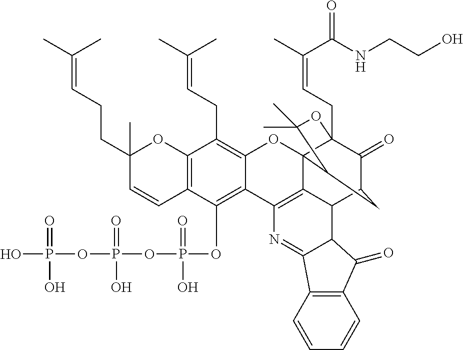 [SMILES: CC(C)=CCCC1(C)C=Cc2c(c(CC=C(C)C)c3c(c2OP(=O)(O)OP(=O)(O)OP(=O)(O)O)C2=C4C(C5CC6C(C)(C)OC(C/C=C(/C)C(=O)NCCO)(C5=O)C46O3)C3C(=O)c4ccccc4C3=N2)O1] | C₄₉H₅₇N₂O₁₇P₃ | 1038.9 |
| 412 | 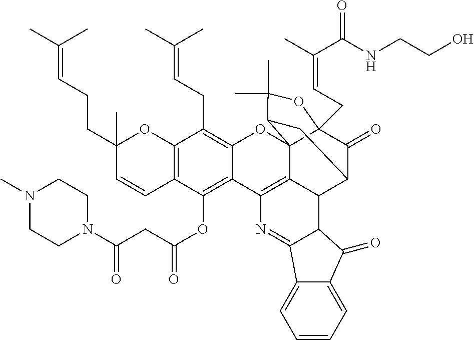 [SMILES: CC(C)=CCCC1(C)C=Cc2c(c(CC=C(C)C)c3c(c2OC(=O)CC(=O)N2CCN(C)CC2)C2=C4C(C5CC6C(C)(C)OC(C/C=C(/C)C(=O)NCCO)(C5=O)C46O3)C3C(=O)c4ccccc4C3=N2)O1] | C₅₇H₆₆N₄O₁₀ | 967.15 |
| 413 | 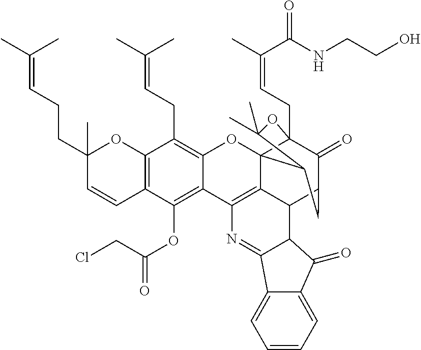 [SMILES: CC(C)=CCCC1(C)C=Cc2c(c(CC=C(C)C)c3c(c2OC(=O)CCl)C2=C4C(C5CC6C(C)(C)OC(C/C=C(/C)C(=O)NCCO)(C5=O)C46O3)C3C(=O)c4ccccc4C3=N2)O1] | C₅₁H₅₅ClN₂O₉ | 875.44 |
| 414 | 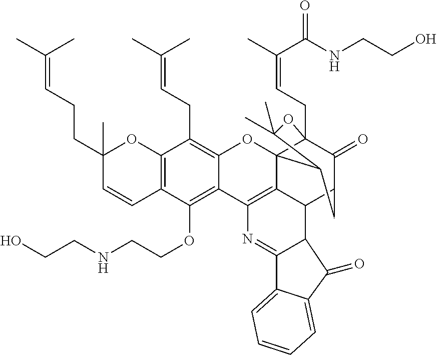 [SMILES: CC(C)=CCCC1(C)C=Cc2c(c(CC=C(C)C)c3c(c2OCCNCCO)C2=C4C(C5CC6C(C)(C)OC(C/C=C(/C)C(=O)NCCO)(C5=O)C46O3)C3C(=O)c4ccccc4C3=N2)O1] | C₅₃H₆₃N₃O₉ | 886.08 |
| 415 | 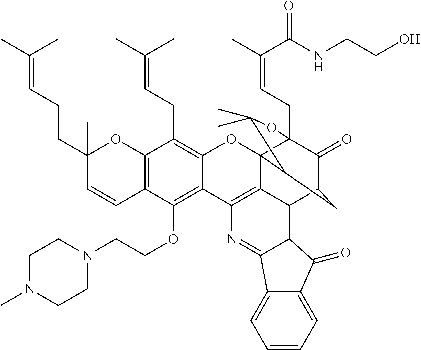 [SMILES: CC(C)=CCCC1(C)C=Cc2c(c(CC=C(C)C)c3c(c2OCCN2CCN(C)CC2)C2=C4C(C5CC6C(C)(C)OC(C/C=C(/C)C(=O)NCCO)(C5=O)C46O3)C3C(=O)c4ccccc4C3=N2)O1] | C₅₆H₆₈N₄O₈ | 925.16 |
| 416 | 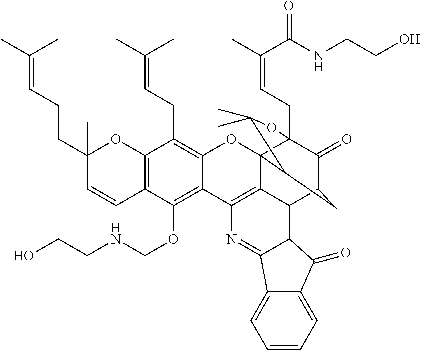 [SMILES: CC(C)=CCCC1(C)C=Cc2c(c(CC=C(C)C)c3c(c2OCNCCO)C2=C4C(C5CC6C(C)(C)OC(C/C=C(/C)C(=O)NCCO)(C5=O)C46O3)C3C(=O)c4ccccc4C3=N2)O1] | C₅₂H₆₁N₃O₉ | 872.06 |
| 417 | 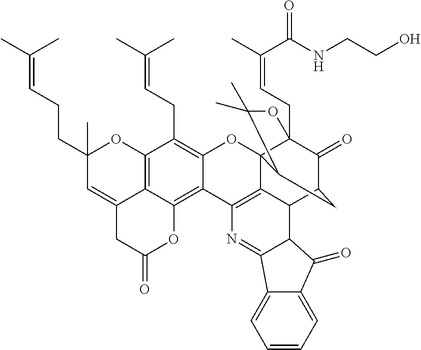 [SMILES: CC(C)=CCCC1(C)C=C2CC(=O)Oc3c2c(c(CC=C(C)C)c2c3C3=C4C(C5CC6C(C)(C)OC(C/C=C(/C)C(=O)NCCO)(C5=O)C46O2)C2C(=O)c4ccccc4C2=N3)O1] | C₅₁H₅₄N₂O₉ | 838.98 |
| 418 | 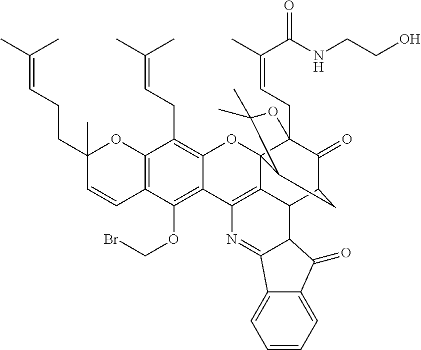 [SMILES: CC(C)=CCCC1(C)C=Cc2c(c(CC=C(C)C)c3c(c2OCBr)C2=C4C(C5CC6C(C)(C)OC(C/C=C(/C)C(=O)NCCO)(C5=O)C46O3)C3C(=O)c4ccccc4C3=N2)O1] | C₅₀H₄₅BrN₂O₈ | 891.88 |
| 419 | 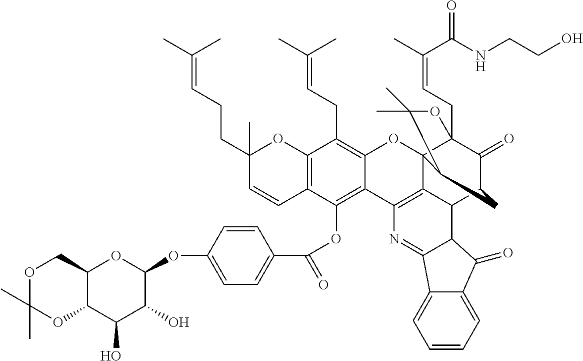 [SMILES: CC(C)=CCCC1(C)C=Cc2c(c(CC=C(C)C)c3c(c2OC(=O)c2ccc(O[C@@H]4O[C@@H]5COC(C)(C)O[C@H]5[C@H](O)[C@H]4O)cc2)C2=C4C(C5C(=O)c6ccccc6C5=N2)[C@@H]2C[C@@H]5C(C)(C)OC(C/C=C(/C)C(=O)NCCO)(C2=O)C45O3)O1] | C₆₅H₇₂N₂O₁₅ | 1121.27 |
| 420 | 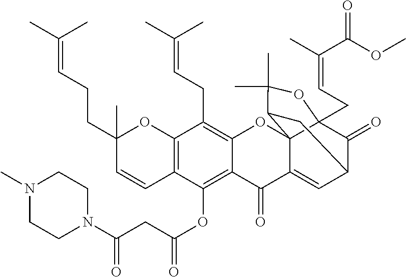 | C₄₇H₅₈N₂O₁₀ | 810.97 |
| 421 | 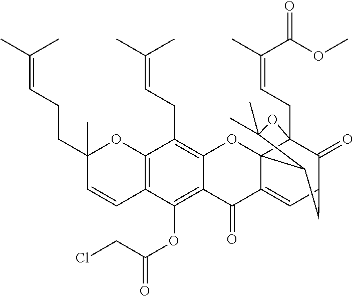 [SMILES: COC(=O)/C(C)=C\CC12OC(C)(C)C3CC(C=C4C(=O)c5c(OC(=O)CCl)c6c(c(CC=C(C)C)c5OC431)OC(C)(CCC=C(C)C)C=C6)C2=O] | C₄₁H₄₇ClO₉ | 719.26 |
| 422 | 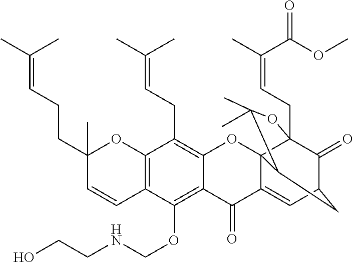 [SMILES: COC(=O)/C(C)=C\CC12OC(C)(C)C3CC(C=C4C(=O)c5c(OCNCCO)c6c(c(CC=C(C)C)c5OC431)OC(C)(CCC=C(C)C)C=C6)C2=O] | C₄₂H₅₃NO₉ | 715.87 |
| 423 | 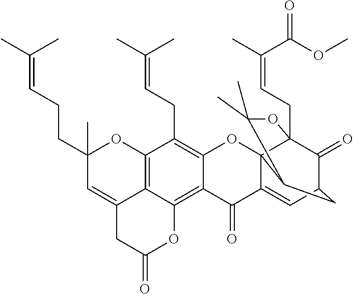 [SMILES: COC(=O)/C(C)=C\CC12OC(C)(C)C3CC(C=C4C(=O)c5c(c(CC=C(C)C)c6c7c5OC(=O)CC7=CC(C)(CCC=C(C)C)O6)OC431)C2=O] | C₄₁H₄₆O₉ | 682.8 |
| 424 | 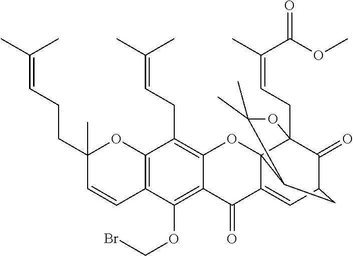 [SMILES: COC(=O)/C(C)=C\CC12OC(C)(C)C3CC(C=C4C(=O)c5c(OCBr)c6c(c(CC=C(C)C)c5OC431)OC(C)(CCC=C(C)C)C=C6)C2=O] | C₄₀H₄₇BrO₈ | 735.7 |
| 425 | 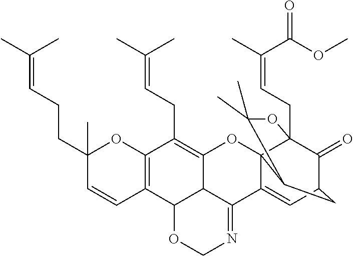 [SMILES: COC(=O)/C(C)=C\CC12OC(C)(C)C3CC(C=C4C5=NCOC6C7=C(OC(C)(CCC=C(C)C)C=C7)C(CC=C(C)C)=C(OC431)C56)C2=O] | C₄₀H₄₉NO₇ | 655.82 |
| 426 | 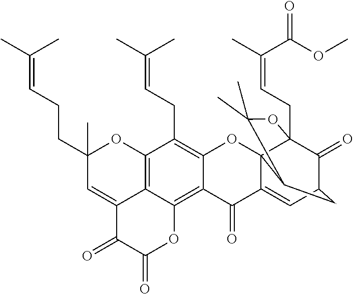 [SMILES: COC(=O)/C(C)=C\CC12OC(C)(C)C3CC(C=C4C(=O)c5c(c(CC=C(C)C)c6c7c5OC(=O)C(=O)C7=CC(C)(CCC=C(C)C)O6)OC431)C2=O] | C₄₁H₄₄O₁₀ | 696.78 |
| 427 | 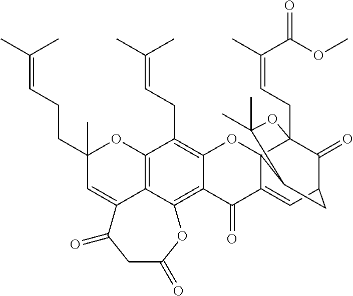 [SMILES: COC(=O)/C(C)=C\CC12OC(C)(C)C3CC(C=C4C(=O)c5c(c(CC=C(C)C)c6c7c5OC(=O)CC(=O)C7=CC(C)(CCC=C(C)C)O6)OC431)C2=O] | C₄₂H₄₆O₁₀ | 710.87 |
| 428 | 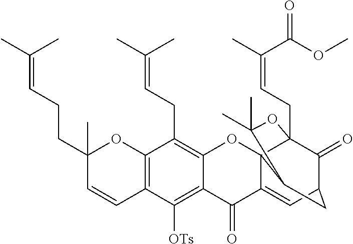 [SMILES: COC(=O)/C(C)=C\CC12OC(C)(C)C3CC(C=C4C(=O)c5c(OS(=O)(=O)c6ccc(C)cc6)c6c(c(CC=C(C)C)c5OC431)OC(C)(CCC=C(C)C)C=C6)C2=O] | C₄₆H₅₂O₁₀S | 796.96 |
| 429 | 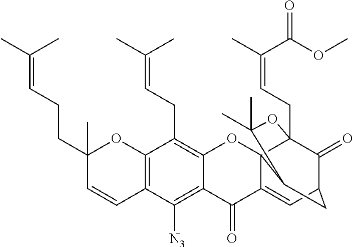 [SMILES: COC(=O)/C(C)=C\CC12OC(C)(C)C3CC(C=C4C(=O)c5c(N=[N+]=[N-])c6c(c(CC=C(C)C)c5OC431)OC(C)(CCC=C(C)C)C=C6)C2=O] | C₃₉H₄₅N₃O₇ | 667.79 |
| 430 | 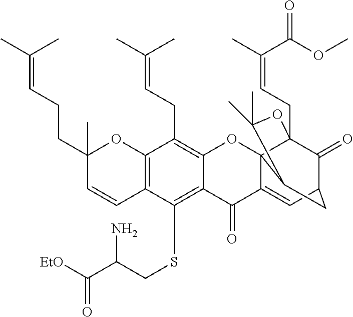 [SMILES: CCOC(=O)C(N)CSc1c2c(c(CC=C(C)C)c3c1C(=O)C1=CC4CC5C(C)(C)OC(C/C=C(/C)C(=O)OC)(C4=O)C15O3)OC(C)(CCC=C(C)C)C=C2] | C₄₄H₅₅NO₉S | 773.97 |
| 431 | 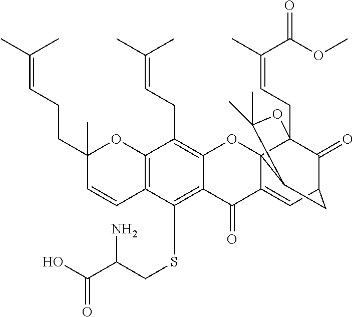 [SMILES: COC(=O)/C(C)=C\CC12OC(C)(C)C3CC(C=C4C(=O)c5c(c(CC=C(C)C)c6c(c5SCC(N)C(=O)O)C=CC(C)(CCC=C(C)C)O6)OC431)C2=O] | C₄₂H₅₁NO₉S | 745.92 |
| 432 | 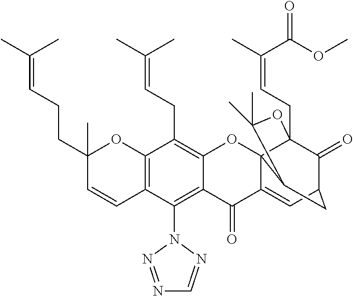 [SMILES: COC(=O)/C(C)=C\CC12OC(C)(C)C3CC(C=C4C(=O)c5c(c(CC=C(C)C)c6c(c5-n5ncnn5)C=CC(C)(CCC=C(C)C)O6)OC431)C2=O] | C₄₀H₄₆N₄O₇ | 694.82 |
| 433 | 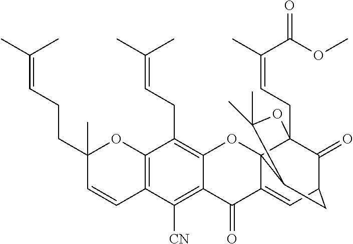 [SMILES: COC(=O)/C(C)=C\CC12OC(C)(C)C3CC(C=C4C(=O)c5c(C#N)c6c(c(CC=C(C)C)c5OC431)OC(C)(CCC=C(C)C)C=C6)C2=O] | C₄₀H₄₅NO₇ | 651.79 |
| 434 | 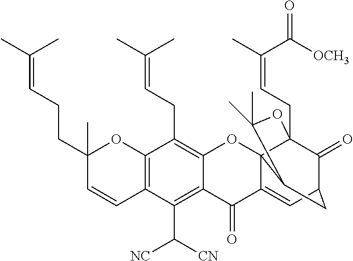 [SMILES: COC(=O)/C(C)=C\CC12OC(C)(C)C3CC(C=C4C(=O)c5c(c(CC=C(C)C)c6c(c5C(C#N)C#N)C=CC(C)(CCC=C(C)C)O6)OC431)C2=O] | C₄₂H₄₆N₂O₇ | 690.82 |
| 435 | 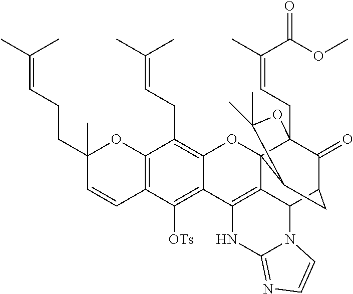 [SMILES: COC(=O)/C(C)=C\CC12OC(C)(C)C3CC(C1=O)C1C4=C(Nc5nccn51)c1c(OS(=O)(=O)c5ccc(C)cc5)c5c(c(CC=C(C)C)c1OC432)OC(C)(CCC=C(C)C)C=C5] | C₄₉H₅₈N₃O₉S | 862.04 |
| 436 | 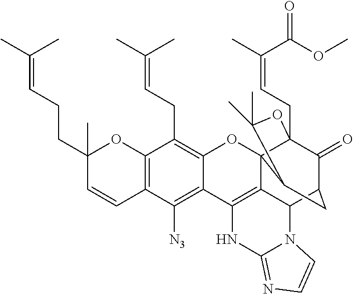 [SMILES: COC(=O)/C(C)=C\CC12OC(C)(C)C3CC(C1=O)C1C4=C(Nc5nccn51)c1c(N=[N+]=[N-])c5c(c(CC=C(C)C)c1OC432)OC(C)(CCC=C(C)C)C=C5] | C₄₂H₄₈N₆O₆ | 732.87 |
| 437 | 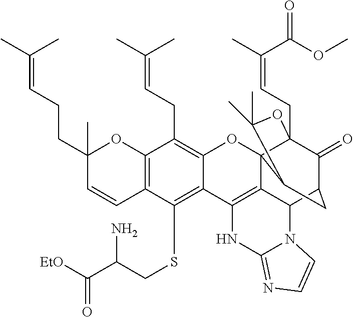 [SMILES: CCOC(=O)C(N)CSc1c2c(c(CC=C(C)C)c3c1C1=C4C(C5CC6C(C)(C)OC(C/C=C(/C)C(=O)OC)(C5=O)C46O3)n3ccnc3N1)OC(C)(CCC=C(C)C)C=C2] | C₄₇H₅₈N₄O₈ | 839.05 |
| 438 | 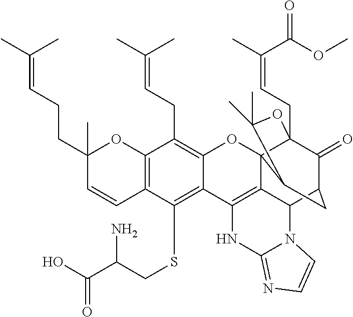 [SMILES: COC(=O)/C(C)=C\CC12OC(C)(C)C3CC(C1=O)C1C4=C(Nc5nccn51)c1c(c(CC=C(C)C)c5c(c1SCC(N)C(=O)O)C=CC(C)(CCC=C(C)C)O5)OC432] | C₄₅H₅₄N₄O₈S | 811 |
| 439 | 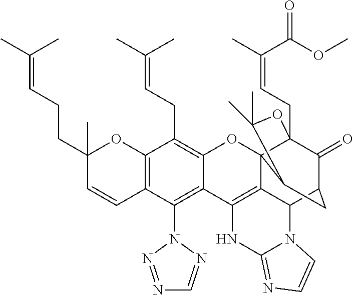 [SMILES: COC(=O)/C(C)=C\CC12OC(C)(C)C3CC(C1=O)C1C4=C(Nc5nccn51)c1c(c(CC=C(C)C)c5c(c1-n1ncnn1)C=CC(C)(CCC=C(C)C)O5)OC432] | C₄₃H₄₉N₇O₆ | 759.89 |
| 440 | 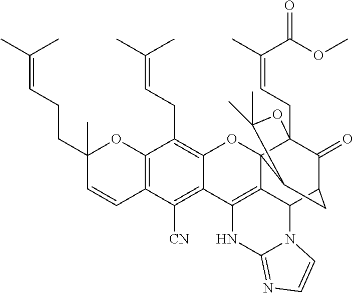 [SMILES: COC(=O)/C(C)=C\CC12OC(C)(C)C3CC(C1=O)C1C4=C(Nc5nccn51)c1c(C#N)c5c(c(CC=C(C)C)c1OC432)OC(C)(CCC=C(C)C)C=C5] | C₄₃H₄₈N₄O₆ | 716.86 |
| 441 | 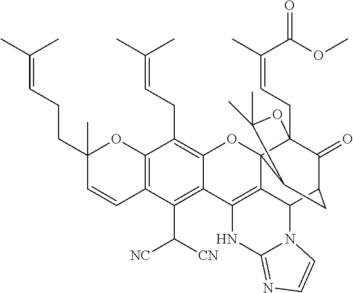 [SMILES: COC(=O)/C(C)=C\CC12OC(C)(C)C3CC(C1=O)C1C4=C(Nc5nccn51)c1c(c(CC=C(C)C)c5c(c1C(C#N)C#N)C=CC(C)(CCC=C(C)C)O5)OC432] | C₄₅H₄₉N₅O₆ | 755.9 |

### Preparation of Injection

### Example 442

### Preparation of Injection 1

Compound 9 (example 9) 5.0 g, ethanol 600 ml, 1,2-propanediol 600 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 5 mg/5 ml.

### Example 443

### Preparation of Injection 2

Compound 2 (example 2) 8.0 g, DMSO 50 ml, 1,2-propanediol 100 ml and Tween 80 100 ml were dissolved and the injection water was added up to total volume of 5000 ml. The solution was filtered with 0.22 μm membrane filter and sterilized 30 min at 100° C. to obtain 1000 preparation of injection 8 mg/5 ml.

### Biological Activity

### Example 444

### In Vitro Anti-Cancer Cell Experiment

### Methods

a. Cell lines: Human pancreatic cancer cell line Panc-1, human colorectal cancer cell line HT₂₉ and human lung cancer cell line NCI—H₄₆₀; the medium: s DMEM (Gibco BRL), containing 10% fetal calf serum (Gibco BRL) and 2 mM L-glutamine (Gibco BRL).

b. Test samples: example compounds 5, 9, 10, 22 and 24. The samples were dissolved in dimethyl sulfoxide (DMSO, Sigma, United States) and medium was added to the final concentration of 0.5%. Cisplatin was as positive control of (CDDP, purity 96%, from Kunming Institute of Precious Metals).

c. Method: cells were digested with trypsin and dispersed into single cells in the medium containing penicillin (25 U/ml) and streptomycin (25 μg/ml). The cells were seeded in 96-well culture plates (Corning Incorporated), at 37° C., in a humidified atmosphere with 5% CO₂ present for 24 hours. The culture medium was removed, 1-100 μm test compounds were added, cultured for 48 hours. Culture medium was removed and thiophene Wow blue (MTT, USA Sigma products) was added. The result was assayed by SK601-based microplate reader (Japan Seikagaku company's products), 570 nm/630 nm optical density (OD).

Calculation of cell viability: (Experimental group OD/control OD)×100%; Positive control CDDP was treated in the same way.

### Results

Inhibition of colorectal cancer: as shown in table 2 five test compounds 5, 9, 10, 22, and 24 showed anti-proliferative effect on HT₂₉. Example compounds 9 and 22 showed significant effect of anti-proliferate on HT₂₉ at low IC₅₀ (the compound concentration producing 50% inhibition of colony formation) values, respectively, 1.03 μg/ml (P<0.05) and 3.62 μg/ml (P<0.05) than conventional 5-FU and Cisplatin.

Inhibition of pancreatic cancer: as shown in table 2 five test compounds 5, 9, 10, 22, and 24 showed anti-proliferative effect on Panc-1. Example compounds 22 showed anti-proliferative effect on Panc-1 at IC₅₀ values 3.26 μg/ml (P<0.05) close to conventional 5-FU.

Inhibition of lung cancer: as shown in table 2 five test compounds showed anti-proliferative effect on NCI—H₄₆₀. Example compounds 9 and 22 showed significant effect of anti-proliferate on NCI—H₄₆₀ at low IC₅₀ (the compound concentration producing 50% inhibition of colony formation) values, respectively, 6.18 μg/ml (P<0.05) and 4.73 μg/ml (P<0.05) than conventional 5-FU.

TABLE 2
IC₅₀ (nM)

| Example | HT₂₉ | MCF7 | Panc-1 | NCI-H₄₆₀ |
|---|---|---|---|---|
| Compound 9 | 1.03 | 2.28 | 3.4 | 3.38 |
| Compound 22 | 3.62 | 6.94 | 3.26 | 4.89 |
| Compound 5 | 35.62 | 19.26 | 5.23 | 7.73 |
| Compound 10 | 38.33 | 56.32 | 17.6 | 17.25 |
| Compound 24 | 54.12 | 44.23 | 26.8 | 13.65 |
| CDDP | 3.69 | 3.92 | 2.17 | 5.40 |
| 5-FU | 14.36 | | 3.33 | |

### Example 445

### Efficacy Studies of Gambogic Acid Glycoside Analogs in Mice

Test samples: example compounds 5, 9, 10, 11, 13, 18, 19, 22, 24, 35 and 306.

Test animals: Kunming kinds of healthy mice (19-21 g), 10 mice (5 male and 5 female)/group, from Beijing Institute of Military Medical Sciences Animal Center.

Tumor strains: mice sarcoma S₁₈₀ for ascites passaged from Beijing Academy of Military Medical Institute of Pharmacology.

### Methods

Xenografts cultured S₁₈₀ tumor cells were implanted subcutaneously into the flank region of mice and tumors were allowed to grow to the desired average size of 100 mg. The mice were randomized into control and treatment groups with 10 mice per group. The control group was injected with the vehicle used to dissolve the drug. Other groups received the test compounds (example compound 5, 9, 10, 11, 13, 18, 19, 22, 24, 35, 306 and positive group, cyclophosphamide (CTX) and 5-fluorouracil (5-FU)) at the dose and schedule as indicated in Table VI. Injections were I.V. via the tail vein. Tumor measurements were taken every other day 20% tumor growth inhibition which was not statistically significant.

### Results

The in vivo experimental data showed anti-tumor efficacy of example compound 9, 22, 24 and 35 are statistically significant.

TABLE 3
Growth Inhibition of S₁₈₀ sarcoma

| | The | | | | Inhibition |
|---|---|---|---|---|---|
| Example | route | body Weight (g) | | Tumor | rate |
| Compound | of ad. | Before ad. | After ad. | weight (g) | (%) |
| Control | — | 23.09 ± 1.37 | 28.77 ± 3.17 | 2.02 ± 0.37 | — |
| CTX | iv | 21.48 ± 1.60 | 26.32 ± 3.58 | 1.02 ± 0.88 | 46.5* |
| 5-FU | iv | 22.59 ± 1.46 | 28.55 ± 2.38 | 1.18 ± 0.49 | 42.8* |
| Compound 5 | iv | 21.53 ± 1.77 | 27.39 ± 2.91 | 1.08 ± 0.63 | 45** |
| Compound 9 | iv | 20.53 ± 1.16 | 23.58 ± 1.86 | 0.56 ± 0.13 | 70.21** |
| Compound 10 | iv | 20.32 ± 1.22 | 25.12 ± 4.64 | 1.24 ± 0.73 | 39.36* |
| Compound 11 | iv | 19.92 ± 1.47 | 26.30 ± 3.62 | 1.35 ± 0.59 | 31.01* |
| Compound 13 | iv | 21.76 ± 1.93 | 27.59 ± 2.52 | 1.06 ± 0.70 | 45** |
| Compound 18 | iv | 20.16 ± 0.90 | 27.37 ± 3.92 | 1.42 ± 0.52 | 25.26 |
| Compound 19 | iv | 20.15 ± 0.88 | 25.89 ± 2.06 | 1.46 ± 0.58 | 25 |
| Compound 22 | iv | 19.34 ± 0.81 | 25.77 ± 2.33 | 0.88 ± 0.37 | 53.19** |
| Compound 24 | iv | 20.71 ± 1.75 | 27.61 ± 2.89 | 0.65 ± 0.37 | 62.87** |
| Compound 35 | iv | 20.08 ± 1.92 | 26.86 ± 1.81 | 0.78 ± 0.33 | 59.25** |
| Compound 306 | iv | 20.75 ± 1.33 | 26.98 ± 3.02 | 1.29 ± 0.49 | 36.77* |

Before ad.: before administration;
After ad.: after administration
*P < 0.01: compared with the control group significantly difference;
** p < 0.001: compared with the control group was very significant difference.
Inhibition rate more than 40% of the sample was statistically significant better than control group.

1. A compound of the formula I, II, III: 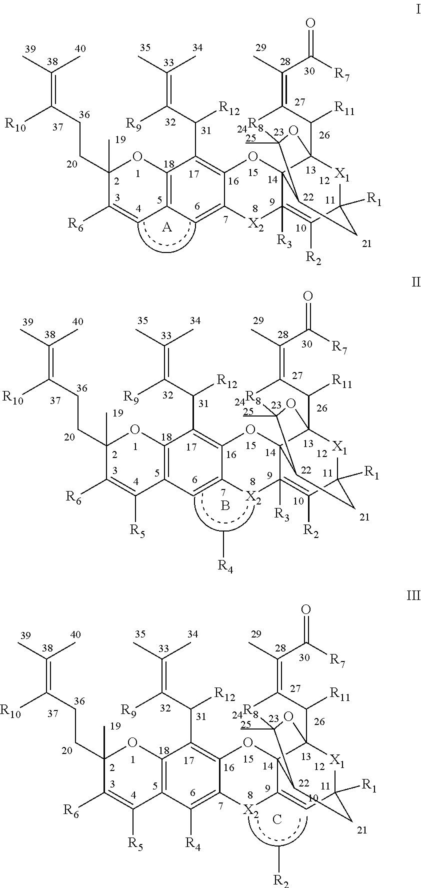
or stereoisomers, tautomers, prodrug, pharmaceutically acceptable salts, complex salts or solvates thereof, wherein:
The dotted lines are optionally substituted single bonds, optionally substituted double bond or a optionally substituted heterocyclic group containing carbon, oxygen, sulfur or nitrogen element;
Ring A, ring B or/and ring C is 4-10 membered saturated or/and unsaturated aliphatic ring aliphatic heterocycle or aryl heterocycle.
R₁, R₂, R₃, R₄, R₅, R₆, R₇, R₈, R₉, R₁₀, R₁₁ or/and R₁₂ is, independently at each occurrence, optionally substituted substituent of glycosyl, optionally substituted multi-hydroxyl, amino acid, acyloxy, phosphoric acid oxy, sulfonyloxy, alkoxy, aryloxy, heterocyclic oxy, thiol, substituted thiol, aliphatic or cyclic containing primary amine secondary amine or/and tertiary amine or substituted primary amine, substituted secondary amine or substituted tertiary amine, where contains optionally substituted one or combination;
Substituent containing oxygen, sulfur, nitrogen or phosphorus element is, independently at each occurrence, optionally substituted one or combination of saturated, unsaturated
C₁₋₁₀ alkyl, optionally substituted 1-4 double bond, optionally substituted triple bond, optional substituent of saturated or unsaturated C₁₋₁₀ alicyclic, arylcyclic and heterocyclic group, where contains a cycle or combination of oxygen, sulfur, nitrogen or phosphorus element, saturated or unsaturated 3-7 membered alicycle, aryl cycle, multi-cycle, aliphatic heterocycle, aryl heterocycle or fused heterocycle;
wherein:
X₁ and X₂ are, independently at each occurrence, C═O, C═Rb—Ra, CHOH, CHORb, CHRb or substituent, where Rb contains, independently at each occurrence, one or combination of C, N or P element; Ra is H, H₂, optionally substituted straight-alkyl, optionally substituted branched-alkyl, C₁₋₁₀ optionally substituted saturated alkyl, optionally substituted 1-4 double bond, optionally substituted 1-4 triple bond, optionally substituted unsaturated alkyl, optionally substituted saturated or unsaturated alicyclic, optionally substituted arylcyclic, optionally substituted aryl or optionally substituted 3-7 membered heterocyclic, optionally substituted aryheterocyclic, fused heterocyclic group where contains hydroxyl, halogen, oxygen, nitrogen, sulfur or phosphorus element;
wherein:
Substituent is, independently at each occurrence, C₁₋₁₀ optionally substituted saturated Glycosyl is D- or L-configuration and its glycoside bond is C—C or C-hetero bond connection, including 1-8 optionally substituted glycosyl or optional substituent glycosyl group;
Multi-hydroxyl is, independently at each occurrence, 1-10 optionally substituted hydroxyl group of alkyl, aryl, cyclic or heterocyclic group, where contains optionally substituted or combination of amino acid, acyloxy, sulfonyloxy, phosphoric acid oxy, alkoxy, aryoxyl or heterocyclicoxyl, thiol, substituted thiol, or heteroatom contained alkyl, alicyclic, aryl ring, aliphatic heterocyclic or aryl heterocyclic group;
Cyclized cyclic substituent is, independently at each occurrence, formed a new one or a combination of A-ring between C-4 and C-6, B-ring between C-6 and C-8, C-ring between C-8 and C-10 positions;
R₁₂, R₁, R₂, R₅, R₆, R₈, R₉, R₁₀, R₁₁ or/and R₁₂ is, independently at each occurrence, H, halogen or XRa; where XRa is unsubstituted or substituted group containing C, O, S, Se, N, and/or P element. R₃ is XaRa electrophilic substituent, where Xa is, independently at each occurrence, unsubstituted or substituted group containing C, S, P, and/or Si element.
R₄ is, independently at each occurrence, optional substituent of 1-8 glycosyl, multi-hydroxyl, substituted multi-hydroxyl, 1-5 amino acid, 1-4 phosphate, acyloxy, phosphoric, sulfonyloxy, alkoxy, aryloxy, heterocyclic oxy, alkyl, alicyclic, aryl cyclic, aliphatic heterocyclic oxyl or aryl heterocyclic oxyl group containing oxygen, sulfur, nitrogen or phosphorus element, where glycosyl, multi-hydroxyl, amino acid, acryloxy, phosphoryloxy, sulfonyloxy, alkoxy, aryloxy, heterocyclic oxy and substituent is the same as above.
1-8 Glycosyl is, independently at each occurrence, optionally substituted C₃₋₈ saccharide, optionally substituted monosaccharide, optionally substituted disaccharide, optionally substituted trisaccharide and/or optionally substituted polysaccharide;
wherein:
C₃₋₈ Saccharide is independently at each occurrence, optionally substituted C₃ saccharide, optionally substituted C₄ saccharide, optionally substituted C₅ saccharide, optionally substituted C₆ saccharide, optionally substituted C₇ saccharide, optionally substituted C₈ saccharide, optionally substituted hydroxyl saccharide, optionally substituted amino saccharide, optionally substituted deoxy saccharide, optionally substituted sulfuric acid saccharide, optionally substituted hetero-element saccharide and/or its glycoside.
R₇ is H or XbRa; Xb is, independently at each occurrence, optional substituent containing H, C, O or N element.
wherein:
When X₁ and/or X₂ is C═O, C═Rb—Ra, CHOH, CHORb or CHRb, X₁ and X₂ are the same or different substituents; when Rb is C, N or P element, Ra is, independently at each occurrence, optionally substituted formation of olefin, alkane, halogenated hydrocarbon, alcohol, ether, oxime, hydrazone or substituted said groups.
A bromo compound at 11-position is selected, independently at each occurrence, from:
gambogic acid, methyl gambogate, ethyl gambogate, gambogyl morpholine, gambogyl.

2. A compound according to the claim 1, wherein:
A compound with A-ring, B-ring or/and C-ring cyclized respectively between 4- and 6-position, 6- and 8-position and 8- and 10-position of gamboge acid is selected, independently at each occurrence, from the example 1 to example 441 and following list but not limiting, of the method and composition of the present invention:
Fused A-ring was formed between 4- and 6-position of gambogate acid analogs, methyl pyrano[4,3,2-d,e]gambogate-5(4H)one, methyl-5-ketone-4,7,12-trihydropyrano[4″,3″,2″:4′,5′,6′]gambogate[10′,9′,8′:4,5,6]pyrimidino[3,2-a]imidazole, methyl-5-ketone-4,7,12-trihydropyrano[4″,3″,2″:4′,5′,6]gambogate[10′,9′,8′:4,5,6]pyrimidino[3,2-b]triazolo(1,2,4), methyl-5-ketone-4,7,10-trihydropyrano[4′,3′,2′:4,5,6]gambogate[10,9,8:d,e]-8-amino-9-cyanopyrimidine, methyl-5-ketone-4,10-dihydropyrano[4′,3′,2′:4,5,6]gambogate[10,9,8:d,e]-8-amino-9-cyanopyrimidine, methyl-5-ketone-4,10-dihydropyrano[4′,3′,2′:4,5,6]gambogate[10,9,8:d,e]-8-amino-9-cyanopyran, methylpyrano[4,3,2-d,e]-5-ketone-4,13-dihydrobenzo[d′]imidazo[2′,1′:2,3]pyrimidino[6,5,4-h,i]gambogate, methyl-5-ketone-4,7,12-trihydro pyrano[4″,3″,2″:4′,5′,6′]gambogate[10′,8′:2,3,4]thiazepino[1,4][6,7-a]benzene, methyl-5-ketone-4,8,14,15-tetrahydropyrano[4″,3″,2″:4′,5′,6′]gambogate[10′,9′,8′:4,5,6]pyrano[3,2-b]indanone-1, methyl-5-ketone-4,14,15-trihydropyrano[4″,3″,2″:4′,5′,6]gambogate[10′,9′,8′:4,5,6]pyrido[3,2-b]indanone-1,methyl-5-ketone-4,8,9-trihydropyran-9-(1-H-3-amino-1,2,4-triazol-1-yl)pyrano[4,3,2:d,e]gambogate, methyl-5-ketone-4,8,9-trihydropyran-9-dicyanomethyl-pyrano[4,3,2:d,e]gambogate, methyl-5-ketone-8,9-dihydro-9-malonicamidoximepyrano[4,3,2-d,e]gambogate, methyl-5-ketone-4,7,11-trihydrogenpyrano[4,3,2-d,e]gambogate[2,4-b,c]-1′,5′,7′,9′-tetrazabicyclo(4,2,1)nonyl-3′,7′-bisene, methyl-4,5-dionepyrano[4,6-b,c]gambogate, methyl-4,6-dioneheptlactono[4,6-bc]gambogate.
Fused B-ring was formed between 6- and 8-position of gambogate acid analogs, methyl-6-H-oxazino[1,3][6,5,4-f,g]gambogate, methyl-6,8,9-trihydrogen-9-(1-H-3-amino-1,2,4-triazol-1-yl)oxazino[1,3][6,5,4-f,g]gambogate, methyl-6,8,9-trihydrogen-9-dicyano-methyleviltriazino[1,3][6,5,4-f,g]gambogate, methyl-8,9-dihydro-9-malonicamidoxime-oxazino[1,3][6,5,4-f,g]gambogate, methyl-6,8,9-trihydrogen-9-morpholinylhydrogen-1,3-oxazino[6,5,4-f,g]gambogate, methyl-6,8,9-trihydrogen-9-(4-methylpyrazine)-1,3-oxazino[6,5,4-f,g]gambogate, methyl-5-H-1,3-oxazino[6,5,4-f,g]gambogate.
Fused C-ring were formed between 8- and 10-position of gambogate acid analogs, methyl-6,11-dihydroimidazo[1′,2′:2,3]pyrimidino[6,5,4-h,i]gambogate, methyl-6,11-dihydro-5-(4-oxo-D-alloglycosyl)imidazo[2′,1′:2,3]pyrimidino[6,5,4-h,i]gambogate, methyl-6,11-dihydro-5-(4-oxo-D-glucosyl)imidazo[2′,1′:2,3]pyrimidino[6,5,4-h,i]gambogate, methyl-6,11-dihydro-5-(7,8-dihydroxy-4,4-dimethylhexahydropyrano[3,2-d]-1,3-dioxane-9-oxyimidazo[2′,1′:2,3]pyrimidino[6,5,4-h,i]gambogate, methyl-6,11-dihydro-5-(4-oxo-D-alloglycosyl)benzoylimidazo[2′,1′:2,3]pyrimidino[6,5,4-h,i]gambogate, methyl-6,11-dihydro-5-(4-oxo-D-glucosyl)benzoylimidazo[2′,1′:2,3]pyrimidino[6,5,4-h,i]gambogate, methyl-6,11-dihydro-5-methylimidazo[2′,1′:2,3]pyrimidino[6,5,4-h,i]gambogate, methyl-6,11-dihydro-5-[4-(4-methylpiperazin-1-yl)-4-oxobutyryl]imidazo[2′1′:2,3]pyrimidino[6,5,4-h,i]gambogate, methyl-6,11-dihydro-5-O-phosphorylimidazo[2′,1′:2,3]pyrimidino[6,5,4-h,i]gambogate, methyl-6,11-dihydro-5-oxygentriphosphorylimidazo[2′,1′:2,3]pyrimidino[6,5,4-h,i]gambogate, methyl-6,11-dihydro-5-[4-(4-methylpiperazin-1-yl)-3-oxo-propionyl]imidazolo[2′,1′:2,3]pyrimidino[6,5,4-h,i]gambogate, methyl-6,11-dihydro-5-(2-chloroacetyl)imidazo[2′,1′:2,3]pyrimidino[6,5,4-h,i]gambogate, methyl-6,11-dihydro-5-(2-hydroxyethylamino)ethylimidazo[2′,1′:2,3]pyrimidino[6,5,4-h,i]gambogate, methyl-6,11-dihydro-5-(2-(4-methylpiperazin-1-yl)ethyl)imidazo[2′,1′:2,3]pyrimidino[6,5,4-h,i]gambogate, methyl-6,11-dihydro-5-(2-hydroxyamino)methyl imidazo[2′,1′:2,3]pyrimidino[6,5,4-h,i]gambogate, methyl-6,11-dihydro-5-(bromomethyl)imidazo[2′,1′:2,3]pyrimidino[6,5,4-h,i]gambogate methyl-6,11-dihydro-5-(4-(7,8-dihydroxy-4,4-dimethylhexahydropyrano[3,2-d][1,3]dioxygenhexacyclo-9-oxy)ethaneformyl)imidazo[2′,1′:2,3]pyrimidino[6,5,4-h,i]gambogate, methyl-6,11-dihydrogentriazolo(1,2,4)[3′,2′:2,3]pyrimidino[6,5,4-h,i]gambogate, methyl-6,11-dihydro-5-(4-oxo-D-alloglycosyl)triazolo(1,2,4)[3′,2′:2,3]pyrimidino[6,5,4-h,i]gambogate, methyl-6,11-dihydro-5-(4-oxo-D-glucosyl)triazolo(1,2,4)[3′,2′:2,3]pyrimidino[6,5,4-h,i]gambogate, methyl-6,11-dihydro-5-(7,8-dihydroxy-4,4-dimethylhexahydropyrano[3,2-d][1,3]dioxane-9-oxy)triazolo(1,2,4)[3′,2′:2,3]pyrimidino[6,5,4-h,i]gambogate, methyl-6,11-dihydro-5-(4-oxo-D-alloglycosyl)benzoicacyltriazolo(1,2,4)[3′,2′:2,3]pyrimidino[6,5,4-h,i]gambogate, methyl-6,11-dihydro-5-(4-oxo-D-glucosyl)benzoyltriazolo(1,2,4)[3′,2′:2,3]pyrimidino[6,5,4-h,i]gambogate methyl-6,11-dihydro-5-methyltriazolo(1,2,4)[3′,2′:2,3]pyrimidino[6,5,4-h,i]gambogate, methyl-6,11-dihydro-5-[4-(4-methylpiperazine-1-yl)-4-oxo-dibutyryl]triazolo(1,2,4)[3′,2′:2,3]pyrimidino[6,5,4-h,i]gambogate, methyl-6,11-dihydro-5-oxygenphosphoryltriazolo(1,2,4)[3′,2′:2,3]pyrimidino[6,5,4-h,i]gambogate, methyl-6,11-dihydro-5-triphosphoryloxygentriazolo(1,2,4)[3′,2′:2,3]pyrimidino[6,5,4-h,i]gambogate, methyl-6,11-dihydro-5-[4-(4-methylpiperazin-1-yl)-3-oxo-propionyl]triazolo(1,2,4)[3′,2′:2,3]pyrimidino[6,5,4-h,i]gambogate, methyl-6,11-dihydro-5-(2-chloroacetyl)triazolo(1,2,4)[3′,2′:2,3]pyrimidino[6,5,4-h,i]gambogate, methyl-6,11-dihydro-5-(2-hydroxyethylamino)ethyltriazolo(1,2,4)[3′,2′:2,3]pyrimidino[6,5,4-h,i]gambogate, methyl-6,11-dihydro-5-(2-(4-methylpiperazine-1-yl)-ethyl)triazolo(1,2,4)[3′,2′:2,3]pyrimidino[6,5,4-h,i]gambogate, methyl-6,11-dihydro-5-(2-hydroxyamino)methyltriazolo(1,2,4)[3′,2′:2,3]pyrimidino[6,5,4-h,i]gambogate, methyl-6,11-dihydro-5-bromomethyl-triazolo(1,2,4)[3′,2′:2,3]pyrimidino[6,5,4-h,i]gambogate, methyl-6,11-dihydro-5-(4-(7,8-dihydroxy-4,4-dimethylhexahydropyrano[3,2-d][1,3]dioxane-9-oxy)benzoyl)triazolo(1,2,4)[3′,2′:2,3]pyrimidino[6,5,4-h,i]gambogate, methyl-7-amino-8-cyano-9-H-pyrido[6,5,4-h,i]gambogate, methyl-5-(4-O-D-alloglycosyl)-7-amino-8-cyano-9-H-pyrido[6,5,4-h,i]gambogate, methyl-5-(4-O-D-glucose)-7-amino-8-cyano-9-H-pyrido[6,5,4-h,i]gambogate, methyl-5-(7,8-dihydroxy-4,4-dimethyl-hexahydropyrano[3,2-d][1,3]dioxane-9-oxy)-7-amino-8-cyano-9-H-pyrido[6,5,4-h,i]gambogate, methyl-5-(4-oxo-D-alloglycosyl)benzoyl-7-amino-8-cyano-9-H-pyrido[6,5,4-h,i]gambogate, methyl-5-(4-oxo-D-glucose)benzoyl-7-amino-8-cyano-9-H-pyrido[6,5,4-h,i]gambogate, methyl-5-methyl-7-amino-8-cyano-9-H-pyrido[6,5,4-h,i]gambogate, methyl-5-[4-(4-methylpiperazin-1-yl)-4-oxobutyryl]-7-amino-8-cyano-9-H-pyrido[6,5,4-h,i]gambogate, methyl-5-O-phosphoryl-7-amino-8-cyano-9-H-pyrido[6,5,4-h,i]gambogate, methyl-5-oxygentriphosphoryl-7-amino-8-cyano-9-H-pyrido[6,5,4-h,i]gambogate, methyl-5-[4-(4-methylpiperazin-1-yl)-3-oxo-propionyl]-7-amino-8-cyano-9-H-pyrido[6,5,4-h,i]gambogate, methyl-5-(2-chloroacetyl)-7-amino-8-cyano-9-H-pyrido[6,5,4-h,i]gambogate, methyl-5-(2-hydroxy)ethyl-7-amino-8-cyano-9-H-pyrido[6,5,4-h,i]gambogate, methyl-5-(2-(4-methyl piperazin-1-yl)ethyl)-7-amino-8-cyano-9-H-pyrido[6,5,4-h,i]gambogate, methyl-5-(2-hydroxyamine) methyl-7-amino-8-cyano-9-H-pyrido[6,5,4-h,i]gambogate, methyl-5-bromomethyl-7-amino-8-cyano-9-H-pyrido[6,5,4-h,i]gambogate, methyl-5-(4-(7,8-dihydroxy-4,4-dimethylhexahydropyrano[3,2-d](1,3)dioxane-9-oxy)benzoyl)-7-amino-8-cyano-9-H-pyrido[6,5,4-h,i]gambogate, methyl-6-methyl-7-amino-8-cyano-9-H-pyrido[6,5,4-h,i]gambogate, methyl-5-(4-oxo-D-alloglycosyl)-6-dimethyl-7-amino-8-cyano-9-H-pyrido[6,5,4-h,i]gambogate, methyl-5-(4-oxo-D-glucosyl)-6-dimethyl-7-amino-8-cyano-9-H-pyrido[6,5,4-h,i]gambogate, methyl-5-(7,8-dihydroxy-4,4-dimethylhexahydropyrano[3,2-d][1,3]dioxane-9-oxy)-6-methyl-7-amino-8-cyano-9-H-pyrido[6,5,4-h,i]gambogate, methyl-5-(4-oxo-D-alloglycosyl)benzoyl-6-dimethyl-7-amino-8-cyano-9-H-pyrido[6,5,4-h,i]gambogate, methyl-5-(4-oxo-D-glucose)benzoyl-6-dimethyl-7-amino-8-cyano-9-H-pyrido[6,5,4-h,i]gambogate, methyl-5,6-dimethyl-7-amino-8-cyano-9-H-pyrido[6,5,4-h,i]gambogate, methyl-5-[4-(4-methylpiperazin-1-yl)-4-oxo-butyryl]-6-dimethyl-7-amino-8-cyano-9-Hpyrido[6,5,4-h,i]gambogate, methyl-5-O-phosphoryl-6-methyl-7-amino-8-cyano-9-H-pyrido[6,5,4-h,i]gambogate, methyl-5-O-triphosphoryl-6-methyl-7-amino-8-cyano-9-H-pyrido[6,5,4-h,i]gambogate, methyl-5-[4-(4-methylpiperazin-1-yl)-3-oxo-propionyl]-6-dimethyl-7-amino-8-cyano-9-H-pyrido[6,5,4-h,i]gambogate, methyl-5-(2-chloroacetyl)-6-methyl-7-amino-8-cyano-9-H-pyrido[6,5,4-h,i]gambogate, methyl-5-(2-hydroxyethylamino)ethyl-6-dimethyl-7-amino-8-cyano-9-H-pyrido[6,5,4-h,i]gambogate, methyl-5-(2-(4-methylpiperazin-1-yl)-ethyl)-6-methyl-7-amino-8-cyano-9-H-pyrido[6,5,4-h,i]gambogate, methyl-5-(2-hydroxyamino)methyl-6-methyl-7-amino-8-cyano-9-H-pyrido[6,5,4-h,i]gambogate, methyl-5-bromomethyl-6-dimethyl-7-amino-8-cyano-9-H-pyrido[6,5,4-h,i]gambogate, methyl-5-(4-(7,8-dihydroxy-4,4-dimethylhexahydropyrano[3,2-d][1,3]dioxane-9-oxy)benzoyl)-6-methyl-7-amino-8-cyano-9-H-pyrido[6,5,4-h,i]gambogate, methyl-7-amino-8-cyano-9-H-pyrano[6,5,4-h,i]gambogate, methyl-5-(4-oxo-D-alloglycosyl)-7-amino-8-cyano-9-H-pyrano[6,5,4-h,i]gambogate, methyl-5-(4-oxo-D-glucose)-7-amino-8-cyano-9-H-pyrano[6,5,4-h,i]gambogate, methyl-5-(7,8-dihydroxy-4,4-dimethyl-hexahydropyrano[3,2-d][1,3]dioxane-9-oxy)-7-amino-8-cyano-9-H-pyrano[6,5,4-h,i]gambogate, methyl-5-(4-oxo-D-alloglycosyl)benzoicacyl-7-amino-8-cyano-9-H-pyrano[6,5,4-h,i]gambogate, methyl-5-(4-oxo-D-glucosyl)benzoyl-7-amino-8-cyano-9-H-pyrano[6,5,4-h,i]gambogate, methyl-5-methyl-7-amino-8-cyano-9-H-pyrano[6,5,4-h,i]gambogate, methyl-5-[4-(4-methyl-piperzin-1-yl)-4-oxobutyryl]-7-amino-8-cyano-9-H-pyrano[6,5,4-h,i]gambogate, methyl-5-O-phosphoryl-7-amino-8-cyano-9-H-pyrano[6,5,4-h,i]gambogate, methyl-5-O-triphosphorusacyl-7-amino-8-cyano-9-H-pyrano[6,5,4-h,i]gambogate, methyl-5-[4-(4-methylpiperazin-1-yl)-3-oxo-propionyl]-7-amino-8-cyano-9-H-pyrano[6,5,4-h,i]gambogate, methyl-5-(2-chloroacetyl)-7-amino-8-cyano-9-H-pyrano[6,5,4-h,i]gambogate, methyl-5-(2-hydroxyethylamino)ethyl-7-amino-8-cyano-9-H-pyrano[6,5,4-h,i]gambogate, methyl-5-(2-(4-methylpiperazin-1-yl)-yl)-7-amino-8-cyano-9-H-pyrano[6,5,4-h,i]gambogate, methyl-5-(2-hydroxyethylamino)methyl-7-amino-8-cyano-9-H-pyrano[6,5,4-h,i]gambogate, methyl-5-bromomethyl-7-amino-8-cyano-9-H-pyrano[6,5,4-h,i]gambogate, methyl-5-(4-(7,8-dihydroxy-4,4-dimethylhexandropyrano[3,2-d][1,3]dioxane-9-oxy)benzoyl)-7-amino-8-cyano-9-H-pyrano[6,5,4-h,i]gambogate, methyl-13-H-benzo[d′]imidazo[2′,1′:2,3]pyrimidino[6,5,4-h,i]gambogate, methyl-5-(4-O-D-alloglycosyl)-13-H-benzo[d′]imidazo[2′,1′:2,3]pyrimidino[6,5,4-h,i]gambogate, methyl-5-(4-oxo-D-glucosyl)-13-hydrogenbenzeno[d′]imidazo[2′,1′:2,3]pyrimidino[6,5,4-h,i]gambogate, methyl-5-(7,8-dihydroxy-4,4-dimethyhexahydropyrano[3,2-d][1,3]dioxane-9-oxy)-13-H-benzo[d′]imidazo[2′,1′:2,3]pyrimidino[6,5,4-h,i]gambogate, methyl-5-(4-O-D-alloglycosyl-) benzoyl-13-H-benzo[d′]imidazo[2′,1′:2,3]pyrimidino[6,5,4-h,i]gambogate, methyl-5-(4-O-D-glucose)benzoyl-13-H-benzo[d′]imidazo[2′,1′:2,3]pyrimidino[6,5,4-h,i]gambogate, methyl-5-methyl-13-H-benzo[d′]imidazo[2′,1′:2,3]pyrimidino[6,5,4-h,i]gambogate, methyl-5-[4-(4-methylpiperazin-1-yl)-4-oxobutyryl]-13-H-benzo[d′]imidazo[2′,1′:2,3]pyrimidino[6,5,4-h,i]gambogate, methyl-5-O-phosphoryl-13-H-benzo[d′]imidazo[2′,1′:2,3]pyrimidino[6,5,4-h,i]gambogate, methyl-5-β-triphosphoryl-13-hydrogenbenzo[d′]imidazo[2′,1′:2,3]pyrimidino[6,5,4-h,i]gambogate, methyl-5-[4-(4-methylpiperazine-1-yl)-3-oxo-propionyl]-13-H-benzo[d′]imidazo[2′,1′:2,3]pyrimidino[6,5,4-h,i]gambogate, methyl-5-(2-chloroacetyl)-13-H-benzo[d′]imidazo[2′,1′:2,3]pyrimidino[6,5,4-h,i]gambogate, methyl-5-(2-hydroxyethylamino)ethyl-13-H-benzo[d′]imidazo[2′,1′:2,3]pyrimidino[6,5,4-h,i]gambogate, methyl-5-(2-(4-methylpiperazin-1-yl)-ethyl)-13-H-benzo[d′]imidazo[2′,1′:2,3]pyrimidino[6,5,4-h,i]gambogate, methyl-5-(2-hydroxyethylamino)methyl-13-H-benzo[d]imidazo[2′,1′:2,3]pyrimidino[6,5,4-h,i]gambogate, methyl-5-(bromomethyl)-13-H-benzo[d′]imidazo[2′,1′:2,3]pyrimidino[6,5,4-h,i]gambogate, methyl-5-(4-(7,8-dihydroxy-4,4-dimethylhexahydropyrano[3,2-d][1,3]dioxane-9-oxygenyl)benzoyl)-13-H-benzo[d′]imidazo[2′,1′:2,3]pyrimidino[6,5,4-h,i]gambogate, methyl-6,12-dihydrobenzo[b][1,4]thiazepino[4,3,2-h,i]gambogate, methyl-5-(4-oxo-D-alloglycosyl)-6,12-dihydrobenzo[b][1,4]thiazepino[4,3,2-h,i]gambogate, methyl-5-(4-oxo-D-glucose)-6,12-dihydrobenzo[b][1,4]thiazepino[4,3,2-h,i]gambogate, methyl-5-(7,8-dihydroxy-4,4-dimethylhexa hydropyrano[3,2-d][1,3]dioxane-9-oxy)-6,12-dihydrobenzo[b][1,4]thiazepino[4,3,2-h,i]gambogate, methyl-5-(4-O-glycosyl-D-allo) benzoyl-6,12-dihydrobenzo[b][1,4]thiazepino[4,3,2-h,i]gambogate, methyl-5-(4-oxo-D-glucosyl)benzeneformyl-6,12-dihydrobenzo[b][1,4]thiazepino[4,3,2-h,i]gambogate, methyl-5-methyl-6,12-dihydrobenzo[b][1,4]thiazepino[4,3,2-h,i]gambogate, methyl-5-[4-(4-methylpiperazin-1-yl)-4-oxobutyryl]-6,12-dihydrobenzo[b][1,4]thiazepino[4,3,2-h,i]gambogate, 5-O-phosphoryl-6,12-dihydrobenzo[b][1,4]thiazepino[4,3,2-h,i]gambogate, methyl-5-O-triphosphorylcholine-6,12-dihydrobenzo[b][1,4]thiazepino[4,3,2-h,i]gambogate, methyl-5-[4-(4-methylpiperzin-1-yl)-3-oxopropionyl]-6,12-dihydrobenzo[b][1,4]thiazepino[4,3,2-h,i]gambogate, methyl-5-(2-chloroacetyl)-6,12-dihydrobenzo[b][1,4]thiazepino[4,3,2-h,i]gambogate, methyl-5-(2-hydroxyethylamino)ethyl-6,12-dihydrobenzo[b][1,4]thiazepino[4,3,2-h,i]gambogate, methyl-5-(2-(4-methylpiperazin-1-yl)ethyl)-6,12-dihydrobenzo[b][1,4]thiazepino[4,3,2-h,i]gambogate, methyl-5-(2-hydroxyethylamino)methyl-6,12-dihydrobenzo[b][1,4]thiazepino[4,3,2-h,i]gambogate, methyl-5-(bromomethyl)-6,12-dihydrobenzo[b][1,4]thiazepino[4,3,2-h,i]gambogate, methyl-5-(4-(7,8-dihydroxy-4,4-dimethyl hexahydropyrano[3,2-d][1,3]dioxane-9-oxy)benzoyl)-6,12-dihydrobenzo[b][1,4]thiazepino[4,3,2-h,i]gambogate, methyl-7,13,14-trihydrogenindanone-1′[3′,2′:2,3]pyrano[6,5,4-h,i]gambogate, methyl-5-(4-oxo-D-alloglycosyl)-7,13,14-trihydrogen-indanone-1′[3′,2′:2,3]pyrano[6,5,4-h,i]gambogate, methyl-5-(4-oxo-D-glucose)-7,13,14-tri-hydrogenindanone-1′[3′,2′:2,3]pyrano[6,5,4-h,i]gambogate, methyl-5-(7,8-dihydroxy-4,4-dimethylhexahydropyrano[3,2-d][1,3]dioxane-9-oxy)-7,13,14-trihydrogenindanone-1′[3′,2′:2,3]pyrano[6,5,4-h,i]gambogate, methyl-5-(4-oxo-D-alloglycosyl)benzeneformyl-7,13,14-trihydrogenindanone-1′[3′,2′:2,3]pyrano[6,5,4-h,i]gambogate, methyl-5-(4-oxo-D-glucosyl)-benzoyl-7,13,14-trihydrogenindanone-1′[3′,2′:2,3]pyrano[6,5,4-h,i]gambogate, methyl-5-methyl-7,13,14-trihydrogenindanone-1′[3′,2′:2,3]pyrano[6,5,4-h,i]gambogate, methyl-5-[4-(4-methylpiperazin-1-yl)-4-oxobutyryl]-7,13,14-trihydrogenindanone-1′[3′,2′:2,3]pyrano[6,5,4-h,i]gambogate, methyl-5-O-phosphoryl-7,13,14-trihydrogenindanone-1′[3′,2′:2,3]pyrano[6,5,4-h,i]gambogate, methyl-5-O-triphosphoryl-7,13,14-trihydrogenindanone-1′[3′,2′:2,3]pyrano[6,5,4-h,i]gambogate, methyl-5-[4-(4-methylpiperzin-1-yl)-3-oxo-propionyl]-7,13,14-trihydrogenindanone-1′[3′,2′:2,3]pyrano[6,5,4-h,i]gambogate, methyl-5-(2-chloroacetyl)-7,13,14-trihydrogenindanone-1′[3′,2′:2,3]pyrano[6,5,4-h,i]gambogate, methyl-5-(2-hydroxyethylamino)ethyl-7,13,14-trihydrogenindanone-1′[3′,2′:2,3]pyrano[6,5,4-h,i]gambogate, methyl-5-(2-(4-methylpiperazin-1-yl)ethyl)-7,13,14-trihydrogenindanone-1′[3′,2′:2,3]pyrano[6,5,4-h,i]gambogate, methyl-5-(2-hydroxyamino)methyl-7,13,14-trihydrogenindanone-1′[3′,2′:2,3]pyrano[6,5,4-h,i]gambogate, methyl-5-(bromomethyl-)-7,13,14-trihydrogenindanone-1′[3′,2′:2,3]pyrano[6,5,4-h,i]gambogate, methyl-5-(4-(7,8-dihydroxy-4,4-dimethylhexahydropyrano[3,2-d][1,3]dioxane-9-oxy)benzoyl)-7,13,14-trihydrogenindanone-1′[3′,2′:2,3]pyrano[6,5,4-h,i]gambogate, methyl-12,13-dihydro-indanone-1′[3′,2′:2,3]pyrido[6,5,4-h,i]gambogate, methyl-5-(4-oxo-D-glucose)-12,13-dihydroindanone-1′[3′,2′:2,3]pyrido[6,5,4-h,i]gambogate, methyl-5-(4-oxo-D-allo-glycosyl)-12,13-dihydroindanone-1′[3′,2′:2,3]pyrido[6,5,4-h,i]gambogate, methyl-5-(7,8-dihydroxy-4,4-dimethylhexahydropyrano[3,2-d][1,3]dioxane-9-oxy)-12,13-dihydro-indanone-1′[3′,2′:2,3]pyrido[6,5,4-h,i]gambogate, methyl-5-(4-oxo-D-alloglycosyl)benzoyl-12,13-dihydroindanone-1′[3′,2′:2,3]pyrido[6,5,4-h,i]gambogate, methyl-5-(4-oxo-D-glucose)benzoyl-12,13-dihydroindanone-1′[3′,2′:2,3]pyrido[6,5,4-h,i]gambogate, methyl-5-methyl-12,13-dihydroindanone-1′[3′,2′:2,3]pyrido[6,5,4-h,i]gambogate, methyl-5-[4-(4-methylpiperazine-1-yl)-4-oxobutyryl]-12,13-dihydroindanone-1′[3′,2′:2,3]pyrido[6,5,4-h,i]gambogate, methyl-5-O-phosphoryl-12,13-dihydroindeneketone-1′[3′,2′:2,3]pyrido[6,5,4-h,i]gambogate methyl-5-O-triphosphoryl-12,13-dihydroindanone-1′[3′,2′:2,3]pyrido[6,5,4-h,i]gambogate, methyl-5-[4-(4-methylpiperazine-1-yl)-3-oxo-propionyl]-12,13-dihydroindanone-1′[3′,2′:2,3]pyrido[6,5,4-h,i]gambogate, methyl-5-(2-chloroacetyl)-12,13-dihydroindanone-1′[3′,2′:2,3]pyrido[6,5,4-h,i]gambogate, methyl-5-(2-hydroxyethylamino)ethyl-12,13-dihydroindanone-1′[3′,2′:2,3]pyrido[6,5,4-h,i]gambogate, methyl-5-(2-(4-methylpiperazin-1-yl)ethyl)-12,13-dihydro-indanone-1′[3′,2′:2,3]pyrido[6,5,4-h,i]gambogate, methyl-5-(2-hydroxyethylamino)methyl-12,13-dihydroindanone-1′[3′,2′:2,3]pyrido[6,5,4-h,i]gambogate, methyl-5-(bromomethyl-)-12,13-dihydroindanone-1′[3′,2′:2,3]pyrido[6,5,4-h,i]gambogate, methyl-5-(4-(7,8-dihydroxy-4,4-dimethylhexahydropyrano[3,2-d][1,3]dioxane-9-oxy)benzoyl)-12,13-dihydrogenindanone-1′[3′,2′:2,3]pyrido[6,5,4-h,i]gambogate, methyl-9,10-dihydro-10-(1-H-3-amino-1,2,4-triazole)gambogate, methyl-6-(4-oxo-D-alloglycosyl)-9,10-dihydro-10-(1-H-3-amino-1,2,4-triazol-1-yl)gambogate, methyl-6-(4-oxo-D-glucose)-9,10-dihydroxy-10-(1-H-3-amino-1,2,4-triazole-1-yl)gambogate, methyl-6-(7,8-dihydroxy-4,4-dimethylhexahydropyrano[3,2-d][1,3]dioxane-9-oxy)-9,10-dihydro-10-(1-H-3-amino-1,2,4-triazol-1-yl)gambogate, methyl-6-(4-oxo-D-alloglycosyl)benzoyl-9,10-dihydro-10-(1-H-3-amino-1,2,4-triazole-1-yl)gambogate, methyl-6-(4-oxo-D-glucosyl)benzoyl-9,10-dihydro-10-(1-H-3-amino-1,2,4-triazol-1-yl)gambogate, methyl-6-methyl-9,10-dihydro-10-(1-H-3-amino-1,2,4-triazole)gambogate, methyl-6-[4-(4-methylpiperazin-1-yl)-4-oxobutyryl]-9,10-dihydro-10-(1-H-3-amino-1,2,4-triazol-1-yl)gambogate, methyl-6-O-phosphoryl-9,10-dihydro-10-(1-H-3-amino-1,2,4-triazol-1-yl)gambogate, methyl-6-O-triphosphoryl-9,10-dihydro-10-(1-H-3-amino-1,2,4-triazole-1-yl)gambogate, methyl-6-[4-(4-methylpiperazin-1-yl)-3-oxo-propionyl]-9,10-dihydro-10-(1-H-3-amino-1,2,4-triazol-1-yl)gambogate, methyl-6-(2-chloroacetyl)-9,10-dihydro-10-(1-H-3-amino-1,2,4-triazol-1-yl)gambogate, methyl-6-(2-hydroxyethylamino)ethyl-9,10-dihydro-10-(1-H-3-amino-1,2,4-triazol-1-yl)gambogate, methyl-6-(2-(4-methylpiperzin-1-yl)-ethyl)-9,10-dihydro-10-(1-H-3-amino-1,2,4-triazole)gambogate, methyl-6-(2-hydroxyamino)methyl-9,10-dihydro-10-(1-H-3-amino-1,2,4-triazol-1-yl)gambogate, methyl-6-(bromomethyl)-9,10-dihydro-10-(1-H-3-amino-1,2,4-tritriazole-1-yl)gambogate, methyl-6-(4-(7,8-dihydroxy-4,4-dimethyl-hexahydropyrano[3,2-d][1,3]dioxane-9-oxy)benzoyl)-9,10-dihydro-10-(1-H-3-amino-1,2,4-triazol-1-yl)gambogate, methyl-9,10-dihydro-10-dicyanomethylgambogate, methyl-6-(4-oxo-D-alloglycosyl)-9,10-dihydro-10-dicyanidegambogate, methyl-6-(4-oxo-D-glucose)-9,10-dihydro-10-dicyanomethylgambogate, methyl-6-(7,8-dihydroxy-4,4-dimethylhexahydropyrano[3,2-d][1,3]dioxane-9-oxy)-9,10-dihydro-10-dicyanomethylgambogate, methyl-6-(4-oxo-D-alloglycosyl)benzoyl-9,10-dihydro-10-dicyanomethylgambogate, 6-(4-oxo-D-glucosyl)benzoyl-9,10-dihydro-10-dicyanomethylgambogate, methyl-6-methyl-9,10-dihydro-10-dicyanidegambogate, methyl-6-[4-(4-methylpiperazin-1-yl)-4-oxobutyryl]-9,10-dihydro-10-dicyanomethylgambogate, 6-O-phosphorylcholine-9,10-dihydro-10-dicyanomethyl-gambogate, 6-O-triphosphoryl-9,10-dihydro-10-dicyanomethylgambogate, methyl-6-[4-(4-methyl-piperazine-1-yl)-3-oxo-propionyl]-9,10-dihydro-10-dicyanomethylgambogate, methyl-6-(2-chloroacetyl)-9,10-dihydro-10-dicyanomethylgambogate, methyl-6-(2-hydroxyethylamino)ethyl-9,10-dihydro-10-dicyanomethylgambogate, methyl-6-(2-(4-methylpiperazin-1-yl)ethyl)-9,10-dihydro-10-dicyanomethylgambogate, methyl-6-(2-hydroxyamino)methyl-9,10-dihydro-10-dicyanomethylgambogate, methyl-6-(bromo-methyl-)-9,10-dihydro-10-dicyanomethylgambogate, methyl-6-(4-(7,8-dihydroxy-4,4-dimethylhexahydropyrano[3,2-d][1,3]dioxane-9-oxygen)benzoyl)-9,10-dihydro-10-dicyanomethylgambogate, methyl-9,10-dihydro-10-malonicamidoximegambogate, methyl-6-(4-O-D-allosugar)-9,10-dihydro-10-malonicamidoximegambogate, methyl-6-(4-oxo-D-glucose)-9,10-dihydro-10-malonylaminooximegambogate, methyl-6-(7,8-dihydroxy-4,4-dimethylhexahydropyrano[3,2-d][1,3]dioxane-9-oxy)-9,10-dihydro-10-malonicamidoximegambogate, methyl-6-(4-oxo-D-alloglycosyl)-benzoyl-9,10-dihydro-10-malonylamidoximegambogate, methyl-6-(4-oxo-D-glucose)benzoicacyl-9,10-dihydro-10-malonicamidoximegambogate, methyl-6-methyl-9,10-dihydro-10-malonicamidoximegambogate, methyl-6-[4-(4-methylpiperazine-1-yl)-4-oxobutyryl]-9,10-dihydro-10-malonicamidoximegambogate, methyl-6-O-phosphoryl-9,10-dihydro-10-malonicamidoximegambogate, methyl-6-O-triphosphoryl-9,10-dihydro-10-malonic-amidoximegambogate, methyl-6-[4-(4-methylpiperazine-1-yl)-3-oxopropionyl]-9,10-dihydro-10-malonicamidoximegambogate, methyl-6-(2-chloroacetyl)-9,10-dihydro-10-malonylamidoximegambogate, methyl-6-(2-hydroxyethyl)ethyl-9,10-dihydro-10-malonicamidoximegambogate, methyl-6-(2-(4-methylpiperazin-1-yl)ethyl)-9,10-dihydro-10-malonylamidoximegambogate, methyl-6-(2-hydroxyethylamino)methyl-9,1-aminodihydro-10-malonicamidoximegambogate, methyl-6-methyl-bromide-9,10-dihydro-10-malonicamidoximegambogate, methyl-6-(4-(7,8-dihydroxy-4,4-dimethyl-hexahydropyrano[3,2-d][1,3]dioxane-9-oxy)benzoyl)-9,10-dihydro-10-malonicamidoximegambogate, methyl-10-H-1′,5′,7′,9′-tetrazabicyclo(4,2,1)nonyl-3′,7′-dieno[2,4-h,i]gambogate, methyl-5-(4-oxo-D-alloglycosyl)-10-H-1′,5′,7′,9′-tetrazabicyclo(4,2,1)nonyl-3′,7′-dieno[2,4-h,i]gambogate, methyl-5-(4-oxo-D-glucose)-10-H-1′,5′,7′,9′-tetrazabicyclo(4,2,1)nonyl-3′,7′-dieno[2,4-h,i]gambogate, methyl-5-(7,8-dihydroxy-4,4-dimethyl-hexahydropyrano[3,2-d][1,3]dioxane-9-oxy)-10-H-1′,5′,7′,9′-tetrazabicyclo(4,2,1)nonyl-3′,7′-dieno[2,4-h,i]gambogate, methyl-5-(4-oxo-D-alloglycosyl)benzoyl-10-H-1′,5′,7′,9′-tetrazabicyclo(4,2,1)nonyl-3′,7′-dieno[2,4-h,i]gambogate, methyl-5-(4-oxo-D-glucosyl)benzoyl-10-H-1′,5′,7′,9′-tetrazabicyclo(4,2,1)nonyl-3′,7′-dieno[2,4-h,i]gambogate, methyl-5-methyl-10-H-1′,5′,7′,9′-tetrazabicyclo(4,2,1)nonyl-3′,7′-dieno[2,4-h,i]gambogate, methyl-5-[4-(4-methylpiperazine-1-ly)-4-oxobutyryl]-10-H-1′,5′,7′,9′-tetrazabicyclo(4,2,1)nonyl-3′,7′-dieno[2,4-h,i]gambogate, methyl-5-O-phosphoryl-10-H-1′,5′,7′,9′-tetrazabicyclo(4,2,1)nonyl-3′,7′-dieno[2,4-h,i]gambogate, methyl-5-O-triphosphoryl-10-H-1′,5′,7′,9′-tetrazabicyclo(4,2,1)nonyl-3′,7′-dieno[2,4-h,i]gambogate, methyl-5-[4-(4-methyl piperazine-1-yl)-3-oxo-propionyl]-10-H-1′,5′,7′,9′-tetrazabicyclo(4,2,1)nonyl-3′,7′-dieno[2,4-h,i]gambogate, methyl-5-(2-chloroacetyl)-10-H-1′,5′,7′,9′-tetrazabicyclo(4,2,1) nonyl-3′,7′-dieno[2,4-h,i]gambogate, methyl-5-(2-hydroxyamino)ethyl-10-H-1′,5′,7′,9′-tetrazabicyclo(4,2,1)nonyl-3′,7′-dieno[2,4-h,i]gambogate, methyl-5-(2-(4-methyl-piperazin-1-yl)-ethyl)-10-H-1′,5′,7′,9′-tetrazabicyclo(4,2,1)nonyl-3′,7′-dieno[2,4-h,i]gambogate, methyl-5-(2-hydroxyethylamino)methyl-10-H-1′,5′,7′,9′-tetrazabicyclo(4,2,1)nonyl-3′,7′-dieno[2,4-h,i]gambogate, methyl-5-bromomethyl-10-H-1′,5′,7′,9′-tetrazabicyclo(4,2,1)nonyl-3′,7′-dieno[2,4-h,i]gambogate, methyl-5-(4-(7,8-dihydroxy-4,4-dimethylhexahydropyrano[3,2-d][1,3]dioxane-9-oxy)benzoyl-10-H-1′,5′,7′,9′-tetrazabicyclo(4,2,1)nonyl-3′,7′-dieno[2,4-h,i]gambogate, methyl-1-cyclopropyl-6-fluoro-4-oxo-7-[4-(9,10-dihydro-10-gambogate)piperazinyl]-1,4-dihydroquinoline-3-carboxylic acid, methyl-1-cyclopropyl-6-fluoro-4-oxo-7-[4-(6-(4-oxo-D-alloglycosyl)-9,10-dihydro-10-gambogate)piperazinyl]-1,4-dihydroquinoline-3-carboxylic acid, methyl-1-cyclopropyl-6-fluoro-4-oxo-7-[4-(6-(4-O-D-glucose)-9,10-dihydro-10-gambogate)piperazinyl]-1,4-dihydroquinoline-3-carboxylic acid, methyl-1-cyclopropyl-6-fluoro-4-oxo-7-[4-(6-methyl-9,10-dihydro-10-gambogate)piperazinyl]-1,4-dihydroquinoline-3-carboxyl acid, methyl-6-aminoethyl-12-hydrogenindanone-1′-[3″,2″:2′,3′]pyrido[6,5,4-h,i]gambogate, methyl-5-(4-oxo-D-alloglycosyl)-6-aminoethyl-12-H-indanone-1′[3′,2′:2,3]pyrido[6,5,4-h,i]gambogate, methyl-5-(4-oxo-D-glucosyl)-6-aminoethyl-12-H-indanone-1′[3′,2′:2,3]pyrido[6,5,4-h,i]gambogate, methyl-5-(7,8-dihydroxy-4,4-dimethylhexahydropyrano[3,2-d][1,3]dioxane-9-oxy)-6-aminoethyl-12-H-indanone-1′[3′,2′:2,3]pyridino[6,5,4-h,i]gambogate, methyl-5-(4-oxo-D-alloglycosyl)benzoyl-6-aminoethyl-12-H-indanone-1′[3′,2′:2,3]pyridino[6,5,4-h,i]gambogate, methyl-5-(4-oxo-D-glucosyl)benzoyl-6-aminoethyl-12-H-indanone-1′[3′,2′:2,3]pyrido[6,5,4-h,i]gambogate, methyl-5-methyl-6-aminoethyl-12-H-indanone-1′[3′,2′:2,3]pyrido[6,5,4-h,i]gambogate, methyl-5-[4-(4-methylpiperzin-1-yl)-4-oxobutyryl]-6-aminoethyl-12-H-indanone-1′[3′,2′:2,3]pyrido[6,5,4-h,i]gambogate, methyl-5-O-phosphoryl-6-aminoethyl-12-H-indanone-1′[3′,2′:2,3]pyrido[6,5,4-h,i]gambogate, methyl-5-O-triphosphoryl-6-aminoethyl-12-H-indanone-1′[3′,2′:2,3]pyrido[6,5,4-h,i]gambogate, methyl-5-[4-(4-methylpiperzin-1-yl)-3-oxopropionyl]-6-aminoethyl-12-H-indanone-1′-[3′,2′:2,3]pyrido[6,5,4-h,i]gambogate, methyl-5-(2-chloro-acetyl)-6-aminoethyl-12-hydrogenindanone-1′[3′,2′:2,3]pyrido[6,5,4-h,i]gambogate, methyl-5-(2-hydroxyethylamino)ethyl-6-aminoethyl-12-H-indanone-1′-[3′,2′:2,3]pyrido[6,5,4-h,i]gambogate, methyl-5-(2-(4-methylpiperzin-1-yl)ethyl)-6-aminoethyl-12-H-indanone-1′[3′,2′:2,3]pyrido[6,5,4-h,i]gambogate, methyl-5-(2-hydroxyethylamino)methyl-6-amino-ethyl-12-H-indanone-1′ [3′,2′:2,3]pyrido[6,5,4-h,i]gambogate, methyl-5-ketone-4,13-dihydro-7-aminoethylpyrano[4″,3″,2″:4′,5′,6′]gambogate[10′,9′,8′:4,5,6]pyrido[3,2-b]indanone-1, methyl-5-bromomethyl-6-aminoethyl-12-H-indanone-1′[3′,2′:2,3]pyrido[6,5,4-h,i]gambogate, methyl-5-(4-(7,8-dihydroxy-4,4-dimethylhexahydropyrano[3,2-d][1,3]dioxane-9-oxy)benzoyl)-6-aminoethyl-12-hydrogenindanone-1′[3′,2′:2,3]pyrido[6,5,4-h,i]gambogate, methyl-6-(4-(7,8-dihydroxy-4,4-dimethylhexahydropyrano[3,2-d][1,3]dioxane-9-oxy))-9,10-dihydro-10-morpholinylgambogate, methyl-6-[4-(4-methyl-piperazine-1-yl)-4-oxobutyryl]-9,10-dihydro-10-morpholinylgambogate, methyl-6-[4-(4-methylpiperazine-1-yl)-3-oxopropionyl]-9,10-dihydro-10-morpholinylgambogate, methyl-6-(2-chloroacetyl)-9,10-dihydro-10-morpholinylgambogate, methyl-6-(2-hydroxyethylamino)ethyl-9,10-dihydro-10-morpholinylgambogate, methyl-6-(2-(4-methylpiperzin-1-yl)ethyl)-9,10-dihydro-10-morpholinylgambogate, methyl-6-(2-hydroxyethylamino)methyl-9,10-dihydro-10-morpholinylgambogate, methyl-5-ketone-4,8,9-trihydrogen-pyrano[4,3,2-d,e]-9-morpholinylgambogate, methyl-5-(bromomethyl)-9,10-dihydro-10-morpholinylgambogate, methyl-9,10-dihydro-10-morpholino-6-(4-(7,8-dihydroxy-4,4-dimethylhexahydropyrano[3,2-d][1,3]dioxane-9-oxy)benzoyl)gambogate, methyl-9,1,0-dihydro-10-(4-methylpyrazine)-6-(7,8-dihydroxy-4,4-dimethylhexahydro pyrano[3,2-d][1,3]dioxane-9-oxy)gambogate, methyl-9,10-dihydro-10-(4-methyl-pyrazinyl)-6-[4-(4-methylpiperazine-1-yl)-4-oxobutyryl]gambogate, methyl-9,10-dihydro-10-(4-methylpyrazinyl)-6-[4-(4-methylpiperazinyl-1-yl)-3-oxopropionyl]gambogate, methyl-6-(2-chloroacetyl)-9,10-dihydro-10-(4-methylpyrazinyl)gambogate, methyl-6-(2-hydroxyethylamino)ethyl-9,10-dihydro-10-(4-methylpyrazinyl)gambogate, methyl-6-(2-(4-methylpiperzin-1-yl)-ethyl)-9,10-dihydro-10-(4-methylpyrazinyl) gambogate, methyl-6-(2-hydroxyethylamino)methyl-9,10-dihydrogen-10-(4-methyl pyrazinyl)gambogate, methyl-5-ketone-4,8,9-trihydrogen-9-(4-methylpyrazinyl)pyrano[4,3,2-d,e]gambogate, methyl-6-(bromomethyl)-9,10-dihydro-10-(4-methylpyrazinyl) gambogate, methyl-9,10-dihydro-10-(4-methylpyrazinyl)-6-(4-(7,8-dihydroxy-4,4-dimethylhexahydropyrano[3,2-d][1,3]dioxane-9-oxy)benzoyl)gambogate, 6,11-dihydro-triazolo(1,2,4)[3′,2′:2,3]pyrimidino[6,5,4-h,i]gambogatehydroxyl-amine, 6,11-dihydro-5-(4-oxo-D-alloglycosyl)triazolo(1,2,4)[3′,2′:2,3]pyrimidino[6,5,4-h,i]gambogatehydroxyl-amine, 6,11-dihydro-5-(4-oxo-D-glucose)triazolo(1,2,4)[3′,2′:2,3]pyrimidino[6,5,4-h,i]gambogatehydroxylamine, 6,11-dihydrogen-5-(7,8-dihydroxy-4,4-dimethylhexahydro-pyrano[3,2-d][1,3]dioxane-9-oxy)triazole[1,2,4][3′,2′:2,3]pyrimidino[6,5,4-h,i]gambogatehydroxylamine, 6,11-dihydro-5-(4-aloroxygen-D-glycosyl)benzoyltriazolo(1,2,4)[3′,2′:2,3]pyrimidino[6,5,4-h,i]gambogatehydroxylamine, 6,11-dihydro-5-(4-oxo-D-glucosyl)benzoyltriazolo(1,2,4)[3′,2′:2,3]pyrimidino[6,5,4-h,i]gambogatehydroxylamine, 6,11-dihydro-5-methyltriazolo(1,2,4)[3′,2′:2,3]pyrimidino[6,5,4-h,i]gambogatehydroxylamine, 6,11-dihydro-5-[4-(4-methylpiperazine-1-yl)-4-oxobutyryl]triazolo(1,2,4)[3′,2′:2,3]pyrimidino[6,5,4-h,i]gambogatehydroxyltriethylamine, 6,11-dihydro-5-O-phosphoryl-triazolo(1,2,4)[3′,2′:2,3]pyrimidino[6,5,4-h,i]gambogatehydroxylamine, 6,11-dihydro-5-oxygentriphosphoryl triazolo(1,2,4)[3′,2′:2,3]pyrimidino[6,5,4-h,i]gambogatehydroxyl-amine, 6,11-dihydro-5-[4-(4-methylpiperazine-1-yl)-3-oxopropionyl]triazolo(1,2,4)[3′,2′:2,3]pyrimidino[6,5,4-h,i]gambogatehydroxylamine, 6,11-dihydro-5-(2-chloroacetyl) triazolo(1,2,4)[3′,2′:2,3]pyrimidino[6,5,4-h,i]gambogatehydroxylamine, 6,11-dihydro-5-(2-hydroxyethylamino)ethyltriazolo(1,2,4)[3′,2′:2,3]pyrimidino[6,5,4-h,i]gambogate-hydroxylamine, 6,11-dihydro-5-(2-(4-methylpiperazine-1-yl)ethyl)triazolo(1,2,4)[3′,2′:2,3]pyrimidino[6,5,4-h,i]gambogatehydroxylamine, 6,11-dihydro-5-(2-hydroxyamino) methyltriazolo(1,2,4)[3′,2′:2,3]pyrimidino[6,5,4-h,i]gambogatehydroxylamine, 5-ketone-4,7,12-trihydropyrano[4″,3″,2″:4′,5′,6′]dihydrohydroxyethylaminogambogate[10′,9′,8′:4,5,6]pyrimidino[3,2-b]-1,2,4-triazole, 5-(bromomethyl)-1,2,4-triazolo[3′,2′:2,3]pyrimidino[6,5,4-h,i]-6,11-dihydro-hydroxyethylaminogambogate, 5-(4-(7,8-dihydroxy-4,4-dimethylhexahydropyrano[3,2-d][1,3]dioxane-9-oxy)benzoyl)[1,2,4]triazolo[3′,2′:2,3]pyrimidino[6,5,4-h,i]-6,11-dihydro-hydroxygambogate, 7-amino-8-cyano-9-H-pyrido[6,5,4-h,i]gambogatehydroxylamine, 5-(4-oxo-D-alloglycosyl)-7-amino-8-cyano-9-H-pyrido[6,5,4-h,i]gambogatehydroxylamine, 5-(4-oxo-D-gamino glucose)-7-amino-8-cyano-9-H-pyrido[6,5,4-h,i]gambogatehydroxylamine, 5-(7,8-dihydroxy-4,4-dimethylhexahydropyrano[3,2-d][1,3]dioxane-9-oxy)-7-amino-8-cyano-9-H-pyrido[6,5,4-h,i]gambogate-hydroxylamine, 5-(4-oxo-D-alosglycosylation)benzoyl-7-amino-8-cyano-9-H-pyrido[6,5,4-h,i]gambogatehydroxyl-amine, 5-(4-oxo-D-glucosyl)benzoyl-7-amino-8-cyano-9-H-pyrido[6,5,4-h,i]gambogatehydroxylamine, 5-methyl-7-amino-8-cyano-9-H-pyrido[6,5,4-h,i]gambogatehydroxylamine, 5-[4-(4-methylpiperzin-1-yl)-4-oxobutyryl]-7-amino-8-cyano-9-H-pyrido[6,5,4-h,i]gambogatehydroxylamine, 5-O-phosphoryl-7-amino-8-cyano-9-H-pyrido[6,5,4-h,i]gambogatehydroxylamine, 5-O-triphosphoryl-7-amino-8-cyano-9-H-pyrido[6,5,4-h,i]gambogatehydroxylamine, 5-[4-(4-methylpiperzin-1-yl)-3-oxopropionyl]-7-amino-8-cyano-9-H-pyrido[6,5,4-h,i]gambogatehydroxylamine, 5-(2-chloroacetyl)-7-amino-8-cyano-9-H-pyrido[6,5,4-h,i]gambogatehydroxylamine, 5-(2-hydroxyethylamino)ethyl-7-amino-8-cyano-9-H-pyrido[6,5,4-h,i]gambogatehydroxyl-amine, 5-(2-(4-methylpiperzin-1-yl)ethyl)-7-amino-8-cyano-9-H-pyrido[6,5,4-h,i]gambogatehydroxylamine, 5-(2-hydroxyethylamino)methyl-7-amino-8-cyano-9-H-pyridino[6,5,4-h,i]gambogatehydroxylamine, 5-ketone-4,7,10-triplehydrogenpyrano[4′,3′,2′:4,5,6]hydroxyethylamino gambogate[10,9,8:d,e]8-amino-9-cyanopyrimidine, 5-bromo-methyl-7-amino-8-cyano-9-H-pyrido[6,5,4-h,i]gambogatehydroxylamine, 5-(4-(7,8-dihydroxy-4,4-dimethylhexa-hydropyrano[3,2-d][1,3]dioxane-9-oxy)benzoyl)-7-amino-8-cyano-9-H-pyrido[6,5,4-h,i]gambogatehydroxyethylamine, 13-H-benzo[d′]imidazo[2′,1′:2,3]pyrimidino[6,5,4-h,i]gambogatehydroxylamine, 5-(4-aloroxygen-D-glycosyl)-13-H-benzo[d′]imidazo[2′,1′:2,3]pyrimidino[6,5,4-h,i]gambogatehydroxylamine, 5-(4-oxo-D-glucose)-13-H-benzo[d′]imidazo[2′,1:2,3]pyrimidino[6,5,4-h,i]gambogatehydroxylamine, 5-(7,8-dihydroxy-4,4-dimethylhexahydropyrano[3,2-d][1,3]dioxane-9-oxy)-13-H-benzo[d]imidazo[2′,1′:2,3]pyrimidino[6,5,4-h,i]gambogatehydroxylamine, 5-(4-oxo-D-alloglycosyl)benzoyl-13-H-benzo[d′]imidazo[2′,1′:2,3]pyrimidino[6,5,4-h,i]gambogate hydroxylamine, 5-(4-oxo-D-glucosyl)-benzoyl-13-H-benzo[d′]imidazo[2′,1′:2,3]pyrimidino[6,5,4-h,i]gambogatehydroxylamine, 5-methyl-13-H-benzo[d′]imidazo[2′,1′:2,3]pyrimidino[6,5,4-h,i]gambogatehydroxylamine, 5-[4-(4-methylpiperzin-1-yl)-4-oxobutyryl]-13-H-benzo[d′]imidazo[2′,1′:2,3]pyrimidino[6,5,4-h,i]gambogatehydroxyl-amine, 5-O-phosphoryl-13-H-benzo[d′]imidazo[2′,1′:2,3]pyrimidino[6,5,4-h,i]gambogatehydroxyl-amine, 5-O-triphosphoryl-13-H-benzo[d′]imidazo[2′,1′:2,3]pyrimidino[6,5,4-h,i]gambogatehydroxylamine, 5-[4-(4-methylpiperazine-1-yl)-3-oxopropionyl]-13-H-benzo[d′]imidazo[2′,1′:2,3]pyrimidino[6,5,4-h,i]gambogatehydroxylamine, 5-(2-chloroacetyl)-13-H-benzo[d′]imidazo[2′,1′:2,3]pyrimidino[6,5,4-h,i]gambogatehydroxylamine, 5-(2-hydroxyethylamino)ethyl-13-H-benzo[d′]imidazo[2,1′:2,3]pyrimidino[6,5,4-h,i]gambogatehydroxylamine, 5-(2-(4-methylpiperzin-1-yl)ethyl)-13-H-benzo[d′]imidazolo[2′,1′:2,3]pyrimidino[6,5,4-h,i]gambogatehydroxylamine, 5-(2-hydroxyethyl)methyl-13-H-benzeno[d′]imidazo[2′,1′:2,3]pyrimidino[6,5,4-h,i]gambogate-hydroxylamine, pyrano[4,3,2-d,e]-5(4H)-ketone-13-H-benzo[d′]imidazo[2′,1′:2,3]pyrimidino[6,5,4-h,i]gambogateaminohydroxylethylamine, 5-(bromomethyl)-13-H-benzo[d′]imidazo[2′,1′:2,3]pyrimidino[6,5,4-h,i]gambogate glycolsylamine, 5-(4-(7,8-dihydroxy-4,4-dimethylhexa-hydropyrano[3,2-d][1,3]dioxane-9-oxy)benzoyl)-13-H-benzo[d′]imidazo[2′,1′:2,3]pyrimidino[6,5,4-h,i]gambogatehydroxylamine, 6,12-dihydrobenzo[b][1,4]thiazepino[4,3,2-h,i]gambogatehydroxyl-amine, 5-(4-oxo-D-allosugar)-6,12-dihydrobenzo[b][1,4]thiazepino[4,3,2-h,i]gambogatehydroxylamine, 5-(4-O-D-glucose)-6,12-dihydrobenzo[b][1,4]thiazepino[4,3,2-h,i]gambogatehydroxylamine, 6,12-dihydro-5-(7,8-dihydroxy-4,4-dimethylhexahydropyrano[3,2-d][1,3]dioxane-9-phenoxy)benzeno[b][1,4]thiazepino[4,3,2-h,i]gambogatehydroxylamine, 5-(4-oxo-D-alloglycosyl)benzoyl-6,12-dihydrobenzo[b][1,4]thiazepino[4,3,2-h,i]gambogatehydroxylamine, 5-(4-oxo-D-glucose)benzoyl-6,12-dihydrobenzo[b][1,4]thiazepino[4,3,2-h,i]gambogatehydroxylamine, 5-methyl-6,12-dihydrobenzo[b][1,4]thiazepino[4,3,2-h,i]gambogatehydroxylamine, 5-[4-(4-methyl-piperzin-1-yl)-4-oxobutyryl]-6,12-dihydrobenzo[b][1,4]thiazepino[4,3,2-h,i]gambogate-hydroxylamine, 5-O-phosphoryl-6,12-dihydrobenzo[b][1,4]thiazepino[4,3,2-h,i]gambogatehydroxylamine, 5-O-triphosphoryl-6,12-dihydrobenzo[b][1,4]thiazepino[4,3,2-h,i]gambogatehydroxylamine, 5-[4-(4-methyl-piperzin-1-yl)-3-oxopropionyl]-6,12-dihydrobenzo[b][1,4]thiazepino[4,3,2-h,i]gambogatehydroxylamine, 5-(2-chloroacetyl)-6,12-dihydrobenzo[b][1,4]thiazepino[4,3,2-h,i]gambogatehydroxylamine, 5-(2-hydroxy-ethylamino)ethyl-6,12-dihydrobenzo[b][1,4]thiazepino[4,3,2-h,i]gambogatehydroxyl-amine, 5-(2-(4-methylpiperzin-1-yl)ethyl)-6,12-dihydrobenzo[b][1,4]thiazepino[4,3,2-h,i]gambogatehydroxylamine, 5-(2-hydroxyethylamino)methyl-6,12-dihydrobenzo[b][1,4]thiazepino[4,3,2-h,i]gambogatehydroxylamine, 5-ketone-4,7,12-trihydropyrano[4″,3″,2″:4′,5′,6′]gambogatehydroxylamino[10′,8′:2,3,4]thiazepino[1,4][6,7-a]benzene, 5-(bromo-methyl-)-6,12-dihydrobenzo[b][1,4]thiazepino[4,3,2-h,i]gambogatehydroxylamine, 5-(4-(7,8-dihydroxy-4,4-dimethylhexahydropyrano[3,2-d][1,3]dioxane-9-oxy)benzoyl)-6,12-dihydrobenzo[b][1,4]thiazepino[4,3,2-h,i]gambogatehydroxylethylamine, 12,13-dihydro-indanone-1′-[3′,2′:2,3]pyrido[6,5,4-h,i]gambogatehydroxylamine, 5-(4-oxo-D-glucose)-12,13-dihydroindanone-1′-[3′,2′:2,3]pyrido[6,5,4-h,i]gambogatehydroxylamine, 5-(4-oxo-D-alloglycosyl)-12,13-dihydroindanone-1′[3′,2′:2,3]pyrido[6,5,4-h,i]gambogatehydroxylamine, 5-(7,8-dihydroxy-4,4-dimethylhexahydropyrano[3,2-d][1,3]dioxane-9-oxy)-12,13-dihydroindanone-1′[3′,2′:2,3]pyrido[6,5,4-h,i]gambogatehydroxylamine, 5-(4-oxo-D-alloglycosyl)benzoyl-12,13-dihydroindanone-1′[3′,2′:2,3]pyrido[6,5,4-h,i]gambogate-hydroxylamine, 5-(4-oxo-D-glucosyl)benzoyl-12,13-dihydroindanone-1′[3′,2′:2,3]pyrido[6,5,4-h,i]gambogatehydroxylamine, 5-methyl-12,13-dihydroindanone-1′[3′,2′:2,3]pyrido[6,5,4-h,i]gambogatehydroxylamine, 5-[4-(4-methylpiperzin-1-yl)-4-oxobutyryl]-12,13-dihydroindanone-1′ [3′,2′:2,3]pyrido[6,5,4-h,i]gambogatehydroxylamine, 5-O-phosphoryl-12,13-dihydroindanone-1′[3′,2′:2,3]pyrido[6,5,4-h,i]gambogatehydroxylamine, 5-O-triphosphoryl-12,13-dihydroindanone-1′ [3′,2′:2,3]pyrido[6,5,4-h,i]gambogatehydroxylamine, 5-[4-(4-methylpiperzin-1-yl)-3-oxopropionyl]-12,13-dihydroindanone-1′[3′,2′:2,3]pyrido[6,5,4-h,i]gambogatehydroxylamine, 5-(2-chloroacetyl)-12,13-dihydroindanone-1′[3′,2′:2,3]pyrido[6,5,4-h,i]gambogatehydroxylamine, 5-(2-hydroxyethylamino)ethyl-12,13-dihydroindanone-1′[3′,2′:2,3]pyrido[6,5,4-h,i]gambogatehydroxylamine, 5-(2-(4-methyl-piperine-1-yl)ethyl)-12,13-dihydroindanone-1′[3′,2′:2,3]pyrido[6,5,4-h,i]gambogatehydroxylamine, 5-(2-hydroxyethylamino)methyl-12,13-dihydroindanone-1′[3′,2′:2,3]pyrido[6,5,4-h,i]gambogatehydroxylamine, 5-ketone-4,14,15-trihydropyrano[4″,3″,2″:4′,5′,6′]gambogatehydroxylamino[10′,9′,8′:4,5,6]pyrido[3,2-b]indanone-1,5-(bromo-methyl)-12,13-dihydroindanone-1′[3′,2′:2,3]pyrido[6,5,4-h,i]gambogatehydroxylamine, 5-(4-(7,8-dihydroxy-4,4-dimethylhexahydropyrano[3,2-d][1,3]dioxane-9-oxy)benzoyl)-12,13-dihydro-indanone-1′-[3′,2′:2,3]pyrido[6,5,4-h,i]gambogatehydroxylamine, methyl-5-[4-(4-methylpiperazine-1-yl)-4-oxobutyryl]gambogate, methyl-5-[4-(4-methylpiperzin-1-yl)-3-oxopropionyl]gambogate, methyl-6-(2-chloroacetyl)gambogate, methyl-5-(2-hydroxyethylamino)ethylgambogate, methyl-6-(2-(4-methylpiperzin-1-yl)ethyl)gambogate, methyl-6-(2-hydroxyamino)gambogate, methyl-4-H-pyrone(5)[4,3,2-d,e]gambogate, methyl-6-(2-bromoethyl)gambogate.

3. A compound according to claim 1, wherein:
The compound is selected from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier.

4. A compound according to the claim 1, wherein:
A process for the manufacture of a compound of formula I, II, III comprises:
For the preparation of compounds of formula I, II, III with A-ring, B-ring, C-ring and salts thereof in which the reaction of a gambogic acid or analog to introduce A-ring lactone at 4-, 6-position, B-ring at 6-, 8-position, and C-ring at 8-, 10-position forms a bond of C—C, C—O, C—S, C—N or C—P under catalysis at −78° C. to 90° C., wherein;
The reactant selected from 2-ethoxy-1-ethoxy carbon acyl-1,2-dihydroquinoline, 2-(7-azabenzotriazole)-N,N,N′,N′-tetramethyl urea hexafluorophosphate, benzotriazole-N,N,N′, N′-tetramethyl urea hexafluoro phosphate, 6-chlorophenyl and triazole-1,1,3,3-tetramethyl urea hexafluoro phosphate, 1-hydroxy-7-azobis benzotriazole, 1-hydroxy-benzotriazole, 3-hydroxy-1,2,3-benzotriazin-4(3H)one, N-hydroxysuccinimide, and triethylamine, Fmoc chloride, acyl succinimide Fmoc, 9-fluorene methanol;
The catalyst selected from palladium, platinum, ruthenium, metal catalyst, organic base or inorganic base, 1-ethyl-3-(3-dimethylpropylamine)carbodiimide, ditertbutyl dicarbonate, bis(2-oxo-3-oxazoline alkyl) times phosphorus chloride, N,N′-carbonyl two pyrrolidine, N,N′-carbonyl bis(1,2,4-triazole), 6-chloro-1-hydroxybenzo triazole, N,N′-dicyclohexyl carbodiimide, 4,5-dicyano imidazole, 3-(diethoxyphosphoryl)oxy-1,2,3-benzotriazine-4-ketone, N,N′-bis isopropyl carbon imide, N,N′-diisopropyl ethylamine, 4-dimethylaminopyridine, 4,4′-dimethoxytriphenyl chloride, 4-(4,6-dimethoxytriazine)-4-methyl morpholine hydrochloride, N,N′-succinimidyl carbonate, 1-ethyl-(3-dimethylaminopropyl)carbodiimide hydrochloride;
The solvent selected from tetrahydrofuran, 1,4-dioxane, acetonitrile, N,N-dimethylformamide, N,N-dimethylacetamide, hexane, toluene, quinoline.

5. A compound according to the claim 1, wherein: For the preparation of compounds of formula I, II, III with A-ring, B-ring, C-ring and salts, the X₁, X₂, R₁, R₂, R₃, R₄, R₅, R₆, R₇, R₈, R₉, R₁₀, R₁₁ or/and R₁₂ is an amino acid, acyloxy, phosphoric acid, phosphoryloxy, sulfonyloxy, alkoxy, aryloxy, heterocyclicoxy, hydrocarbons, alicyclic, glycosyl, multi-hydroxyl, carboxyl, glucosyl, multi-hydroxyl, alkane, aryl, alicyclic, heterocyclic, heteroarylcyclic or substituent modified by acylation, halogenation; electrophilic substituent at 9-position, nucleophilic substituent at 10-position accompanied by 1,4 addition reaction; allylation at 11-position, 26-position, 31-position or 36-position with a bond of C—C, C—O, C—S, C—N or C—P under catalysis at −78 to 90° C.

6. A method according to the claim 3, wherein:
A method for treating cancer, comprising: administration to a compound of the claim 1 and claim 9, in the range of 0.001 mg/kg-250 mg/kg, a pharmaceutically acceptable salt or prodrug from thereof;
a cancer is selected from the lung cancer, stomach cancer, colon cancer, small cell lung cancer, thyroid cancer, esophageal cancer, pancreatic cancer, endometrial cancer, adrenal cortical carcinoma, head and neck cancer, Osteogenic sarcoma, breast cancer, ovarian cancer, Vail Williams tumors, cervical tumors, testicular cancer, genitourinary cancer, skin cancer, renal cell carcinoma, bladder cancer, primary brain cancer, prostate cancer, soft tissue sarcoma, neuroblastoma, rhabdomyosarcoma, Kaposi sarcoma, malignant melanoma, malignant pancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, or Hodgkin's disease.

7. The method according to claim 3, wherein said compounds is administered together with at least one known cancer, chemotherapeutic and immune agent selected from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, tamoxifen, doxazocin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoro-methyl ornithine, fenretinide, N-4-carboxyphenyl retinamide, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, Roscovitine, Olonoucine, Valecoxib.

8. A method according to the claim 3, wherein:
a compound for treating, preventing or slowing the progression of neoplasia and cancer, and infection diseases by virus, bacterial or fungi, including bacterial infections and fungal infections of the drug application, which comprises administration together with at least one known chemotherapeutic agent selected from the group consisting of antibacterial and antifungal drugs to a patient in need of such treatment.

9. A compound according to the claim 3, wherein:
the administration may be by oral route, parenteral, subcutaneous, intravenous, intramuscular, intra-peritoneal, transdermal, buccal, intrathecal, intracranial, intranasal or topical routes.
